(19) United States
(12) Patent Application Publication
(10) Pub. No.: US 2023/0183179 A1
(43) Pub. Date: Jun. 15, 2023

(54) NOVEL CELL METABOLISM MODULATING COMPOUNDS AND USES THEREOF

(71) Applicant: CRESCENTA BIOSCIENCES, UNION, NJ (US)

(72) Inventors: EMRE KOYUNCU, Irvine, CA (US); HAHN KIM, PRINCETON, NJ (US)

(73) Assignee: CRESCENTA BIOSCIENCES, UNION, NJ (US)

(21) Appl. No.: 17/793,670

(22) PCT Filed: Jan. 20, 2021
(86) PCT No.: PCT/US21/14250

§ 371 Date: Jul. 19, 2022

Related U.S. Application Data
(60) Provisional application No. 62/963,508, filed on Jan. 20, 2020.

(51) Int. Cl. C07D 215/38; C07D 401/12; C07D 401/04; C07D 413/04; C07D 235/30; C07D 401/08; C07D 417/04; C07D 471/04; C07D 417/08; C07D 401/14

(52) U.S. Cl. CPC C07D 215/38; C07D 401/12; C07D 401/04; C07D 413/04; C07D 235/30; C07D 401/08; C07D 417/04; C07D 471/04; C07D 417/08; C07D 401/14

(57) ABSTRACT

A class of compounds that bind to fatty acid binding protein (FABP4) and modulate adipocyte metabolism to drive enhanced glucose utilization, as well as pharmaceutical compositions comprising the class of compounds, in combination with a pharmaceutically acceptable diluent or carrier, and optionally, further in combination with a therapeutically active agent, and the use of these compounds in medicine and for the preparation of a medicament in the treatment of disorders acting on the FABP4.

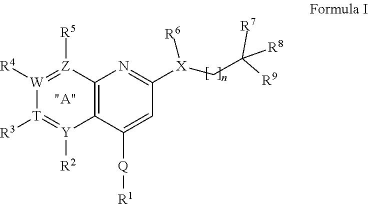

### CROSS-REFERENCE TO RELATED APPLICATIONS SECTION

This application claims priority to International Application S/N 17/742,899 filed on Jan. 20, 2021 which claims priority of U.S. Provisional Patent Application Ser. No. 62/963,508 filed on Jan. 20, 2020, the entire contents of which are hereby incorporated by reference in their entirety.

### FIELD OF THE EMBODIMENTS

The field of the embodiments of the present invention relate to novel compounds for treatment or prophylaxis of diseases related to metabolism and inflammation, including, but not limited to, type-2 diabetes, atherosclerosis, intracranial atherosclerotic disease, Alzheimer's disease, non-alcoholic steatohepatitis, obesity, cardiovascular disease, asthma, cancer and other diseases. Compounds in this invention particularly interact with fatty acid binding protein 4 (FABP4) and improve glucose consumption in adipocytes.

### BACKGROUND OF THE EMBODIMENTS

Fatty acid binding proteins (FABP) are a family of proteins that reversibly bind free fatty acids and other lipid molecules and facilitate their transport in cells. To date, nine different FABP isoforms have been identified in mammals. FABP isoforms display differential expression patterns in different tissues. Fatty acid binding protein 4 (FABP4), also often referred to as aP2 in literature, is primarily expressed in adipocytes and macrophages, and mediates key metabolic and inflammatory pathways in these cells, such as lipid storage and degradation, signaling, and eicosanoid production. In addition, FABP4 is also secreted to plasma and has been proposed to act as an adipose-derived factor regulating systemic glucose homeostasis.

Genetic knockout studies in mice provided insights into tissue-specific and systemic functions of FABP4. Importantly, when mice bearing a homozygous deletion of the FABP4 gene are subjected to a prolonged high-fat diet, they gained weight comparable to the wild-type, but were protected from hyperglycemia and insulin-resistance. See, G. S. Hotamisligil, et al., “Uncoupling of Obesity from Insulin Resistance Through a Targeted Mutation in Ap2, the Adipocyte Fatty Acid Binding Protein,” Science, 1996, 274(5291), Pages 1377-9, doi: 10.1126/science.274.5291.1377. In addition, FABP4-deficiency significantly protected apoliprotein E (ApoE) knockout mice from atherosclerosis, a phenotype attributed to FABP4 modulation of inflammatory pathways in macrophages. See, L. Makowski, et al., “Lack of Macrophage Fatty-Acid-Binding Protein aP2 Protects Mice Deficient in Apolipoprotein E Against Atherosclerosis,” Nat. Med. 2001, 7(6), Pages 699-705, doi: 10.1038/89076. FABP4 expression has also been detected in airway epithelial cells and FABP4-deficiency was demonstrated to be protective in a mouse model of allergic lung inflammation. See, B. O. V. Shum, et al., “The Adipocyte Fatty Acid-Binding Protein aP2 is Required in Allergic Airway Inflammation,” J. Clin. Invest., 2006, 116(8), Pages 2183-2192, doi: 10.1172/JCI24767.

Several reports have been published in literature linking FABP4 expression level and functions with a number of pathologies in humans. For instance, reduced risk of type-2 diabetes and coronary heart disease was observed in individuals bearing genetic variations in the promoter region of FABP4 (rs77878271) that resulted in reduced expression of this gene. See, G. Tuncman, et al., “A Genetic Variant at the Fatty Acid-binding Protein aP2 Locus Reduces the Risk for Hypertriglyceridemia, Type 2 Diabetes, and Cardiovascular Disease,” Proc. Natl. Acad. Sci. USA, 2006, 103(18), Pages 6970-5, doi: 10.1073/pnas.0602178103. The same polymorphism was also associated with reduced atherosclerotic disease manifestations in an independent study. See, J. Saksi, et al., “Low-Expression Variant of Fatty Acid-Binding Protein 4 Favors Reduced Manifestations of Atherosclerotic Disease and Increased Plaque Stability,” Circ. Cardiovasc. Genet., 2014, 7(5), Pages 588-98, doi: 10.1161/CIRCGENETICS.113.000499. Furthermore, triple-negative breast cancer patients with a single nucleotide polymorphism in the 3′ untranslated region (UTR) of FABP4 (rs1054135-GG genotype) that also results in reduced FAB4 expression was associated with a reduced risk of disease progression and a prolonged disease-free survival time. See, W. Wang, et al., “A Single-Nucleotide Polymorphism in the 3′-UTR Region of the Adipocyte Fatty Acid Binding Protein 4 Gene is Associated with Prognosis of Triple-Negative Breast Cancer,” Oncotarget., 2016, 7(14), Pages 18984-18998, doi: 10.18632/oncotarget.7920. Moreover, increased expression of FABP4 has been observed in preeclamptic placenta and is proposed to have a role in the pathogenesis of preeclampsia. See, Y. Yan, et al., “Increased Expression of Fatty Acid Binding Protein 4 in Preeclamptic Placenta and its Relevance to Preeclampsia,” Placenta, 2016, 39, Pages 94-100, https://doi.org/10.1016/j.placenta.2016.01.014. Similarly, granulosa cells of polycystic ovary syndrome patients also show increased FABP4 expression, which was related to the clinical characterization of the disease. See, W. Hu, et al., “Expression and Regulation of Adipocyte Fatty Acid Binding Protein in Granulosa Cells and its Relation with Clinical Characteristics of Polycystic Ovary Syndrome,” Endocrine, 2011, 40(2), Pages 196-202, doi: 10.1007/s12020-011-9495-9. Collectively, these studies demonstrated the active role of FAPB4 in regulating systemic metabolism and inflammation, and suggested that pharmacological targeting of FABP4 can be used as a strategy for the treatment of a variety of diseases linked to FABP4 functions.

Adipocytes play a central role in the systemic glucose homeostasis. One of their primary roles is taking up excess glucose in plasma and storing it in the form of lipids. Adipocyte dysfunction due to metabolic stress and inflammation often leads to complications, such as hyperglycemia and insulin resistance. It is noteworthy that FABP4-deficient mice showed increased glucose deposition to adipose tissue. Adipocytes isolated from these animals showed a significantly elevated rate of glucose conversion into fatty acids as compared to their wild-type counterparts. See, S. Shaughnessy, et al., “Adipocyte Metabolism in Adipocyte Fatty Acid Binding Protein Knockout Mice (Ap2−/−) After Short-Term High-Fat Feeding: Functional Compensation by the Keratinocyte [Correction Of Keritinocyte] Fatty Acid Binding Protein,” Diabetes, 2000, 49(6), Pages 904-911, https://doi.org/10.2337/diabetes.49.6.904. Thus, increasing glucose consumption in adipocytes can be achieved by targeting FABP4.

While the literature and certain prior art patent applications (e.g., WO 00/47734, WO 00/15229, WO 00/15230, WO 02/40448, WO 01/54694, WO 00/59506, and WO 2004/063156) have provided various presentations of the concept of the FABP inhibition, in general, and that of the FABP4 inhibition, in particular, none of the discussions in these prior art documents provides solution(s) of all of the unmet needs, as does the present invention. Specifically, the present invention describes a novel class of compounds that bind to FABP4 and modulate adipocyte metabolism to drive enhanced glucose utilization.

### SUMMARY OF THE EMBODIMENTS

The present invention, in one of its embodiments, relates to a compound of Formula (I), or pharmaceutically acceptable salts or esters thereof:

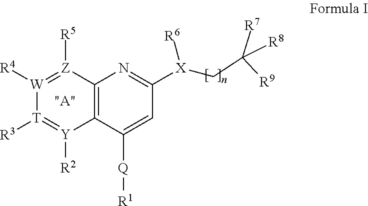

wherein each of R¹ and R⁶-R⁹ are independently —H, —CN, —COOH, —CONH₂, B(ORₐ)₂, an acid isostere, a halo, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl,

wherein Rₐ of the B(ORₐ)₂ is H or an alkyl,

wherein B of the B(ORₐ)₂ is boron,

wherein n of the Cₙ is 1-10,

wherein each of R²-R⁵ are independently —H, —CN, —COOH, —COOMe, —CONH₂, B(ORₐ)₂, the acid isostere, the halo, —CONHOH, —NH—SO₂—Cι-C₆-alkyl, —NHSO₂Ar, the Cₙ alkyl, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein the Ar of —NHSO₂Ar is selected from the group consisting of: phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5-naphthyridine, 1,8-naphthyridine, acridine, phenazine, and xanthene,

wherein each of the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, and the Cₙ heterocycloalkyl is unsubstituted or substituted with a quantity of substituents being between 1-5,

wherein each of the substituents is the same or different,

wherein each of the substituents is selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, and —C_{q}—U—C_{q},

wherein each q of —C_{q}—U—C_{q} is independently 0 to 10,

wherein the U of —C_{q}—U—C_{q} is any one of aryl, heteroaryl, cycloalkyl, heterocycloalkyl, O, S, SO₂, or N(R₁)(R₁),

wherein each R₁ of N(R₁)(R₁) is independently hydrogen, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein each R₁ of N(R₁)(R₁) is unsubstituted or substituted with 1, 2, 3, 4 or 5 substituents which can be the same or different and are independently selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein Q is a bond or O,

wherein X is C, N, O, or S such that R⁶ is not present if X is O or S,

wherein “A” is a saturated or unsaturated ring depicted by

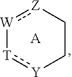

wherein Y, T, W, and Z are independently a bond, C, N, O, an alkyl having 1 to 4 carbon atoms, or an alkenyl having 1 to 4 carbon atoms, and

wherein n is 0, 1, 2, or 3.

In one embodiment, the invention comprises the compound of Formula I where at least one of R¹ and R², R² and R³, R³ and R⁴, R⁴ and R⁵, R⁶ and R⁷, R⁷ and R⁸, R⁸ and R⁹ are bonded forming a fused heteroaryl or fused heterocycloalkyl.

In a further embodiment, the invention may be a compound of Formula II or pharmaceutically acceptable salts or esters thereof:

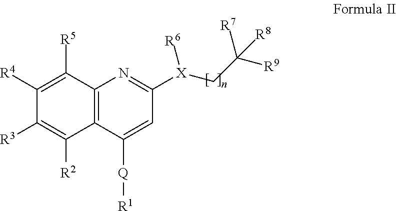

wherein R¹ and R⁶-R⁹ is independently —H, —CN, —COOH, —CONH₂, B(ORₐ)₂, an acid isostere, a halo, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl,

wherein Rₐ of the B(ORₐ)₂ is H or an alkyl,

wherein B of the B(ORₐ)₂ is boron,

wherein n of the Cₙ is 1-10,

wherein each of R²-R⁵ are independently —H, —CN, —COOH, —COOMe, —CONH₂, B(ORₐ)₂, the acid isostere, the halo, —CONHOH, —NH—SO₂—C₁-C₆-alkyl, —NHSO₂Ar, the Cₙ alkyl, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein the Ar of —NHSO₂Ar is selected from the group consisting of: phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5-naphthyridine, 1,8-naphthyridine, acridine, phenazine, and xanthene,

wherein each of the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl is unsubstituted or substituted with a quantity of substituents being between 1-5,

wherein each of the substituents is the same or different,

wherein each of the substituents is selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, and —C_{q}—U—C_{q},

wherein each q of —C_{q}—U—C_{q} is independently 0 to 10,

wherein the U of —C_{q}—U—C_{q} is any one of aryl, heteroaryl, cycloalkyl, heterocycloalkyl, O, S, SO₂, or N(R₁)(R₁),

wherein each R₁ of N(R₁)(R₁) is independently hydrogen, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein each R₁ of N(R₁)(R₁) is unsubstituted or substituted with 1, 2, 3, 4 or 5 substituents which can be the same or different and are independently selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein Q is a bond or O,

wherein X is C, N, O, or S such that R⁶ is not present if X is O or S,

wherein X is C, N, O, or S, and

wherein n is 0, 1, 2, or 3.

In some embodiments, the invention includes compounds of Formula II, wherein a heterocycloalkyl group is formed by bonding two of R⁷, R⁸, or R⁹ to form:

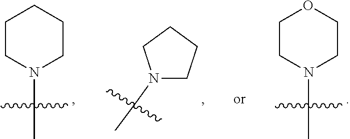 [SMILES: *N1CCCCC1 ; *N1CCCC1 ; *N1CCOCC1]

Additional embodiments of the invention include compounds of Formula III or pharmaceutically acceptable salts or esters thereof:

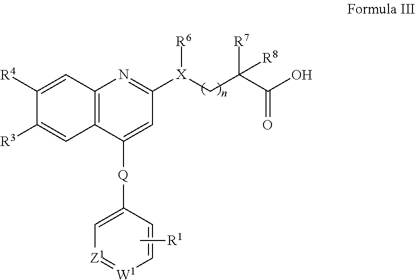

wherein R¹ is selected from the group consisting of: —CN, alkyl, —H, halo, ²H, amino, alkoxy, aminoalkyl, (amino)alkoxy, alkenyl, alkynyl, alkoxy, hydroxy, alkylhydroxy, aryloxy, alkyl(aryl), (alkoxyalkyl)amino, aryl, aryl(halo), heteroaryl, hydroxyl-alkyl, hydroxyl-aryl, (aryl)alkyl, C(O)OH, —S(O)₂-alkyl, —S(O)₂-aryl, —C(O)alkyl, and C(O)NH₂,

wherein each of R³ and R⁴ are independently —H, the halo, Cₙ alkyl, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, Cₙ heterocycloalkyl, and —C_{q}—U—C_{q},

wherein n of Cₙ is 1-10,

wherein each q of —C_{q}—U—C_{q} is independently 0 to 10,

wherein the U of —C_{q}—U—C_{q} is any one of O, S, SO₂, or N(R₁)(R₁),

wherein each R₁ of N(R₁)(R₁) are independently hydrogen,

wherein each of the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, and the Cₙ heterocycloalkyl are unsubstituted or substituted with a quantity of substituents being between 1-5,

wherein each of the substituents are the same or different,

wherein each of the substituents is selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein R⁶ is independently H or alkyl,

wherein each of R⁷ and R⁸ are independently hydrogen, ²H, fluoro or alkyl,

wherein R⁶ and R⁷ are bonded with an adjoining R group to form a fused group,

wherein the fused group is selected from the group consisting of: a fused cycloalkyl, a fused heterocycloalkyl, a fused aryl, and a fused heteroaryl ring,

wherein the fused group comprises between 4 to 10 carbon atoms,

wherein n of Formula III is 0 or 1,

wherein Q is a bond or O,

wherein X is C, N, O, or S such that R⁶ is not present if X is O or S, and

wherein Z¹ or W¹ are independently C, N, O, or S.

Another embodiment of the invention includes compounds of Formula III wherein X is N.

An embodiment the invention includes compounds of Formula III wherein R⁶ is hydrogen and X is N.

A further embodiment the invention includes compounds of Formula III wherein R³ is alkyl and R¹ is cyano.

An additional embodiment the invention includes compounds of Formula III wherein X is N and n=0.

An embodiment the invention includes compounds of Formula III wherein X is N and n=1.

In general, the present invention succeeds in conferring the following benefits and objectives.

It is an object of the present invention to utilize the compound according to Formula (I), (II) or (III) or a pharmaceutically acceptable salt or ester thereof to treat disorders acting on the fatty acid binding protein (FABP4).

It is an object of the present invention to provide a pharmaceutical composition comprising a compound according to Formula (I), (II) or (III) as an active ingredient, in combination with a pharmaceutically acceptable diluent or carrier, for use in the treatment of disorders acting on the FABP4. Here, the pharmaceutical composition can further comprise an additional therapeutically active agent.

It is an object of the present invention to provide a method for the treatment of disorders acting on the FABP4. Such method comprises numerous process steps, such as: administering an effective amount of a compound according to Formula (I), (II) or (III) to a subject in need of such treatment (preferably, a human). The method optionally includes a process step regarding co-administration with other therapeutic agents, either as a single (or multiple) dosing, and either simultaneously or sequentially.

It is an object of the present invention to provide a method for inhibiting FABP4, which comprises a process step of administering an effective amount of a compound according to Formula (I), (II) or (III) to a subject in need of such treatment (preferably, a human).

It is an object of the present invention to provide a method to use a compound according to Formula (I), (II) or (III) for the manufacture of a medicament for use in the treatment of disorders acting on the FABP4. Examples of such disorders include type 2 diabetes, hyperglycemia, metabolic syndrome, obesity, atherosclerosis, intracranial atherosclerotic disease, non-alcoholic steatohepatitis, asthma, multiple sclerosis, Alzheimer's disease, other chronic inflammatory and autoimmune/inflammatory diseases, chronic heart disease, polycystic ovary syndrome, preeclampsia, and cancer.

Other features and advantages of the invention will be apparent from the detailed description and the claims.

### DESCRIPTION OF THE PREFERRED EMBODIMENTS

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be.

### Definitions

As used herein, the term “acid isostere” includes, but is not limited to, the following functional groups, where R is H or alkyl.

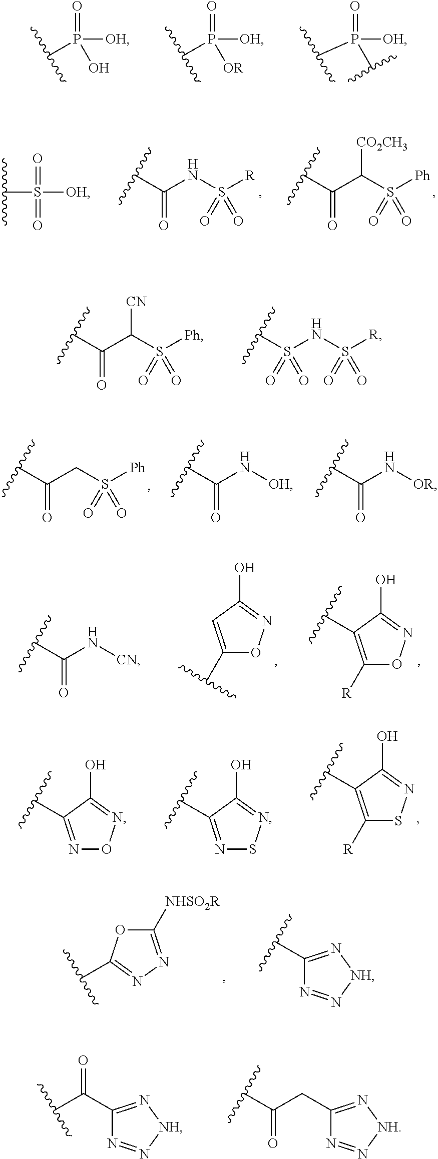

The term “alkyl” refers to a saturated, straight-chain hydrocarbon group, or branched-chain hydrocarbon group having from 1 to 20 carbon atoms. Representative alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, and the like, and longer alkyl groups, such as heptyl, octyl, and the like. Moreover, the number of carbons on an alkyl chain can be defined in association with the C atom such as C₁₋₁₀, where C₁₋₁₀ is a carbon chain having 1 to 10 carbon atoms.

The term “alkoxy” as used herein refers to an alkyl group singularly bonded to oxygen and includes —O-(alkyl), wherein alkyl is defined above.

The term “amino” as used herein refers to an —NH₂ group.

The term “alkenyl” refers to a hydrocarbon group formed when a hydrogen atom is removed from an alkene group. Alkenyl compounds are named by replacing the -e from the parent alkene's name with -yl. An example includes H2C═CH— (e.g., ethenyl or vinyl).

The term “alkynyl” refers to a fragment containing an open point of attachment on a carbon atom, that would form if a hydrogen atom bonded to a triply bonded carbon is removed from the molecule of an alkyne.

The term “haloalkyl” refers to any alkyl radical having one or more hydrogen atoms replaced by a halogen atom.

The term “phenyl” refers to a univalent hydrocarbon radical (C₆H₅) formally derived from benzene by the removal of a hydrogen atom.

The term “naphthyl” refers to an isomeric univalent radical formally derived from naphthalene by removal of a hydrogen atom.

The term “pyrrole” refers to a heterocyclic aromatic organic compound that is a five-membered ring with the formula C₄H₄NH.

The term “imidazole” refers to an organic compound with the formula C₃N₂H₄.

The term “thiophene” refers to a heterocyclic compound with the formula C₄H₄S.

The term “furan” refers to a heterocyclic organic compound consisting of a five-membered aromatic ring with four carbon atoms and one oxygen.

The term “thiazole” refers to a heterocyclic compound that contains both sulfur and nitrogen and contains the molecular formula C₃H₃NS.

The term “isothiazole” or 1,2-thiazole refers to an organic compound containing a five-membered aromatic ring that consists of three carbon atoms, one nitrogen atom, and one sulfur atom.

The term “thiadiazole” refers to a sub-family of azole compounds, that are five-membered heterocyclic compounds containing one sulfur and two nitrogen atoms.

The term “oxazole” refers to the parent compound for a class of heterocyclic aromatic organic compounds. Oxazoles are azoles with an oxygen and a nitrogen separated by one carbon.

The term “isoxazole” refers to an azole with an oxygen atom next to the nitrogen.

The term “oxadiazole” refers to a class of heterocyclic aromatic chemical compounds of the azole family with the molecular formula C₂H₂N₂O.

The term “pyridine” refers to a basic heterocyclic organic compound with the chemical formula C₅H₅N. Pyridine is structurally related to benzene, with one methine group replaced by a nitrogen atom.

The term “pyrazine” refers to a heterocyclic organic compound with the chemical formula C₄H₄N₂. Pyrazine is less basic than pyridine, pyridazine and pyrimidine.

The term “pyrimidine” refers to a heterocyclic organic compound similar to pyridine. One of the three diazines, pyrimidine has the nitrogen atoms at positions 1 and 3 in the ring.

The term “pyridazine” is a heterocyclic organic compound with the molecular formula (CH)₄N₂. Pyrimidine contains a six-membered ring with two adjacent nitrogen atoms.

The term “pyrazole” refers to an organic compound with the formula C₃H₃N₂H. Pyrazole is a heterocycle characterized by a 5-membered ring of three carbon atoms and two adjacent nitrogen atoms.

The term “trizole” refers to any of the heterocyclic compounds with molecular formula C₂H₃N₃, having a five-membered ring of two carbon atoms and three nitrogen atoms.

The term “tetrazole” refers to a class of synthetic organic heterocyclic compounds, consisting of a 5-member ring of four nitrogen atoms and one carbon atom. The name tetrazole also refers to the parent compound with formula CH₂N₄.

The term “chroman” or “chromane” refers to a heterocyclic chemical compound with the chemical formula C₉H₁₀O.

The term “quinoline” refers to a heterocyclic organic compound with the chemical formula C₉H₇N.

The term “quinoxaline” or “benzopyrazine” refers to a heterocyclic compound containing a ring complex made up of a benzene ring and a pyrazine ring.

The term “isoquinoline” refers to a heterocyclic aromatic organic compound. Isoquinoline is a structural isomer of quinoline. Isoquinoline and quinoline are benzopyridines, which are composed of a benzene ring fused to a pyridine ring.

The term “phthalazine,” “benzo-orthodiazine,” or “benzopyridazine” is a heterocyclic organic compound with the molecular formula C₈H₆N₂. It is isomeric with other naphthyridines, including quinoxaline, cinnoline and quinazoline.

The term “cinnoline” is a heterocyclic compound with the formula C₈H₆N₂. It is isomeric with other naphthyridines, including quinoxaline, phthalazine and quinazoline.

The term “quinazoline” refers to an organic compound with the formula C₈H₆N₂. Quinazoline is a heterocycle with a bicyclic structure consisting of two fused six-membered aromatic rings, a benzene ring and a pyrimidine ring.

The term “indole” is a heterocyclic organic compound with formula C₈H₇N. Indole has a bicyclic structure, consisting of a six-membered benzene ring fused to a five-membered pyrrole ring.

The term “isoindole” is a benzo-fused pyrrole and is an isomer of indole.

The term “indoline” is a heterocyclic organic compound with the chemical formula C₈H₉N. Indoline has a bicyclic structure, consisting of a six-membered benzene ring fused to a five-membered nitrogen-containing ring.

The term “isoindoline” refers to a heterocyclic organic compound with the molecular formula C₈H₉N. The parent compound has a bicyclic structure, consisting of a six-membered benzene ring fused to a five-membered nitrogen-containing ring.

The term “benzothiophene” refers to an organic compound with a molecular formula C₈H₆S.

The term “benzofuran” refers to a heterocyclic compound consisting of fused benzene and furan rings.

The term “isobenzofuran” refers to a heterocyclic compound consisting of fused benzene and furan rings. Isobenzofuran is isomeric with benzofuran.

The term “benzoxazole” refers to an organic compound with a molecular formula C₇H₅NO and includes a benzene-fused oxazole ring structure.

The term “benzothiazole” refers to a heterocyclic compound with the chemical formula C₇H₅NS.

The term “benzimidazole” refers to a heterocyclic organic compound. Benzimidazole is a bicyclic compound that consists of the fusion of benzene and imidazole.

The term “indazole” or “isoindazole” refers to a heterocyclic organic compound that consists of the fusion of benzene and pyrazole.

The term “benzodioxane” refers to isomeric chemical compounds with the molecular formula C₈H₈O₂.

The term “indane” or “indan” refers to an organic compound with the formula C₆H₄(CH₂)₃.

The term “acridine” refers to an organic compound and a nitrogen heterocycle with the formula C₁₃H₉N. Acridines are substituted derivatives of the parent ring. It is a planar molecule that is structurally related to anthracene with one of the central CH groups replaced by nitrogen.

The term “phenazine” refers to an organic compound with the formula (C₆H₄)₂N₂. Phenazine is a dibenzo annulated pyrazine.

The term “xanthene” refers to an organic compound with the formula CH₂[C₆H₄]₂O.

The term “isochroman” refers to a compound having the formula C₉H₁₀O and the following structure:

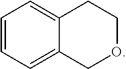

The term “2,1,3-benzoxadiazole” refers to an organic compound having the following structure:

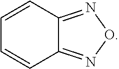

The term “2,1,3-benzothiazole” refers to a heterocyclic compound with the chemical formula C₇H₅NS.

The term “2,1,3-benzoselenadiazole” refers to an organic compound having the following structure:

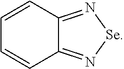

The term “tetrahydroquinoline” refers to an organic compound that is the semi-hydrogenated derivative of quinoline.

The term “3,4-dihydro-2H-1,4-benzoxazine” refers to an organic compound having the following structure:

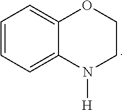

The term “1,5-naphthyridine” refers to an organic compound having the following structure:

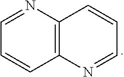

The term “1,8-naphthyridine” refers to an organic compound with the formula C₈H₆N₂.

The term “aryl” means a monocyclic, bicyclic, or tricyclic aromatic group, wherein all rings of the group are aromatic and all ring atoms are carbon atoms. For bicyclic or tricyclic systems, the individual aromatic rings are fused to one another. Examples of aryl groups are 6 and 10 membered aryls. Further examples of aryl groups include, but are not limited to, phenyl, naphthalene, and anthracene.

The term “cyano” as used herein means a substituent having a carbon atom joined to a nitrogen atom by a triple bond.

The term “deuterium” as used herein means a stable isotope of hydrogen having one proton and one neutron.

The term “halogen” as used herein refers to fluorine, chlorine, bromine, iodine, astatine, or tennessine. The term “halo” represents a group comprising a halogen.

The term “hydroxy” means an —OH group.

The term “oxo” means an ═O group and may be attached to a carbon atom or a sulfur atom.

The term “N-oxide” refers to the oxidized form of a nitrogen atom.

The term “cycloalkyl” refers to a saturated or partially saturated, monocyclic, fused polycyclic, bridged polycyclic, or spiro polycyclic carbocycle having from 3 to 15 carbon ring atoms. A non-limiting category of cycloalkyl groups are saturated or partially saturated, monocyclic carbocycles having from 3 to 6 carbon atoms. Illustrative examples of cycloalkyl groups include, but are not limited to, the following moieties:

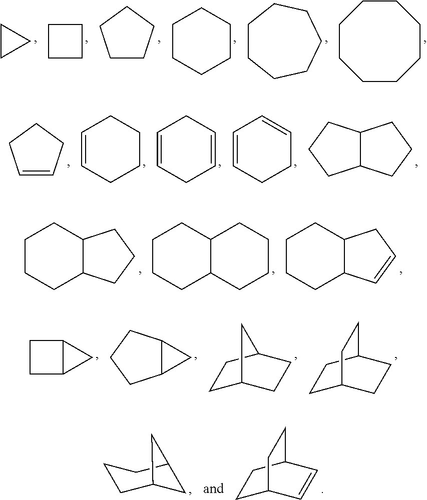

The term “heterocycloalkyl” as used herein refers to a monocyclic, or fused, bridged, or spiro polycyclic ring structure that is saturated or partially saturated and has from three to 12 ring atoms selected from carbon atoms and up to three heteroatoms selected from nitrogen, oxygen, and sulfur. The ring structure may optionally contain up to two oxo groups on carbon or sulfur ring members, or an N-oxide. Illustrative heterocycloalkyl entities include, but are not limited to the following:

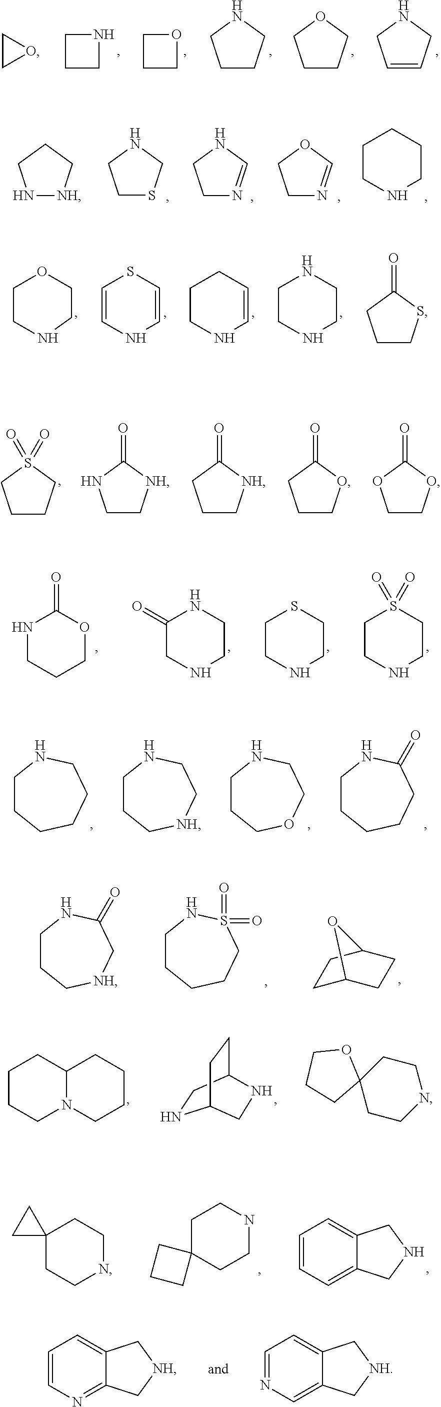

The term “heteroaryl” refers to a monocyclic, or fused polycyclic, aromatic heterocycle having from three to 15 ring atoms that are selected from carbon, oxygen, nitrogen, and sulfur. Suitable heteroaryl groups do not include ring systems that must be charged to be aromatic, such as pyrylium. Suitable 5-membered heteroaryl rings (as a monocyclic heteroaryl or as part of a polycyclic heteroaryl) have one oxygen, sulfur, or nitrogen ring atom, or one nitrogen plus one oxygen or sulfur, or 2, 3, or 4 nitrogen ring atoms. Suitable 6-membered heteroaryl rings (as a monocyclic heteroaryl or as part of a polycyclic heteroaryl) have 1, 2, or 3 nitrogen ring atoms. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The term “fused heteroaryl” refers to a heteroaryl as defined above, having two constituent aromatic rings, wherein the two rings are fused to one another and at least one of the rings is a heteroaryl as defined above. Fused heteroaryls include fused heteroaryl groups comprising 1, 2, 3, or 4 heteroatom ring atoms selected from O, N or S. In certain embodiments, wherein the heteroatom is N it can be an N-oxide. Fused heteroaryls also include 8-, 9-, or 10-membered fused heteroaryl groups. Fused heteroaryls also include 8-, 9-, or 10-membered fused heteroaryl groups that have 1, 2, 3, or 4 heteroatom ring atoms selected from O, N or S. Illustrative examples of fused heteroaryls include, but are not limited to, the following:

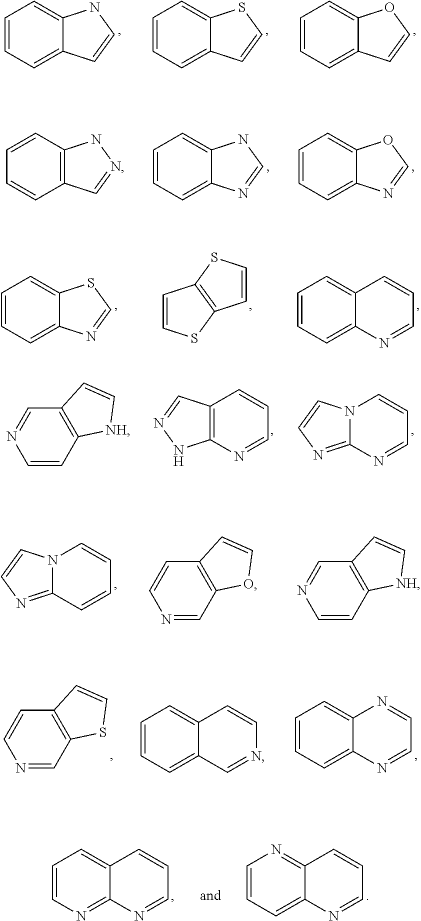

Those skilled in the art will recognize that the species of heteroaryl, cycloalkyl, and heterocycloalkyl groups listed or illustrated above are not exhaustive, and that additional species within the scope of these defined terms may also be selected.

As used herein, “liquid chromatography-mass spectrometry” or LC-MS is an analytical chemistry technique that combines the physical separation capabilities of liquid chromatography (or HPLC) with the mass analysis capabilities of mass spectrometry (MS).

As used herein, “proton nuclear magnetic resonance” or 1H NMR is the application of nuclear magnetic resonance in NMR spectroscopy with respect to hydrogen-1 nuclei within the molecules of a substance, in order to determine the structure of its molecules.

As used herein, the term “substituted” means that the specified group or moiety bears one or more suitable substituents. As used herein, the term “unsubstituted” means that the specified group bears no substituents. As used herein, the term “optionally substituted” means that the specified group is unsubstituted or substituted by the specified number of substituents. Where the term “substituted” is used to describe a structural system, the substitution is meant to occur at any valency-allowed position on the system.

The term “substituent” refers to an atom or group of atoms that replaces one or more hydrogen atoms on the parent chain of a hydrocarbon, becoming a moiety of the resultant new molecule.

Any atom that is represented herein with an unsatisfied valence is assumed to have the sufficient number of hydrogen atoms to satisfy the atom's valence.

When any variable (e.g., alkyl, R^{a}, R¹, etc.) appears in more than one place in any formula or description provided herein, the definition of that variable on each occurrence is independent of its definition at every other occurrence.

Numerical ranges, as used herein, are intended to include sequential whole numbers. For example, a range expressed as “from 0 to 4” or “0-4” includes 0, 1, 2, 3 and 4.

When a multifunctional moiety is shown, the point of attachment to the remainder of the formula can be at any point on the multifunctional moiety. In some embodiments, the point of attachment is indicated by a line or hyphen. For example, aryloxy- refers to a moiety in which an oxygen atom is the point of attachment to the core molecule while aryl is attached to the oxygen atom.

As used herein, the term “subject” encompasses mammals and non-mammals. Examples of mammals include, but are not limited to, any member of the Mammalian class, such as: humans, non-human primates (e.g., chimpanzees, apes, and monkey species), farm animals (e.g., cattle, horses, sheep, goats, swine, etc.), domestic animals (e.g., rabbits, dogs, cats, etc.), and laboratory animals (e.g., rodents, such as rats, mice and guinea pigs, and the like). Examples of non-mammals include, but are not limited to, birds, fish, and the like. In one embodiment of the present invention, the mammal is a human.

The term “patient” or “subject” includes both human and animals.

The terms “effective amount” or “therapeutically effective amount” refer to a sufficient amount of the agent to provide the desired biological result. That result can be reduction and/or alleviation of the signs, symptoms, or causes of a disease or medical condition, or any other desired alteration of a biological system. For example, an “effective amount” for therapeutic use is the amount of a compound, or of a composition comprising the compound, that is required to provide a clinically relevant change in a disease state, symptom, or medical condition. An appropriate “effective” amount in any individual case may be determined by one of ordinary skill in the art using routine experimentation. Thus, the expression “effective amount” generally refers to the quantity for which the active substance has a therapeutically desired effect.

As used herein, the terms “treat” or “treatment” encompass both “preventative” and “curative” treatment. As used herein, the phrase “preventative treatment” is meant to indicate a postponement of development of a disease, a symptom of a disease, or medical condition, suppressing symptoms that may appear, or reducing the risk of developing or recurrence of a disease or symptom. As used herein, the phrase “curative treatment” refers to reducing the severity of or suppressing the worsening of an existing disease, symptom, or condition. Thus, “treatment” includes ameliorating or preventing the worsening of existing disease symptoms, preventing additional symptoms from occurring, ameliorating or preventing the underlying metabolic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

### Specific Embodiments

### Formula I

The present invention, in one of its embodiments, relates to a compound of Formula (I), or pharmaceutically acceptable salts or esters thereof:

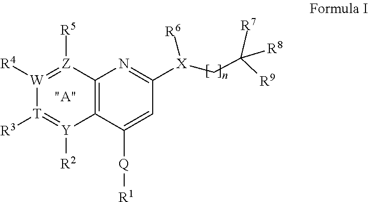

wherein each of R¹ and R⁶-R⁹ are independently —H, —CN, —COOH, —CONH₂, B(ORₐ)₂, an acid isostere, a halo, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl,

wherein Rₐ of the B(ORₐ)₂ is H or an alkyl,

wherein B of the B(ORₐ)₂ is boron,

wherein n of Cₙ is 1-10,

wherein each of R²-R⁵ are independently —H, —CN, —COOH, —COOMe, —CONH₂, B(ORₐ)₂, the acid isostere, the halo, —CONHOH, —NH—SO₂—C₁-C₆-alkyl, —NHSO₂Ar, the Cₙ alkyl, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein the Ar of —NHSO₂Ar is selected from the group consisting of: phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5-naphthyridine, 1,8-naphthyridine, acridine, phenazine, and xanthene,

wherein each of the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, and the Cₙ heterocycloalkyl are unsubstituted or substituted with a quantity of substituents being between 1-5,

wherein each of the substituents is the same or different,

wherein each of the substituents is selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, and —C_{q}—U—C_{q},

wherein each q of —C_{q}—U—C_{q} is independently 0 to 10,

wherein the U of —C_{q}—U—C_{q} is one of: aryl, heteroaryl, cycloalkyl, heterocycloalkyl, O, S, SO₂, and N(R₁)(R₁),
wherein each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl,

wherein each R₁ of N(R₁)(R₁) is unsubstituted or substituted with 1, 2, 3, 4 or 5 substituents which can be the same or different and are independently selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein Q is a bond or O,

wherein X is C, N, O, or S such that R⁶ is not present if X is O or S,

wherein “A” is a saturated or unsaturated ring depicted by

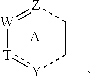

wherein Y, T, W, and Z are independently a bond, C, N, O, an alkyl having 1 to 4 carbon atoms or an alkenyl having 1 to 4 carbon atoms, and

wherein n is 0, 1, 2, or 3.

In one embodiment, the invention comprises the compound of Formula I where at least one of R¹ and R² are bonded forming a fused heteroaryl.

In one embodiment, the invention comprises the compound of Formula I where at least one of R² and R³ are bonded forming a fused heteroaryl.

In one embodiment, the invention comprises the compound of Formula I where at least one of R³ and R⁴ are bonded forming a fused heteroaryl.

In one embodiment, the invention comprises the compound of Formula I where at least one of R⁴ and R⁵ are bonded forming a fused heteroaryl.

In one embodiment, the invention comprises the compound of Formula I where at least one of R⁵ and R⁶ are bonded forming a fused heteroaryl.

In one embodiment, the invention comprises the compound of Formula I where at least one of R⁶ and R⁷ are bonded forming a fused heteroaryl.

In one embodiment, the invention comprises the compound of Formula I where at least one of R⁷ and R⁸ are bonded forming a fused heteroaryl.

In one embodiment, the invention comprises the compound of Formula I where at least one of R⁸ and R⁹ are bonded forming a fused heteroaryl.

In one embodiment, the invention comprises the compound of Formula I where at least one of R¹ and R² are bonded forming a fused heterocycloalkyl.

In one embodiment, the invention comprises the compound of Formula I where at least one of R² and R³ are bonded forming a fused heterocycloalkyl.

In one embodiment, the invention comprises the compound of Formula I where at least one of R³ and R⁴ are bonded forming a fused heterocycloalkyl.

In one embodiment, the invention comprises the compound of Formula I where at least one of R⁴ and R⁵ are bonded forming a fused heterocycloalkyl.

In one embodiment, the invention comprises the compound of Formula I where at least one of R⁵ and R⁶ are bonded forming a fused heterocycloalkyl.

In one embodiment, the invention comprises the compound of Formula I where at least one of R⁶ and R⁷ are bonded forming a fused heterocycloalkyl.

In one embodiment, the invention comprises the compound of Formula I where at least one of R⁷ and R⁸ are bonded forming a fused heterocycloalkyl.

In one embodiment, the invention comprises the compound of Formula I where at least one of R⁸ and R⁹ are bonded forming a fused heterocycloalkyl.

An embodiment of the invention includes a compound of Formula (I), or pharmaceutically acceptable salts or esters thereof, where the various moieties are independently selected, and where R¹-R⁹, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is —H, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is —CN, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is —COOH, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is —CONH₂, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is B(ORₐ)₂, where B of B(ORₐ)₂ is boron, where Rₐ of B(ORₐ)₂ is H, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is B(ORₐ)₂, where B of B(ORₐ)₂ is boron, where Rₐ of B(ORₐ)₂ is an alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is an acid isostere as disclosed herein, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a halo, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl, where n of the Cₙ alkyl is 1-10, where the Cₙ alkyl is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl, where n of the Cₙ alkyl is 1-10, where the Cₙ alkyl is substituted with 1 substituent, where the substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl, where n of the Cₙ alkyl is 1-10, where the Cₙ alkyl is substituted with 2-5 substituents, where the substituents are the same, and where each of the substituents is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl, where n of the Cₙ alkyl is 1-10, where the Cₙ alkyl is substituted with 2-5 substituents, where the substituents differ, where each of the substituents is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ alkyl is 1-10, where a quantity of the substituents is 1, where the substituent is H, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is ²H, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a halo, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an amino group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an alkoxy group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a cyano group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an aminoalkyl-, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an (amino)alkoxy-, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an -alkyl group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an -alkenyl group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an -alkynyl group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an alkoxy- group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a hydroxy group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an -alkylhydroxy group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an aryloxy- group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an -alkyl(aryl) group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an (alkoxyalkyl)amino-group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an aryl group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an -aryl(halo) group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a -heteroaryl group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a hydroxyl-alkyl- group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a hydroxyl-aryl- group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is an (aryl)alkyl- group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a —S(O)₂-alkyl group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a —S(O)₂-aryl group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is a —C(O)alkyl group, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is aryl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is heteroaryl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where the n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is cycloalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is heterocycloalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is O, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is S, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is SO₂, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ alkenyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ alkynyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ aryl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ aminoalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ haloalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ heteroaryl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ cycloalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently the Cₙ heterocycloalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of the Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with one substituent, where the substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents, where each substituent is the same, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n of Cₙ is 1-10, where a quantity of the substituents is 1, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents, where each substituent differs, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where each of the substituents are the same, where each of the substituents are H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where each of the substituents are different, where each of the substituents are H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where each of the substituents are the same, where each of the substituents are H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, or —C_{q}—U—C_{q}, and where R²-R¹, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where each of the substituents are different, where each of the substituents are H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, or —C_{q}—U—C_{q}, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is aryl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is heteroaryl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is cycloalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is heterocycloalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is O, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is S, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is SO₂, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is N(R₁)(R₁), wherein each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is N(R₁)(R₁), wherein each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is N(R₁)(R₁), wherein each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, each R₁ of N(R₁)(R₁) is substituted with 1-5 substituents, where each substituent is the same, where each substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkyl that is substituted, where n is 1-10, where a quantity of the substituents is 2-5, where at least one of the substituents is —C_{q}—U—C_{q}, wherein each q of —C_{q}—U—C_{q} is independently 0 to 10, where the U of —C_{q}—U—C_{q} is N(R₁)(R₁), wherein each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, each R₁ of N(R₁)(R₁) is substituted with 1-5 substituents, where each substituent differs, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 1 substituent, where the substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 2-5 substituents, where the substituents are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 2-5 substituents, where the substituents differ and are independently: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ alkenyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 1 substituent, where the substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 2-5 substituents, where the substituents are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 2-5 substituents, where the substituents differ and are independently: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aryl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 1 substituent, where the substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 2-5 substituents, where the substituents are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 2-5 substituents, where the substituents differ and are independently: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 1 substituent, where the substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 2-5 substituents, where the substituents are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 2-5 substituents, where the substituents differ and are independently: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ haloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 1 substituent, where the substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 2-5 substituents, where the substituents are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 2-5 substituents, where the substituents differ and are independently: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heteroalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl where n of Cₙ is 1-10, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 1 substituent, where the substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 2-5 substituents, where the substituents are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 2-5 substituents, where the substituents differ and are independently: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl where n of Cₙ is 1-10, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ aminoaryl, where n of Cₙ is 1-10, where the Cₙ alkenyl is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 1 substituent, where the substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 1 substituent, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 2-5 substituents, where the substituents are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 2-5 substituents, where the substituents differ and are independently: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1 substituent comprising: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ heterocycloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that are the same and include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, where at least one of R¹ and R⁶-R⁹ is a Cₙ cycloalkyl, where n of Cₙ is 1-10, where the Cₙ alkenyl is substituted with 2-5 substituents, where at least one of the substituents is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 2-5 substituents that differ and may include: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, W, T, Y, Z, Q and X are as defined.

An embodiment of the invention includes a compound of Formula (I), or pharmaceutically acceptable salts or esters thereof, where the various moieties are independently selected, each of R²-R⁵ are independently —H, —CN, —COOH, —COOMe, —CONH₂, B(ORₐ)₂, the acid isostere, the halo, —CONHOH, —NH—SO₂—C₁-C₆-alkyl, —NHSO₂Ar, the Cₙ alkyl, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl, where the B of B(ORₐ)₂ is Boron, where the Rₐ of B(ORₐ)₂ is H or alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where the Ar of —NHSO₂Ar is selected from the group consisting of: phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5-naphthyridine, 1,8-naphthyridine, acridine, phenazine, and xanthene, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is naphthyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is pyrrole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is imidazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is thiophene, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is isothiazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is thiadiazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is oxazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is isoxazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is oxadiazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is pyridine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is pyrazine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is pyrimidine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is pyridazine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is pyrazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is triazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is tetrazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is chroman, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is isochroman, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is quinoline, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is quinoxaline, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is isoquinoline, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is phthalazine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is cinnoline, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is quinazoline, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is indole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is isoindole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is indoline, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is isoindoline, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is benzothiophene, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is benzofuran, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is isobenzofuran, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is benzoxazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is 2,1,3-benzoxadiazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is benzothiazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is 2,1,3-benzothiazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is 2,1,3-benzoselenadiazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is benzimidazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is indazole, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is benzodioxane, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is indane, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is 1,2,3,4-tetrahydroquinoline, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is 3,4-dihydro-2H-1,4-benzoxazine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is 1,5-naphthyridine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is 1,8-naphthyridine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is acridine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is phenazine, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is —NHSO₂Ar, where Ar is xanthene, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl that is unsubstituted, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl that is substituted with 1 substituent, where n of Cₙ is 1-10, where the substituent is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, or —C_{q}—U—C_{q}, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl that is substituted with 1 substituent, where n of Cₙ is 1-10, where the substituent is —C_{q}—U—C_{q}, where each q of —C_{q}—U—C_{q} is independently 0 to 10, where the U of —C_{q}—U—C_{q} is one of: aryl, heteroaryl, cycloalkyl, heterocycloalkyl, O, S, SO₂, and N(R₁)(R₁), and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl that is substituted with 1 substituent, where n of Cₙ is 1-10, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl that is substituted with 1 substituent, where n of Cₙ is 1-10, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents that are the same, where the substituents are H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl that is substituted with 1 substituent, where n of Cₙ is 1-10, where the substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents that differ, where each of the 1-5 substituents are H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkyl that is substituted, where the quantity of substituents is 1, where the substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkyl that is substituted, where the quantity of substituents is 2-5, where each of the substituents is the same and is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkyl that is substituted, where the quantity of substituents is 2-5, where each of the substituents differs and is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkyl that is substituted, where the quantity of substituents is 1, where the substituent is —C_{q}—U—C_{q}, where each q of —C_{q}—U—C_{q} is independently 0 to 10, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkyl that is substituted, where the quantity of substituents is 1, where the substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkyl that is substituted, where n of Cₙ is 1-10, where the quantity of substituents is 1, where the substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkyl that is substituted, where n of Cₙ is 1-10, where the quantity of substituents is 1, where the substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where each of the 1-5 substituents is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkenyl that is unsubstituted, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkenyl that is substituted with 1-5 substituents, where n of Cₙ is 1-10, where each of the substituents are the same or different and include H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkenyl that is substituted, where n of Cₙ is 1-10, where the quantity of substituents is 2-5, where each at least one of the substituents is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkynyl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkynyl that is unsubstituted, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkynyl that is substituted with 1-5 substituents that are the same or are different, where n of Cₙ is 1-10, where the substituent(s) is/are H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ alkynyl that is substituted, where n of Cₙ is 1-10, where the quantity of substituents is 1-5, where the substituent(s) is/are —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ aryl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ aryl that is unsubstituted, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ aryl that is substituted, where n of Cₙ is 1-10, where the quantity of substituents is 1-5, where the substituent(s) are the same or are different, where each of the substituent(s) is/are: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, or —C_{q}—U—C_{q}, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ aryl that is substituted, where n of Cₙ is 1-10, where the quantity of substituents is 1-5, where at least one the substituents is —C_{q}—U—C_{q}, where each q of —C_{q}—U—C_{q} is independently 0 to 10, where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ aminoalkyl, where n of the Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ aminoalkyl that is unsubstituted, where n of the Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ aminoalkyl that is substituted with 1-5 substituents, where n of the Cₙ is 1-10, where each of the substituents is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, or —C_{q}—U—C_{q}, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ aminoalkyl that is substituted, where n of the Cₙ is 1-10, where the quantity of substituents is 1-5, where at least one of the substituents is —C_{q}—U—C_{q}, where each q of —C_{q}—U—C_{q} is independently 0 to 10, where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ haloalkyl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ haloalkyl that is unsubstituted, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ haloalkyl that is substituted with 1-5 substituents, where n of Cₙ is 1-10, where each of the substituents is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C_{q}—U—C_{q}, or —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ haloalkyl that is substituted, where n of Cₙ is 1-10, where the quantity of substituents is 1-5, where at least one of the substituents is —C_{q}—U—C_{q}, where each q of —C_{q}—U—C_{q} is independently 0 to 10, where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ heteroaryl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ heteroaryl that is unsubstituted, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ heteroaryl that is substituted with 1-5 substituents, where n of Cₙ is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, or —C_{q}—U—C_{q}, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ heteroaryl that is substituted, where n of Cₙ is 1-10, where the quantity of substituents is 1-5, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ cycloalkyl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ cycloalkyl that is unsubstituted, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ cycloalkyl that is substituted with 1-5 substituents, where n of Cₙ is 1-10, where each of the substituents is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ cycloalkyl that is substituted, where n of Cₙ is 1-10, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ heterocycloalkyl, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ heterocycloalkyl that is unsubstituted, where n of Cₙ is 1-10, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ heterocycloalkyl that is substituted with 1-5 substituents, where n of Cₙ is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, or —C_{q}—U—C_{q}, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where at least one of R²-R⁵ is the Cₙ heterocycloalkyl that is substituted with 1-5 substituents, where n of Cₙ is 1-10, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, W, T, Y, Z, Q and X are as defined.

An embodiment of the invention includes a compound of Formula (I), or pharmaceutically acceptable salts or esters thereof, where the various moieties are independently selected, where Q is a bond, and where R¹-R⁹, W, T, Y, Z, and X are as defined.

An embodiment of the invention includes a compound of Formula (I), or pharmaceutically acceptable salts or esters thereof, where the various moieties are independently selected, where Q is O, and where R¹-R⁹, W, T, Y, Z, and X are as defined.

An embodiment of the invention includes a compound of Formula (I), or pharmaceutically acceptable salts or esters thereof, where the various moieties are independently selected, where X is C, and where R¹-R⁹, W, T, Y, Z, and Q are as defined.

An embodiment of the invention includes a compound of Formula (I), or pharmaceutically acceptable salts or esters thereof, where the various moieties are independently selected, where X is N, and where R¹-R⁹, W, T, Y, Z, and Q are as defined.

An embodiment of the invention includes a compound of Formula (I), or pharmaceutically acceptable salts or esters thereof, where the various moieties are independently selected, where X is O, R⁶ is not present, and where R¹-R⁵, R⁷-R⁹, W, T, Y, Z, and Q are as defined.

An embodiment of the invention includes a compound of Formula (I), or pharmaceutically acceptable salts or esters thereof, where the various moieties are independently selected, where X is S, R⁶ is not present, and where R¹-R⁵, R⁷-R⁹, W, T, Y, Z, and Q are as defined.

An embodiment of the invention includes a compound of Formula (I), or pharmaceutically acceptable salts or esters thereof, where the various moieties are independently selected, where A is unsaturated, and where R¹-R⁹, W, T, Y, Z, X, and Q are as defined.

An embodiment of the invention includes a compound of Formula (I), or pharmaceutically acceptable salts or esters thereof, where the various moieties are independently selected, where A is saturated, and where R¹-R⁹, W, T, Y, Z, X, and Q are as defined.

An embodiment of the invention includes a compound of Formula (I), or pharmaceutically acceptable salts or esters thereof, where the various moieties are independently selected, where Y is a bond, and where R¹-R⁹, W, T, Z, X, and Q are as defined.

An embodiment of the invention includes a compound of Formula (I), or pharmaceutically acceptable salts or esters thereof, where the various moieties are independently selected, where Y is a C, and where R¹-R⁹, W, T, Z, X, and Q are as defined.

An embodiment of the invention includes a compound of Formula (I), or pharmaceutically acceptable salts or esters thereof, where the various moieties are independently selected, where Y is a N, and where R¹-R⁹, W, T, Z, X, and Q are as defined.

An embodiment of the invention includes a compound of Formula (I), or pharmaceutically acceptable salts or esters thereof, where the various moieties are independently selected, where Y is a O, and where R¹-R⁹, W, T, Z, X, and Q are as defined.

An embodiment of the invention includes a compound of Formula (I), or pharmaceutically acceptable salts or esters thereof, where the various moieties are independently selected, where Y is a an alkyl having 1 to 4 carbon atoms, and where R¹-R⁹, W, T, Z, X, and Q are as defined.

An embodiment of the invention includes a compound of Formula (I), or pharmaceutically acceptable salts or esters thereof, where the various moieties are independently selected, where Y is a an alkenyl having 1 to 4 carbon atoms, and where R¹-R⁹, W, T, Z, X, and Q are as defined.

An embodiment of the invention includes a compound of Formula (I), or pharmaceutically acceptable salts or esters thereof, where the various moieties are independently selected, where n is 0-3, and where R¹-R⁹, W, T, Z, Y, X, and Q are as defined.

An embodiment of the invention includes a compound of Formula (I), or pharmaceutically acceptable salts or esters thereof, where the various moieties are independently selected, where T is a bond, and where R¹-R⁹, W, Y, Z, X, and Q are as defined.

An embodiment of the invention includes a compound of Formula (I), or pharmaceutically acceptable salts or esters thereof, where the various moieties are independently selected, where T is a C, and where R¹-R⁹, W, Y, Z, X, and Q are as defined.

An embodiment of the invention includes a compound of Formula (I), or pharmaceutically acceptable salts or esters thereof, where the various moieties are independently selected, where T is a N, and where R¹-R⁹, W, Y, Z, X, and Q are as defined.

An embodiment of the invention includes a compound of Formula (I), or pharmaceutically acceptable salts or esters thereof, where the various moieties are independently selected, where T is a O, and where R¹-R⁹, W, Y, Z, X, and Q are as defined.

An embodiment of the invention includes a compound of Formula (I), or pharmaceutically acceptable salts or esters thereof, where the various moieties are independently selected, where T is a an alkyl having 1 to 4 carbon atoms, and where R¹-R⁹, W, Y, Z, X, and Q are as defined.

An embodiment of the invention includes a compound of Formula (I), or pharmaceutically acceptable salts or esters thereof, where the various moieties are independently selected, where T is a an alkenyl having 1 to 4 carbon atoms, and where R¹-R⁹, W, Y, Z, X, and Q are as defined.

An embodiment of the invention includes a compound of Formula (I), or pharmaceutically acceptable salts or esters thereof, where the various moieties are independently selected, where W is a bond, and where R¹-R⁹, Y, T, Z, X, and Q are as defined.

An embodiment of the invention includes a compound of Formula (I), or pharmaceutically acceptable salts or esters thereof, where the various moieties are independently selected, where W is a C, and where R¹-R⁹, Y, T, Z, X, and Q are as defined.

An embodiment of the invention includes a compound of Formula (I), or pharmaceutically acceptable salts or esters thereof, where the various moieties are independently selected, where W is a N, and where R¹-R⁹, Y, T, Z, X, and Q are as defined.

An embodiment of the invention includes a compound of Formula (I), or pharmaceutically acceptable salts or esters thereof, where the various moieties are independently selected, where W is a O, and where R¹-R⁹, Y, T, Z, X, and Q are as defined.

An embodiment of the invention includes a compound of Formula (I), or pharmaceutically acceptable salts or esters thereof, where the various moieties are independently selected, where W is a an alkyl having 1 to 4 carbon atoms, and where R¹-R⁹, Y, T, Z, X, and Q are as defined.

An embodiment of the invention includes a compound of Formula (I), or pharmaceutically acceptable salts or esters thereof, where the various moieties are independently selected, where W is a an alkenyl having 1 to 4 carbon atoms, and where R¹-R⁹, Y, T, Z, X, and Q are as defined.

An embodiment of the invention includes a compound of Formula (I), or pharmaceutically acceptable salts or esters thereof, where the various moieties are independently selected, where Z is a bond, and where R¹-R⁹, W, T, Y, X, and Q are as defined.

An embodiment of the invention includes a compound of Formula (I), or pharmaceutically acceptable salts or esters thereof, where the various moieties are independently selected, where Z is a C, and where R¹-R⁹, W, T, Y, X, and Q are as defined.

An embodiment of the invention includes a compound of Formula (I), or pharmaceutically acceptable salts or esters thereof, where the various moieties are independently selected, where Z is a N, and where R¹-R⁹, W, T, Y, X, and Q are as defined.

An embodiment of the invention includes a compound of Formula (I), or pharmaceutically acceptable salts or esters thereof, where the various moieties are independently selected, where Z is a O, and where R¹-R⁹, W, T, Y, X, and Q are as defined.

An embodiment of the invention includes a compound of Formula (I), or pharmaceutically acceptable salts or esters thereof, where the various moieties are independently selected, where Z is a an alkyl having 1 to 4 carbon atoms, and where R¹-R⁹, W, T, Y, X, and Q are as defined.

An embodiment of the invention includes a compound of Formula (I), or pharmaceutically acceptable salts or esters thereof, where the various moieties are independently selected, where Z is a an alkenyl having 1 to 4 carbon atoms, and where R¹-R⁹, W, T, Y, X, and Q are as defined.

### Formula II

In a further embodiment, the invention may be a compound of Formula II or pharmaceutically acceptable salts or esters thereof:

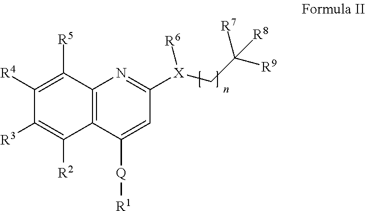

wherein R¹ and R⁶-R⁹ is independently —H, —CN, —COOH, —CONH₂, B(ORₐ)₂, an acid isostere, a halo, Cₙ alkyl, Cₙ alkenyl, Cₙ alkynyl, aryl, aminoalkyl, haloalkyl, heteroaryl, cycloalkyl, or heterocycloalkyl,

wherein the halo is selected from the group consisting of —F, —Cl, —Br, —I, —At, and -Ts,

wherein the Rₐ is H or an alkyl,

wherein the B is boron,

wherein n is 1-10,

wherein each of R²-R⁵ are independently —H, —CN, —COOH, —COOMe, —CONH₂, B(ORₐ)₂, the acid isostere, the halo, —CONHOH, —NH—SO₂-Cι-C₆-alkyl, —NHSO₂Ar, the Cₙ alkyl, the Cₙ alkyl, the Cₙ alkenyl, the Cₙ alkynyl, aryl, aminoalkyl, haloalkyl, heteroaryl, cycloalkyl, or heterocycloalkyl,

wherein the Ar of —NHSO₂Ar is selected from phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5-naphthyridine, 1,8-naphthyridine, acridine, phenazine, and xanthene,

wherein each of the Cₙ alkyl, the Cₙ alkenyl, the Cₙ alkynyl, aryl, aminoalkyl, haloalkyl, heteroaryl, cycloalkyl, or heterocycloalkyl is unsubstituted or substituted with a quantity of substituents being between 1-5,

wherein each of the substituents is the same or different,

wherein each of the substituents is selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, and —C_{q}—U—C_{q},

wherein each q of —C_{q}—U—C_{q} is independently 0 to 10,

wherein the U of —C_{q}—U—C_{q} is any one of aryl, heteroaryl, cycloalkyl, heterocycloalkyl, O, S, SO₂, or N(R₁)(R₁),

wherein each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl,

wherein each R₁ of N(R₁)(R₁) is unsubstituted or substituted with 1, 2, 3, 4 or 5 substituents which can be the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein Q is a bond or O,

wherein X is C, N, O, or S such that R⁶ is not present if X is O or S,

wherein X is C, N, O, or S, and wherein

n is 0, 1, 2, or 3.

In some embodiments, the invention includes compounds of Formula II, wherein a heterocycloalkyl group is formed by bonding two of R⁷, R⁸, or R⁹ to form:

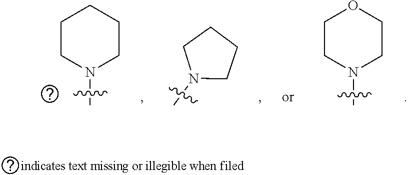

An embodiment of the invention includes a compound of Formula (II), or pharmaceutically acceptable salts or esters thereof, where the various moieties are independently selected, where at least one of R¹ and R⁶-R⁹ is —CN, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is —COOH, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is —CONH₂, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is B(ORₐ)₂, where B of B(ORₐ)₂ is boron, where Rₐ of B(ORₐ)₂ is H, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is B(ORₐ)₂, where B of B(ORₐ)₂ is boron, where Rₐ of B(ORₐ)₂ is an alkyl, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is an acid isostere as disclosed herein, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a halo, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl, where n of the Cₙ alkyl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is unsubstituted, where n of the Cₙ alkyl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted with 1-5 substituents, where n of Cₙ alkyl is 1-10, where each substituent is the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, or —C_{q}—U—C_{q}, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted with 1-5 substituents, where n of Cₙ alkyl is 1-10, where at least one substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted with 1-5 substituents, where n of Cₙ alkyl is 1-10, where at least one substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is H, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is ²H, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where the substituent is a halo, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an amino group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an alkoxy group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is a cyano group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an aminoalkyl-, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an (amino)alkoxy-, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an -alkyl group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an -alkenyl group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an -alkynyl group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an alkoxy-group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is a hydroxy group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an -alkylhydroxy group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an aryloxy-group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an -alkyl(aryl) group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is an (alkoxyalkyl)amino- group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is an aryl group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is an -aryl(halo) group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is a -heteroaryl group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is a hydroxyl-alkyl- group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is a hydroxyl-aryl- group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is an (aryl)alkyl- group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is a —S(O)₂-alkyl group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is a —S(O)₂-aryl group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one the substituent is a —C(O)alkyl group, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is heteroaryl, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is cycloalkyl, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is heterocycloalkyl, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is O, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is S, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is SO₂, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted, where n of Cₙ alkyl is 1-10, where a quantity of the substituents is 1-5, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where each substituent is the same or is different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkyl that is substituted with 1-5 substituents that are the same or are different, where n of Cₙ alkyl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ alkenyl that is unsubstituted or is substituted with 1-5 substituents that are the same or are different, where n of Cₙ alkenyl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ aryl that is unsubstituted or is substituted with 1-5 substituents that are the same or are different, where n of Cₙ aryl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ aminoalkyl that is unsubstituted or is substituted with 1-5 substituents that are the same or are different, where n of Cₙ aminoalkyl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ haloalkyl that is unsubstituted or is substituted with 1-5 substituents that are the same or are different, where n of Cₙ haloalkyl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ heteroaryl that is unsubstituted or is substituted with 1-5 substituents that are the same or are different, where n of Cₙ heteroaryl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ cycloalkyl that is unsubstituted or is substituted with 1-5 substituents that are the same or are different, where n of Cₙ cycloalkyl is 1-10, and where R²-R⁵, Q and X are as defined.

An embodiment of the invention includes a compound of Formula (II), or pharmaceutically acceptable salts or esters thereof, where the various moieties are independently selected, where at least one of R¹ and R⁶-R⁹ is a Cₙ heterocycloalkyl that is unsubstituted or is substituted with 1-5 substituents that are the same or are different, where n of Cₙ heterocycloalkyl is 1-10, and where R²-R⁵, Q and X are as defined.

An embodiment of the invention includes a compound of Formula (II), or pharmaceutically acceptable salts or esters thereof, where the various moieties are independently selected, where at least one of R²-R⁵ is —H, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —CN, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —COOH, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —COOMe, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —CONH₂, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is B(ORₐ)₂, where B is boron, where Rₐ is H, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is B(ORₐ)₂, where B is boron, where Rₐ is an alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the acid isostere, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the halo, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —CONHOH, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NH—SO₂-C₁-C₆-alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is phenyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is naphthyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is pyrrole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is imidazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is thiophene, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is isothiazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is thiadiazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is oxazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is isoxazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is oxadiazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is pyridine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is pyrazine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is pyrimidine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is pyridazine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is pyrazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is triazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is tetrazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is chroman, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is isochroman, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is quinoline, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is quinoxaline, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is isoquinoline, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is phthalazine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is cinnoline, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is quinazoline, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is indole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is isoindole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is indoline, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is isoindoline, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is benzothiophene, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is benzofuran, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is isobenzofuran, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is benzoxazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is 2,1,3-benzoxadiazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is benzothiazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is 2,1,3-benzothiazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is 2,1,3-benzoselenadiazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is benzimidazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is indazole, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is benzodioxane, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is indane, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is 1,2,3,4-tetrahydroquinoline, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is 3,4-dihydro-2H-1,4-benzoxazine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is 1,5-naphthyridine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is 1,8-naphthyridine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is acridine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is phenazine, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is —NHSO₂Ar, where Ar is xanthene, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkyl that is unsubstituted, where n is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkyl that is substituted with 1-5 substituents that are the same or are different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where n of Cₙ alkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkyl that is substituted, where the quantity of substituents is 1-5, where at least one substituent is —C_{q}—U—C_{q}, where each q of —C_{q}—U—C_{q} is independently 0 to 10, where the U is aryl, heteroaryl, cycloalkyl, heterocycloalkyl, O, S, SO₂, or N(R₁)(R₁), where n of Cₙ alkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkyl that is substituted with 1-5 substituents that are the same or are different, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where n of Cₙ alkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkyl that is substituted, where n of Cₙ alkyl is 1-10, where the quantity of substituents is 1-5, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is unsubstituted, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkyl that is substituted, where n of Cₙ alkyl is 1-10, where the quantity of substituents is 1-5, where at least one substituent is —C_{q}—U—C_{q}, where each q of —C_{q}—U—C_{q} is independently 0 to 10, where the U is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents that are the same or are different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkenyl that is unsubstituted, where n of Cₙ alkenyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkenyl that is substituted, where n of Cₙ alkenyl is 1-10, where the quantity of substituents is 1-5 that are the same or are different, where each substituted is: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkenyl that is substituted with 1-5 substituents, where n of Cₙ alkenyl is 1-10, where at least one substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkenyl that is substituted with 1-5 substituents, where n of Cₙ alkenyl is 1-10, where at least one substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkynyl, where n of Cₙ alkynyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkynyl that is unsubstituted, where n of Cₙ alkynyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkynyl that is substituted with 1-5 substituents that are the same or are different, where n of Cₙ alkynyl is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkynyl that is substituted, where n of the Cₙ alkynyl is 1-10, where the quantity of substituents is 1-5, where each substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ alkynyl that is substituted, where n of the Cₙ alkynyl is 1-10, where the quantity of substituents is 1-5, where each substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aryl, where n of Cₙ aryl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aryl that is unsubstituted, where n of Cₙ aryl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aryl that is substituted with 1-5 substituents that are the same or are different, where n of Cₙ aryl is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aryl that is substituted with 1-5 substituents that are the same or are different, where n of Cₙ aryl is 1-10, where at least one substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aryl that is substituted with 1-5 substituents that are the same or are different, where n of Cₙ aryl is 1-10, where at least one substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aminoalkyl, where n of Cₙ aminoalkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aminoalkyl that is unsubstituted, where n of Cₙ aminoalkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aminoalkyl that is substituted with 1-5 substituents that are the same or are different, where n of Cₙ aminoalkyl is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aminoalkyl that is substituted with 1-5 substituents that are the same or are different, where n of Cₙ aminoalkyl is 1-10, where at least one substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ aminoalkyl that is substituted with 1-5 substituents that are the same or are different, where n of Cₙ aminoalkyl is 1-10, where at least one substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ haloalkyl, where n of Cₙ haloalkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ haloalkyl that is unsubstituted, where n of Cₙ haloalkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ haloalkyl that is substituted with 1-5 substituents that are the same or are different, where n of Cₙ haloalkyl is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ haloalkyl that is substituted with 1-5 substituents that are the same or are different, where n of Cₙ haloalkyl is 1-10, where at least one substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ haloalkyl that is substituted with 1-5 substituents that are the same or are different, where n of Cₙ haloalkyl is 1-10, where at least one substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heteroaryl, where n of the Cₙ heteroaryl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heteroaryl that is unsubstituted, where n of the Cₙ heteroaryl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heteroaryl that is substituted with 1-5 substituents that are the same or are different, where n of Cₙ heteroaryl is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heteroaryl that is substituted with 1-5 substituents that are the same or are different, where n of Cₙ heteroaryl is 1-10, where at least one substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heteroaryl that is substituted with 1-5 substituents that are the same or are different, where n of Cₙ heteroaryl is 1-10, where at least one substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ cycloalkyl, where n of Cₙ cycloalkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ cycloalkyl that is unsubstituted, where n of Cₙ cycloalkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ cycloalkyl that is substituted with 1-5 substituents that are the same or are different, where n of Cₙ cycloalkyl is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ cycloalkyl that is substituted with 1-5 substituents that are the same or are different, where n of Cₙ cycloalkyl is 1-10, where at least one substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ cycloalkyl that is substituted with 1-5 substituents that are the same or are different, where n of Cₙ cycloalkyl is 1-10, where at least one substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heterocycloalkyl, where n of the Cₙ heterocycloalkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heterocycloalkyl that is unsubstituted, where n of the Cₙ heterocycloalkyl is 1-10, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heterocycloalkyl that is substituted with 1-5 substituents that are the same or are different, where n of Cₙ heterocycloalkyl is 1-10, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heterocycloalkyl that is substituted with 1-5 subsistent that are the same or are different, where n of Cₙ heterocycloalkyl is 1-10, where at least one substituent is —C_{q}—U—C_{q}, where each q of —C_{q}—U—C_{q} is independently 0 to 10, where the U of —C_{q}—U—C_{q} is any one of aryl, heteroaryl, cycloalkyl, heterocycloalkyl, O, S, SO₂, or N(R₁)(R₁), and where R¹, R⁶-R⁹, Q and X are as defined.

An embodiment of the invention includes a compound of Formula (II), or pharmaceutically acceptable salts or esters thereof, where the various moieties are independently selected, where at least one of R²-R⁵ is the Cₙ heterocycloalkyl that is substituted with 1-5 subsistent that are the same or are different, where n of Cₙ heterocycloalkyl is 1-10, where at least one substituent is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, the Cₙ alkyl, the Cₙ alkenyl, the Cₙ alkynyl, aryl, aminoalkyl, haloalkyl, heteroaryl, cycloalkyl, or heterocycloalkyl, where each R₁ of N(R₁)(R₁) is substituted with 1-5 substituents, where the substituent of each R₁ of N(R₁)(R₁) is the same or different and are independently selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl, and where R¹, R⁶-R⁹, Q and X are as defined.

An embodiment of the invention includes a compound of Formula (II), or pharmaceutically acceptable salts or esters thereof, where the various moieties are independently selected, where Q is a bond, and where R¹-R⁹ and X are as defined.

An embodiment of the invention includes a compound of Formula (II), or pharmaceutically acceptable salts or esters thereof, where the various moieties are independently selected, where Q is O, and where R¹-R⁹ and X are as defined.

An embodiment of the invention includes a compound of Formula (II), or pharmaceutically acceptable salts or esters thereof, where the various moieties are independently selected, where X is C, and where R¹-R⁹ and X are as defined.

An embodiment of the invention includes a compound of Formula (II), or pharmaceutically acceptable salts or esters thereof, where the various moieties are independently selected, where X is N, and where R¹-R⁹, R¹-R⁹ and X are as defined.

An embodiment of the invention includes a compound of Formula (II), or pharmaceutically acceptable salts or esters thereof, where the various moieties are independently selected, where X is O, R⁶ is not present, and where R¹-R⁵, R⁷-R⁹, R¹-R⁹ and X are as defined. An embodiment of the invention includes a compound of Formula (II), or pharmaceutically acceptable salts or esters thereof, where the various moieties are independently selected, where X is S, R⁶ is not present, and where R¹-R⁵, R⁷-R⁹, R¹-R⁹ and X are as defined.

An embodiment of the invention includes a compound of Formula (II), or pharmaceutically acceptable salts or esters thereof, where the various moieties are independently selected, where n is 0-3, and where R¹-R⁹, Q and X are as defined.

### Formula III

Additional embodiments of the invention include compounds of Formula III or pharmaceutically acceptable salts or esters thereof:

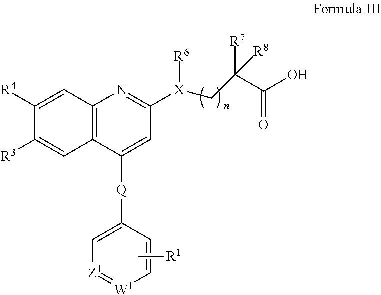

wherein R¹ is selected from the group consisting of: —CN, alkyl, —H, halo, ²H, amino, alkoxy, aminoalkyl, (amino)alkoxy, alkenyl, alkynyl, alkoxy, hydroxy, alkylhydroxy, aryloxy, alkyl(aryl), (alkoxyalkyl)amino, aryl, aryl(halo), heteroaryl, hydroxyl-alkyl, hydroxyl-aryl, (aryl)alkyl, C(O)OH, —S(O)₂-alkyl, —S(O)₂-aryl, —C(O)alkyl, and C(O)NH₂,

wherein each of R³ and R⁴ are independently —H, the halo, Cₙ alkyl, Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, the Cₙ heterocycloalkyl, and —C_{q}—U—C_{q},

wherein n is 1-10,

wherein each q of —C_{q}—U—C_{q} is independently 0 to 10,

wherein the U of —C_{q}—U—C_{q} is any one of O, S, SO₂, or N(R₁)(R₁),

wherein each R₁ of N(R₁)(R₁) independently hydrogen,

wherein each of the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl is unsubstituted or substituted with a quantity of substituents being between 1-5,

wherein each of the substituents is the same or different,

wherein each of the substituents is selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein R⁶ is independently H or alkyl,

wherein each of R⁷ and R⁸ are independently hydrogen, ²H, fluoro or alkyl,

wherein R⁶ and R⁷ are bonded with an adjoining R group to form a fused group,

wherein the fused group is selected from the group consisting of: a fused cycloalkyl, a fused heterocycloalkyl, a fused aryl, and a fused heteroaryl ring,

wherein the fused group comprises between 4 to 10 carbon atoms,

wherein n of Formula III is 0 or 1,

wherein Q is a bond or O,

wherein X is C, N, O, or S such that R⁶ is not present if X is O or S, and

wherein Z¹ or W¹ are independently C, N, O, or S.

An embodiment of the invention includes a compound of Formula (III), or pharmaceutically acceptable salts or esters thereof, where the various moieties are independently selected, where n is 0, and where R¹, R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 1, and where R¹, R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where Q is a bond, and where R¹, R³-R⁴, R⁶-R⁸, X, Z¹, and W¹ are as defined.

An embodiment of the invention includes a compound of Formula (III), or pharmaceutically acceptable salts or esters thereof, where the various moieties are independently selected, where n is 0 or 1, where Q is O, and where R¹, R³-R⁴, R⁶-R⁸, X, Z¹, and W¹ are as defined.

An embodiment of the invention includes a compound of Formula (III), or pharmaceutically acceptable salts or esters thereof, where the various moieties are independently selected, where n is 0 or 1, where X is C, and where R¹, R³-R⁴, R⁶-R⁸, Q, Z¹, and W¹ are as defined.

An embodiment of the invention includes a compound of Formula (III), or pharmaceutically acceptable salts or esters thereof, where the various moieties are independently selected, where n is 0 or 1, where X is N, and where R¹, R³-R⁴, R⁶-R⁸, Q, Z¹, and W¹ are as defined.

An embodiment of the invention includes a compound of Formula (III), or pharmaceutically acceptable salts or esters thereof, where the various moieties are independently selected, where n is 0 or 1, where X is O, where R⁶ is not present, and where R¹, R³-R⁴, R⁷-R⁸, Q, Z¹, and W¹ are as defined.

An embodiment of the invention includes a compound of Formula (III), or pharmaceutically acceptable salts or esters thereof, where the various moieties are independently selected, where n is 0 or 1, where X is S, where R⁶ is not present, and where R¹, R³-R⁴, R⁷-R⁸, Q, Z¹, and W¹ are as defined.

An embodiment of the invention includes a compound of Formula (III), or pharmaceutically acceptable salts or esters thereof, where the various moieties are independently selected, where n is 0 or 1, where Z¹ is C, and where R¹, R³-R⁴, R⁷-R⁸, Q, X, and W¹ are as defined.

An embodiment of the invention includes a compound of Formula (III), or pharmaceutically acceptable salts or esters thereof, where the various moieties are independently selected, where n is 0 or 1, where Z¹ is N, and where R¹, R³-R⁴, R⁷-R⁸, Q, X, and W¹ are as defined.

An embodiment of the invention includes a compound of Formula (III), or pharmaceutically acceptable salts or esters thereof, where the various moieties are independently selected, where n is 0 or 1, where Z¹ is O, and where R¹, R³-R⁴, R⁷-R⁸, Q, X, and W¹ are as defined.

An embodiment of the invention includes a compound of Formula (III), or pharmaceutically acceptable salts or esters thereof, where the various moieties are independently selected, where n is 0 or 1, where Z¹ is S, and where R¹, R³-R⁴, R⁷-R⁸, Q, X, and W¹ are as defined.

An embodiment of the invention includes a compound of Formula (III), or pharmaceutically acceptable salts or esters thereof, where the various moieties are independently selected, where n is 0 or 1, where W¹ is C, and where R¹, R³-R⁴, R⁷-R⁸, Q, X, and Z¹ are as defined.

An embodiment of the invention includes a compound of Formula (III), or pharmaceutically acceptable salts or esters thereof, where the various moieties are independently selected, where n is 0 or 1, where W¹ is N, and where R¹, R³-R⁴, R⁷-R⁸, Q, X, and Z¹ are as defined.

An embodiment of the invention includes a compound of Formula (III), or pharmaceutically acceptable salts or esters thereof, where the various moieties are independently selected, where n is 0 or 1, where W¹ is O, and where R¹, R³-R⁴, R⁷-R⁸, Q, X, and Z¹ are as defined.

An embodiment of the invention includes a compound of Formula (III), or pharmaceutically acceptable salts or esters thereof, where the various moieties are independently selected, where n is 0 or 1, where W¹ is S, and R¹, R³-R⁴, R⁷-R⁸, Q, X, and Z¹ are as defined.

An embodiment of the invention includes a compound of Formula (III), or pharmaceutically acceptable salts or esters thereof, where the various moieties are independently selected, where n is 0 or 1, where R¹ is —CN, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is alkyl, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is —H, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is a halo, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is ²H, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an amino group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an alkoxy group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an aminoalkyl group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an (amino)alkoxy group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an alkenyl group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an alkynyl group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an alkoxy group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is a hydroxy group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an alkylhydroxy group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an aryloxy group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an alkyl(aryl) group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an (alkoxyalkyl)amino group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an aryl group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an aryl(halo) group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is a heteroaryl group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is a hydroxyl-alkyl group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is a hydroxyl-aryl group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is an (aryl)alkyl group, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is C(O)OH, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is —S(O)₂-alkyl, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is —S(O)₂-aryl, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is C(O)alkyl, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where R¹ is C(O)NH₂, and where R³-R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is —H, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a halo, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ alkyl, where the n of the Cₙ alkyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ alkyl, where the Cₙ alkyl is unsubstituted, where the n of the Cₙ alkyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ alkenyl, where the Cₙ alkenyl is unsubstituted, where the n of the Cₙ alkenyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ alkynyl, where the Cₙ alkynyl is unsubstituted, where the n of the Cₙ alkynyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ aryl, where the Cₙ aryl is unsubstituted, where the n of the Cₙ aryl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ aminoalkyl, where the Cₙ aminoalkyl is unsubstituted, where the n of the Cₙ aminoaryl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ haloalkyl, where the Cₙ haloalkyl is unsubstituted, where the n of the Cₙ haloalkyl is 1-10, and where R¹, R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ heteroaryl, where the Cₙ heteroaryl is unsubstituted, where the n of the Cₙ heteroaryl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ cycloalkyl, where the Cₙ cycloalkyl is unsubstituted, where the n of the Cₙ cycloalkyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ heterocycloalkyl, where the Cₙ heterocycloalkyl is unsubstituted, where the n of the Cₙ heterocycloalkyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is —C_{q}—U—C_{q}, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is —C_{q}—U—C_{q}, where each q of —C_{q}—U—C_{q} is independently 0 to 10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is —C_{q}—U—C_{q}, where each q of —C_{q}—U—C_{q} is independently 0 to 10, where the U of —C_{q}—U—C_{q} is O, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is —C_{q}—U—C_{q}, where each q of —C_{q}—U—C_{q} is independently 0 to 10, where the U of —C_{q}—U—C_{q} is S, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is —C_{q}—U—C_{q}, where each q of —C_{q}—U—C_{q} is independently 0 to 10, where the U of —C_{q}—U—C_{q} is SO₂, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is —C_{q}—U—C_{q}, where each q of —C_{q}—U—C_{q} is independently 0 to 10, where the U of —C_{q}—U—C_{q} is N(R₁)(R₁), where each R₁ of N(R₁)(R₁) is independently hydrogen, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ alkyl, where the Cₙ alkyl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where the n of the Cₙ alkyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ alkenyl, where the Cₙ alkenyl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where the n of the Cₙ alkenyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ alkynyl, where the Cₙ alkynyl is unsubstituted, where the n of the Cₙ alkynyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ alkynyl, where the Cₙ alkynyl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where the n of the Cₙ alkynyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ aryl, where the Cₙ aryl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where the n of the Cₙ aryl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ aminoalkyl, where the Cₙ aminoalkyl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl where the n of the Cₙ aminoaryl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ haloalkyl, where the Cₙ haloalkyl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where the n of the Cₙ haloalkyl is 1-10, and where R¹, R⁴, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ heteroaryl, where the Cₙ heteroaryl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where the n of the Cₙ heteroaryl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ cycloalkyl, where the Cₙ cycloalkyl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where the n of the Cₙ cycloalkyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is a Cₙ heterocycloalkyl, where the Cₙ heterocycloalkyl is substituted with 1-5 substituents that are the same or different, where each substituent is H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, or —C(O)alkyl, where the n of the Cₙ heterocycloalkyl is 1-10, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where n is 0 or 1, where at least one of R³ and R⁴ is —C_{q}—U—C_{q}, where each q of —C_{q}—U—C_{q} is independently 0 to 10, where the U of —C_{q}—U—C_{q} is any one of O, S, SO₂, or N(R₁)(R₁), where each R₁ of N(R₁)(R₁) independently hydrogen, and where R¹, R⁶-R⁸, Q, X, Z¹, and W¹ are as defined.

An embodiment of the invention includes a compound of Formula (III), or pharmaceutically acceptable salts or esters thereof, where the various moieties are independently selected, where R⁶ is H, and where R¹, R³-R⁴, R⁷-R⁸, Q, X, Z¹, and W¹ are as defined.

An embodiment of the invention includes a compound of Formula (III), or pharmaceutically acceptable salts or esters thereof, where the various moieties are independently selected, where R⁶ is alkyl, and where R¹, R³-R⁴, R⁷-R⁸, Q, X, Z¹, and W¹ are as defined.

An embodiment of the invention includes a compound of Formula (III), or pharmaceutically acceptable salts or esters thereof, where the various moieties are independently selected, where R⁷ is H, and where R¹, R³-R⁴, R⁶, R⁸, Q, X, Z¹, and W¹ are as defined.

An embodiment of the invention includes a compound of Formula (III), or pharmaceutically acceptable salts or esters thereof, where the various moieties are independently selected, where R⁷ is ²H, and where R¹, R³-R⁴, R⁶, R⁸, Q, X, Z¹, and W¹ are as defined.

An embodiment of the invention includes a compound of Formula (III), or pharmaceutically acceptable salts or esters thereof, where the various moieties are independently selected, where R⁷ is fluoro, and where R¹, R³-R⁴, R⁶, R⁸, Q, X, Z¹, and W¹ are as defined.

An embodiment of the invention includes a compound of Formula (III), or pharmaceutically acceptable salts or esters thereof, where the various moieties are independently selected, where R⁷ is alkyl, and where R¹, R³-R⁴, R⁶, R⁸, Q, X, Z¹, and W¹ are as defined.

An embodiment of the invention includes a compound of Formula (III), or pharmaceutically acceptable salts or esters thereof, where the various moieties are independently selected, where R⁸ is H, and where R¹, R³-R⁴, R⁶, R⁷, Q, X, Z¹, and W¹ are as defined.

An embodiment of the invention includes a compound of Formula (III), or pharmaceutically acceptable salts or esters thereof, where the various moieties are independently selected, where R⁸ is ²H, and where R¹, R³-R⁴, R⁶, R⁷, Q, X, Z¹, and W¹ are as defined.

An embodiment of the invention includes a compound of Formula (III), or pharmaceutically acceptable salts or esters thereof, where the various moieties are independently selected, where R⁸ is fluoro, and where R¹, R³-R⁴, R⁶, R⁷, Q, X, Z¹, and W¹ are as defined.

An embodiment of the invention includes a compound of Formula (III), or pharmaceutically acceptable salts or esters thereof, where the various moieties are independently selected, where R⁸ is alkyl, and where R¹, R³-R⁴, R⁶, R⁷, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group comprises 4 carbon atoms, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group comprises 5 carbon atoms, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group comprises 6 carbon atoms, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group comprises 7 carbon atoms, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group comprises 8 carbon atoms, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group comprises 9 carbon atoms, and where R¹, R³-R⁴, R¹, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group comprises 10 carbon atoms, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group is a fused cycloalkyl ring, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group is a fused heterocycloalkyl ring, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group is a fused aryl ring, and where R¹, R³-R⁴, R⁸, Q, X, Z¹, and W¹ are as defined.

An embodiment of the invention includes a compound of Formula (III), or pharmaceutically acceptable salts or esters thereof, where the various moieties are independently selected, where R⁶ and R⁷ are bonded with an adjoining R group to form a fused group, where the fused group is a fused heteroaryl ring, and where R¹, R³-R⁴, R⁸, Q, X, Z¹, and W¹ are as defined.

In one embodiment of the present invention, there is a method of inhibiting the fatty acid binding protein FABP4 in a mammal, which comprises administering an effective amount of a compound of Formula (I) to a mammal.

In one embodiment of the present invention, the subject is a human.

In one embodiment of the present invention, the compound according to Formula (I) is used in the prophylaxis or treatment of disorders acting on the fatty acid binding protein FABP4.

In one embodiment of the present invention, the disorders are: type 2 diabetes, hyperglycemia, Alzheimer's disease, metabolic syndrome, obesity, atherosclerosis, intracranial atherosclerotic disease, non-alcoholic steatohepatitis, asthma, multiple sclerosis, Alzheimer's disease, other chronic inflammatory and autoimmune/inflammatory diseases, chronic heart disease, polycystic ovary syndrome, preeclampsia, or cancer.

In one embodiment of the present invention, there is a pharmaceutical composition comprising a compound according to Formula (I) as the active ingredient.

In one embodiment of the present invention, there is a pharmaceutical composition that further includes at least one additional active ingredient or a pharmaceutically acceptable carrier.

In one embodiment of the present invention, there is a method for the prophylaxis or treatment of disorders acting on the FABP4, which comprises administering an effective amount of a compound according to Formula (I) to a subject in need of treatment.

Any formula given herein is intended to represent compounds having structures depicted by the structural formula as well as certain variations or forms. For example, compounds of any formula given herein may have asymmetric or chiral centers and therefore exist in different stereoisomeric forms. All stereoisomers, including optical isomers, enantiomers, and diastereomers, of the compounds of the general formula, and mixtures thereof, are considered to fall within the scope of the formula. Furthermore, certain structures may exist as geometric isomers (e.g., cis and trans isomers), as tautomers, or as atropisomers. All such isomeric forms, and mixtures thereof, are contemplated herein as part of the present invention. Thus, any formula given herein is intended to represent a racemate, one or more enantiomeric forms, one or more diastereomeric forms, one or more tautomeric or atropisomeric forms, and mixtures thereof.

Diastereomeric mixtures may be separated into their individual diastereomers on the basis of their physical chemical differences by methods well known to those skilled in the art, such as, for example, by chromatography and/or fractional crystallization. Enantiomers may be separated by converting the enantiomeric mixture into a diastereomeric mixture by reaction with an appropriate optically active compound (e.g., chiral auxiliary such as a chiral alcohol or Mosher's acid chloride, or formation of a mixture of diastereomeric salts), separating the diastereomers and converting (e.g., hydrolyzing or de-salting) the individual diastereomers to the corresponding pure enantiomers. Enantiomers may also be separated by use of chiral HPLC column. The chiral centers of compounds of the present invention may be designated as “R” or “S” as defined by the IUPAC 1974 Recommendations.

The compounds of the invention can form pharmaceutically acceptable salts, which are also within the scope of this invention. As described herein, a “pharmaceutically acceptable salt” refers to a salt of a free acid or base of a compound of Formula I, II, or III that is non-toxic, is physiologically tolerable, is compatible with the pharmaceutical composition in which it is formulated, and is otherwise suitable for formulation and/or administration to a subject. Reference to a compound herein is understood to include reference to a pharmaceutically acceptable salt of said compound unless otherwise indicated.

Compound salts include acidic salts formed with inorganic and/or organic acids, as well as basic salts formed with inorganic and/or organic bases. In addition, where a given compound contains both a basic moiety, such as, but not limited to, a pyridine or imidazole, and an acidic moiety, such as, but not limited to, a carboxylic acid, one of skill in the art will recognize that the compound may exist as a zwitterion (“inner salt”); such salts are included within the term “salt” as used herein. Salts of the compounds of the invention may be prepared, for example, by reacting a compound with an amount of a suitable acid or base, such as an equivalent amount, in a medium such as one in which the salt precipitates or in an aqueous medium followed by lyophilization.

Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate (“mesylate”), ethanesulfonate, benzenesulfonate, p-toluenesulfonate, and pamoate (e.g., 1,1′-methylene-bis(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counter ion.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

Additionally, acids and bases which are generally considered suitable for the formation of pharmaceutically useful salts from pharmaceutical compounds are discussed, for example, by P. Stahl et al., Camille G. (eds.) Handbook of Pharmaceutical Salts: Properties, Selection and Use. (2002) Zurich: Wiley-VCH; S. Berge et al., J. Pharm. Sci. (1977) 66(1) 1-19; P. Gould, Int. J. Pharm. (1986) 33 201-217; Anderson et al., The Practice of Medicinal Chemistry (1996), Academic Press, New York; and in The Orange Book (Food & Drug Administration, MD, available from FDA). These disclosures are incorporated herein by reference thereto.

Additionally, any compound described herein is intended to refer also to any unsolvated form, or a hydrate, solvate, or polymorph of such a compound, and mixtures thereof, even if such forms are not listed explicitly. As described herein, “solvate” means a physical association of a compound of the invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of a crystalline solid. As described herein, “solvate” encompasses both solution-phase and isolatable solvates. Suitable solvates include those formed with pharmaceutically acceptable solvents such as water, ethanol, and the like. In some embodiments, the solvent is water and the solvates are hydrates.

One or more compounds of the invention may optionally be converted to a solvate. Methods for the preparation of solvates are generally known. Thus, one group describes the preparation of the solvates of the antifungal fluconazole in ethyl acetate as well as from water. See, M. Caira, et al., “Preparation and Crystal Characterization of a Polymorph, a Monohydrate, and an Ethyl Acetate Solvate of the Antifungal Fluconazole,” Journal of Pharmaceutical Sciences, 2004, 93(3), Pages 601-611, https://doi.org/10.1002/jps.10541. Similar preparations of solvates, hemisolvate, hydrates, and the like are also described. See, E. Tonder, et al., “Preparation and Physicochemical Characterization of 5 Niclosamide Solvates and 1 Hemisolvate,” AAPS PharmSciTech., 5(1), E12, DOI: 10.1208/pt050112; and A. Bingham, et al., “Over One Hundred Solvates of Sulfathiazoleelectronic Supplementary Information (ESI) Available: Solvates and Adducts of Sulfathiazole,” Chemical Communications, 2001, 7, Pages 603-604. A typical, non-limiting process involves dissolving the inventive compound in a suitable amounts of the solvent (organic solvent or water or a mixture thereof) at a higher than ambient temperature, and cooling the solution at a rate sufficient to form crystals which are then isolated by standard methods. Analytical techniques such as, for example, infrared spectroscopy, show the presence of the solvent (or water) in the crystals as a solvate (or hydrate).

The invention also relates to pharmaceutically acceptable prodrugs of the compounds of Formula I, II, or III, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula I). A “pharmaceutically acceptable prodrug” is a prodrug that is non-toxic, biologically tolerable, and otherwise suitable for formulation and/or administration to the subject. Illustrative procedures for the selection and preparation of suitable prodrug derivatives are described, for example, in “Design of Prodrugs”, ed. H. Bundgaard, Elsevier, 1985.

Examples of prodrugs include pharmaceutically acceptable esters of the compounds of the invention, which are also considered to be part of the invention. Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, C₁₋₄alkoxy, or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol. Additional discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press.

For example, if a compound of Formula I, II, or III contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholine (C₂-C₃)alkyl, and the like.

Similarly, if a compound of Formula I, II, or III contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I, II, or III incorporates an amine functional group, a prodrug can be formed by the replacement of a hydrogen atom in the amine group with a group such as, for example, R″-carbonyl, R″O-carbonyl, NR″R′-carbonyl where R″ and R′ are each independently (C₁-C₂₀)alkyl, (C₃-C₇) cycloalkyl, benzyl, or R″-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

The present invention also relates to pharmaceutically active metabolites of compounds of Formula I, II, or III, and uses of such metabolites in the methods of the invention. A “pharmaceutically active metabolite” means a pharmacologically active product of metabolism in the body of a compound of Formula I, II, or III, or salt thereof. Prodrugs and active metabolites of a compound may be determined using routine techniques known or available in the art. See, G. Bertolini, et al., “A New Rational Hypothesis for the Pharmacophore of the Active Metabolite of Leflunomide, a Potent Immunosuppressive Drug,” Journal of Medicinal Chemistry, 1997, 40(13), Pages 2011-2016, https://doi.org/10.1021/jm970039n; D. Shan, et al., “Prodrug Strategies Based on Intramolecular Cyclization Reactions,” Journal of Pharmaceutical Sciences, 1997, 86(7), Pages 765-767, https://doi.org/10.1021/js970069d; K. Bagshawe, “Antibody-Directed Enzyme Prodrug Therapy: A Review,” Drug Development Research, 1995, 34(2), Pages 220-230, https://doi.org/10.1002/ddr.430340211; N. Bodor, Advanced Drug Delivery Reviews, 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (for example with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly suitable for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

The use of the terms “salt,” “solvate,” “polymorph,” “prodrug,” and the like, with respect to the compounds described herein is intended to apply equally to the salt, solvate, polymorph, and prodrug forms of enantiomers, stereoisomers, rotamers, tautomers, atropisomers, and racemates of the inventive compounds.

The embodiments of the present invention can also be a compound selected from the compounds listed below in Table 1.

TABLE 1

| Structure | IUPAC Name |
|---|---|
| 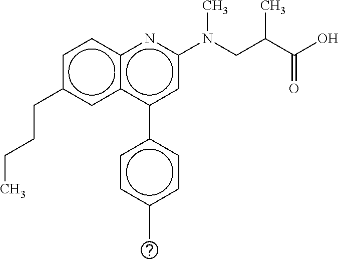 | 3-{[6-butyl-4-(4- fluorophenyl)quinolin-2- yl](methyl)amino}-2- methylpropanoic acid |
| 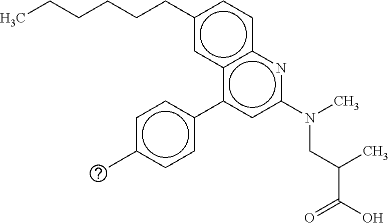 | 3-{[4-(4-fluorophenyl)-6- hexylquinolin-2- yl](methyl)amino}-2- methylpropanoic acid |
| 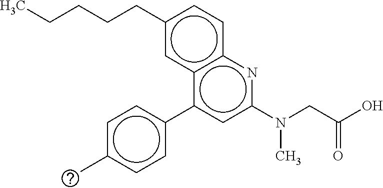 | 2-{[4-(4-fluorophenyl)-6- pentylquinolin-2- yl](methyl)amino}acetic acid |
| 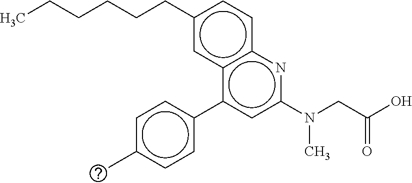 | 2-{[4-(4-fluorophenyl)-6- hexylquinolin-2- yl](methyl)amino}acetic acid |
| 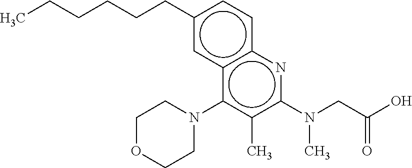 | 2-{[6-hexyl-3-methyl-4- (morpholin-4-yl)quinolin-2- yl](methyl)amino}acetic acid |
| 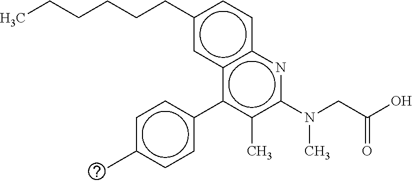 | 2-{[4-(4-fluorophenyl)-6-hexyl- 3-methylquinolin-2- yl](methyl)amino}acetic acid |
| 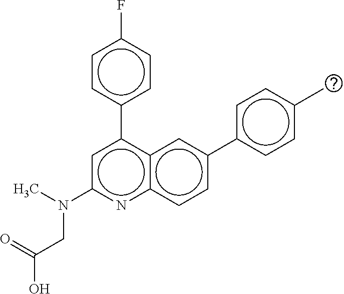 | 2-{[4,6-bis(4- fluorophenyl)quinolin-2- yl](methyl)amino}acetic acid |
| 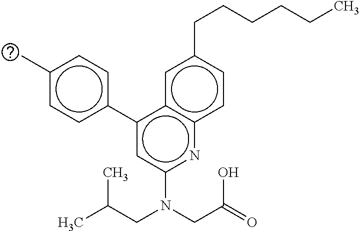 | 2-{[4-(4-fluorophenyl)-6- hexylquinolin-2-yl](2- methylpropyl)amino}acetic acid |
| 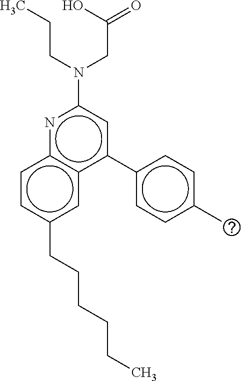 | 2-{[4-(4-fluorophenyl)-6- hexylquinolin-2- yl](propyl)amino}acetic acid |
| 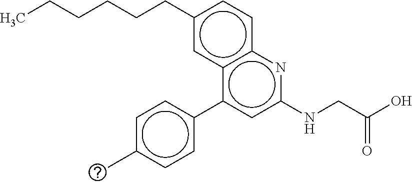 | 2-{[4-(4-fluorophenyl)-6- hexylquinolin-2-yl]amino}acetic acid |
| 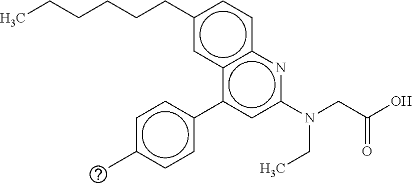 | 2-{ethyl[4-(4-fluorophenyl)-6- hexylquinolin-2-yl]amino}acetic acid |
| 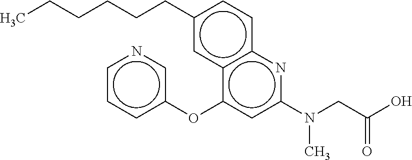 | 2-{[6-hexyl-4-(pyridin-3- yloxy)quinolin-2- yl](methyl)amino}acetic acid |
| 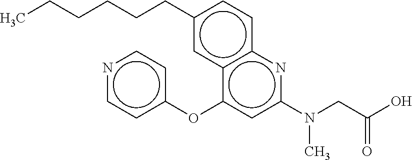 | 2-{[6-hexyl-4-(pyridin-4- yloxy)quinolin-2- yl](methyl)amino}acetic acid |
| 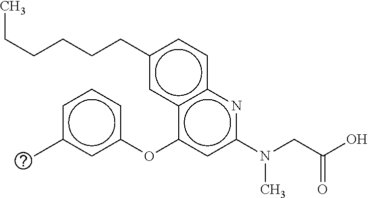 | 2-{[4-(3-fluorophenoxy)-6- hexylquinolin-2- yl](methyl)amino}acetic acid |
| 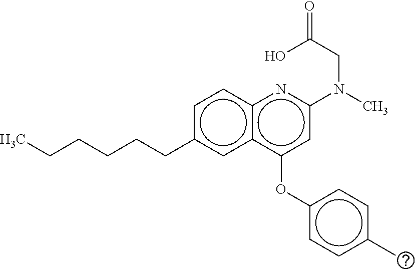 | 2-{[4-(4-fluorophenoxy)-6- hexylquinolin-2- yl](methyl)amino}acetic acid |
| 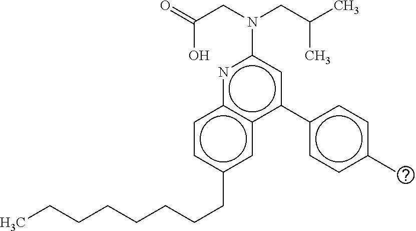 | 2-{[4-(4-fluorophenyl)-6- octylquinolin-2-yl](2- methylpropyl)amino}acetic acid |
| 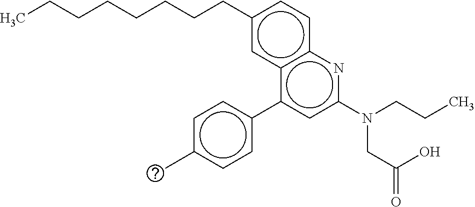 | 2-{[4-(4-fluorophenyl)-6- octylquinolin-2- yl](propyl)amino}acetic acid |
| 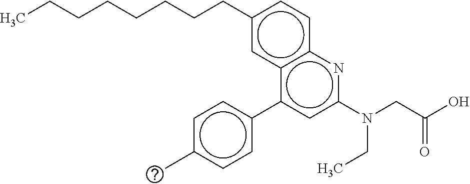 | 2-{ethyl[4-(4-fluorophenyl)-6- octylquinolin-2-yl]amino)acetic acid |
| 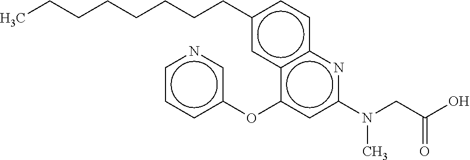 | 2-{methyl[6-octyl-4-(pyridin-3- yloxy)quinolin-2- yl]amino}acetic acid |
| 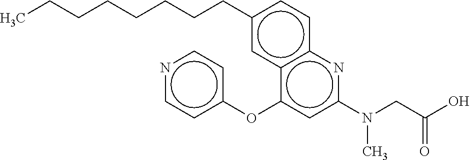 | 2-{methyl[6-octyl-4-(pyridin-4- yloxy)quinolin-2- yl]amino}acetic acid |
| 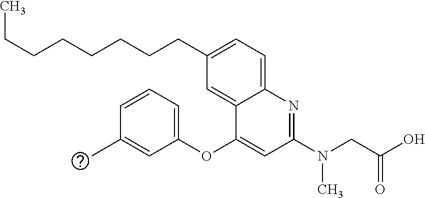 [SMILES: CCCCCCCCc1ccc2nc(N(C)CC(=O)O)cc(Oc3cccc(C)c3)c2c1] | 2-{[4-(3-fluorophenoxy)-6- octylquinolin-2- yl](methyl)amino}acetic acid |
| 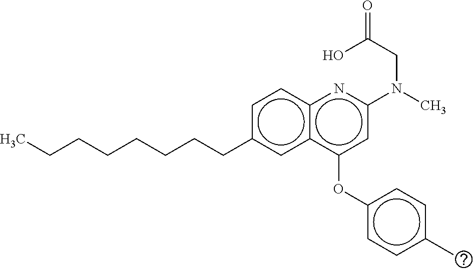 | 2-{[4-(4-fluorophenoxy)-6- octylquinolin-2- yl](methyl)amino}acetic acid |
| 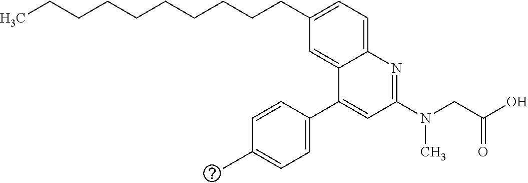 [SMILES: CCCCCCCCCCc1ccc2nc(N(C)CC(=O)O)cc(-c3ccc(C)cc3)c2c1] | 2-{[6-decyl-4-(4- fluorophenyl)quinolin-2- yl](methyl)amino}acetic acid |
| 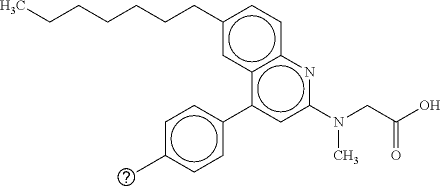 | 2-{[4-(4-fluorophenyl)-6- heptylquinolin-2- yl](methyl)amino}acetic acid |
| 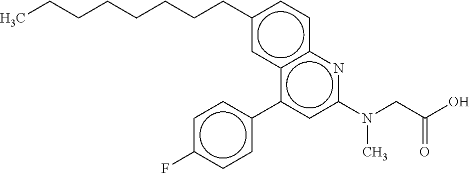 | 2-{[4-(4-fluorophenyl)-6- octylquinolin-2- yl](methyl)amino}acetic acid |
| 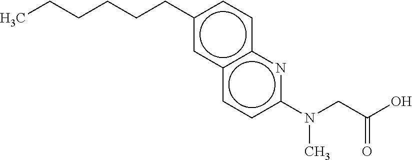 | 2-[(6-hexylquinolin-2- yl)(methyl)amino]acetic acid |
| 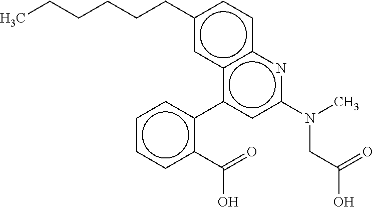 | 2-{2- [{carboxymethyl)(methyl)amino]- 6-hexylquinolin-4-yl}benzoic acid |
| 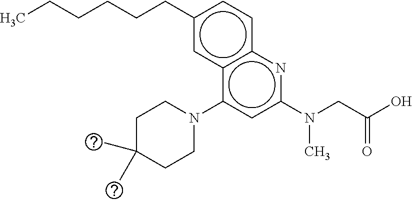 | 2-{[4-(4,4-difluoropiperidin-1- yl)-6-hexylquinolin-2- yl](methyl)amino}acetic acid |
| 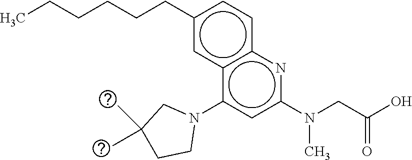 | 2-{[4-(3,3-difluoropyrrolidin-1- yl)-6-hexylquinolin-2- yl](methyl)amino}acetic acid |
| 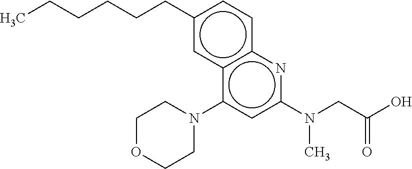 | 2-{[6-hexyl-4-(morpholin-4- yl)quinolin-2- yl](methyl)amino}acetic acid |
| 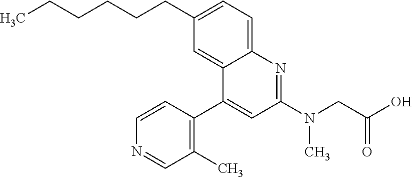 [SMILES: CCCCCCc1ccc2nc(N(C)CC(=O)O)cc(-c3ccncc3C)c2c1] | 2-{[6-butyl-4-(2-methyl-pyridin- 4-yl)quinolin-2- yl](methyl)amino}acetic acid |
| 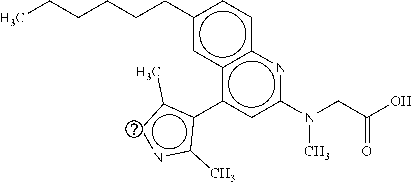 | 2-{[4-(3,5-dimethyl-1,2-oxazol- 4-yl)-6-hexylquinolin-2- yl](methyl)amino}acetic acid |
| 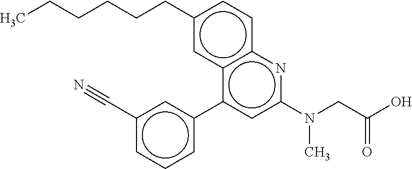 | 2-{[4-(3-cyanophenyl)-6- hexylquinolin-2- yl](methyl)amino}acetic acid |
| 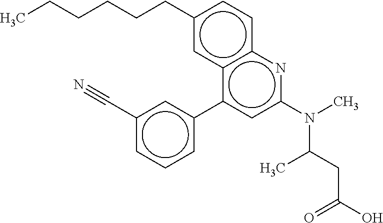 | 3-{[4-(3-cyanophenyl)-6- hexylquinolin-2- yl](methyl)amino}acetic acid |
| 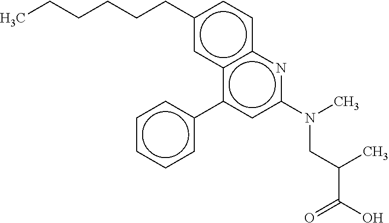 | 3-[(6-hexyl-4-phenylquinolin-2- yl)(methyl)amino]-2- methylpropanoic acid |
| 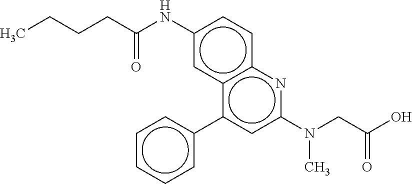 | 2-[methyl(6-pentanamido-4- phenylquinolin-2- yl)amino]acetic acid |
| 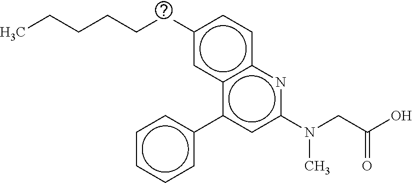 | 2-{methyl[6-(pentyloxy)-4- phenylquinolin-2- yl]amino}acetic acid |
| 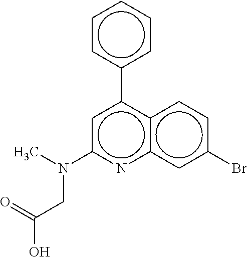 | 2-[(7-bromo-4-phenylquinolin-2- yl)(methyl)amino]acetic acid |
| 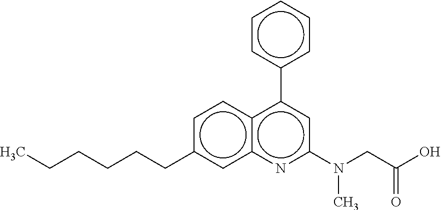 | 2-[(7-hexyl-4-phenylquinolin-2- yl)(methyl)amino]acetic acid |
| 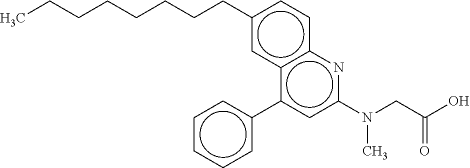 | 2-[methyl(6-octyl-4- phenylquinolin-2- yl)amino]acetic acid |
| 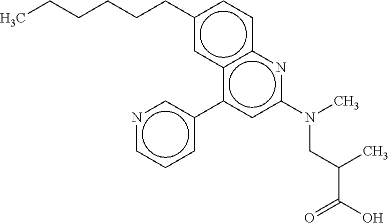 | 3-{[6-hexyl-4-(pyridin-3- yl)quinolin-2-yl](methyl)amino}- 2-methylpropanoic acid |
| 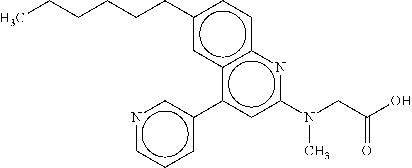 | 2-{[6-hexyl-4-(pyridin-3- yl)quinolin-2- yl](methyl)amino}acetic acid |
| 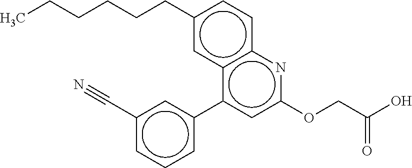 | 2-{[4-(3-cyanophenyl)-6- hexylquinolin-2-yl]oxy}acetic acid |
| 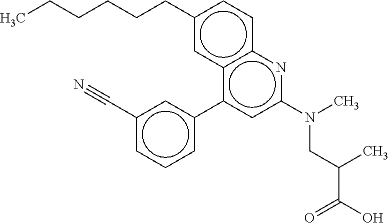 | 3-{[4-(3-cyanophenyl)-6- hexylquinolin-2- yl](methyl)amino}-2- methylpropanoic acid |
| 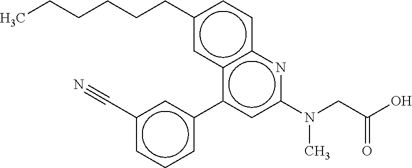 | 2-{[4-(3-cyanophenyl)-6- hexylquinolin-2- yl](methyl)amino}acetic acid |
| 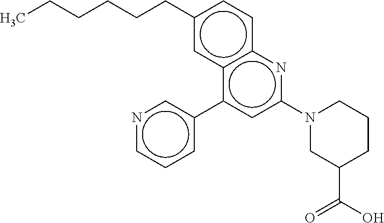 | 1-[6-hexyl-4-(pyridin-3- yl)quinolin-2-yl]piperidine-3- carboxylic acid |
| 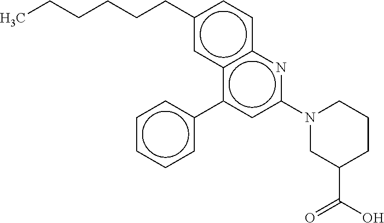 | 1-(6-hexyl-4-phenylquinolin-2- yl)piperidine-3-carboxylic acid |
| 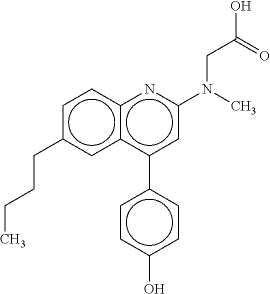 | 2-{[6-butyl-4-(4- hydroxyphenyl)quinolin-2- yl](methyl)amino}acetic acid |
| 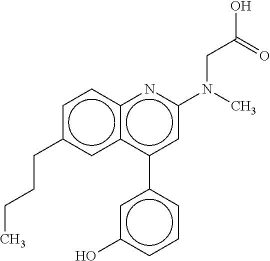 | 2-{[6-butyl-4-(3- hydroxyphenyl)quinolin-2- yl](methyl)amino}acetic acid |
| 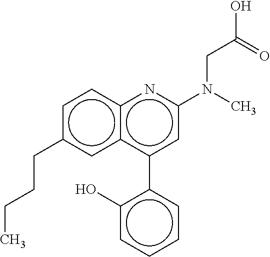 | 2-{[6-butyl-4-(2- hydroxyphenyl)quinolin-2- yl](methyl)amino}acetic acid |
| 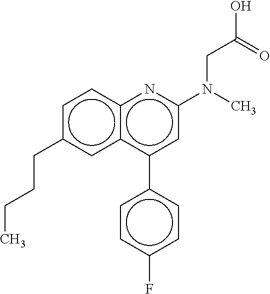 | 2-{[6-butyl-4-(4- fluorophenyl)quinolin-2- yl](methyl)amino}acetic acid |
| 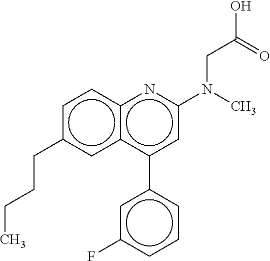 | 2-{[6-butyl-4-(3- fluorophenyl)quinolin-2- yl](methyl)amino}acetic acid |
| 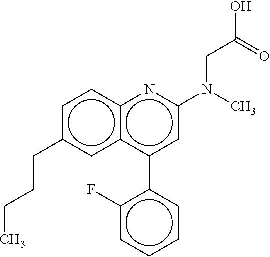 | 2-{[6-butyl-4-(2- fluorophenyl)quinolin-2- yl](methyl)amino}acetic acid |
| 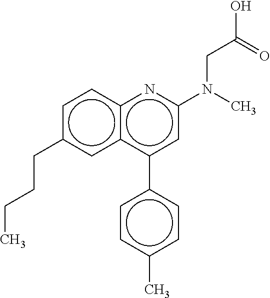 | 2-{[6-butyl-4-(4- methylphenyl)quinolin-2- yl](methyl)amino}acetic acid |
| 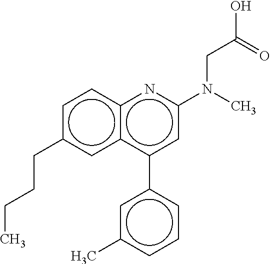 | 2-{[6-butyl-4-(3- methylphenyl)quinolin-2- yl](methyl)amino}acetic acid |
| 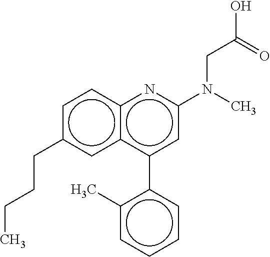 | 2-{[6-butyl-4-(2- methylphenyl)quinolin-2- yl](methyl)amino}acetic acid |
| 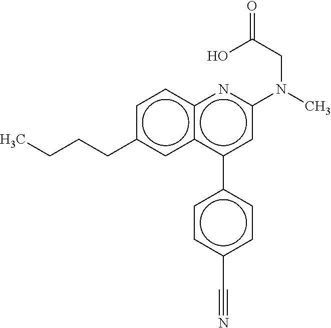 | 2-{[6-butyl-4-(4- cyanophenyl)quinolin-2- yl](methyl)amino}acetic acid |
| 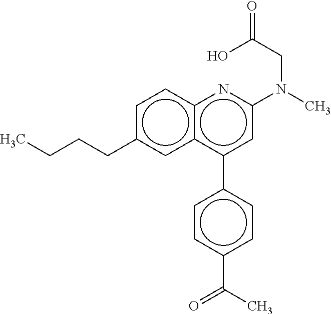 | 2-{[6-butyl-4-(4- carbamoylphenyl)quinolin-2- yl](methyl)amino}acetic acid |
| 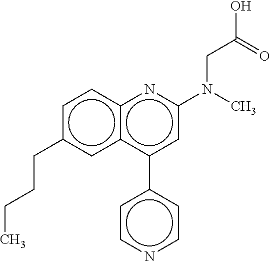 | 2-{[6-butyl-4-(pyridin-4- yl)quinolin-2- yl](methyl)amino}acetic acid |
| 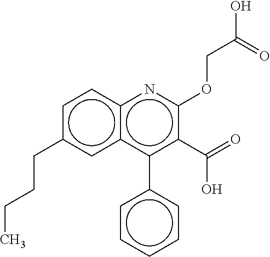 | 6-butyl-2-(carboxymethoxy)-4- phenylquinoline-3-carboxylic acid |
| 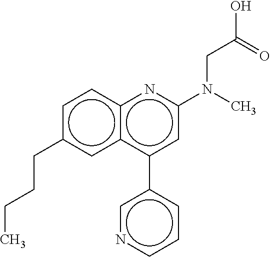 | 2-{[6-butyl-4-(pyridin-3- yl)quinolin-2- yl](methyl)amino}acetic acid |
| 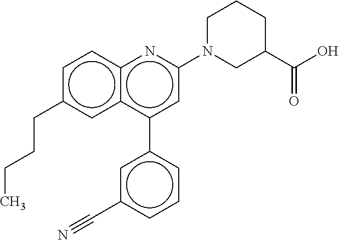 | 1-[6-butyl-4-(3- cyanophenyl)quinolin-2- yl]piperidine-3-carboxylic acid |
| 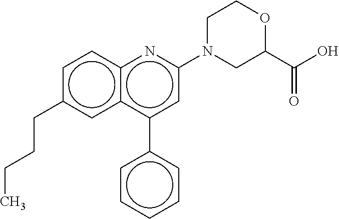 | 4-(6-butyl-4-phenylquinolin-2- yl)morpholine-2-carboxylic acid |
| 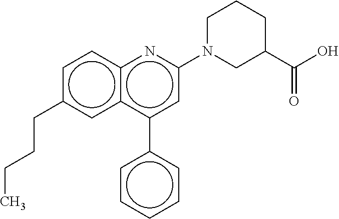 | 1-(6-butyl-4-phenylquinolin-2- yl)piperidine-3-carboxylic acid |
| 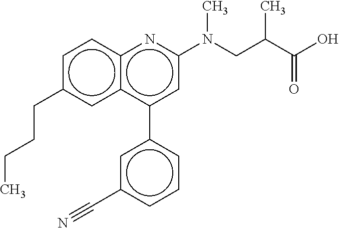 | 3-{[6-butyl-4-(3- cyanophenyl)quinolin-2- yl](methyl)amino}-2- methylpropanoic acid |
| 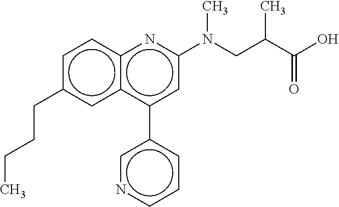 | 3-{[6-butyl-4-(pyridin-3- yl)quinolin-2-yl](methyl)amino}- 2-methylpropanoic acid |
| 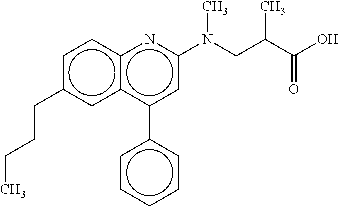 | 3-[(6-butyl-4-phenylquinolin-2- yl)(methyl)amino]-2- methylpropanoic acid |
| 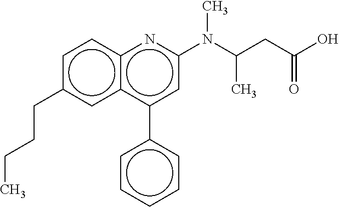 | 3-[(6-butyl-4-phenylquinolin-2- yl)(methyl)amino]butanoic acid |
| 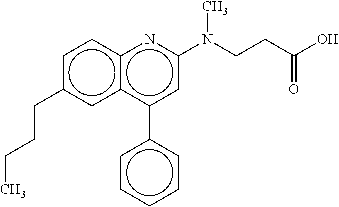 | 3-[(6-butyl-4-phenylquinolin-2- yl)(methyl)amino]propanoic acid |
| 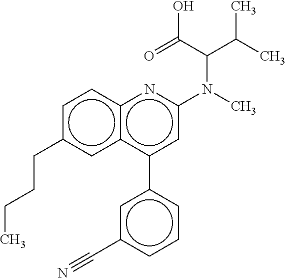 | N-(6-butyl-4-(3- cyanophenyl)quinolin-2-yl)-N- methylvaline? |
| 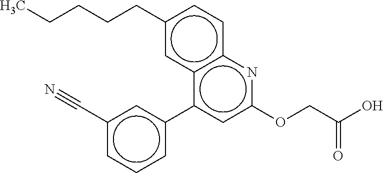 | 2-{[4-(3-cyanophenyl)-6- pentylquinolin-2-yl]oxy}acetic acid |
| 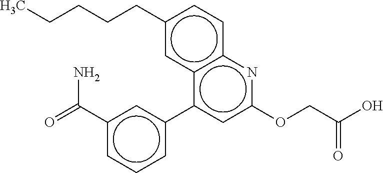 | 2-{[4-(3-carbamoylphenyl)-6- pentylquinolin-2-yl]oxy}acetic acid |
| 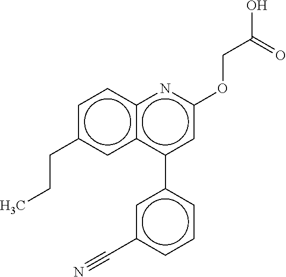 | 2-{[4-(3-cyanophenyl)-6- propylquinolin-2-yl]oxy}acetic acid |
| 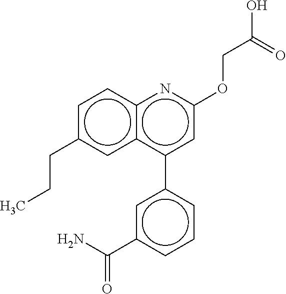 | 2-{[4-(3-carbamoylphenyl)-6- propylquinolin-2-yl]oxy}acetic acid |
| 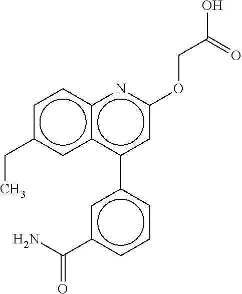 | 2-{[4-(3-carbamoylphenyl)-6- ethylquinolin-2-yl]oxy}acetic acid |
| 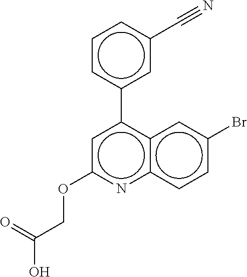 | 2-{[6-bromo-4-(3- cyanophenyl)quinolin-2- yl]oxy}acetic acid |
| 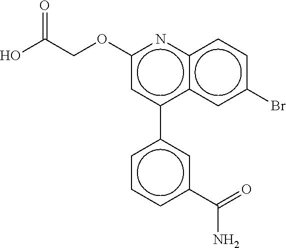 | 2-{[6-bromo-4-(3- carbamoylphenyl)quinolin-2- yl]oxy}acetic acid |
| 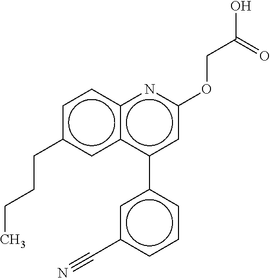 | 2-{[6-butyl-4-(3- cyanophenyl)quinolin-2- yl]oxy}acetic acid |
| 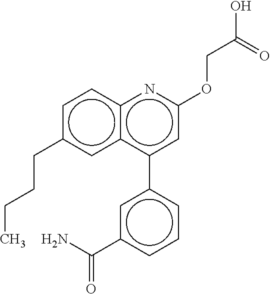 | 2-{[6-butyl-4-(3- carbamoylphenyl)quinolin-2- yl]oxy}acetic acid |
| 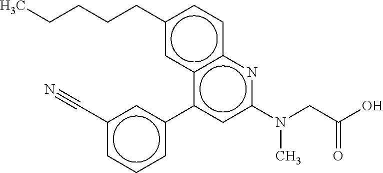 | 2-{[4-(3-cyanophenyl)-6- pentylquinolin-2- yl](methyl)amino}acetic acid |
| 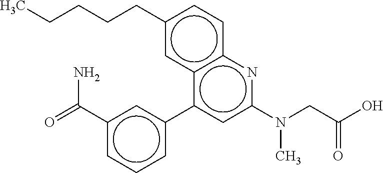 | 2-{[4-(3-carbamoylphenyl)-6- pentylquinolin-2- yl](methyl)amino}acetic acid |
| 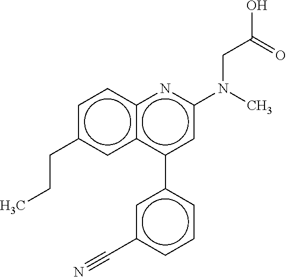 | 2-{[4-(3-cyanophenyl)-6- propylquinolin-2- yl](methyl)amino}acetic acid |
| 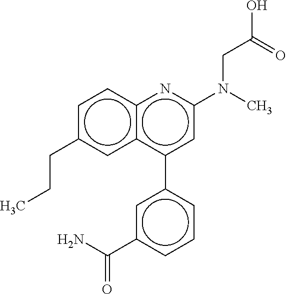 | 2-{[4-(3-carbamoylphenyl)-6- propylquinolin-2- yl](methyl)amino}acetic acid |
| 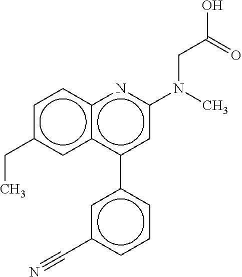 | 2-{[4-(3-cyanophenyl)-6- ethylquinolin-2- yl](methyl)amino}acetic acid |
| 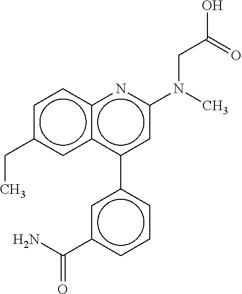 | 2-{[4-(3-carbamoylphenyl)-6- ethylquinolin-2- yl](methyl)amino}acetic acid |
| 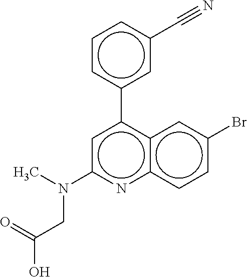 | 2-{[6-bromo-4-(3- cyanophenyl)quinolin-2- yl](methyl)amino}acetic acid |
| 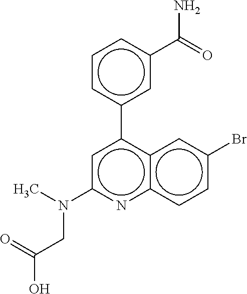 | 2-{[6-bromo-4-(3- carbamoylphenyl)quinolin-2- yl](methyl)amino}acetic acid |
| 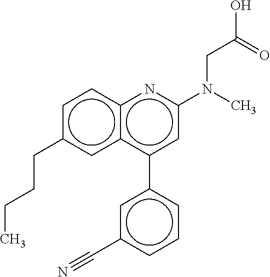 | 2-{[6-butyl-4-(3- cyanophenyl)quinolin-2- yl](methyl)amino}acetic acid |
| 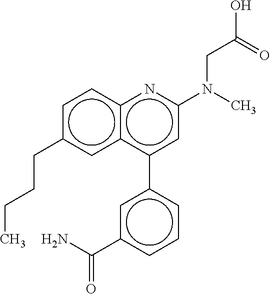 | 2-{[6-butyl-4-(3- carbamoylphenyl)quinolin-2- yl](methyl)amino}acetic acid |
| 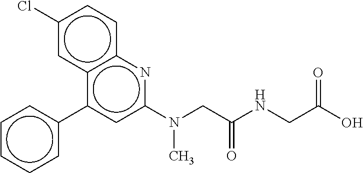 | 2-{2-[(6-chloro-4- phenylquinolin-2-yl) (methyl)amino]acetamino]acetic acid |
| 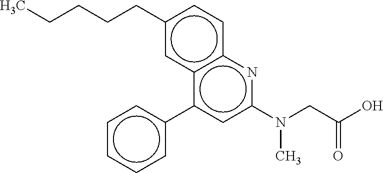 | 2-[methyl(6-pentyl-4- phenylquinolin-2- yl)amino]acetic acid |
| 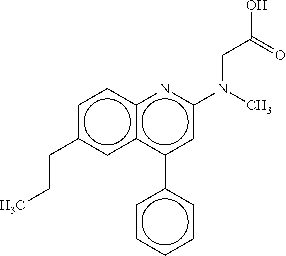 | 2-[methyl(4-phenyl-6- propylquinolin-2- yl)amino]acetic acid |
| 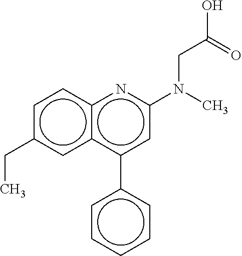 | 2-[(6-ethyl-4-phenylquinolin-2- yl)(methyl)amino]acetic acid |
| 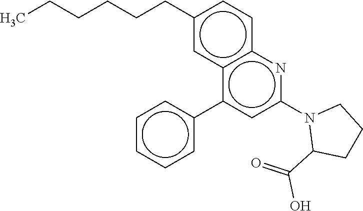 | 1-(6-hexyl-4-phenylquinolin-2- yl)pyrrolidine-2-carboxylic acid |
| 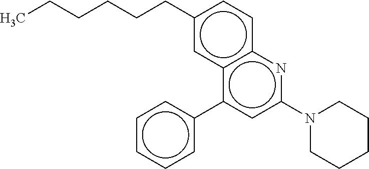 | 6-hexyl-4-phenyl-2-(piperidin-1- yl)quinoline |
| 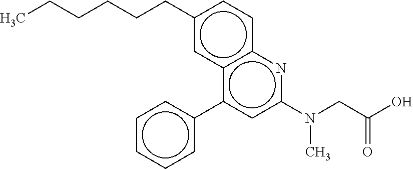 | 2-[(6-hexyl-4-phenylquinolin-2- yl)(methyl)amino]acetic acid |
| 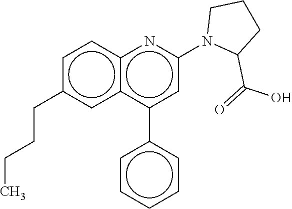 | 1-(6-butyl-4-phenylquinolin-2- yl)pyrrolidine-2-carboxylic acid |
| 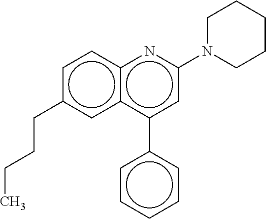 | 6-butyl-4-phenyl-2-(piperidin-1- yl)quinoline |
| 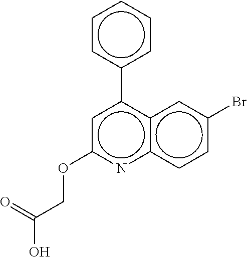 | 2-[(6-bromo-4-phenylquinolin-2- yl)oxy]acetic acid |
| 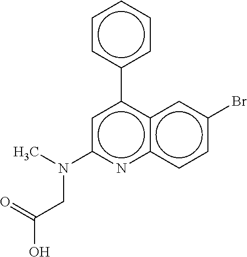 | 2-[(6-bromo-4-phenylquinolin-2- yl)(methyl)amino]acetic acid |
| 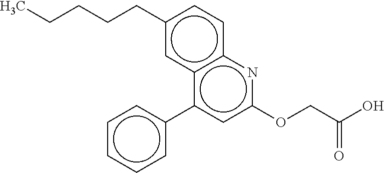 | 2-[(6-pentyl-4-phenylquinolin-2- yl)oxy]acetic acid |
| 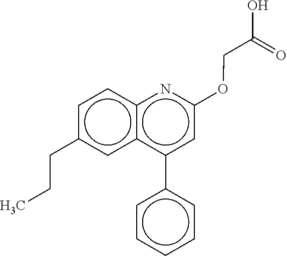 | 2-[(4-phenyl-6-propylquinolin-2- yl)oxy]acetic acid |
| 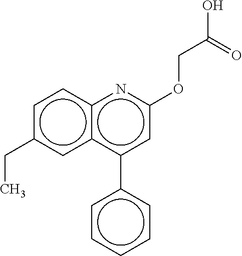 | 2-[(6-ethyl-4-phenylquinolin-2- yl)oxy]acetic acid |
| 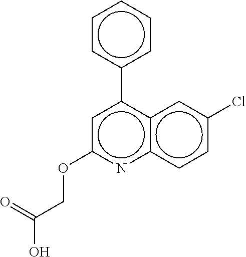 | 2-[(6-chloro-4-phenylquinolin-2- yl)oxy]acetic acid |
| 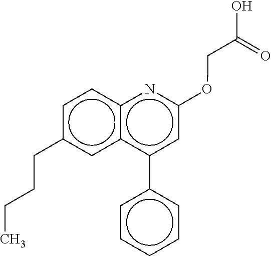 | 2-[(6-butyl-4-phenylquinolin-2- yl)oxy]acetic acid |
| 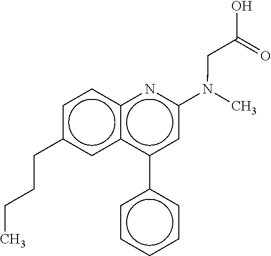 | 2-[(6-butyl-4-phenylquinolin-2- yl)(methyl)amino]acetic acid |
| 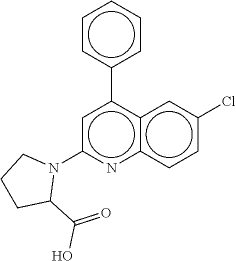 | 1-(6-chloro-4-phenylquinolin-2- yl)pyrrolidine-2-carboxylic acid |
| 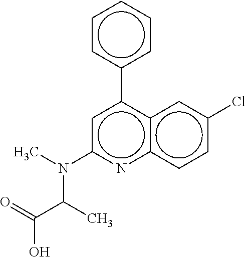 | 2-[(6-chloro-4-phenylquinolin-2- yl)(methyl)amino]propanoic acid |
| 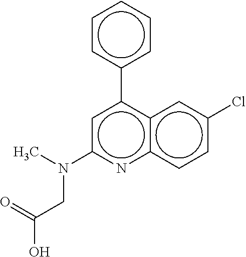 | 2-[(6-chloro-4-phenylquinolin-2- yl)(methyl)amino]acetic acid |
| 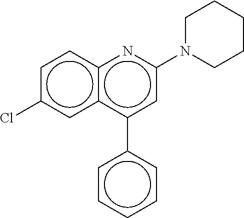 | 6-chloro-4-phenyl-2-(piperidin-1- yl)quinoline |
| 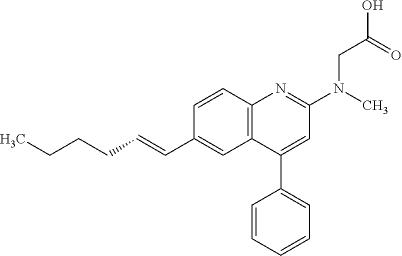 | 2-({6-[(1E)-hex-1-en-1-yl]-4- phenylquinolin-2- yl}(methyl)amino)acetic acid |
| 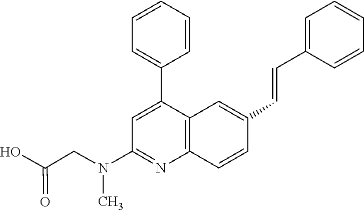 | 2-[methyl({4-phenyl-6-[(1E)-2- phenylethenyl]quinolin-2- yl})amino]acetic acid |
| 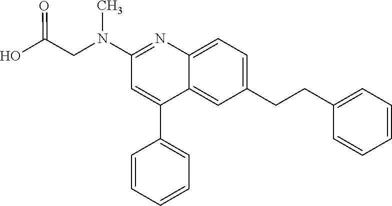 [SMILES: CN(CC(=O)O)c1cc(-c2ccccc2)c2cc(CCc3ccccc3)ccc2n1] | 2-{methyl[4-phenyl-6-(2- phenylethyl)quinolin-2- yl]amino}acetic acid |
| 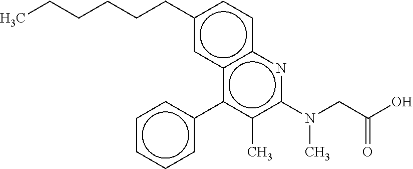 | 2-[(6-hexyl-3-methyl-4- phenylquinolin-2- yl)(methyl)amino]acetic acid |
| 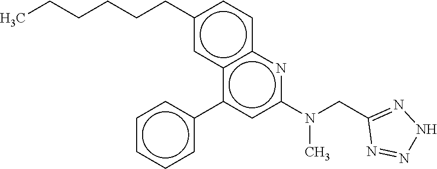 | 6-hexyl-N-methyl-4-phenyl-N- [(2H-1,2,3,4-tetrazol-5- yl)methyl]quinolin-2-amine |
| 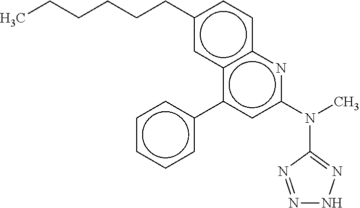 | 6-hexyl-N-methyl-4-phenyl-N- (2H-1,2,3,4-tetrazol-5- yl)quinolin-2-amine |
| 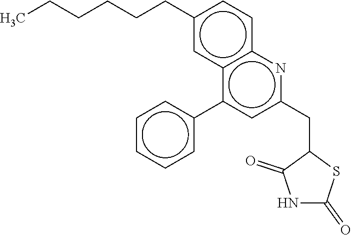 | 5-[(6-hexyl-4-phenylquinolin-2- yl)methyl]-1,3-thiazolidine-2,4- dione |
| 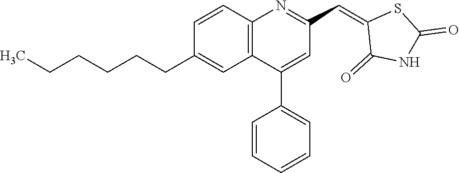 | 5-[(6-hexyl-4-phenylquinolin-2- yl)methylidene]-1,3-thiazolidine- 2,4-dione |
| 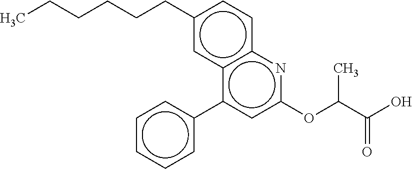 | 2-[(6-hexyl-4-phenylquinolin-2- yl)oxy]propanoic acid |
| 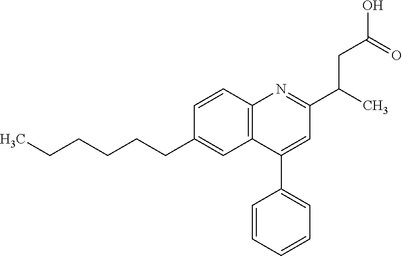 [SMILES: CCCCCCc1ccc2nc(C(C)CC(=O)O)cc(-c3ccccc3)c2c1] | 3-(6-hexyl-4-phenylquinolin-2- yl)butanoic acid |
| 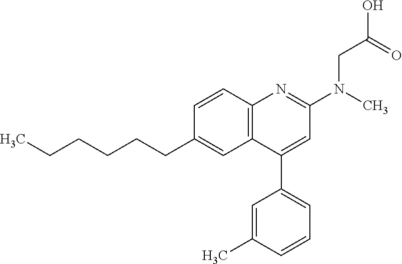 [SMILES: CCCCCCc1ccc2nc(N(C)CC(=O)O)cc(-c3cccc(C)c3)c2c1] | 2-{[6-hexyl-4-(3- methylphenyl)quinolin-2- yl](methyl)amino}acetic acid |
| 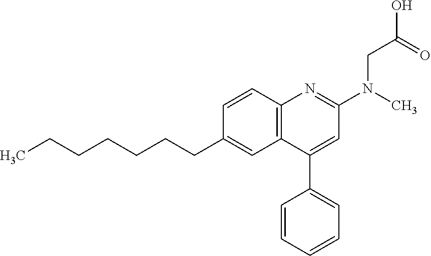 [SMILES: CCCCCCCc1ccc2nc(N(C)CC(=O)O)cc(-c3ccccc3)c2c1] | 2-[(6-heptyl-4-phenylquinolin-2- yl)(methyl)amino]acetic acid |
| 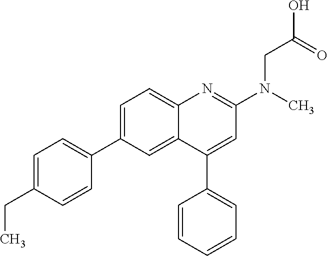 [SMILES: CCc1ccc(-c2ccc3nc(N(C)CC(=O)O)cc(-c4ccccc4)c3c2)cc1] | 2-{[6-(4-ethylphenyl)-4- phenylquinolin-2- yl](methyl)amino}acetic acid |
| 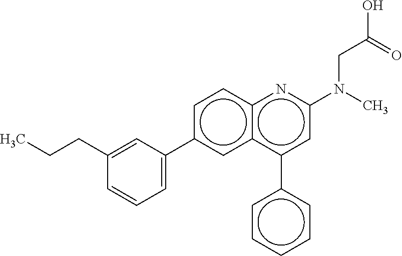 | 2-{methyl[4-phenyl-6-(3- propylphenyl)quinolin-2- yl]amino}acetic acid |
| 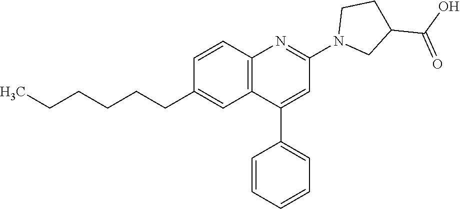 [SMILES: CCCCCCc1ccc2nc(N3CCC(C(=O)O)C3)cc(-c3ccccc3)c2c1] | 1-(6-hexyl-4-phenylquinolin-2- yl)pyrrolidine-3-carboxylic acid |
| 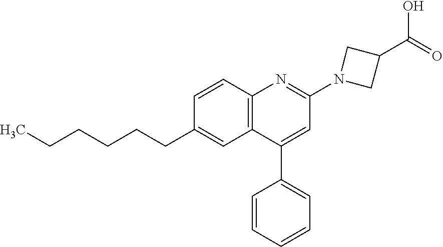 [SMILES: CCCCCCc1ccc2nc(N3CC(C(=O)O)C3)cc(-c3ccccc3)c2c1] | 1-(6-hexyl-4-phenylquinolin-2- yl)azetidine-3-carboxylic acid |
| 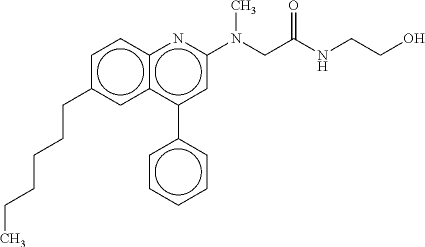 | 2-[(6-hexyl-4-phenylquinolin-2- yl)methyl)amino]-N-(2- hydroxyethyl)acetamide |
| 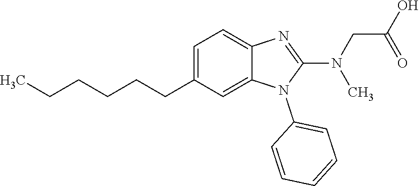 [SMILES: CCCCCCc1ccc2nc(N(C)CC(=O)O)n(-c3ccccc3)c2c1] | 2-[(6-hexyl-1-phenyl-1H-1,3- benzodiazol-2- yl)(methyl)amino]acetic acid |
| 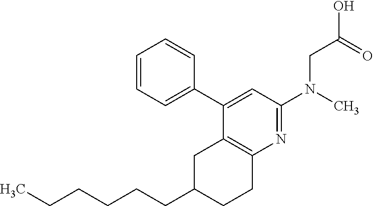 [SMILES: CCCCCCC1CCc2nc(N(C)CC(=O)O)cc(-c3ccccc3)c2C1] | 2-[(6-hexyl-4-phenyl-5,6,7,8- tetrahydroquinolin-2- yl)(methyl)amino]acetic acid |
| 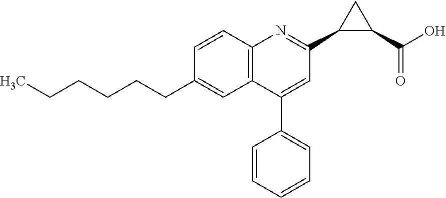 [SMILES: CCCCCCc1ccc2nc([C@H]3C[C@H]3C(=O)O)cc(-c3ccccc3)c2c1] | cis-2-(6-hexyl-4-phenylquinolin- 2-yl)cyclopropane-1-carboxylic acid |
| 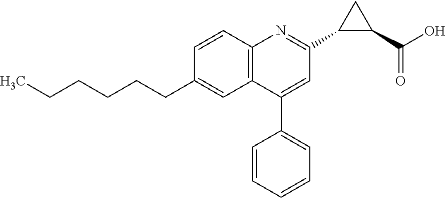 [SMILES: CCCCCCc1ccc2nc([C@@H]3C[C@H]3C(=O)O)cc(-c3ccccc3)c2c1] | trans-2-(6-hexyl-4- phenylquinolin-2- yl)cyclopropane-1-carboxylic acid |
| 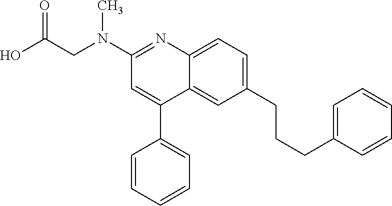 [SMILES: CN(CC(=O)O)c1cc(-c2ccccc2)c2cc(CCCc3ccccc3)ccc2n1] | 2-{methyl[4-phenyl-6-(3- phenylpropyl)quinolin-2- yl]amino}acetic acid |
| 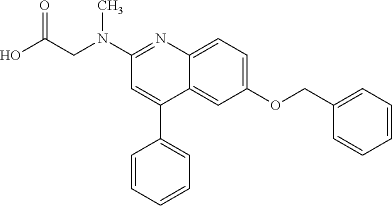 [SMILES: CN(CC(=O)O)c1cc(-c2ccccc2)c2cc(OCc3ccccc3)ccc2n1] | 2-{[6-(benzyloxy)-4- phenylquinolin-2- yl](methyl)amino}acetic acid |
| 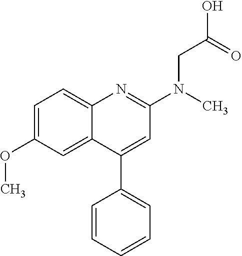 [SMILES: COc1ccc2nc(N(C)CC(=O)O)cc(-c3ccccc3)c2c1] | 2-[(6-methoxy-4-phenylquinolin- 2-yl)(methyl)amino]acetic acid |
| 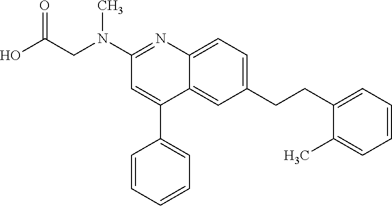 [SMILES: Cc1ccccc1CCc1ccc2nc(N(C)CC(=O)O)cc(-c3ccccc3)c2c1] | 2-[methyl({6-[2-(2- methylphenyl)ethyl]-4- phenylquinolin-2- yl})amino]acetic acid |
| 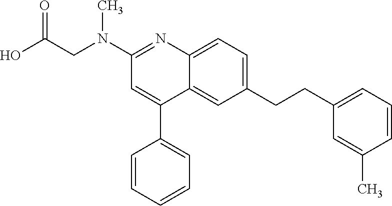 [SMILES: Cc1cccc(CCc2ccc3nc(N(C)CC(=O)O)cc(-c4ccccc4)c3c2)c1] | 2-[methyl({6-[2-(3- methylphenyl)ethyl]-4- phenylquinolin-2- yl})amino]acetic acid |
| 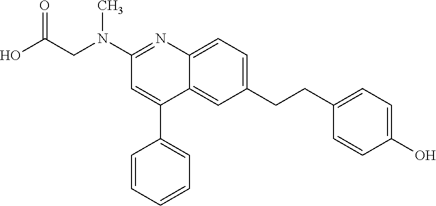 [SMILES: CN(CC(=O)O)c1cc(-c2ccccc2)c2cc(CCc3ccc(O)cc3)ccc2n1] | 2-[methyl({6-[2-(4- methylphenyl)ethyl]-4- phenylquinolin-2- yl})amino]acetic acid |
| 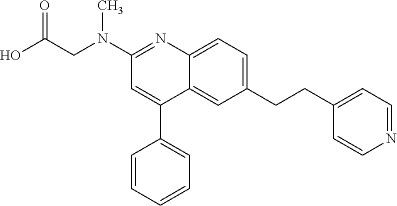 [SMILES: CN(CC(=O)O)c1cc(-c2ccccc2)c2cc(CCc3ccncc3)ccc2n1] | 2-[methyl({4-phenyl-6-[2- (pyridin-4-yl)ethyl]quinolin-2- yl})amino]acetic acid |
| 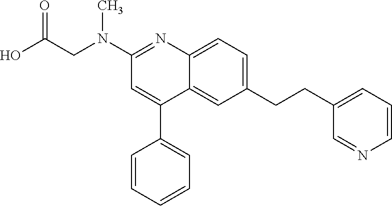 [SMILES: CN(CC(=O)O)c1cc(-c2ccccc2)c2cc(CCc3cccnc3)ccc2n1] | 2-[methyl({4-phenyl-6-[2- (pyridin-3-yl)ethyl]quinolin-2- yl})amino]acetic acid |
| 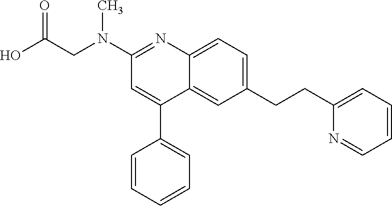 [SMILES: CN(CC(=O)O)c1cc(-c2ccccc2)c2cc(CCc3ccccn3)ccc2n1] | 2-[methyl({4-phenyl-6-[2- (pyridin-2-yl)ethyl]quinolin-2- yl})amino]acetic acid |
| 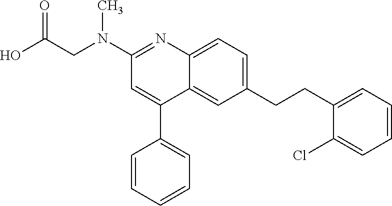 [SMILES: CN(CC(=O)O)c1cc(-c2ccccc2)c2cc(CCc3ccccc3Cl)ccc2n1] | 2-({6-[2-(2-chlorophenyl)ethyl]- 4-phenylquinolin-2- yl}(methyl)amino)acetic acid |
| 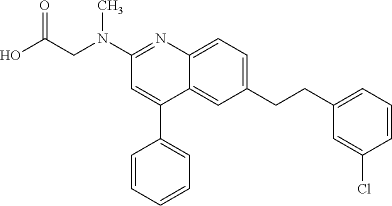 [SMILES: CN(CC(=O)O)c1cc(-c2ccccc2)c2cc(CCc3cccc(Cl)c3)ccc2n1] | 2-({6-[2-(3-chlorophenyl)ethyl]- 4-phenylquinolin-2- yl}(methyl)amino)acetic acid |
| 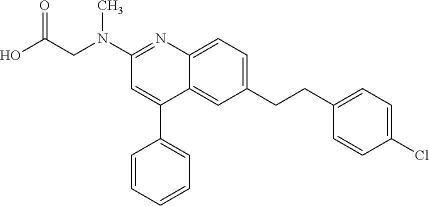 [SMILES: CN(CC(=O)O)c1cc(-c2ccccc2)c2cc(CCc3ccc(Cl)cc3)ccc2n1] | 2-({6-[2-(4-chlorophenyl)ethyl]- 4-phenylquinolin-2- yl}(methyl)amino)acetic acid |
| 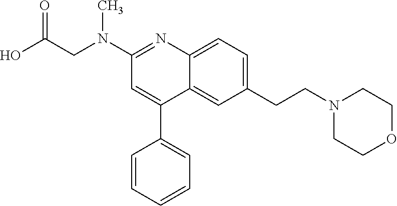 [SMILES: CN(CC(=O)O)c1cc(-c2ccccc2)c2cc(CCN3CCOCC3)ccc2n1] | 2-(methyl({6-[2-(morpholin-4- yl)ethyl]-4-phenylquinolin-2- yl})amino]acetic acid |
| 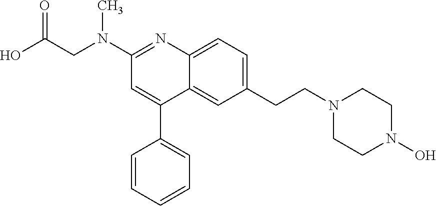 [SMILES: CN(CC(=O)O)c1cc(-c2ccccc2)c2cc(CCN3CCN(O)CC3)ccc2n1] | 2-[methyl({6-[2-(4- methylpiperazin-1-yl)ethyl]-4- phenylquinolin-2- yl})amino]acetic acid |
| 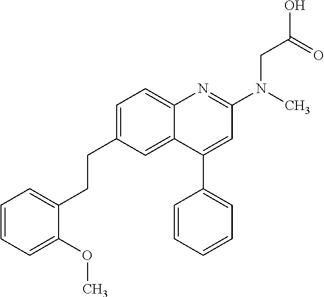 [SMILES: COc1ccccc1CCc1ccc2nc(N(C)CC(=O)O)cc(-c3ccccc3)c2c1] | 2-({6-[2-(2- methoxyphenyl)ethyl]-4- phenylquinolin-2- yl}(methyl)amino)acetic acid |
| 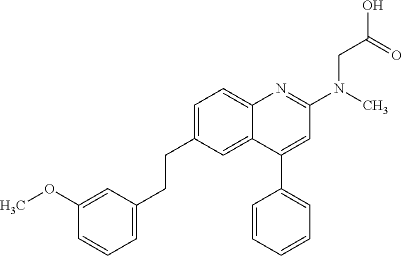 [SMILES: COc1cccc(CCc2ccc3nc(N(C)CC(=O)O)cc(-c4ccccc4)c3c2)c1] | 2-({6-[2-(3- methoxyphenyl)ethyl]-4- phenylquinolin-2- yl}(methyl)amino)acetic acid |
| 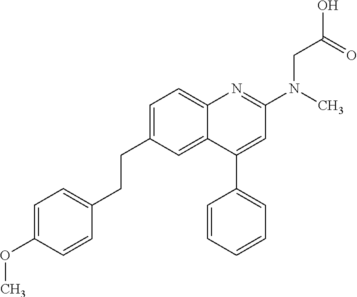 [SMILES: COc1ccc(CCc2ccc3nc(N(C)CC(=O)O)cc(-c4ccccc4)c3c2)cc1] | 2-({6-[2-(4- methoxyphenyl)ethyl]-4- phenylquinolin-2- yl}(methyl)amino)acetic acid |
| 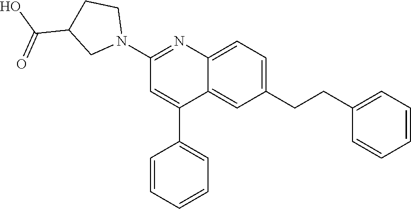 [SMILES: O=C(O)C1CCN(c2cc(-c3ccccc3)c3cc(CCc4ccccc4)ccc3n2)C1] | 1-[4-phenyl-6-(2- phenylethyl)quinolin-2- yl]pyrrolidine-3-carboxylic acid |
| 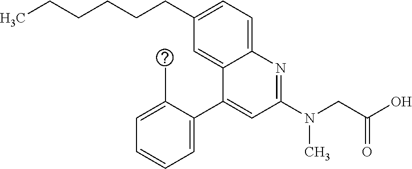 [SMILES: CCCCCCc1ccc2nc(N(C)CC(=O)O)cc(-c3ccccc3C)c2c1] | 2-{[4-(2-fluorophenyl)-6- hexylquinolin-2- yl](methyl)amino}acetic acid |
| 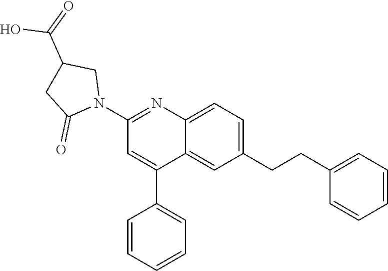 [SMILES: O=C(O)C1CC(=O)N(c2cc(-c3ccccc3)c3cc(CCc4ccccc4)ccc3n2)C1] | 5-oxo-1-[4-phenyl-6-(2- phenylethyl)quinolin-2- yl]pyrrolidine-3-carboxylic acid |
| 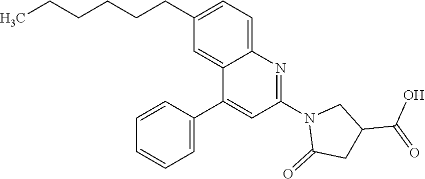 [SMILES: CCCCCCc1ccc2nc(N3CC(C(=O)O)CC3=O)cc(-c3ccccc3)c2c1] | 1-(6-hexyl-4-phenylquinolin-2- yl)-5-oxopyrrolidine-3- carboxylic acid |
| 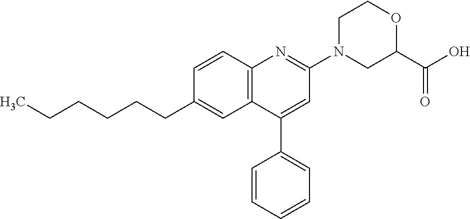 [SMILES: CCCCCCc1ccc2nc(N3CCOC(C(=O)O)C3)cc(-c3ccccc3)c2c1] | 4-(6-hexyl-4-phenylquinolin-2- yl)morpholine-2-carboxylic acid |
| 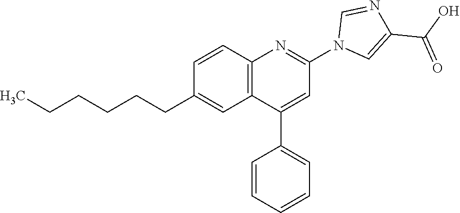 [SMILES: CCCCCCc1ccc2nc(-n3cnc(C(=O)O)c3)cc(-c3ccccc3)c2c1] | 1-(6-hexyl-4-phenylquinolin-2- yl)-1H-imidazole-4-carboxylic acid |
| 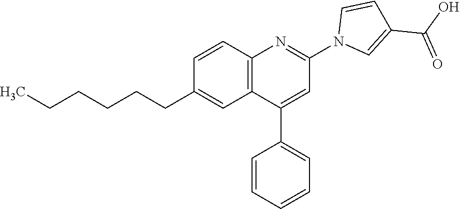 [SMILES: CCCCCCc1ccc2nc(-n3ccc(C(=O)O)c3)cc(-c3ccccc3)c2c1] | 1-(6-hexyl-4-phenylquinolin-2- yl)-1H-pyrrole-3-carboxylic acid |
| 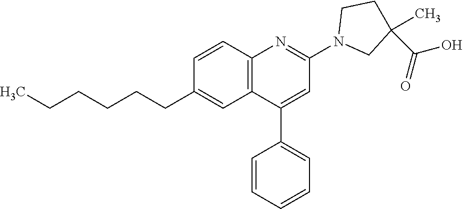 [SMILES: CCCCCCc1ccc2nc(N3CCC(C)(C(=O)O)C3)cc(-c3ccccc3)c2c1] | 1-(6-hexyl-4-phenylquinolin-2- yl)-3-methylpyrrolidine-3- carboxylic acid |
| 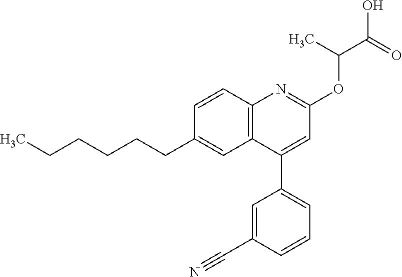 [SMILES: CCCCCCc1ccc2nc(OC(C)C(=O)O)cc(-c3cccc(C#N)c3)c2c1] | 2-{[4-(3-cyanophenyl)-6- hexylquinolin-2- yl]oxy}propanoic acid |
| 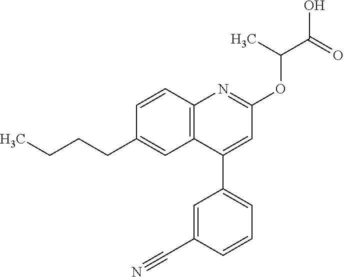 [SMILES: CCCCc1ccc2nc(OC(C)C(=O)O)cc(-c3cccc(C#N)c3)c2c1] | 2-{[6-butyl-4-(3- cyanophenyl)quinolin-2- yl]oxy}propanoic acid |
| 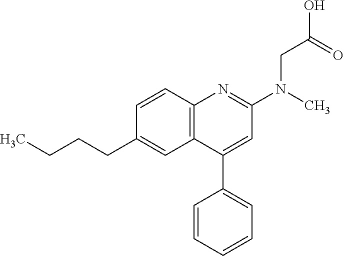 [SMILES: CCCCc1ccc2nc(N(C)CC(=O)O)cc(-c3ccccc3)c2c1] | 2-[(6-butyl-4-phenylquinolin-2- yl)(methyl)amino]acetic acid |
| 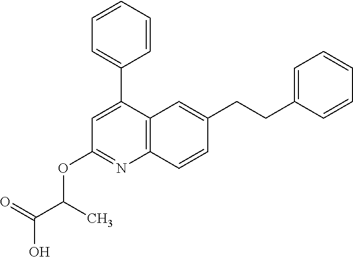 [SMILES: CC(Oc1cc(-c2ccccc2)c2cc(CCc3ccccc3)ccc2n1)C(=O)O] | 2-{[4-phenyl-6-(2- phenylethyl)quinolin-2- yl]oxy}propanoic acid |
| 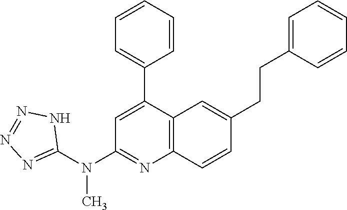 [SMILES: CN(c1cc(-c2ccccc2)c2cc(CCc3ccccc3)ccc2n1)c1nnn[nH]1] | N-methyl-4-phenyl-6-(2- phenylethyl)-N-(1H-1,2,3,4- tetrazol-5-yl)quinolin-2-amine |
| 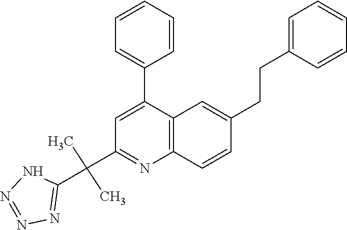 [SMILES: CC(C)(c1cc(-c2ccccc2)c2cc(CCc3ccccc3)ccc2n1)c1nnn[nH]1] | 4-phenyl-6-(2-phenylethyl)-2-[2- (1H-1,2,3,4-tetrazol-5-yl)propan- 2-yl]quinoline |
| 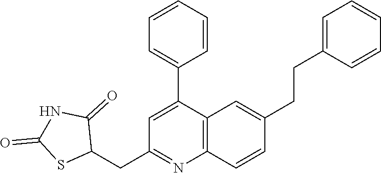 [SMILES: O=C1NC(=O)C(Cc2cc(-c3ccccc3)c3cc(CCc4ccccc4)ccc3n2)S1] | 5-{[4-phenyl-6-(2- phenylethyl)quinolin-2- yl]methyl}-1,3-thiazolidine-2,4- dione |
| 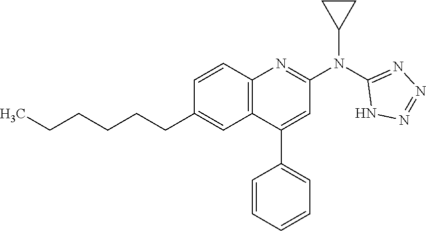 [SMILES: CCCCCCc1ccc2nc(N(c3nnn[nH]3)C3CC3)cc(-c3ccccc3)c2c1] | N-cyclopropyl-6-hexyl-4-phenyl- N-(1H-1,2,3,4-tetrazol-5- yl)quinolin-2-amine |
| 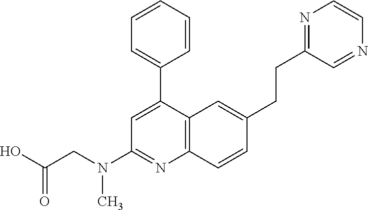 [SMILES: CN(CC(=O)O)c1cc(-c2ccccc2)c2cc(CCc3cnccn3)ccc2n1] | 2-[methyl({4-phenyl-6-[2- (pyrazin-2-yl)ethyl]quinolin-2- yl})amino]acetic acid |
| 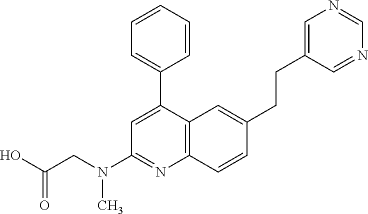 [SMILES: CN(CC(=O)O)c1cc(-c2ccccc2)c2cc(CCc3cncnc3)ccc2n1] | 2-[methyl({4-phenyl-6-[2- (pyrimidin-5-yl)ethyl]quinolin-2- yl})amino]acetic acid |
| 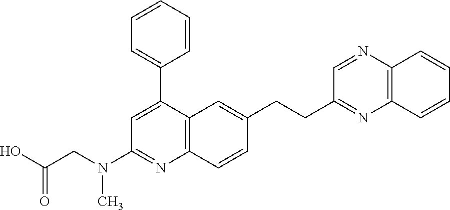 [SMILES: CN(CC(=O)O)c1cc(-c2ccccc2)c2cc(CCc3cnc4ccccc4n3)ccc2n1] | 2-[methyl({4-phenyl-6-[2- (quinoxalin-2-yl)ethyl]quinolin- 2-yl})amino]acetic acid |
| 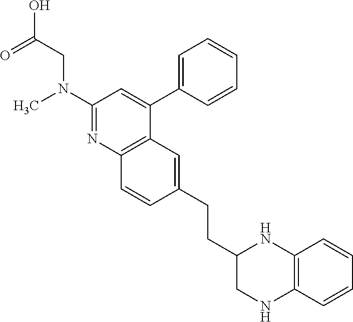 [SMILES: CN(CC(=O)O)c1cc(-c2ccccc2)c2cc(CCC3CNc4ccccc4N3)ccc2n1] | 2-[methyl({4-phenyl-6-[2- (1,2,3,4-tetrahydroquinoxalin-2- yl)ethyl]quinolin-2- yl})amino]acetic acid |
| 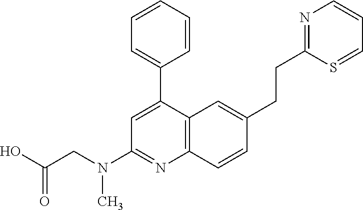 | 2-[methyl({4-phenyl-6-[2- (pyrimidin-2-yl)ethyl]quinolin-2- yl})amino]acetic acid |
| 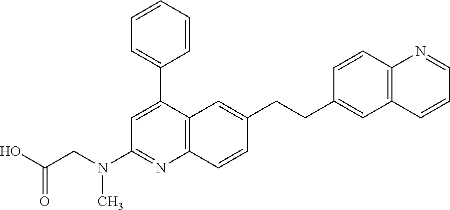 [SMILES: CN(CC(=O)O)c1cc(-c2ccccc2)c2cc(CCc3ccc4ncccc4c3)ccc2n1] | 2-[methyl({4-phenyl-6-[2- (quinolin-6-yl)ethyl]quinolin-2- yl})amino]acetic acid |
| 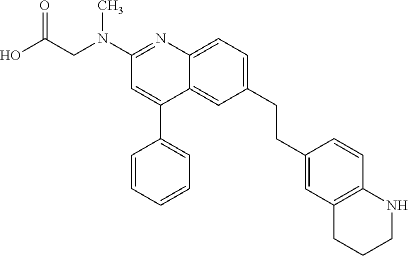 [SMILES: CN(CC(=O)O)c1cc(-c2ccccc2)c2cc(CCc3ccc4c(c3)CCCN4)ccc2n1] | 2-[methyl({4-phenyl-6-[2- (1,2,3,4-tetrahydroquinolin-6- yl)ethyl]quinolin-2- yl})amino]acetic acid |
| 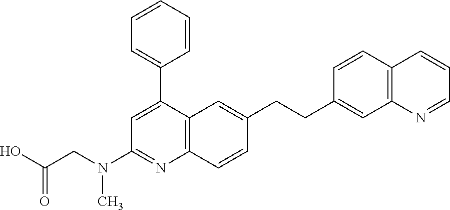 [SMILES: CN(CC(=O)O)c1cc(-c2ccccc2)c2cc(CCc3ccc4cccnc4c3)ccc2n1] | 2-[methyl({4-phenyl-6-[2- (quinolin-7-yl)ethyl]quinolin-2- yl})amino]acetic acid |
| 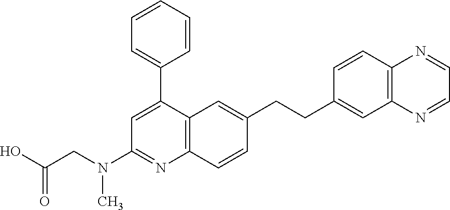 [SMILES: CN(CC(=O)O)c1cc(-c2ccccc2)c2cc(CCc3ccc4nccnc4c3)ccc2n1] | 2-[methyl({4-phenyl-6-[2- (quinoxalin-6-yl)ethyl]quinolin- 2-yl})amino]acetic acid |
| 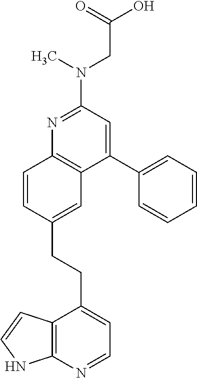 [SMILES: CN(CC(=O)O)c1cc(-c2ccccc2)c2cc(CCc3ccnc4[nH]ccc34)ccc2n1] | 2-{methyl[4-phenyl-6-(2-{1H- pyrrolo[2,3-b]pyridin-4- yl}ethyl)quinolin-2- yl]amino}acetic acid |
| 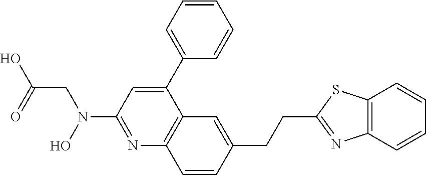 [SMILES: O=C(O)CN(O)c1cc(-c2ccccc2)c2cc(CCc3nc4ccccc4s3)ccc2n1] | 2-({6-[2-(1,3-benzothiazol-2- yl)ethyl]-4-phenylquinolin-2- yl}(methyl)amino)acetic acid |
| 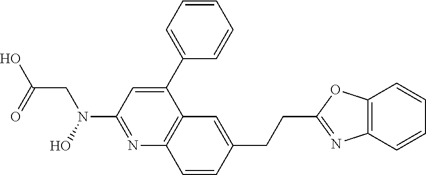 | 2-({6-[2-(1,3-benzoxazol-2- yl)ethyl]-4-phenylquinolin-2- yl}(methyl)amino)acetic acid |
| 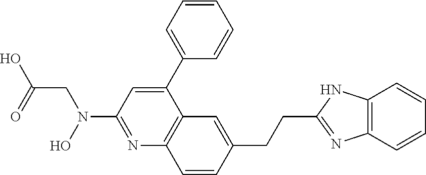 [SMILES: O=C(O)CN(O)c1cc(-c2ccccc2)c2cc(CCc3nc4ccccc4[nH]3)ccc2n1] | 2-({6-[2-(1H-1,3-benzodiazol-2- yl)ethyl]-4-phenylquinolin-2- yl}(methyl)amino)acetic acid |
| 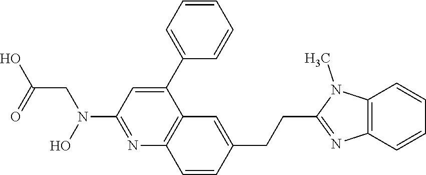 [SMILES: Cn1c(CCc2ccc3nc(N(O)CC(=O)O)cc(-c4ccccc4)c3c2)nc2ccccc21] | 2-[methyl({6-[2-(1-methyl-1H- 1,3-benzodiazol-2-yl)ethyl]-4- phenylquinolin-2- yl})amino)acetic acid |
| 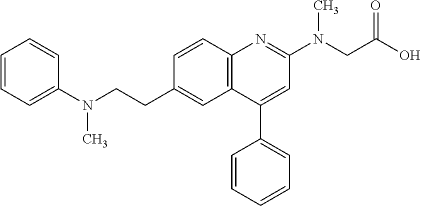 [SMILES: CN(CCc1ccc2nc(N(C)CC(=O)O)cc(-c3ccccc3)c2c1)c1ccccc1] | 2-[methyl(6-{2- [methyl(phenyl)amino]ethyl}-4- phenylquinolin-2- yl)amino]acetic acid |
| 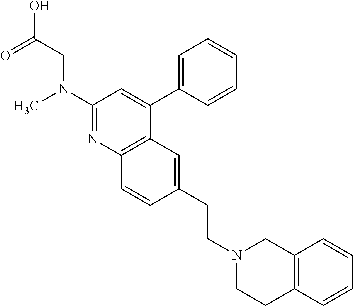 [SMILES: CN(CC(=O)O)c1cc(-c2ccccc2)c2cc(CCN3CCc4ccccc4C3)ccc2n1] | 2-[methyl({4-phenyl-6-[2- (1,2,3,4-tetrahydroisoquinolin-2- yl)ethyl]quinolin-2- yl})amino]acetic acid |
| 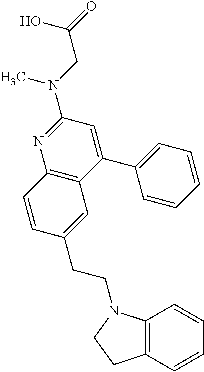 [SMILES: CN(CC(=O)O)c1cc(-c2ccccc2)c2cc(CCN3CCc4ccccc43)ccc2n1] | 2-({6-[2-(2,3-dihydro-1H-indol- 1-yl)ethyl]-4-phenylquinolin-2- yl}(methyl)amino)acetic acid |
| 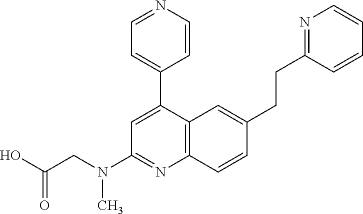 [SMILES: CN(CC(=O)O)c1cc(-c2ccncc2)c2cc(CCc3ccccn3)ccc2n1] | 2-[methyl({6-[2-(pyridin-2- yl)ethyl]-4-(pyridin-4- yl)quinolin-2-yl})amino]acetic acid |
| 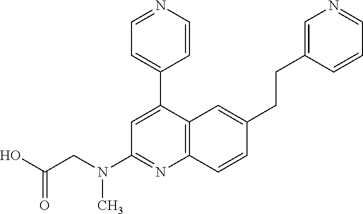 [SMILES: CN(CC(=O)O)c1cc(-c2ccncc2)c2cc(CCc3cccnc3)ccc2n1] | 2-[methyl({6-[2-(pyridin-3- yl)ethyl]-4-(pyridin-4- yl)quinolin-2-yl})amino]acetic acid |
| 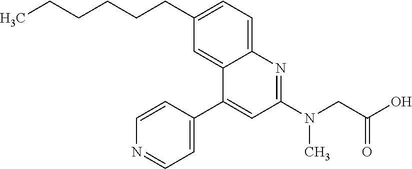 [SMILES: CCCCCCc1ccc2nc(N(C)CC(=O)O)cc(-c3ccncc3)c2c1] | 2-{[6-hexyl-4-(pyridin-3- yl)quinolin-2- yl](methyl)amino}acetic acid |
| 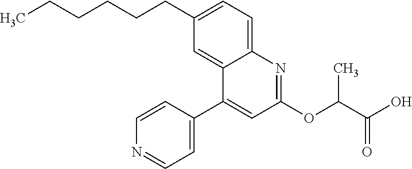 [SMILES: CCCCCCc1ccc2nc(OC(C)C(=O)O)cc(-c3ccncc3)c2c1] | 2-{[6-hexyl-4-(pyridin-4- yl)quinolin-2-yl]oxy}propanoic acid |
| 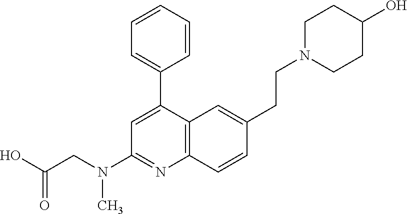 [SMILES: CN(CC(=O)O)c1cc(-c2ccccc2)c2cc(CCN3CCC(O)CC3)ccc2n1] | 2-({6-[2-(4-hydroxypiperidin-1- yl)ethyl]-4-phenylquinolin-2- yl}(methyl)amino)acetic acid |
| 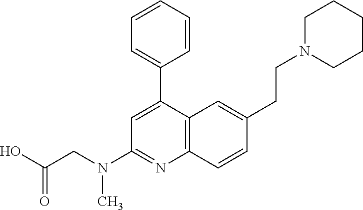 [SMILES: CN(CC(=O)O)c1cc(-c2ccccc2)c2cc(CCN3CCCCC3)ccc2n1] | 2-[methyl({4-phenyl-6-[2- (piperidin-1-yl)ethyl]quinolin-2- yl})amino]acetic acid |
| 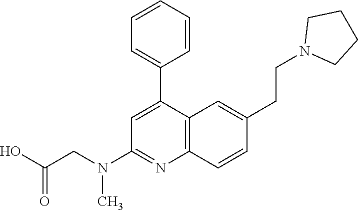 [SMILES: CN(CC(=O)O)c1cc(-c2ccccc2)c2cc(CCN3CCCC3)ccc2n1] | 2-[methyl({4-phenyl-6-[2- (pyrrolidin-1-yl)ethyl]quinolin-2- yl})amino]acetic acid |
| 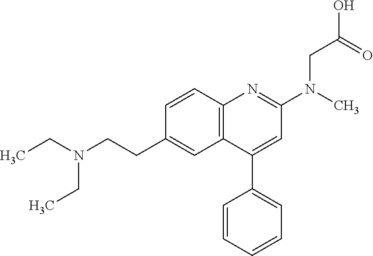 [SMILES: CCN(CC)CCc1ccc2nc(N(C)CC(=O)O)cc(-c3ccccc3)c2c1] | 2-({6-[2-(diethylamino)ethyl]-4- phenylquinolin-2- yl}(methyl)amino)acetic acid |
| 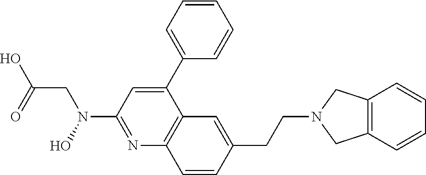 | 2-({6-[2-(2,3-dihydro-1H- isoindol-2-yl)ethyl]-4- phenylquinolin-2- yl}(methyl)amino)acetic acid |

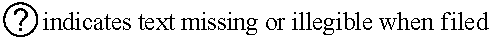

### Detailed Description of Experiments

### Synthetic Method A:

### Representative Example: 2-{[4-(3-cyanophenyl)-6-hexylquinolin-2-yl](methyl)amino}acetic acid

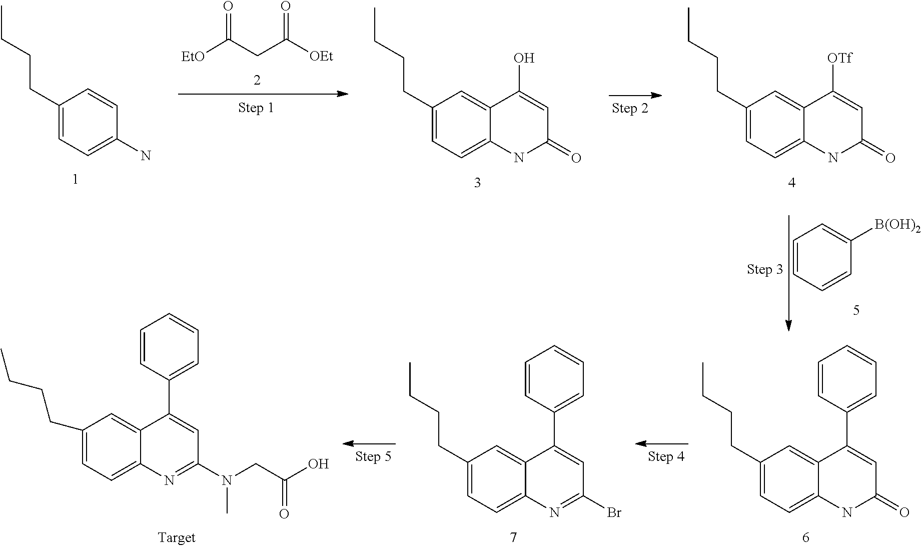

4-hexyl aniline (5 g, 28.24 mmol) in CH₂Cl₂ (50 mL) was cooled to 0° C. and then diethyl malonate 2 was added at stirred for 2 h at room temperature. Reaction mixture was quenched with ice and neutralized with saturated NaHCO₃ solution. Resulting solution was then extracted with CH₂Cl₂ (×2) then dried over Na₂SO₄ and concentrated. 5 g of desired ester amide product was obtained by purification by flash column chromatography which was taken to next step.

5 g of ester amide in PolyPhosphoricAcid (50 mL) was heated to 150° C. and stirred. After 4 h, the reaction was poured into ice and stirred. Pale brown solid was formed after 30 minutes which was filtered and dried to yield 3.5 g of the desired bicyclic product, as determined by LCMS and HNMR.

Bicyclic beta-keto amide (3.5 g, 14.285 mmol) in 15 mL of DMF was cooled to 0° C. and triethylamine (2.48 mL, 17.142 mmol) was added and stirred for 5 min. Comin's reagent (3.78 g, 17.142 mmol) was added and the reaction mixture was stirred for 2 h at room temperature. Reaction mixture was then poured into ice and pale yellow solid was formed which was filtered and dried to yield 3.0 g of the desired product, as determined by LCMS and HNMR.

Bicyclic triflate (0.3 g, 0.797 mmol), 3-cyano-phenyl boronic acid (0.177 g, 1.196 mmol) and Na₂CO₃ (0.168 g, 1.594 mmol) was added in DMF+H₂O (10 mL:2 mL) solution. Argon gas was bubbled and Pd(dppf)Cl₂ (0.058 g, 0.0797 mmol) was added at which point the reaction was moved to pre-heated 100° C. oil bath and stirred for 1 h. Reaction mixture was diluted with water, resulting in formation of brown precipitation which was then filtered and dried to yield the desired product, as determined by LCMS and HNMR.

Tricyclic amide (0.2 g, 0.606 mmol) in toluene was added POBr₃ (0.52 g, 1.818 mmol) and stirred for 3 h at 140° C. The reaction mixture was poured into ice and the aqueous layer was extract with EtOAc (×2). The organic layer was then dried over Na₂SO₄ and concentrated. Purification by flash column chromatography gave the desired bromide product as determined by LCMS and HNMR.

Cyano bromide (0.18 g, 0.459 mmol) in DMSO (5 mL) was added N-Methyl glycine (0.32 g, 2.295 mmol) and K₂CO₃ (0.19 g, 1.377 mmol). Reaction mixture was then stirred at 100° C. for 16 h before quenched with H₂O. The mixture was acidified with 1N HCl to adjust the pH˜6 before being extracted with EtOAc (×2). Organic layer was dried over Na₂SO₄ and concentrated before purification by reverse phase prep-HPLC to yield 2-{[4-(3-cyanophenyl)-6-hexylquinolin-2-yl](methyl)amino}acetic acid.

### Synthetic Method B:

### Representative Example: 2-{[4-(3-cyanophenyl)-6-hexylquinolin-2-yl]oxy}acetic acid

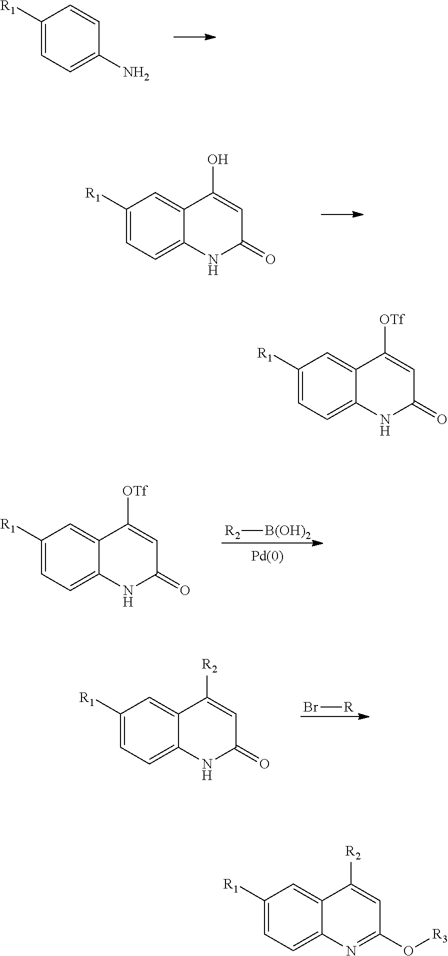

4-hexyl aniline (5 g, 28.24 mmol) in CH₂Cl₂ (50 mL) was cooled to 0° C. and then diethyl malonate 2 was added at stirred for 2 h at room temperature. Reaction mixture was quenched with ice and neutralized with saturated NaHCO₃ solution. Resulting solution was then extracted with CH₂Cl₂ (×2) then dried over Na₂SO₄ and concentrated. 5 g of desired ester amide product was obtained by purification by flash column chromatography which was taken to next step.

5 g of ester amide in PolyPhosphoricAcid (50 mL) was heated to 150° C. and stirred. After 4 h, the reaction was poured into ice and stirred. Pale brown solid was formed after 30 minutes which was filtered and dried to yield 3.5 g of the desired bicyclic product, as determined by LCMS and HNMR.

Bicyclic beta-keto amide (3.5 g, 14.285 mmol) in 15 mL of DMF was cooled to 0° C. and triethylamine (2.48 mL, 17.142 mmol) was added and stirred for 5 min. Comin's reagent (3.78 g, 17.142 mmol) was added and the reaction mixture was stirred for 2 h at room temperature. Reaction mixture was then poured into ice and pale yellow solid was formed which was filtered and dried to yield 3.0 g of the desired product, as determined by LCMS and HNMR.

Bicyclic triflate (1.0 g, 2.652 mmol), 3-cyano-phenyl boronic acid (0.580 g, 3.978 mmol) and Na₂CO₃ (0.562 g, 5.304 mmol) was added in DMF+H₂O (10 mL:2 mL) solution. Argon gas was bubbled and Pd(dppf)Cl₂ (0.193 g, 0.265 mmol) was added at which point the reaction was moved to pre-heated 100° C. oil bath and stirred for 1 h. Reaction mixture was diluted with water, resulting in formation of brown precipitation which was then filtered and dried to yield the desired product, as determined by LCMS and HNMR.

Tricyclic amide (0.4 g, 1.212 mmol) in DMF (5 mL) was cooled to 0° C. and NaH (60% in Mineral Oil, 0.145 g, 3.636 mmol) was added portion wise and stirred for 30 minutes. Bromo-methyl acetate (0.1303 g, 1.818 mmol) was added at 0° C. and the reaction mixture, after removal of ice bath, was stirred for 4 h. Reaction was quenched with ice and organic layer was extracted with EtOAc (×2), dried over Na₂SO₄ and concentrated by flash column chromatography gave the desired bromide product as determined by LCMS and HNMR.

Cyano ester bromide (0.17 g, 0.4096 mmol) in EtOH:H₂O (5 mL:1 mL) was added KOH (0.07 g, 1.2289 mmol) and the reaction mixture was then stirred for 2 h at room temperature. After evaporating EtOH, crude reaction was diluted with 1 mL of H₂O and pH adjusted to 4 with 1N HCl, pale yellow precipitation was formed which was dried and washed with Et₂O and n-pentane to yield 2-{[4-(3-cyanophenyl)-6-hexylquinolin-2-yl]oxy}acetic acid, as determined by LCMS and HNMR.

### Synthetic Method C:

### Representative Example: 2-{[6-hexyl-4-(morpholin-4-yl)quinolin-2-yl](methyl)amino}acetic acid

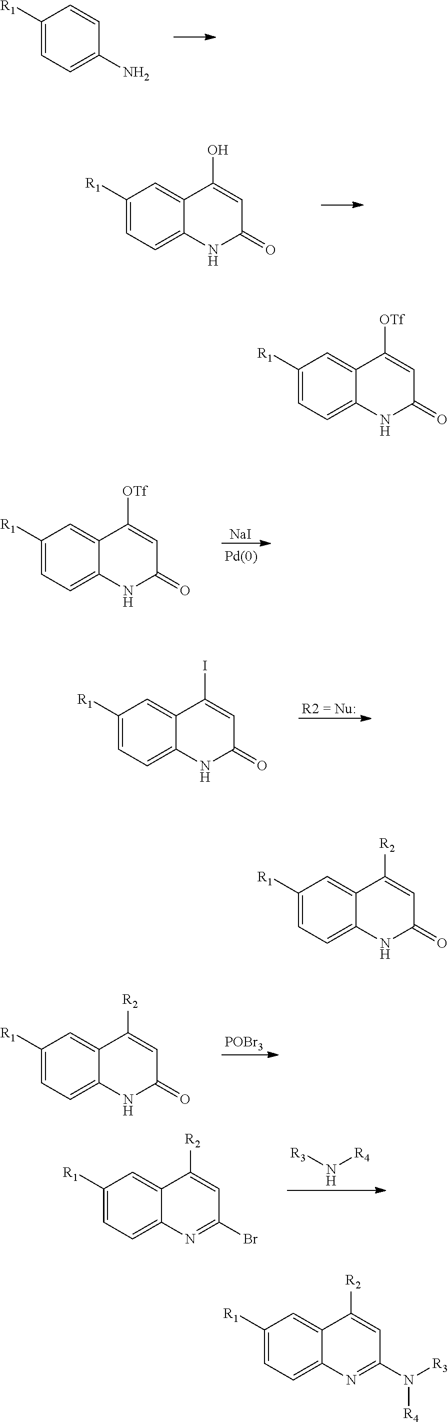

4-hexyl aniline (5 g, 28.24 mmol) in CH₂Cl₂ (50 mL) was cooled to 0° C. and then 2 was added at stirred for 2 h at room temperature. Reaction mixture was quenched with ice and neutralized with saturated NaHCO₃ solution. Resulting solution was then extracted with CH₂Cl₂ (×2) then dried over Na₂SO₄ and concentrated. 5 g of desired ester amide product was obtained by purification by flash column chromatography which was taken to next step.

5 g of ester amide in PolyPhosphoricAcid (50 mL) was heated to 150° C. and stirred. After 4 h, the reaction was poured into ice and stirred. Pale brown solid was formed after 30 minutes which was filtered and dried to yield 3.5 g of the desired bicyclic product, as determined by LCMS and HNMR.

Bicyclic beta-keto amide (3.5 g, 14.285 mmol) in 15 mL of DMF was cooled to 0° C. and triethylamine (2.48 mL, 17.142 mmol) was added and stirred for 5 min. Comin's reagent (3.78 g, 17.142 mmol) was added and the reaction mixture was stirred for 2 h at room temperature. Reaction mixture was then poured into ice and pale yellow solid was formed which was filtered and dried to yield 3.0 g of the desired product, as determined by LCMS and HNMR.

To bicyclic triflate (0.5 g, 1.3262 mmol), in CH₃CN (10 mL) was added conc. HCl (0.1 mL, 1.452 mmol) followed by Sodium Iodide (1.989 g, 13.262 mmol) at 0° C. and stirred at room temperature for 16 h. Reaction mixture was diluted with water and extracted with EtOAc (×2), dried over Na₂SO₄ and concentrated. Purification by flash column chromatography yielded the desired product, as determined by LCMS and HNMR.

Iodo amide (0.2 g, 0.5633 mmol) in NMP (1 mL) was added morpholine (490 mg, 5.633 mmol) and triethylamine (0.4 mL, 02.816 mmol). The reaction mixture was stirred for 1 h at 160° C. Reaction was diluted with water and organic layer was extracted with EtOAc (×2), dried over Na₂SO₄ and concentrated by flash column chromatography gave the desired bromide product as determined by LCMS and HNMR.

Morpholino amide (0.1 g, 0.3184 mmol) in toluene (3 mL) was added POBr₃ (0.273 g, 0.955 mmol) and stirred for 3 h at 140° C. The reaction mixture was poured into ice and the aqueous layer was extract with EtOAc (×2). The organic layer was then dried over Na₂SO₄ and concentrated. Purification by flash column chromatography gave the desired bromide product as determined by LCMS and HNMR.

Morpholino bromide (0.08 g, 0.212 mmol) in DMSO (3 mL) was added N-Methyl glycine methyl ester (0.145 g, 1.063 mmol) and K₂CO₃ (0.090 g, 0.636 mmol). Reaction mixture was then stirred at 100° C. for 16 h before quenched with H₂O. The mixture was acidified with 1N HCl to adjust the pH˜6 before being extracted with EtOAc (×2). Organic layer was dried over Na₂SO₄ and concentrated before purification by reverse phase prep-HPLC to yield 2-{[6-hexyl-4-(morpholin-4-yl)quinolin-2-yl](methyl)amino}acetic acid as confirmed by H-NMR and LCMS.

### Experimental Protocols

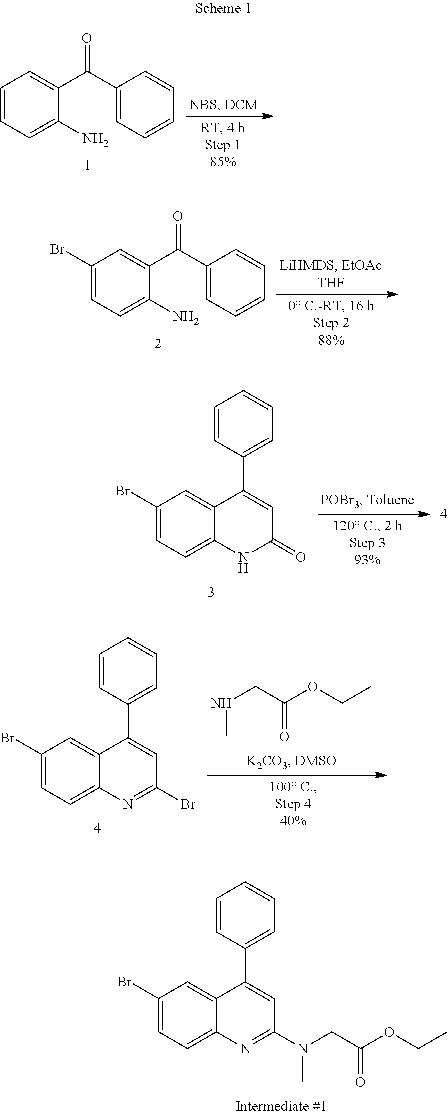

### Step-1: Synthesis of (2-amino-5-bromophenyl) (phenyl)methanone

To a stirred solution of (2-aminophenyl) (phenyl)methanone (10 g, 0.0507 mmol) in DCM (50 mL) at 0° C. was added NBS (8.98 g, 0.0507 mmol). The reaction mixture was slowly warmed to room temperature and stirred at RT for a period of 4 h. After completion of the reaction (monitored by T.L.C, Eluent: 10% EtOAc/Hexane, R.f: 0.5), the reaction mixture was diluted with water (50 mL), and the DCM layer was separated out. The aqueous layer was re-extracted with DCM (2×100 mL). The combined organic layers were dried over Na₂SO₄, filtered and concentrated under reduced pressure to obtain the crude compound. The crude compound was purified by column chromatography over silica gel (100-200 mesh) using a solvent gradient of 3-5% EtOAC in hexane as an eluent to afford (2-amino-5-bromophenyl) (phenyl)methanone) (16 g, 85%) as a yellow solid. MS (ESI) m/z: 275.2 [M+H]⁺.

### Step 2: Synthesis of 6-bromo-4-phenylquinolin-2(1H)-one

To a stirred solution of (2-amino-5-Bromophenyl) (phenyl)methanone (12 g, 0.0436 mmol) in THF (100 mL) at 0° C. was added LiHMDS (260 mL, 0.261 mmol, 1M in THF) and stirred for 0.5 h before the addition of ethyl acetate (12.79 mL). The reaction mixture was continued and stirred at 0° C. for a period of 4 h. The reaction mixture was slowly warmed to room temperature and water (50 mL) was added to the reaction mixture. Resultant mass was stirred at RT for a period of 16 h. After completion of the reaction (monitored by T.L.C, Eluent: 30% EtOAc/Hexane, R.f: 0.3), hexane (200 mL) was added to the reaction mixture, and the precipitated solid was filtered, washed with water and dried under vacuum to afford 6-bromo-4-phenylquinolin-2(1H)-one (11.5 g, 88%) as an off white solid. MS (ESI) m/z: 399.0 [M+H]⁺.

### Step 3: Synthesis of 2,6-dibromo-4-phenylquinoline

To a stirred solution of 6-bromo-4-phenylquinolin-2(1H)-one (11.5 g, 0.0384 mmol) in toluene (100 mL) was added POBr₃ (33.0 g, 0.115 mmol), and resultant reaction mass was heated at 140° C. for 4 h. After completion of the reaction (monitored by T.L.C, Eluent: 30% EtOAc/Hexane, R.f: 0.9), the reaction mixture was neutralized with saturated solution of sodium bicarbonate (250 mL) and the product was extracted with ethyl acetate (2×200 mL). The combined organic layers were washed with brine (100 mL), dried over sodium sulphate, filtered and concentrated under reduced pressure to obtain the crude compound. The crude compound was purified by column chromatography over silica gel using a solvent gradient of 2-3% EtOAC in hexane as an eluent to afford 2,6-dibromo-4-phenylquinoline (9 g, 93%) as off white solid. MS (ESI) m/z: 361.0 [M+H]⁺.

### Step 4: Synthesis of ethyl N-(6-bromo-4-phenylquinolin-2-yl)-N-methyl glycinate (Int #1)

To a stirred solution of 2,6-dibromo-4-phenylquinoline (9 g, 0.0249 mmol) in DMSO (90 mL) at room temperature was added potassium carbonate (10.32 g, 0.747 mmol) and sarcosine ethyl ester (5.9 g, 0.0373 mmol), heated the resultant reaction mixture at 100° C. for 16 h. After completion of the reaction (monitored by T.L.C, Eluent: 20% EtOAc: Hexane, R.f: 0.2), the reaction mixture was cooled to room temperature, partioned between ice-water (100 mL) and EtOAc (100 mL). The organic layer was separated out, and the aqueous layer was acidified with 1N HCl (15 mL). The product was extracted with ethyl acetate (2×120 mL). The combined organic layer was dried over sodium sulphate, filtered and concentrated under reduced pressure to obtain crude compound. The crude compound was purified by column chromatography over silica gel using a solvent gradient of 10% EtOAC in hexane as an eluent to afford (3 g, 40%). MS (ESI) m/z: 398.0 [M+H]⁺.

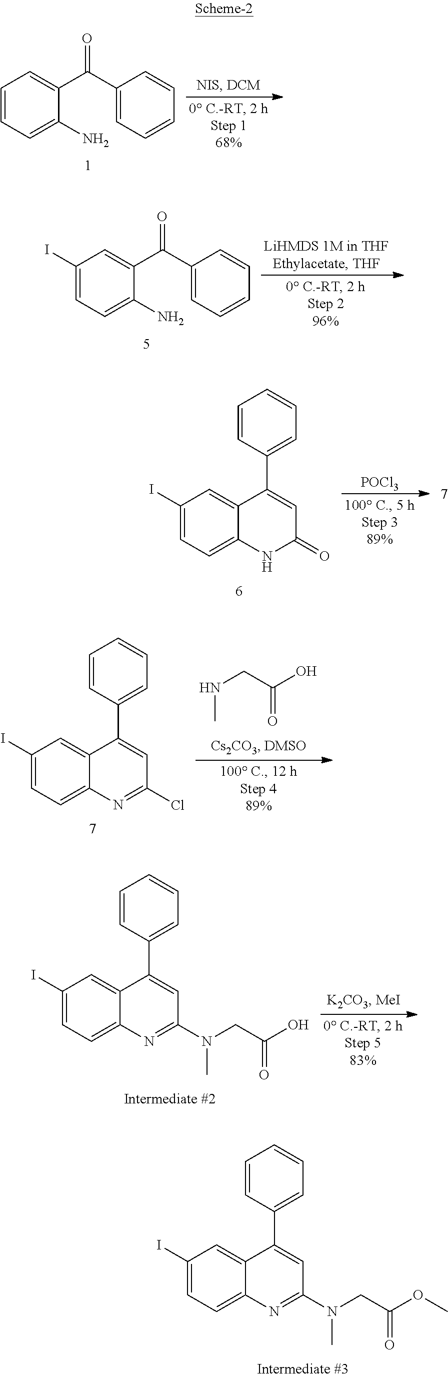

### Step 1: Synthesis of (2-amino-5-iodophenyl) (Phenyl)methanone

To a stirred solution of (2-aminophenyl) (phenyl)methanone (25 g, 0.126 mol) in DCM (250 mL) was added N-iodosuccinimide portion wise at 0° C. The resultant reaction mixture was warmed to room temperature and stirred for a period of 2 h. After completion of reaction (monitored by TLC, Eluent: 10% EtOAc/Hexane, R f; 0.3), the reaction mixture was poured in to ice-water (100 mL) and extracted with EtOAc (2×200 mL), washed with brine (50 mL). The combined organic layer was dried over Na₂SO₄, filtered and concentrated under reduced pressure to afford a crude material. The obtained crude material purified by column chromatography over silica gel (100-200 mesh) using a solvent gradient of 15-20% EtOAc in Hexane as an eluent to afford (2-amino-5-iodophenyl) (Phenyl)methanone (28 g, 68%). MS (ESI) m/z: 324 [M+H]⁺

### Step 2: Synthesis of 6-iodo-4-phenylquinolin-2(1H)-one

To a stirred solution of (2-amino-5-iodophenyl) (Phenyl)methanone (28 g, 0.086 mol) in THF (280 mL) was added LiHMDS (520 mL, 0.519 mol, 1M in THF) at 0° C. and the reaction mixture was stirred for a period of 0.5 h, before the addition of ethyl acetate (28 mL, 1 Vol). The reaction mixture was warmed to room temperature and stirred for a period of 2 h. After completion of reaction (monitored by TLC, Eluent: 30% EtOAc/Hexane, R.f; 0.15), the reaction mixture was quenched with sat. NH₄Cl solution (100 mL). The precipitated solid was collected by filtration and dried under Vacuum to afford 6-iodo-4-phenylquinolin-2-ol (29 g, 96%). MS (ESI) m/z: 348 [M+H]⁺

### Step 3: Synthesis of 2-chloro-6-iodo-4-Phenylquinoline

To a stirred solution of 6-iodo-4-phenylquinolin-2-ol (29 g, 0.083 mol) in POCl₃ (200 mL) was added N, N-dimethyl aniline (29 mL, 1 Vol) and heated to 110° C. for a period of 4 h. After completion of reaction (monitored by TLC, Eluent: 10% EtOAc/Hexane, R.f; 0.7), the reaction mixture was concentrated under reduced pressure. The resulting residue was poured on to ice-water (100 mL), and the precipitated solid was collected by filtration and dried under vacuum to afford 2-chloro-6-iodo-4-Phenylquinoline (27.3 g, 89.5%). MS (ESI) m/z: 366 [M+H]⁺

### Step 4: Synthesis of N-(6-iodo-4-Phenylquinolin-2-yl)-N-methyl glycine (Int #2)

To a stirred solution of 2-chloro-6-iodo-4-Phenylquinoline (27.3 g, 0.074 mol) in DMSO (200 mL) was added cesium carbonate (48.8 g, 0.149 mol) and sarcosine (8 g, 0.089 mol). The reaction mixture was heated at 100° C. for a period of 16 h. After completion of reaction (monitored by TLC, Eluent: 80% EtOAc/Hexane, R.f; 0.25), the reaction mixture was quenched with 1N HCl solution, and the solid formed was filtered and dried under vacuum to afford N-(6-iodo-4-Phenylquinolin-2-yl)-N-methyl glycine (29 g, 89.5%). MS (ESI) m/z: 417 [M−H]⁻

### Step 5: Synthesis of methyl N-(6-iodo-4-Phenylquinolin-2-yl)-N-methyl glycine (Int #3)

To a stirred solution of N-(6-iodo-4-Phenylquinolin-2-yl)-N-methyl glycine (29 g, 0.069 mol) in DMF (200 mL) at room temperature was added potassium carbonate (19.2 g, 0.139 mol) and methyl iodide (8.7 mL, 0.139 mol) and stirred at room temperature for a period of 2 h. After completion of reaction (monitored by TLC, eluent: 70% EtOAc/Hexane, R.f; 0.65), the reaction mixture was quenched with ice cold water, the obtained solid was filtered and dried under vacuum to afford methyl N-(6-iodo-4-Phenylquinolin-2-yl)-N-methyl glycine (25 g, 83%). MS (ESI) m/z: 433 [M+H]⁺

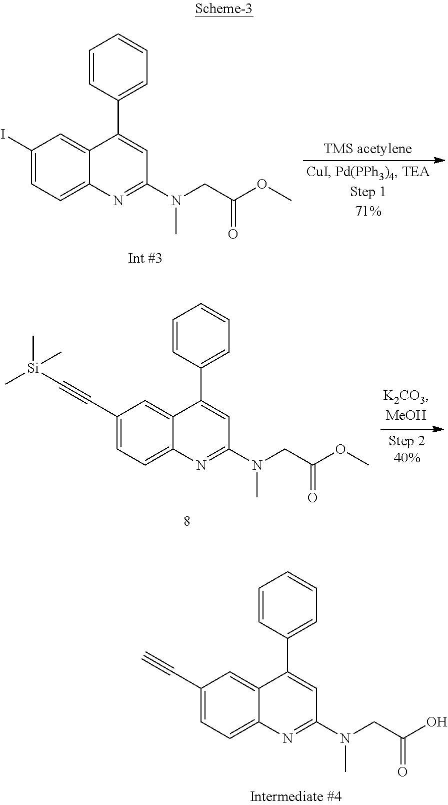

### Step-1: Synthesis of Methyl N-methyl-N-(4-phenyl-6-((trimethylsilyl)ethynyl) quinolin-2-yl) glycinate

To a stirred solution of methyl N-(6-iodo-4-phenylquinolin-2-yl)-N-methyl glycinate (4.8 g, 0.011 mmol) in THF (45 mL) was added CuI (0.21 g, 0.0011 mmol), Et₃N (0.21 g, 0.11 mmol) and TMS acetylene (2.18 g, 0.022 mmol). The reaction mixture was degassed for 10 min with nitrogen and then Pd(PPh₃)₂Cl₂ (0.39 g, 0.05 mmol) was added and again degassed with nitrogen for 10 min. The reaction mixture was degassed with N₂ for 15 min and stirred at room temperature for 2 h. After completion of the reaction (monitored by T.L.C, Eluent: 20% EtOAc: Hexane, R.f: 0.4), the reaction mixture was filtered through a celite bed, the celite bed was further washed with THF (50 mL) and concentrated under reduced to afford a residue. The residue was purified by flash column chromatography on silica gel (100-200) eluting with a gradient of 5-7% ethyl acetate in hexane to obtain methyl N-methyl-N-(4-phenyl-6-((trimethylsilyl)ethynyl) quinolin-2-yl) glycinate (3.2 g, 71%). MS (ESI) m/z: 403 [M+H]⁺.

### Step-2: Synthesis of N-(6-ethynyl-4-phenylquinolin-2-yl)-N-methyl glycine

To a stirred solution of methyl N-methyl-N-(4-phenyl-6-((trimethylsilyl)ethynyl) quinolin-2-yl) glycinate (3.2 g, 0.0079 mmol) in MeOH-DCM (25 mL) at room temperature was added potassium carbonate (2.2 g, 0.015 mmol) and continued the stirring for 2 h. After completion of the reaction (monitored by T.L.C, Eluent: 20% EtOAc: Hexane, R.f: 0.5), the reaction mixture was quenched with Ice-cold water (25 mL) and the product was extracted with 10% MeOH in DCM (2×25 mL). The combined organic layers were dried over sodium sulphate, filtered and concentrated under reduced pressure to afford crude material. The resulting crude material was triturated with ether (10 mL) and pentane (20 mL) to afford N-(6-ethynyl-4-phenylquinolin-2-yl)-N-methyl glycine (500 mg, 40%). MS (ESI) m/z: 317.2 [M+H]⁺.

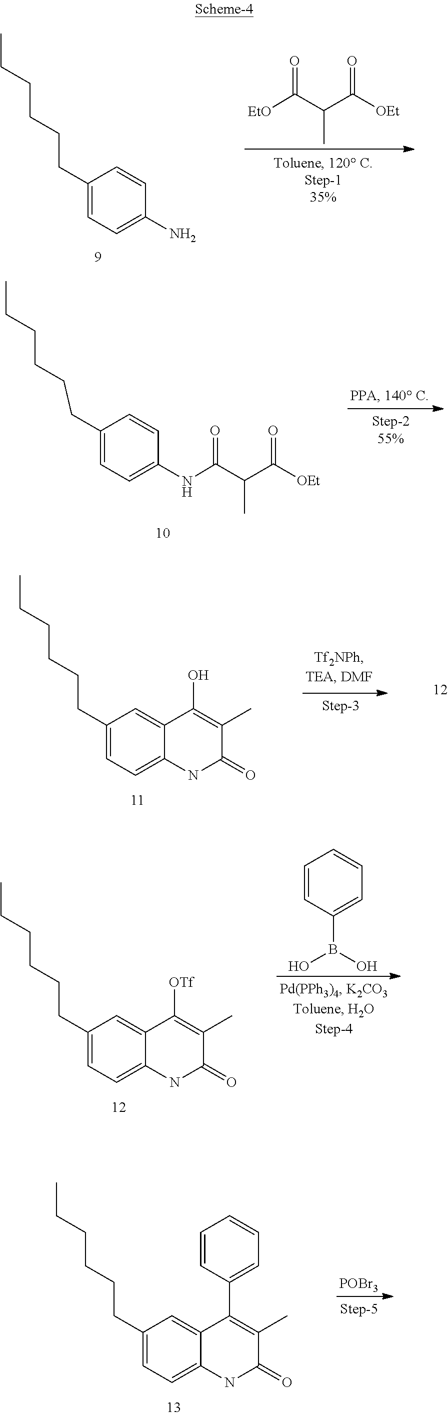

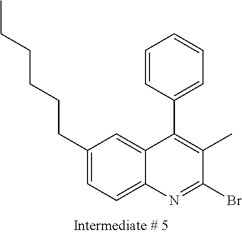 [SMILES: CCCCCCc1ccc2nc(Br)c(C)c(-c3ccccc3)c2c1]

### Step 1: Synthesis of ethyl 3-((4-hexylphenyl) amino)-2-methyl-3-oxopropanoate

A mixture of 4-hexyl aniline (2.0 g, 11.28 Mmol) and diethyl 2-methylmalonate (2.30 g, 13.2 mmol) was heated at 120° C. for 5 h. After completion of the reaction (monitored by T.L.C, Eluent: 50% EtOAc/Hexane, R.f: 0.4), reaction mixture was cooled to room temperature and diluted with ice-water (50 mL), extracted with ethyl acetate (2×100 mL) and washed with brine (50 mL). The combined organic layer was dried over Na₂SO₄, filtered and concentrated under reduced pressure to obtain crude compound. The crude compound was triturated with n-hexane (4×25 mL), filtered and dried to obtain ethyl 3-((4-hexylphenyl) amino)-2-methyl-3-oxopropanoate (1.2 g, 35%) as a white solid. MS (ESI) m/z: 306.0 [M+H]⁺.

### Step 2: Synthesis of 6-hexyl-4-hydroxy-3-methylquinolin-2(1H)-one

A mixture of N-(4-hexylphenyl)-3-oxo-3-phenylpropanamide (3.0 g, 9.84 mmol) in PPA (15 g) was heated at 130° C. for 1.5 h. After completion of the reaction (monitored by T.L.C, Eluent: 40% EtOAc/Hexane, R.f: 0.35), it was cooled to room temperature, poured onto ice-water (100 mL), extracted with DCM (50 mL) and washed with brine (10 mL). The combined organic layer was dried over Na₂SO₄, filtered and concentrated under reduced pressure to obtain crude compound. The crude compound was triturated with n-hexane (2×10 mL) to afford 6-hexyl-4-hydroxy-3-methylquinolin-2(1H)-one (1.4 g, 55%) as off-white solid. MS (ESI) m/z: 260.0 [M+H]⁺.

### Step 3: 6-hexyl-3-methyl-2-oxo-1,2-dihydroquinolin-4-yl trifluoromethanesulfonate

To a stirred solution of 6-hexyl-4-hydroxy-3-methylquinolin-2(1H)-one (250 mg, 0.965 mmol) in DMF (4.0 mL), cooled to 0-5° C. was added triethyl amine (0.4 mL, 2.895 mmol) followed by N-Phenyl-bis(trifluoromethanesulfonimide) (413 mg, 1.16 mmol). The resultant reaction mixture was stirred at room temperature for 1 h. After completion of the reaction (monitored by T.L.C, Eluent: 40% EtOAc/Hexane, R.f: 0.55), it was cooled to room temperature, poured onto ice-water (100 mL), extracted with ethyl acetate (25 mL) and washed with brine (2×10 mL). The combined organic layer was dried over Na₂SO₄, filtered and concentrated under reduced pressure to obtain crude compound as a reddish-brown syrupy liquid (300 mg). The crude compound was 6-hexyl-3-methyl-2-oxo-1,2-dihydroquinolin-4-yl trifluoromethanesulfonate was utilized in the subsequent step without further purification.

### Step 4: 6-hexyl-3-methyl-4-phenylquinolin-2 (1H)-one

To a stirred solution of 6-hexyl-3-methyl-2-oxo-1,2-dihydroquinolin-4-yl trifluoromethanesulfonate (300 mg, 0.766 mmol), Phenyl boronic acid (94 mg, 0.766 mmol) and PdCl₂(dppf) (62 mg, 0.0766 mmol) were sequentially added to degassed DMF (5 mL), and stirred at rt for 20 min. To the resulting mixture was added triethyl amine (0.32 mL, 2.298 mmol) and heated at 130° C. for 3 h. After completion of the reaction (monitored by T.L.C, Eluent: 40% EtOAc/Hexane, R.f: 0.6), it was cooled to room temperature, diluted with ethyl acetate (25 mL), filtered through celite bed and was washed with brine (2×10 mL). The organic layer was dried over Na₂SO₄, filtered and concentrated under reduced pressure to obtain crude compound as a dark brown solid. The crude compound was 6-hexyl-3-methyl-4-phenylquinolin-2(1H)-one (200 mg) was utilized in the subsequent step without further purification. MS (ESI) m/z: 320 [M+H]⁺.

### Step 5: Synthesis of 2-bromo-6-hexyl-4-phenylquinoline (Intermediate #5)

To a stirred solution of 6-hexyl-3-methyl-4-phenylquinolin-2(1H)-one (200 mg, 0.626 mmol) in Toluene (1.0 mL) was added POBr₃ (320 mg, 0.182 mmol) and heated at 120° C. for 2 h. After completion of the reaction (monitored by T.L.C, Eluent: 40% EtOAc/Hexane, R.f: 0.7), the reaction mixture was poured onto ice-water (10 mL) and the product was extracted with EtOAc (2×10 mL) and washed with brine (2×5 mL). The combined organic layers were dried over Na₂SO₄, filtered and concentrated under reduced pressure to obtain 2-bromo-6-hexyl-4-phenylquinoline as a brown syrupy liquid (240 mg). The crude compound was utilized in the next step without further purification. MS (ESI) m/z: 382.0 [M+H]⁺.
Similarly, below intermediate #6 were prepared by following the same or analogous process described above.

| Int # | Structure | Analytical data |
|---|---|---|
| 6 | 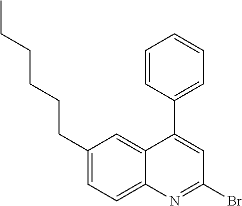 [SMILES: CCCCCCc1ccc2nc(Br)cc(-c3ccccc3)c2c1] | MS (ESI) m/z: m/z 368 [M + H]⁺. |

Intermediate #6 also synthesized by following an alternative scheme and the experimental protocol is mentioned below.

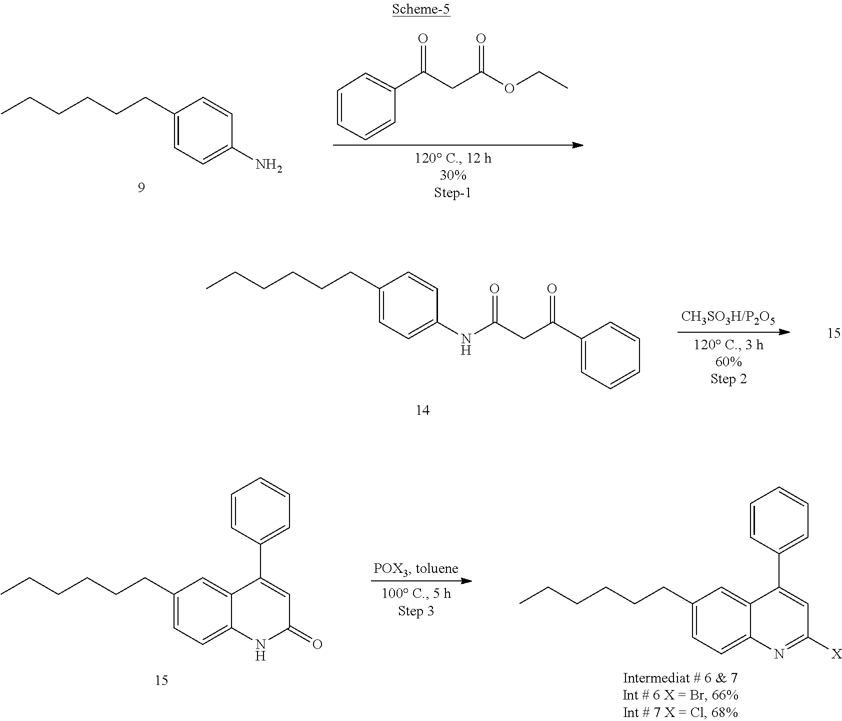

### Step 1: Synthesis of N-(4-hexylphenyl)-3-oxo-3-phenylpropanamide

A mixture of 4-hexyl aniline (30 g, 0.170 mol) and ethyl 3-oxo-3-phenylpropanoate (37 g, 0.186 mol) was heated at 120° C. for 12 h. After completion of the reaction (monitored by T.L.C, Eluent: 10% EtOAc/Hexane, R.f: 0.3), it was cooled to room temperature and diluted with ice-water (100 mL), extracted with ethyl acetate (3×100 mL) and washed with brine (2×50 mL). The combined organic layer was dried over Na₂SO₄ (35 g), filtered and concentrated under reduced pressure to obtain crude compound. The crude compound was purified by column chromatography over silica gel (100-200 mesh) using a solvent gradient of 4% EtOAC in hexane as eluent to afford N-(4-hexylphenyl)-3-oxo-3-phenylpropanamide as a white solid (16.5 g, 30%) MS (ESI) m/z: 324.0 [M+H]⁺.

### Step 2: Synthesis of 6-hexyl-4-phenylquinolin-2(1H)-one

To a stirred solution of N-(4-hexylphenyl)-3-oxo-3-phenylpropanamide (16.5 g, 0.051 mol) in methanesulphonic acid (66.3 mL, 1.022 mol) at room temperature was added phosphorus pentoxide (14.4 g, 0.102 mol) under nitrogen atmosphere. The reaction mixture was heated at 100° C. for 3 h. After completion of the reaction (monitored by T.L.C, Eluent: 30% EtOAc/Hexane, R.f: 0.3), it was cooled to room temperature, poured onto ice-water (350 mL), extracted with EtOAc (3×150 mL) and washed with brine (1×50 mL). The combined organic layer was dried over Na₂SO₄ (12 g), filtered and concentrated under reduced pressure to obtain crude compound. The crude compound was triturated with diethyl ether (2×50 mL) to afford 6-hexyl-4-phenylquinolin-2(1H)-one (10 g, 60%) as off-white solid. MS (ESI) m/z: m/z 306 [M+H]⁺.

### Step 3: Synthesis of 2-Bromo-6-hexyl-4-phenylquinoline (Int #6)

The process of this step was adopted from step-3 of scheme-1. The desired compound was obtained as pale-brown liquid (400 mg, 66%). MS (ESI) m/z: m/z 368.3 [M+H]⁺.

### Step 3A: Synthesis of 2-chloro-6-hexyl-4-phenylquinoline (Int-7)

To a stirred solution of 6-hexyl-4-phenylquinolin-2(1H)-one (10 g, 0.032 mol) in POCl₃ (61.30 mL, 0.65 mol) was added N,N-Dimethylaniline (10.3 mL, 0.082 mol) and heated at 100° C. for 5 h. After completion of the reaction (monitored by T.L.C, Eluent: 10% EtOAc/Hexane, R.f: 0.7), excess POCl₃ was concentrated under reduced pressure and the residue was poured onto ice-water (150 mL) and the product was extracted with EtOAc (3×120 mL) and washed with brine (1×50 mL). The combined organic layers were dried over Na₂SO₄ (8 g), filtered and concentrated under reduced pressure to obtain crude compound. The crude compound was purified by column chromatography over silica gel (100-200 mesh) using a solvent gradient of 3% EtOAC in Hexane as eluent to afford 2-chloro-6-hexyl-4-phenylquinoline as a colorless liquid (7.2 g, 68%). MS (ESI) m/z: 324.0 [M+H]⁺.

### Example #1: Synthesis of (E)-N-methyl-N-(4-phenyl-6-styrylquinolin-2-yl) glycine

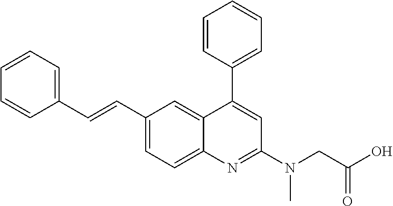 [SMILES: CN(CC(=O)O)c1cc(-c2ccccc2)c2cc(/C=C/c3ccccc3)ccc2n1]

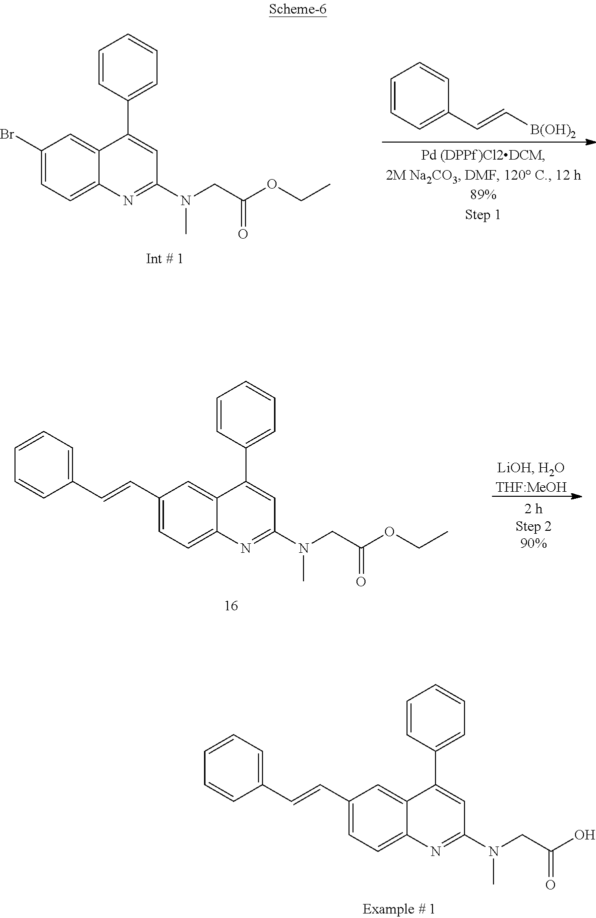 [SMILES: CCOC(=O)CN(C)c1cc(-c2ccccc2)c2cc(Br)ccc2n1 ; OB(O)/C=C/c1ccccc1 ; CCOC(=O)CN(C)c1cc(-c2ccccc2)c2cc(/C=C/c3ccccc3)ccc2n1 ; "LiOH, H2OTHF:MeOH" ; CN(CC(=O)O)c1cc(-c2ccccc2)c2cc(/C=C/c3ccccc3)ccc2n1]

### Step 1: synthesis of ethyl (E)-N-methyl-N-(4-phenyl-6-styrylquinolin-2-yl) glycinate

To a stirred solution of ethyl N-(6-bromo-4-phenylquinolin-2-yl)-N-methyl glycinate (0.2 g, 0.501 mmol) in DMF (2 mL) was added (E)-styryl boronic acid (0.11 g, 0.753 mmol), 2M solution of sodium carbonate (2 mL) (degassed under nitrogen atmosphere for 10 mins), and Pd (DPPf)Cl₂. DCM adduct (0.0041 g, 0.05 mmol) and heated the reaction mixture at 120° C. for 12 h. After completion of the reaction (monitored by T.L.C, Eluent: 20% EtOAc: Hexane, R.f: 0.3), the reaction mixture was cooled to room temperature, diluted the reaction mixture with water (10 mL) and the product was extracted with ethyl acetate (2×15 mL). The combined organic layer was dried over sodium sulphate, filtered and concentrated under reduced pressure to obtain crude product. The crude compound was purified by column chromatography over silica gel using a solvent gradient of 10% EtOAC in Hexane as eluent to afford ethyl (E)-N-methyl-N-(4-phenyl-6-styrylquinolin-2-yl) glycinate (210 mg, 89%). MS (ESI) m/z: 423.0 [M+H]⁺

### Step-2: Synthesis of (E)-N-methyl-N-(4-phenyl-6-styrylquinolin-2-yl) glycine

To a stirred solution of ethyl (E)-N-methyl-N-(4-phenyl-6-styrylquinolin-2-yl) glycinate (0.035 g, 0.082 mmol) in THF (2 mL) and water (1 mL) at 0° C. was added lithium hydroxide monohydrate (0.0017 g). The reaction mixture was slowly warmed to room temperature and stirred for 2 h. After completion of the reaction (monitored by T.L.C, Eluent: 10% MeOH:DCM, R.f: 0.2), the excess solvents was distilled under reaction mixture. The resulting residue was acidified with 1N HCl (2 mL) and the product was extracted with ethyl acetate (2×15 mL). The combined organic layer was dried over sodium sulphate, filtered and concentrated under reduced pressure to obtain crude material. The obtained material was triturated with ether (10 ml) and pentane (5 mL) to afford (E)-N-methyl-N-(4-phenyl-6-styrylquinolin-2-yl)glycine (10.6 mg, 90%) as an yellow colour solid. ¹H-NMR (400 MHz, DMSO-d6): δ 12.57 (bs, 1H), 7.97 (d, J=7.20 Hz, 1H), 7.63-7.55 (m, 9H), 7.35-7.31 (m, 2H), 7.27-7.20 (m, 2H), 7.18-7.13 (m, 1H), 6.95 (s, 1H), 4.46 (s, 2H), 3.22 (s, 3H), MS (ESI) m/z: 395.0 (M+H)⁺.

### Example #2: Synthesis of N-methyl-N-(6-phenethyl-4-phenylquinolin-2-yl) glycine

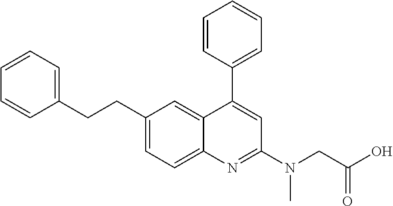 [SMILES: CN(CC(=O)O)c1cc(-c2ccccc2)c2cc(CCc3ccccc3)ccc2n1]

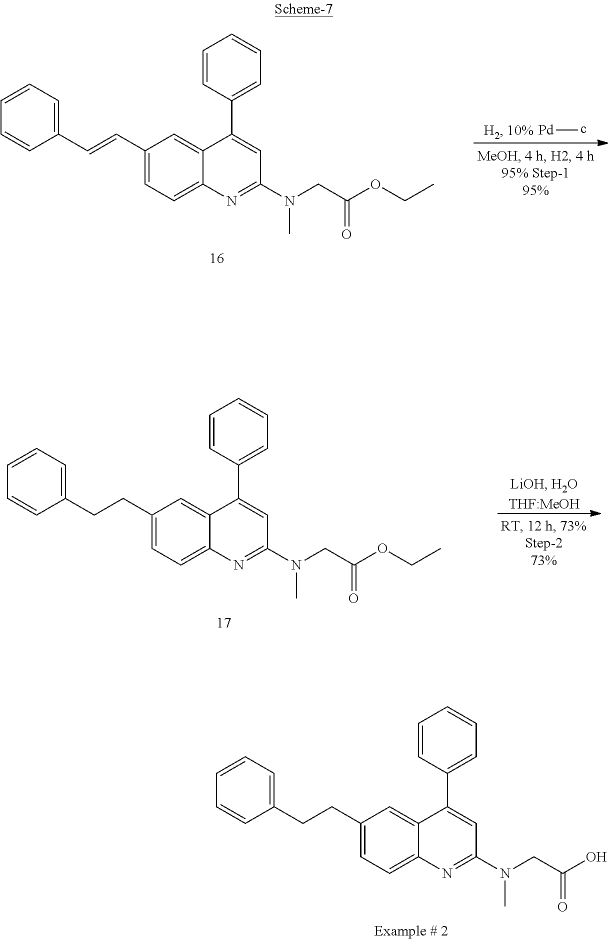

### Step-1: Synthesis of Ethyl N-methyl-N-(6-phenethyl-4-phenylquinolin-2-yl) glycinate

To a stirred solution of ethyl (E)-N-methyl-N-(4-phenyl-6-styrylquinolin-2-yl) glycinate (0.1 g, 0.23 mmol) in MeOH (10 mL) under nitrogen atmosphere was added 10% Pd/C (10 mg). The reaction mixture was hydrogenated under balloon pressure at room temperature for 4 h. After completion of the reaction (monitored by T.L.C, Eluent: 10% EtOAc: Hexane, R.f: 0.5), the reaction mixture was filtered through a celite pad, washed with MeOH (20 mL). Filtrate was concentrated under reduced pressure to afford ethyl N-methyl-N-(6-phenethyl-4-phenylquinolin-2-yl) glycinate (100 mg, 95%). MS (ESI) m/z: 425.0 [M+H]⁺.

### Step-2: Synthesis of N-methyl-N-(6-phenethyl-4-phenylquinolin-2-yl) glycine

The process of this step was adopted similar to step-2 of Example #1 and the desired compound obtained as an off-white solid (20.8 mg, 73%). ¹H-NMR (400 MHz, DMSO-d6): δ 12.53 (bs, 1H), 7.55-7.49 (m, 4H), 7.45 (dd, J=1.60, 8.60 Hz, 1H), 7.37-7.34 (m, 2H), 7.26-7.22 (m, 2H), 7.19-7.15 (m, 2H), 7.11 (d, J=6.80 Hz, 2H), 6.88 (s, 1H), 4.41 (s, 2H), 3.19 (s, 3H), 2.89 (d, J=6.00 Hz, 2H), 2.85 (d, J=5.60 Hz, 2H), MS (ESI) m/z: 397.0 [M+H]⁺.

### Example #3: Synthesis of N-(6-(4-ethylphenyl)-4-phenylquinolin-2-yl)-N-methyl glycine

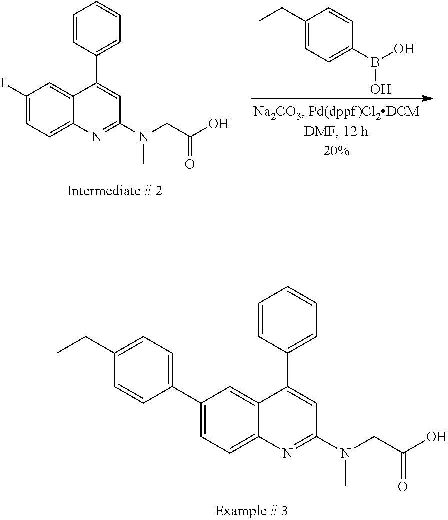 [SMILES: CN(CC(=O)O)c1cc(-c2ccccc2)c2cc(I)ccc2n1 ; CCc1ccc(B(O)O)cc1 ; CCc1ccc(-c2ccc3nc(N(C)CC(=O)O)cc(-c4ccccc4)c3c2)cc1]

The process of this step was adopted similar to step-1 of Example #1 and the desired compound obtained as pale-yellow solid (11 mg, 20%). ¹H-NMR (400 MHz, DMSO-d₆): δ 12.57 (s, 1H), 7.87-7.83 (m, 1H), 7.74-7.74 (m, 1H), 7.70-7.68 (m, 1H), 7.60-7.58 (m, 4H), 7.57-7.51 (m, 1H), 7.46 (d, J=8.40 Hz, 2H), 7.26 (d, J=8.40 Hz, 2H), 6.99 (s, 1H), 4.47 (s, 2H), 3.23 (s, 3H), 2.68-2.58 (m, 2H), 1.18 (t, J=15.20 Hz, 3H), MS (ESI) m/z: 397.1 [M+H]⁺.

### Example #4 Synthesis of N-(6-hexyl-3-methyl-4-phenylquinolin-2-yl)-N-methyl glycine

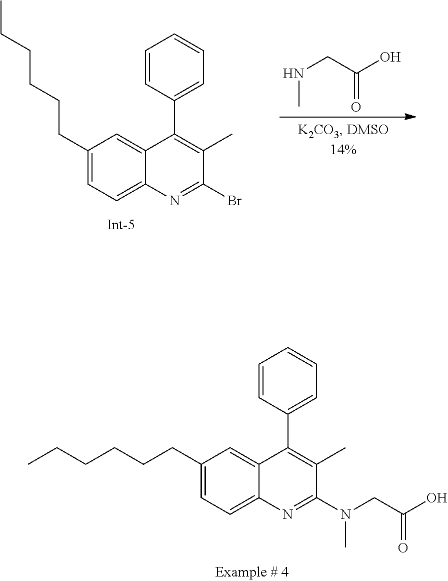

To a stirred solution of 2-bromo-6-hexyl-3-methyl-4-phenylquinoline (100 mg, 0.261 mmol) in DMSO (1.5 mL) at room temperature was added potassium carbonate (73 mg, 0.523 mmol) followed by sarcosine (46 mg, 0.525 mmol). The reaction mixture was heated at 120° C. for 16 h. After completion of the reaction (monitored by T.L.C, Eluent: 10% Methanol: DCM, R.f: 0.3), the reaction mixture was cooled to room temperature and portioned between ice-water (5 mL) and EtOAc (10 mL). The aqueous layer was acidified with 1N HCl and the precipitated solid was collected by filtration, washed with ice-water (10 mL) and dried under vacuum to afford N-(6-hexyl-3-methyl-4-phenylquinolin-2-yl)-N-methyl glycine as a Pale-green solid (15 mg, 14%). ¹H-NMR (400 MHz, DMSO-d₆): δ 7.81 (s, 1H), 7.65-7.53 (m, 4H), 7.33-7.28 (m, 2H), 6.93 (s, 1H), 4.25 (s, 2H), 3.19 (s, 3H), 2.57-2.54 (m, 2H), 2.09 (s, 3H), 1.50-1.47 (m, 2H), 1.15-1.25 (m, 6H), 0.81 (t, J=13.20 Hz, 3H), MS (ESI) m/z: 391.0 [M+H]⁺.

### Example #5 Synthesis of N-((2H-tetrazol-5-yl) methyl)-6-hexyl-N-methyl-4-phenylquinolin-2-amine

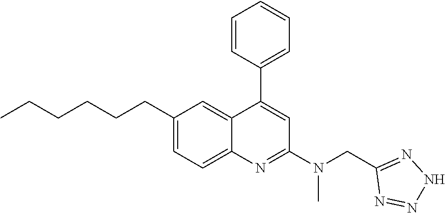 [SMILES: CCCCCCc1ccc2nc(N(C)Cc3nn[nH]n3)cc(-c3ccccc3)c2c1]

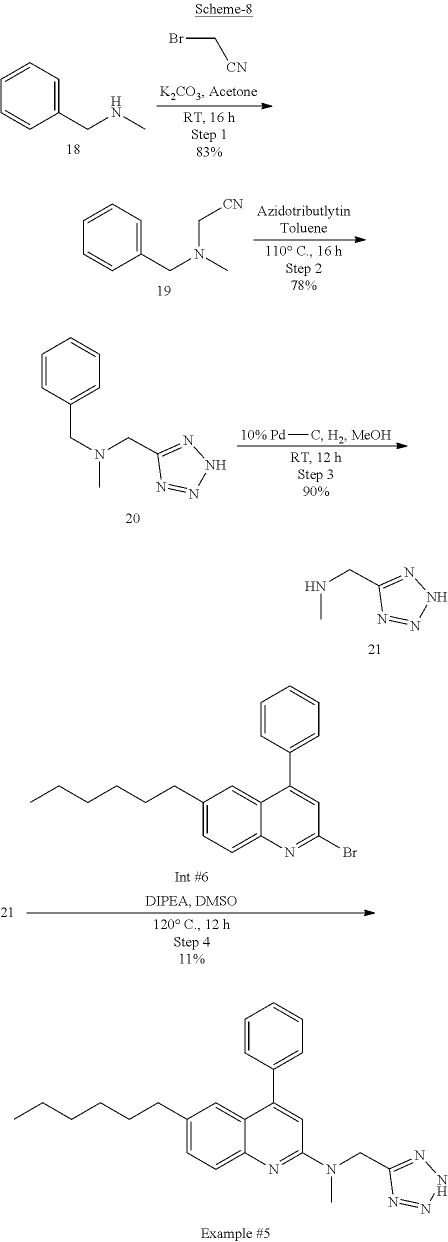

### Step 1: Synthesis of 2-(benzyl(methyl)amino) acetonitrile

To a stirred solution of N-methyl-1-phenylmethanamine (2.0 g, 0.0165 mol) in acetone (20 mL) was added K₂CO₃ (5.7 g, 0.041 mol) followed by bromo acetonitrile (2.37 g, 0.0198 mol). The reaction mixture was stirred at room temperature for 16 h. After completion of the reaction (monitored by T.L.C, Eluent: 10% EtOAc/Hexane, R.f: 0.5), it was filtered to remove the inorganic salts. Organic layer was evaporated under reduced pressure to obtain the crude compound. The crude compound was purified by column chromatography over silica gel (100-200 mesh) using a solvent gradient of 5% EtOAC in Hexane as eluent to afford 2-(benzyl(methyl)amino) acetonitrile as a pale-yellow liquid (2.20 g, 83%). MS (ESI) m/z: 161.0 [M+H]⁺.

### Step 2: Synthesis of N-((2H-tetrazol-5-yl) methyl)-N-methyl-1-phenylmethanamine

To a stirred solution of 2-(benzyl(methyl)amino)acetonitrile (200 mg, 0.125 mmol) in toluene (5 mL) was added azidotributyl tin (625 mg, 1.88 mmol) and heated at 110° C. for 16 h. After completion of the reaction (monitored by T.L.C, Eluent: 30% EtOAc/Hexane, R.f: 0.30), it was cooled to room temperature and concentrated under reduced pressure to obtain the crude compound. The crude compound was purified by column chromatography over silica gel (100-200 mesh) using a solvent gradient of 15% MeOH in DCM as eluent to afford N-((2H-tetrazol-5-yl) methyl)-N-methyl-1-phenylmethanamine (200 mg, 78%) as a yellow liquid. MS (ESI) m/z: 204.0 [M+H]⁺.

### Step 3: Synthesis N-methyl-1-(2H-tetrazol-5-yl) methanamine

To a stirred solution of N-((2H-tetrazol-5-yl)methyl)-N-methyl-1-phenylmethanamine (200 mg, 0.985 mmol) in MeOH (5 mL) was added 10% Pd—C (25 mg) and hydrogenated under balloon pressure for 12 h. After completion of the reaction (monitored by T.L.C, Eluent: 10% MeOH/DCM, R.f: 0.30), it was filtered over celite bed and the celite was once again washed with additional quantity of methanol. The organic layer was concentrated under reduced pressure to obtain the crude product. The crude compound was further triturated with diethylether and pentane and isolated N-methyl-1-(2H-tetrazol-5-yl) methanamine (200 mg, 90%) as a pale-yellow oily liquid. MS (ESI) m/z: 114.0 [M+H]⁺.

### Step 4: Synthesis of N-((2H-tetrazol-5-yl) methyl)-6-hexyl-N-methyl-4-phenylquinolin-2-amine

To a stirred solution of sarcosine (92 mg, 0.818 mmol) in DMSO (5 mL) was added int #6 (250 mg, 0.681 mmol) followed by Cs₂CO₃ (440 mg, 1.350 mmol). The reaction mixture was heated to 120° C. for 12 h. After completion of the reaction (monitored by T.L.C, Eluent: 30% EtOAc/Hexane, R.f: 0.50), it was cooled to room temperature and diluted with water and extracted with ethyl acetate (2×10 mL). The organic layer was separated and dried over Na₂SO₄, filtered and concentrated under reduced pressure to obtain crude compound. The crude compound was purified by column chromatography over silica gel (100-200 mesh) using a solvent gradient of 30% EtOAc in Hexane as eluent to N-((2H-tetrazol-5-yl)methyl)-6-hexyl-N-methyl-4-phenylquinolin-2-amine (30 mg, 11%) as a pale yellow solid. ¹H-NMR (400 MHz, DMSO-d₆): δ 7.59-7.54 (m, 4H), 7.52-7.49 (m, 2H), 7.42-7.39 (m, 1H), 7.33 (br.s, 1H), 7.02 (s, 1H), 5.22 (s, 2H), 3.27 (s, 3H), 2.67-2.56 (m, 2H), 1.51-1.49 (m, 2H), 1.23-1.15 (m, 7H), 0.82 (t, J=12.80 Hz, 3H), MS (ESI) m/z: m/z 401.0 [M+H]⁺.

### Example #6 Synthesis of 6-hexyl-N-methyl-4-phenyl-N-(2H-tetrazol-5-yl) quinolin-2-amine

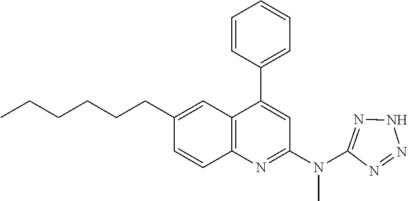 [SMILES: CCCCCCc1ccc2nc(N(C)c3nn[nH]n3)cc(-c3ccccc3)c2c1]

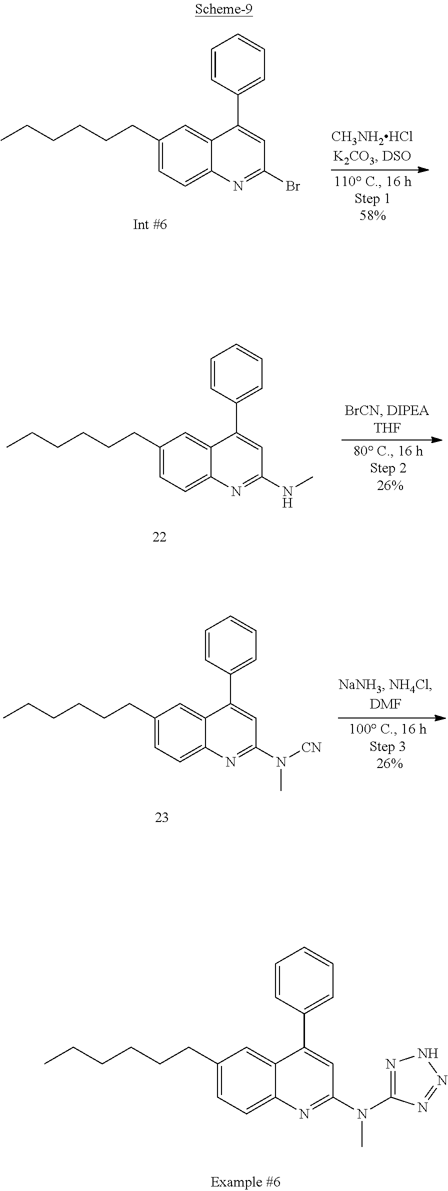

### Step 1: Synthesis of 6-hexyl-N-methyl-4-phenylquinolin-2-amine

To a stirred solution of Int #6 (500 mg, 1.358 mmol) in DMSO (10 mL) was added K₂CO₃ (940 mg, 6.794 mmol) followed by methylamine. HCl (465 mg, 6.794 mmol). The reaction mixture was heated to 110° C. for 16 h. After completion of the reaction (monitored by T.L.C, Eluent: 30% EtOAc/Hexane, R.f: 0.2), reaction mixture was cooled to room temperature and diluted with water (15 mL) and extracted with ethyl acetate (2×50 mL). The organic layer was dried over Na₂SO₄, filtered and concentrated under reduced pressure to obtain the crude compound. The crude compound was purified by column chromatography over silica gel (100-200 mesh) using a solvent gradient of 20% EtOAC in hexane as eluent to afford 2-(benzyl(methyl)amino)acetonitrile (250 mg, 58%) as a pale brown liquid. MS (ESI) m/z: 319.0 [M+H]⁺.

### Step 2: Synthesis of N-(6-hexyl-4-phenylquinolin-2-yl)-N-methylcyanamide

To a stirred solution of 6-hexyl-N-methyl-4-phenylquinolin-2-amine (250 mg, 0.787 mmol) in THF (10 mL) was added K₂CO₃ (660 mg, 4.717 mmol) followed by BrCN (340 mg, 3.145 mmol). The reaction mixture was heated to 80° C. for 16 h. After completion of the reaction (monitored by T.L.C, Eluent: 10% EtOAc/Hexane, R.f: 0.7), it was cooled to room temperature and diluted with water (15 mL) and extracted with ethyl acetate (2×50 mL). The organic layer was dried over Na₂SO₄, filtered and concentrated under reduced pressure to obtain the crude compound. The crude compound was purified by column chromatography over silica gel (100-200 mesh) using a solvent gradient of 5% EtOAC in hexane as eluent to afford 2-(benzyl(methyl)amino)acetonitrile (70 mg, 26%) as a pale brown solid. MS (ESI) m/z: 343.0 [M+H]⁺.

### Step 3: Synthesis 6-hexyl-N-methyl-4-phenyl-N-(2H-tetrazol-5-yl)quinolin-2-amine

To a stirred solution of N-(6-hexyl-4-phenylquinolin-2-yl)-N-methylcyanamide (40 mg, 0.116 mmol) in DMF (4 mL) was added NaN₃ (9 mg, 0.134 mmol) followed by NH₄Cl (13 mg, 0.232 mmol). The reaction mixture was heated to 100° C. for 16 h. After completion of the reaction (monitored by T.L.C, Eluent: 50% EtOAc/Hexane, R.f: 0.25), it was cooled to room temperature and diluted with water (20 mL) and extracted with ethyl acetate (2×50 mL). The organic layer was dried over Na₂SO₄, filtered and concentrated under reduced pressure to obtain the crude compound. The crude compound was purified by column chromatography over silica gel (100-200 mesh) using a solvent gradient of 50% EtOAC in Hexane as eluent to afford 6-hexyl-N-methyl-4-phenyl-N-(2H-tetrazol-5-yl)quinolin-2-amine (12 mg, 26%) as an off-white solid. ¹H-NMR (400 MHz, DMSO-d₆): δ 15.71 (s, 1H), 8.26 (d, J=8.40 Hz, 1H), 7.64-7.55 (m, 7H), 7.50 (s, 1H), 3.83 (s, 3H), 2.67 (t, J=15.20 Hz, 2H), 1.62-1.55 (m, 2H), 1.31-1.20 (m, 6H), 0.83 (t, J=14.00 Hz, 3H), MS (ESI) m/z: 387.0 [M+H]⁺.

### Example #7 Synthesis of (E)-5-((6-hexyl-4-phenylquinolin-2-yl)methylene)thiazolidine-2,4-dione

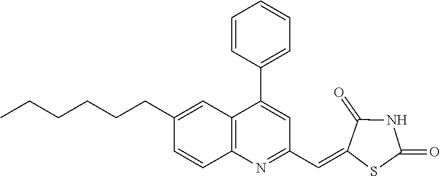 [SMILES: CCCCCCc1ccc2nc(/C=C3/SC(=O)NC3=O)cc(-c3ccccc3)c2c1]

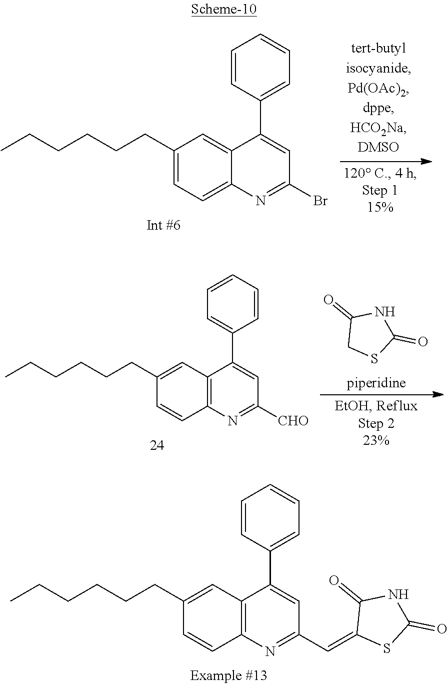 [SMILES: CCCCCCc1ccc2nc(Br)cc(-c3ccccc3)c2c1 ; CCCCCCc1ccc2nc(C=O)cc(-c3ccccc3)c2c1 ; O=C1CSC(=O)N1 ; CCCCCCc1ccc2nc(/C=C3/SC(=O)NC3=O)cc(-c3ccccc3)c2c1]

### Step 1: Synthesis of 6-hexyl-4-phenylquinoline-2-carbaldehyde

To a stirred solution of 2-bromo-6-hexyl-4-phenylquinoline (1 g, 2.72 mmol) in DMSO (10 mL) at RT under nitrogen atmosphere was added HCO₂Na (370 mg, 5.42 mmol) and dppe (98 mg, 0.245 mmol). The reaction mixture was degassed with N₂ gas and added t-butyl isocyanide (0.37 ml, 3.26 mmol), followed by Pd (OAc)₂ (31 mg, 0.136 mmol). The reaction mixture was heated at 120° C. for 4 h. After completion of the reaction (monitored by T.L.C, Eluent: 10% EtOAc: Hexane, R.f: 0.4), it was cooled to room temperature, poured onto ice-water (30 mL) and the product was extracted with EtOAc (2×300 mL). The combined organic layer was dried over Na₂SO₄ filtered and concentrated under reduced pressure to obtain crude compound. The crude compound was purified by column chromatography over silica gel (100-200 mesh) using a solvent gradient of 3-4% EtOAc in Hexane as eluent to afford 6-hexyl-4-phenylquinoline-2-carbaldehyde (130 mg, 15%) as an orange oil. MS (ESI) m/z: 318.0 [M+H]⁺.

### Step 2: 5-((6-hexyl-4-phenylquinolin-2-yl) methylene) thiazolidine-2,4-dione

A mixture of 6-hexyl-4-phenylquinoline-2-carbaldehyde (130 mg, 0.41 mmol) and thiazolidine-2,4-dione (52 mg, 0.45 mmol) in EtOH at RT, was added piperidine (0.1 mL, 0.284 mmol) and heated up to reflux for 24 h. After completion of the reaction (monitored by T.L.C, Eluent: 20% EtOAc/Hexane, R.f: 0.4), it was cooled to room temperature and the solvent was removed under reduced pressure. The residue was diluted with water (20 mL), the compound was extracted with ethyl acetate (3×20 mL). The combined organic layer was dried over Na₂SO₄ filtered and concentrated under reduced pressure to obtain crude compound. The crude compound was purified by column chromatography over silica gel (100-200 mesh) using a solvent gradient of 10% EtOAc in hexane as eluent to afford 5-((6-hexyl-4-phenylquinolin-2-yl) methylene) thiazolidine-2,4-dione (40 mg, 23%) as a beige solid. ¹H-NMR (400 MHz, DMSO-d6): δ 12.49 (s, 1H), 8.12 (d, J=8.8 Hz, 1H), 7.96 (br.s, 1H), 7.86 (s, 1H), 7.75-7.72 (m, 1H), 7.61-7.56 (m, 6H), 2.72 (t, J=14.4 Hz, 2H), 1.62-1.55 (m, 2H), 1.31-1.20 (m, 6H), 0.84-0.82 (m, 3H), MS (ESI) m/z: 417.4 [M+H]⁺

### Example #8 Synthesis of 5-((6-hexyl-4-phenylquinolin-2-yl)methyl)thiazolidine-2,4-dione

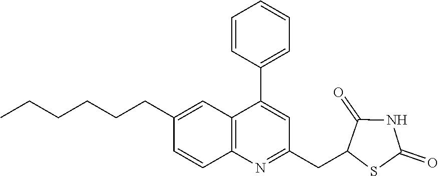 [SMILES: CCCCCCc1ccc2nc(CC3SC(=O)NC3=O)cc(-c3ccccc3)c2c1]

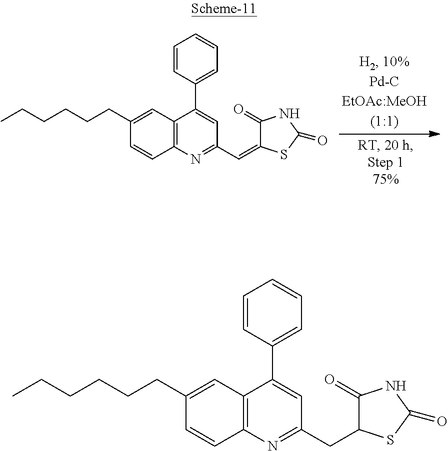 [SMILES: CCCCCCc1ccc2nc(/C=C3/SC(=O)NC3=O)cc(-c3ccccc3)c2c1 ; CCCCCCc1ccc2nc(CC3SC(=O)NC3=O)cc(-c3ccccc3)c2c1]

### Step 1: 5-((6-hexyl-4-phenylquinolin-2-yl) methyl) thiazolidine-2,4-dione

To a stirred solution of 5-((6-hexyl-4-phenylquinolin-2-yl) methylene) thiazolidine-2,4-dione (40 mg, 0.0962 mmol) in EtOAc:MeOH (1:1) (4 mL) at RT, was added Pd/C (20 mg). The reaction mixture was kept under hydrogen atmosphere for 24 h. After completion of the reaction (monitored by T.L.C, Eluent: 20% EtOAc/Hexane, R.f: 0.3), reaction mass was filtered through celite and washed with EtOAc. The filtrate was evaporated to obtain crude compound. The crude compound was purified by column chromatography over silica gel (100-200 mesh) using a solvent gradient of 10% EtOAc in hexane as eluent to afford 5-((6-hexyl-4-phenylquinolin-2-yl) methyl) thiazolidine-2,4-dione) (30 mg, 75%) as a Pale-yellow solid. ¹H-NMR (400 MHz, CDCl₃): δ 7.99 (s, 1H), 7.97 (s, 1H), 7.61 (br.s, 1H), 7.58-7.26 (m, 6H), 7.18 (s, 1H), 5.061 (dd, J=3.6 Hz, 1H), 4.04-3.99 (m, 1H), 3.60-3.53 (m, 1H), 2.70 (t, J=7.6 Hz, 2H), 1.63-1.55 (m, 2H), 1.33-1.26 (m, 6H) 0.88-0.84 (t, J=6.8 Hz, 3H),

MS (ESI) m/z: 419.0 [M+H]⁺.

### Example #9 Synthesis of Synthesis of 2-((6-hexyl-4-phenylquinolin-2-yl)oxy)propanoic acid

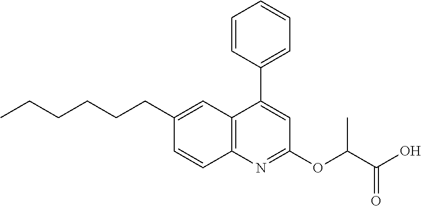 [SMILES: CCCCCCc1ccc2nc(OC(C)C(=O)O)cc(-c3ccccc3)c2c1]

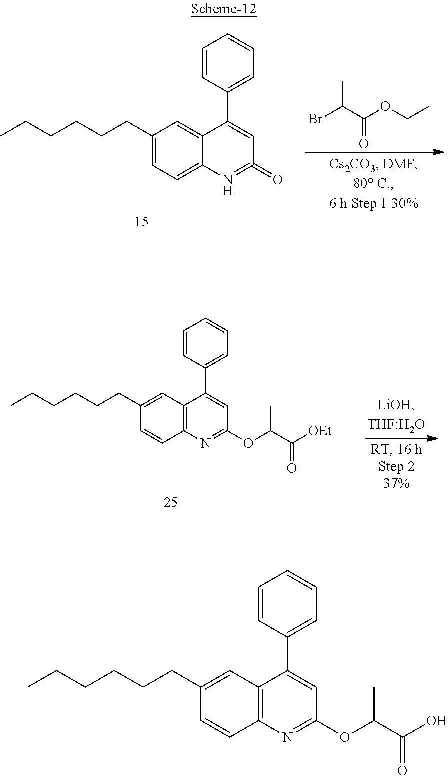

### Step 1: Synthesis of ethyl 2-((6-hexyl-4-phenylquinolin-2-yl)oxy)propanoate

To a stirred solution of 6-hexyl-4-phenylquinolin-2(1H)-one (100 mg, 0.328 mmol) in DMF (1 mL) was added ethyl 2-bromopropanoate (148 mg, 0.820 mmol) followed by Cs₂CO₃ (320 mg, 0.984 mmol). The reaction mixture was heated to 80° C. for 6 h. After completion of the reaction (monitored by T.L.C, Eluent: 30% EtOAc/n-Hexane, R.f: 0.60), it was diluted with water and extracted with ethyl acetate (2×10 mL). The organic layer was separated and dried over Na₂SO₄, filtered and concentrated under reduced pressure to obtain ethyl 2-((6-hexyl-4-phenylquinolin-2-yl)oxy)propanoate (40 mg, 30%) as an off-white solid. MS (ESI) m/z: 406.0 [M+H]⁺.

### Step 2: Synthesis of 2-((6-hexyl-4-phenylquinolin-2-yl)oxy)propanoic acid

To a stirred solution of ethyl 2-((6-hexyl-4-phenylquinolin-2-yl)oxy)propanoate (40 mg, 0.098 mmol) in THF:H₂O (5 mL, 4:1) was added LiOH·H₂O (12 mg, 0.494 mmol) and stirred at room temperature for 16 h. After completion of the reaction (monitored by T.L.C, Eluent: 30% EtOAc/Hexane, R.f: 0.30), it was diluted with water (10 mL) and acidified with 2M HCl (pH˜5-6) and extracted with DCM (2×10 mL). The organic layer was dried over Na₂SO₄, filtered and concentrated under reduced pressure. The obtained residue was triturated with Ether (10 mL) and pentane (10 mL) afforded 2-((6-hexyl-4-phenylquinolin-2-yl)oxy)propanoic acid (12 mg, 37%) as off-white solid. ¹H-NMR (400 MHz, DMSO-d₆): δ 7.69-7.67 (m, 1H), 7.61-7.51 (m, 6H), 7.46-7.45 (m, 1H), 6.93 (s, 1H), 5.50 (m, 1H), 2.68-2.64 (m, 2H), 1.57-1.52 (m, 5H), 1.29-1.20 (m, 6H), 0.82 (t, J=14.00 Hz, 3H). MS (ESI) m/z: 378.0 [M+H]⁺.

### Example #10 Synthesis of 3-(6-hexyl-4-phenylquinolin-2-yl) butanoic acid

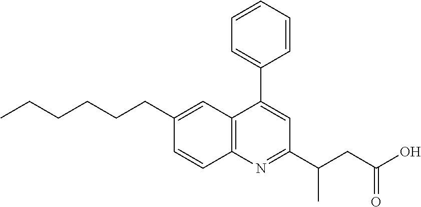 [SMILES: CCCCCCc1ccc2nc(C(C)CC(=O)O)cc(-c3ccccc3)c2c1]

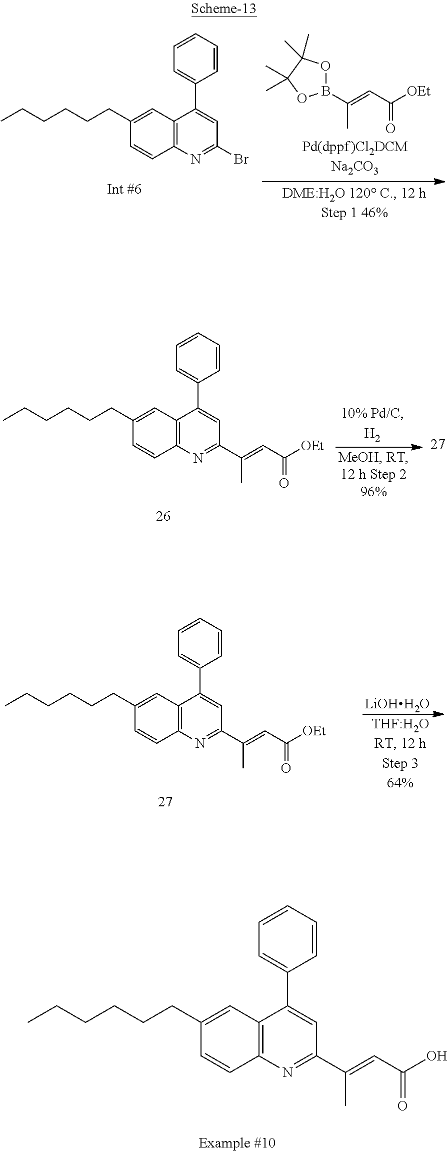 [SMILES: CCCCCCc1ccc2nc(Br)cc(-c3ccccc3)c2c1 ; CCOC(=O)/C=C(\C)B1OC(C)(C)C(C)(C)O1 ; CCCCCCc1ccc2nc(/C(C)=C/C(=O)OCC)cc(-c3ccccc3)c2c1 ; CCCCCCc1ccc2nc(/C(C)=C/C(=O)OCC)cc(-c3ccccc3)c2c1 ; "LiOH·H2O" ; CCCCCCc1ccc2nc(/C(C)=C/C(=O)O)cc(-c3ccccc3)c2c1]

### Step 1: Synthesis of ethyl (E)-3-(6-hexyl-4-phenylquinolin-2-yl) but-2-enoate

The process of this step was adopted from step #1 of example #1 to obtain the title compound (250 mg, 46%) as a white solid. MS (ESI) m/z: 402 [M+H]⁺

### Step 2: Synthesis of ethyl 3-(6-hexyl-4-phenylquinolin-2-yl) butanoate

The process of this step was obtained from step-1 of Example #2 to obtain the title compound (240 mg, 96%) as Grey color liquid. MS (ESI) m/z: 404 (M+H)⁺.

### Step 3: Synthesis of 3-(6-hexyl-4-phenylquinolin-2-yl) butanoic acid

The process of this step was obtained from step-2 of Example #1 to obtain 3-(6-hexyl-4-phenylquinolin-2-yl) butanoic acid (143 mg, 64%) as a Pale-yellow thick liquid. ¹H-NMR (400 MHz, DMSO-d6): δ 8.03 (d, J=8.40 Hz, 1H), 7.66-7.63 (m, 2H), 7.58-7.54 (m, 3H), 7.52-7.49 (m, 2H), 7.29 (s, 1H), 3.52-3.48 (m, 1H), 3.15 (dd, J=1.60, 14.80 Hz, 1H), 2.91 (dd, J=7.20, 14.80 Hz, 1H), 2.73 (t, J=8.00 Hz, 2H), 1.64-1.60 (m, 2H), 1.54 (d, J=7.20 Hz, 3H), 1.33-1.22 (m, 6H), 0.88-0.85 (m, 3H), MS (ESI) m/z: 376 [M+H]⁺.

### Example #11 Synthesis of N-(6-(benzyloxy)-4-phenylquinolin-2-yl)-N-methylglycine

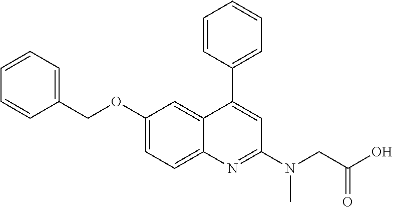 [SMILES: CN(CC(=O)O)c1cc(-c2ccccc2)c2cc(OCc3ccccc3)ccc2n1]

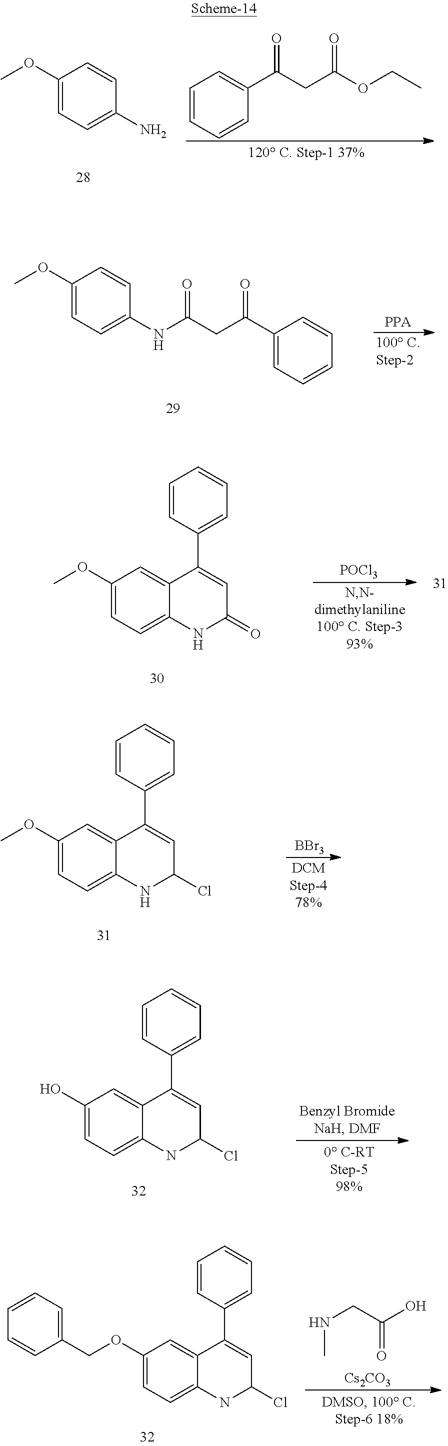

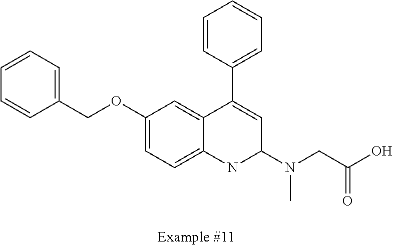 [SMILES: CN(CC(=O)O)C1C=C(c2ccccc2)c2cc(OCc3ccccc3)ccc2N1]

### Step 1: Synthesis of N-(4-methoxyphenyl)-3-oxo-3-phenylpropanamide

To a stirred solution of 4-methoxy aniline (5 g, 40.60 mmol) in xylene (50 mL) was added ethyl 3-oxo-3-phenylpropanoate (11.7 g, 60.90 mmol) and heated at 120° C. for 16 h. After completion of the reaction (monitored by T.L.C, Eluent: 20% EtOAc/Hexane, R.f: 0.6), it was cooled to room temperature, diluted with ice-water (100 mL) and extracted with ethyl acetate (2×100 mL). The combined organic layer was dried over Na₂SO₄, filtered and concentrated under reduced pressure to obtain crude compound. The crude compound was purified by column chromatography over silica gel (100-200 mesh) using a solvent gradient of 8% EtOAc in Hexane to afford N-(4-methoxyphenyl)-3-oxo-3-phenylpropanamide (4 g 37%) as a light-yellow solid. MS (ESI) m/z: 268 [M−H]⁺.

### Step 2: Synthesis of 6-methoxy-4-phenylquinolin-2(1H)-one

To a stirred solution of N-(4-methoxyphenyl)-3-oxo-3-phenylpropanamide (500 mg, 1.86 mmol) in toluene (10 mL) at room temperature was added PPA (3 g, 6 vol). The reaction mixture was heated at 100° C. for 7 h. After completion of the reaction (monitored by T.L.C, Eluent: 50% EtOAc/Hexane, R.f: 0.3), it was cooled to room temperature, poured onto ice-water (100 mL), and extracted with EtOAc (3×150 mL) and washed with brine (1×100 mL). The combined organic layer was dried over Na₂SO₄, filtered and concentrated under reduced pressure to obtain 6-methoxy-4-phenylquinolin-2(1H)-one (400 mg, 86%) as off-white solid. MS (ESI) m/z: 252 [M+H]⁺.

### Step 3: Synthesis of 2-chloro-6-methoxy-4-phenylquinoline

To a stirred solution of 6-methoxy-4-phenylquinolin-2(1H)-one (300 mg, 1.19 mmol) in POCl₃ (3 mL, 10 vol) was added N,N-Dimethylaniline (0.3 mL, 1 vol) and heated at 100° C. for 2 h. After completion of the reaction (monitored by T.L.C, Eluent: 10% EtOAc/Hexane, R.f: 0.8), excess POCl₃ was concentrated under reduced pressure and the residue was poured onto ice-water (10 mL) and the product was extracted with EtOAc (2×10 mL), washed with brine (1×20 mL). The combined organic layers were dried over Na₂SO₄, filtered and concentrated under reduced pressure to afford 2-chloro-6-methoxy-4-phenylquinoline (300 mg, 93%) as a colorless liquid. MS (ESI) m/z: 270 [M+H]⁺.

### Step 4: Synthesis of 2-chloro-4-phenylquinolin-6-ol

To a stirred solution of 2-chloro-6-methoxy-4-phenylquinoline (300 mg, 1.11 mmol) in DCM (5 mL) at 0° C. was added BBr₃ (3.3 mL, 3.33 mmol, 1M in DCM) and the reaction mixture was allowed to room temperature for 4 h. After completion of the reaction (monitored by T.L.C, Eluent: 10% EtOAc/Hexane, R.f: 0.2), the reaction was quenched with ice-water (10 mL) and the product was extracted with DCM (3×15 mL) and washed with brine (1×20 mL). The combined organic layers were dried over Na₂SO₄, filtered and concentrated under reduced pressure to afford 2-chloro-4-phenylquinolin-6-ol (220 mg, 78%) as a colorless liquid. MS (ESI) m/z: 256 (M+H)⁺.

### Step 5: Synthesis of 6-(benzyloxy)-2-chloro-4-phenylquinoline

To a stirred solution of 2-chloro-4-phenylquinolin-6-ol (220 mg, 0.863 mmol) in DMF (4 mL) cooled to 0° C. was added NaH (55% in paraffin oil) (80 mg, 1.8 mmol), followed by benzyl bromide (0.15 mL, 1.3 mmol). The reaction mixture was stirred at room temperature for 2 h. After completion of the reaction (monitored by T.L.C, Eluent: 10% EtOAc: Hexane, R.f: 0.8), the reaction mixture was quenched with ice-water (10 mL) and the product was extracted with EtOAc (2×10 mL). The combined organic layers were dried over Na₂SO₄, filtered and concentrated under reduced pressure to afford 6-(benzyloxy)-2-chloro-4-phenylquinoline (300 mg, 98%) as colorless oil. MS (ESI) m/z: 344 [M−H]⁺.

### Step 6: Synthesis of 2-((6-(benzyloxy)-4-phenylquinolin-2-yl) (methyl)amino) acetic acid

To a stirred solution of 6-(benzyloxy)-2-chloro-4-phenylquinoline (300 mg, 0.86 mmol) in DMSO (5 mL) at room temperature was added cesium carbonate (560 mg, 1.72 mmol) and sarcosine (117 mg, 1.28 mmol). The reaction mixture was heated at 100° C. for 18 h. After completion of the reaction (monitored by T.L.C, Eluent: EtOAc, R.f: 0.3), the reaction mixture was cooled to room temperature and partioned between ice-water (15 mL) and EtOAc (20 mL). The aqueous layer was acidified with 1N HCl and the precipitated solid was collected by filtration, washed with ice-water (10 mL) and dried under vacuum to afford 2-((6-(benzyloxy)-4-phenylquinolin-2-yl) (methyl)amino) acetic acid (60 mg, 18%) as a pale-Yellow solid. ¹H-NMR (400 MHz, DMSO-d₆): δ 7.57-7.51 (m, 4H), 7.44-7.42 (m, 2H), 7.37-7.36 (m, 4H), 7.34-7.29 (m, 2H), 7.00 (d, J=2.40 Hz, 1H), 6.88 (s, 1H), 5.02 (s, 2H), 4.32 (s, 2H), 3.16 (s, 3H), MS (ESI) m/z: 397 [M−H]⁺.

### Example #12 Synthesis of N-methyl-N-(6-(3-methylphenethyl)-4-phenylquinolin-2-yl)glycine

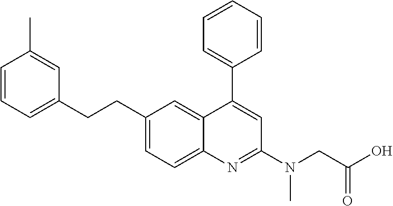 [SMILES: Cc1cccc(CCc2ccc3nc(N(C)CC(=O)O)cc(-c4ccccc4)c3c2)c1]

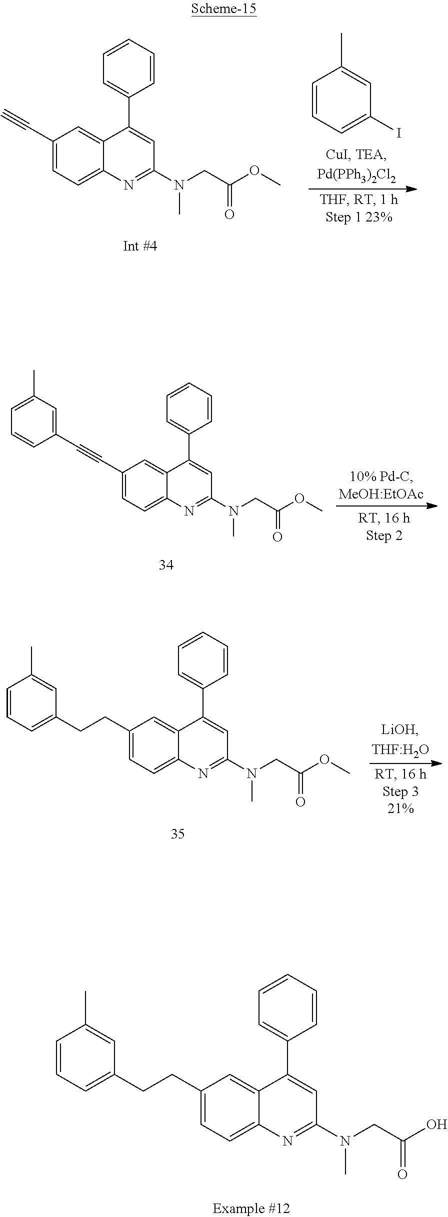

### Step 1: Synthesis of methyl N-methyl-N-(4-phenyl-6-(m-tolylethynyl) quinolin-2-yl) glycinate

To a stirred solution of Int #4 (400 mg, 1.212 mmol) and 1-iodo-3-methylbenzene (320 mg, 1.454) in THF (20 mL) was added CuI (23 mg, 0.121 mmol) followed by Et₃N (2.0 ML, 12.12 mmol). The reaction mixture was degassed for 10 min with Argon and then Pd(PPh₃)₂Cl₂ (42 mg, 0.0606 mmol) was added and again degassed with Argon for 10 min. The resultant reaction mixture was stirred at room temperature for 1 h. After completion of the reaction (monitored by T.L.C, Eluent: 20% EtOAc/Hexane, R.f: 0.5), it was diluted with water (15 mL) and extracted with ethyl acetate (2×50 mL). The organic layer was dried over Na₂SO₄, filtered and concentrated under reduced pressure to obtain the crude compound. The crude compound was purified by column chromatography over silica gel (100-200 mesh) using a solvent gradient of 10% EtOAC in Hexane as eluent to afford methyl N-methyl-N-(4-phenyl-6-(m-tolylethynyl) quinolin-2-yl) glycinate (120 mg, 23%) as a pale-yellow solid. MS (ESI) m/z: 421.0 [M+H]⁺.

### Step 2: Synthesis of methyl N-methyl-N-(6-(3-methylphenethyl)-4-phenylquinolin-2-yl)glycinate

To a stirred solution of methyl N-methyl-N-(4-phenyl-6-(m-tolylethynyl) quinolin-2-yl) glycinate (120 mg, 0.285 mmol) in EtOAc:MeOH (20 mL, 10 mL) was added 10% Pd—C (100 mg) and hydrogenated under balloon pressure for 16 h. After completion of the reaction (monitored by T.L.C, Eluent: 20% EtOAc/Hexane, R.f: 0.55), it was filtered over celite bed and washed with ethyl acetate (25 mL). The organic layer was concentrated under reduced pressure to obtain the crude product (120 mg). The crude compound was utilized in the subsequent step without further purification. MS (ESI) m/z: 426.0 (M+H)⁺.

### Step 3: Synthesis N-methyl-N-(6-(3-methylphenethyl)-4-phenylquinolin-2-yl) glycine

To a stirred solution of N-methyl-N-(6-(3-methylphenethyl)-4-phenylquinolin-2-yl)glycine (200 mg, 0.460 mmol) in THF:H₂O (5 mL, 4:1) was added LiOH·H₂O (80 mg, 1.88 mmol) and stirred at room temperature for 16 h. After completion of the reaction (monitored by T.L.C, Eluent: 10% MeO/DCM, R.f: 0.2), it was diluted with water (5 mL) and acidified with 1N HCl (pH˜5-6) and extracted with 10% MeOH in DCM (2×10 mL). The organic layer was dried over Na₂SO₄, filtered and concentrated under reduced pressure to obtain the crude compound. The crude compound was purified by prep-TLC and isolated N-methyl-N-(6-(3-methylphenethyl)-4-phenylquinolin-2-yl) glycine (40 mg, 21%) as off-white solid. ¹H-NMR (400 MHz, DMSO-d₆): δ 12.50 (s, 1H), 7.55-7.48 (m, 4H), 7.46-7.44 (m, 1H), 7.42-7.35 (m, 2H), 7.22 (s, 1H), 7.11 (t, J=15.60 Hz, 1H), 7.01-6.95 (m, 2H), 6.92-6.86 (m, 2H), 4.41 (s, 2H), 3.19 (s, 3H), 2.90-2.86 (m, 2H), 2.81-2.67 (m, 2H), 2.24 (s, 3H), MS (ESI) m/z: 411.0 [M+H]⁺.

### Example #13 Synthesis of 1-(6-hexyl-4-phenylquinolin-2-yl)-5-oxopyrrolidine-3-carboxylic acid

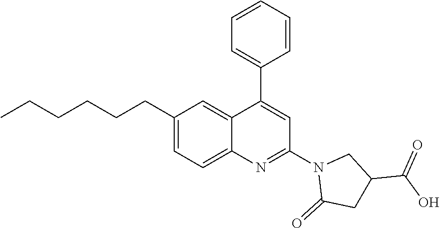 [SMILES: CCCCCCc1ccc2nc(N3CC(C(=O)O)CC3=O)cc(-c3ccccc3)c2c1]

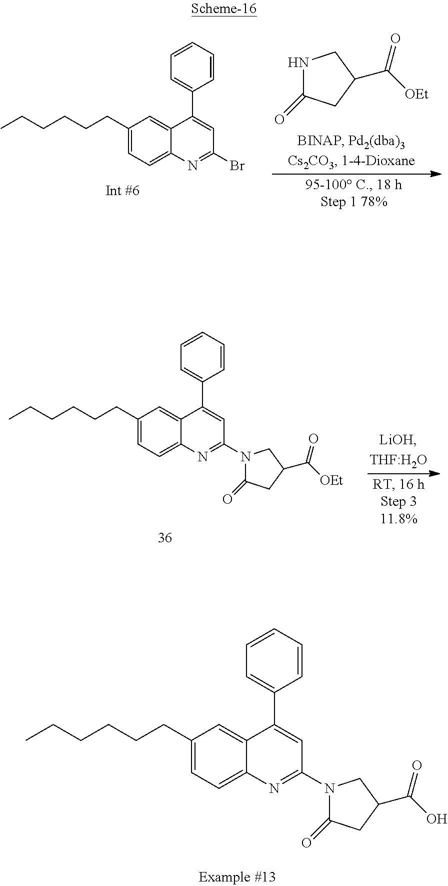 [SMILES: CCCCCCc1ccc2nc(Br)cc(-c3ccccc3)c2c1 ; CCOC(=O)C1CNC(=O)C1 ; CCCCCCc1ccc2nc(N3CC(C(=O)OCC)CC3=O)cc(-c3ccccc3)c2c1 ; CCCCCCc1ccc2nc(N3CC(C(=O)O)CC3=O)cc(-c3ccccc3)c2c1]

### Step 1: Synthesis of ethyl 1-(6-hexyl-4-phenylquinolin-2-yl)-5-oxopyrrolidine-3-carboxylate

To a solution of Int #6 (100 mg, 0.272 mmol) and 1,4-Dioxane (10 mL) in sealed tube was added Cs₂CO₃ (221 mg, 0.68 mmol) followed by the addition of BINAP (10 mg, 0.016 mmol), and Pd₂(dba)₃ (8 mg, 0.008 mmol), under continuous nitrogen purging. After stirring for 10 min, ethyl 5-oxopyrrolidine-3-carboxylate (51 mg, 0.326 mmol) was added and the reaction mixture was heated to 95-100° C. for 18 h. After completion of the reaction (monitored by T.L.C, Eluent: 10% EtOAc/Hexane, R.f: 0.5), it was cooled to room temperature and filtered over celite bed. The celite bed was further washed with ethyl acetate (2×10 mL) and the combined organic layer was with water (10 mL), followed by brine (10 mL). The organic layer was separated and dried over Na₂SO₄, filtered and concentrated under reduced pressure to obtain crude compound. The crude compound was purified by column chromatography over silica gel (100-200 mesh) using a solvent gradient of 5% EtOAC in Hexane as eluent to afford ethyl 1-(6-hexyl-4-phenylquinolin-2-yl)-5-oxopyrrolidine-3-carboxylate (95 mg, 78%) as a brown syrupy liquid. MS (ESI) m/z: 445.0 [M+H]⁺.

### Step 2: Synthesis of 1-(6-hexyl-4-phenylquinolin-2-yl)-5-oxopyrrolidine-3-carboxylic acid

To a stirred solution of ethyl 1-(6-hexyl-4-phenylquinolin-2-yl)-5-oxopyrrolidine-3-carboxylate (95 mg, 0.214 mmol) in THF:H₂O (15 mL, 2:1) was added LiOH·H₂O (36 mg, 0.856 mmol) and stirred at room temperature for 16 h. After completion of the reaction (monitored by T.L.C, Eluent: 20% EtOAc/Hexane, R.f: 0.20), it was diluted with water (10 mL) and acidified with 1N HCl (pH˜5-6) and extracted with 10% MeOH:DCM (3×10 mL). The organic layer was dried over Na₂SO₄, filtered and concentrated under reduced pressure to obtain crude compound (127 mg). The crude compound was purified by Prep-HPLC and isolated 1-(6-hexyl-4-phenylquinolin-2-yl)-5-oxopyrrolidine-3-carboxylic acid (11 mg, 11.8%) as off-white solid. ¹H-NMR (400 MHz, MeOD): δ 8.39 (s, 1H), 7.92-7.90 (m, 1H), 7.59-7.51 (m, 7H), 4.52-4.48 (m, 2H), 3.49-3.40 (m, 1H), 2.99-2.97 (m, 2H), 2.70 (t, J=15.20 Hz, 2H), 1.69-1.59 (m, 2H), 1.32-1.27 (m, 6H), 0.87 (t, J=14.00 Hz, 3H), MS (ESI) m/z: 417.0 [M+H]⁺.

### Example #14 Synthesis of 1-(6-hexyl-4-phenylquinolin-2-yl)-1H-pyrrole-3-carboxylic acid

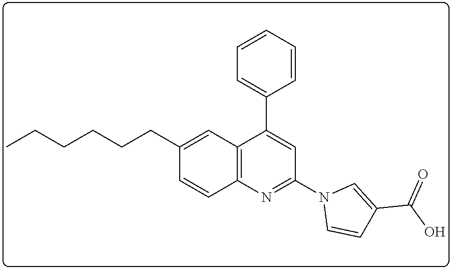 [SMILES: CCCCCCc1ccc2nc(-n3ccc(C(=O)O)c3)cc(-c3ccccc3)c2c1]

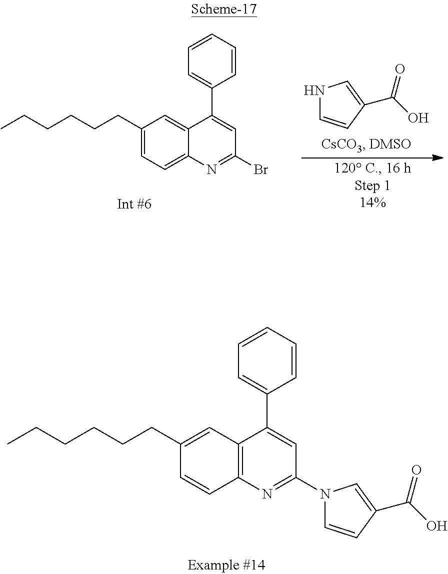 [SMILES: CCCCCCc1ccc2nc(Br)cc(-c3ccccc3)c2c1 ; O=C(O)c1cc[nH]c1 ; CCCCCCc1ccc2nc(-n3ccc(C(=O)O)c3)cc(-c3ccccc3)c2c1]

### Step 1: Synthesis of 1-(6-hexyl-4-phenylquinolin-2-yl)-1H-pyrrole-3-carboxylic acid

To a stirred solution of Int #6 (100 mg, 0.272 mmol) in DMSO (5 mL) was added 1H-pyrrole-3-carboxylic acid (60 mg, 0.540 mmol) followed by Cs₂CO₃ (176 mg, 0.541 mmol). The reaction mixture was heated to 120° C. for 16 h. After completion of the reaction (monitored by T.L.C, Eluent: 10% MeOH/DCM, R.f: 0.30), it was cooled to room temperature and diluted with water and extracted with 10% MeOH:CHCl₃ (2×10 mL). The organic layer was separated and dried over Na₂SO₄, filtered and concentrated under reduced pressure to obtain crude compound. The crude compound was purified by prep-HPLC and isolated ethyl 1-(6-hexyl-4-phenylquinolin-2-yl)-5-oxopyrrolidine-3-carboxylate (15 mg, 14%) as an off-White solid. ¹H NMR (400 MHz, DMSO-d6): δ 12.20 (bs, 1H), 8.49 (s, 1H), 7.96-8.00 (m, 3H), 7.70 (dd, J=1.60, 8.60 Hz, 1H), 7.66-7.58 (m, 6H), 6.65 (dd, J=1.60, 3.20 Hz, 1H), 2.72-2.71 (m, 2H), 1.60-1.57 (m, 2H), 1.30-1.20 (m, 6H), 0.83 (t, J=6.80 Hz, 3H), MS (ESI) m/z: 399 [M+H]⁺

### Example #15 Synthesis of (1R,2S)-2-(6-hexyl-4-phenylquinolin-2-yl) cyclopropane-1-carboxylic acid

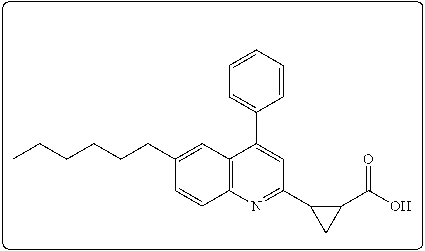 [SMILES: CCCCCCc1ccc2nc(C3CC3C(=O)O)cc(-c3ccccc3)c2c1]

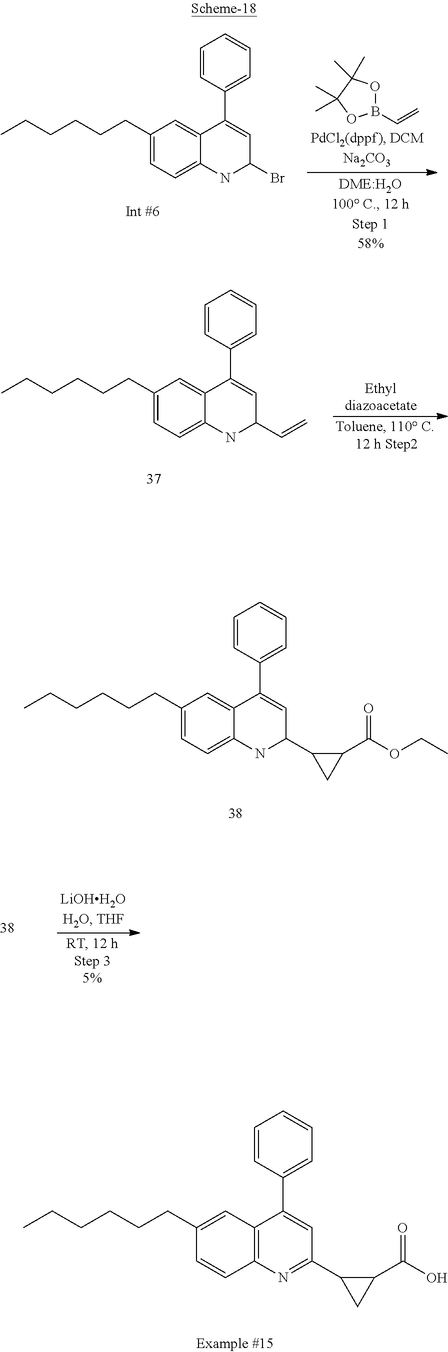

### Step 1: Synthesis of 6-hexyl-4-phenyl-2-vinylquinoline

A sealed tube was charged with 2-bromo-6-hexyl-4-phenylquinoline Int #6 (1 g, 2.715 mmol), 4,4,5,5-tetramethyl-2-vinyl-1,3,2-dioxaborolane (0.501 g, 3.258 mmol) and sodium carbonate (0.862 g, 8.145 mmol) in DME (8 mL):H₂O (8 mL). The reaction mixture was bubbled with nitrogen for 10 minutes and Pd (dppf)Cl₂·DCM (0.011 g, 0.135 mmol) was added to the reaction mixture, the sealed tube was tightened the cap and heated at 100° C. for 12 h. After completion of reaction (monitored by T.L.C, Eluent: 10% EtOAc/Hexane, R.f: 0.5), the reaction mixture was cooled to room temperature and then water (30 mL) was added to the reaction mixture followed by extraction with EtOAc (3×50 mL). The combined organic layer was dried over Na₂SO₄, filtered and concentrated under reduced pressure to afford to crude material. The obtained crude material was purified by column chromatography over silica gel (100-200 mesh) using a solvent gradient of 2-3% EtOAc in hexane as eluent to afford (6-hexyl-4-phenyl-2-vinylquinoline (500 mg, 58%) as a pale red liquid. MS (ESI) M/z: 316 [M+H]⁺

### Step 2: Synthesis of ethyl 2-(6-hexyl-4-phenylquinolin-2-yl) cyclopropane-1-carboxylate

To a stirred solution of (6-hexyl-4-phenyl-2-vinylquinoline (500 mg 1.584 mol) in toluene (8 mL) at room temperature, was added ethyl diazoacetate (0.25 mL g, 2.377 mmol) under nitrogen atmosphere and the reaction mixture was heated to 110° C. for 12 h. After completion of the reaction (monitored by T.L.C, Eluent: 10% EtOAc/Hexane, R.f: 0.7), the reaction mixture was cooled to room temperature, ice-cold water (20 mL) was added to the reaction mixture and the product was extracted with EtOAc (3×50 mL). The combined organic layers were dried over Na₂SO₄, filtered and concentrated under reduced pressure to afford ethyl (1R,2S)-2-(6-hexyl-4-phenylquinolin-2-yl) cyclopropane-1-carboxylate (700 mg, Crude) as grey color liquid. The Crude compound was taken forward to next step without purification. MS (ESI) m/z: 402 [M+H]⁺

### Step 3: Synthesis of 2-(6-hexyl-4-phenylquinolin-2-yl) cyclopropane-1-carboxylic acid

The process of this step was obtained from Step-2 of Example #1 and the desired compound obtained as (35 mg, 5%) brown color solid. ¹H-NMR (400 MHz, DMSO-d6): δ 8.08 (d, J=8.40 Hz, 1H), 7.83 (d, J=8.80 Hz, 1H), 7.77 (s, 1H), 7.67 (s, 1H), 7.63-7.57 (m, 5H), 3.03 (q, J=8.40 Hz, 1H), 2.78 (t, J=8.00 Hz, 2H), 2.43 (q, J=8.40 Hz, 1H), 2.02-1.98 (m, 1H), 1.77-1.72 (m, 1H), 1.67-1.61 (m, 2H), 1.39-1.25 (m, 6H), 0.89-0.85 (m, 3H), MS (ESI) m/z: 374.5 [M+H]⁺

### Example #16 Synthesis of N-(6-hexyl-4-(m-tolyl)quinolin-2-yl)-N-methylglycine

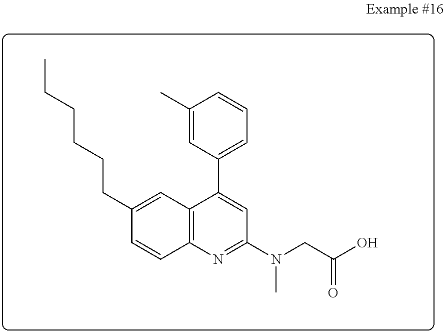 [SMILES: CCCCCCc1ccc2nc(N(C)CC(=O)O)cc(-c3cccc(C)c3)c2c1]

The process of this step was adopted from step-2 of Example #1. The desired compound was obtained as off-white solid (10.6 mg, 10%). ¹H-NMR (400 MHz, DMSO-d₆): δ 12.58 (s, 1H), 7.54-7.52 (m, 1H), 7.46-7.42 (m, 1H), 7.40-7.40 (m, 1H), 7.38-7.27 (m, 4H), 6.88 (s, 1H), 4.40 (s, 2H), 3.19 (s, 3H), 2.68-2.60 (m, 2H), 2.41 (s, 3H), 1.54-1.51 (m, 2H), 1.29-1.20 (m, 6H), 0.82 (t, J=13.20 Hz, 3H), MS (ESI) m/z: 391 [M+H]⁺.

### Example #17 Synthesis of N-methyl-6-phenethyl-4-phenyl-N-(1H-tetrazol-5-yl) quinolin-2-amine

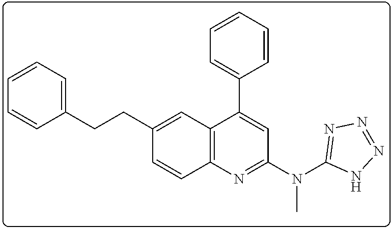 [SMILES: CN(c1cc(-c2ccccc2)c2cc(CCc3ccccc3)ccc2n1)c1nnn[nH]1]

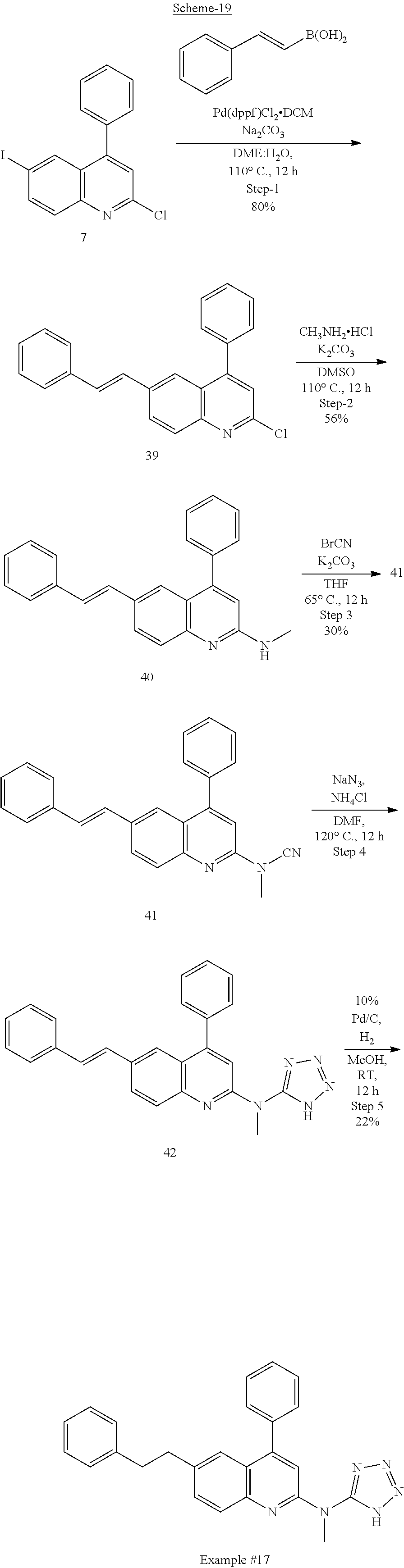

### Step 1: Synthesis of (E)-2-chloro-4-phenyl-6-styrylquinoline

The process of this step was obtained from step-1 of Example #1 and obtained the title compound (1.5 g, 80%) as a white solid. MS (ESI) m/z: 342 [M+H]⁺

### Step 2: Synthesis of (E)-N-methyl-4-phenyl-6-styrylquinolin-2-amine

To a stirred solution of (E)-2-chloro-4-phenyl-6-styrylquinoline (0.45 g, 1.31 mmol) in DMSO (5 mL) at room temperature was added potassium carbonate (910 mg, 6.598 mmol) and methyl amine hydrochloride (445 mg, 6.598 mmol). The reaction mixture was heated at 110° C. for 12 h. After completion of the reaction (monitored by TLC, Eluent: 50% EtOAc/Hexane, R.f: 0.3), the reaction mixture was cooled to room temperature, ice-cold water (30 mL) was added and the product was extracted with EtOAc (3×30 mL). The combined organic layers were dried over Na₂SO₄, filtered and concentrated under reduced pressure to afford crude compound. The crude compound was purified by column chromatography over silica gel (100-200 mesh) using a solvent gradient of 30-40% EtOAC in hexane as eluent to afford (E)-N-methyl-4-phenyl-6-styrylquinolin-2-amine (250 mg, 56%) as off-green solid. MS (ESI) m/z: 337 [M+H]⁺.

### Step 3: Synthesis of (E)-N-methyl-N-(4-phenyl-6-styrylquinolin-2-yl) cyanamide

To a stirred solution of (E)-N-methyl-4-phenyl-6-styrylquinolin-2-amine (250 mg, 0.744 mmol) in THF (5 mL) at room temperature was added potassium carbonate (308 mg, 2.232 mmol) and cyanogen bromide (157.5 mg 1.488 mmol). The reaction mixture was stirred at 65° C. for 12 h. After completion of reaction (monitored by TLC, Eluent: 50% EtOAc/Hexane, R.f: 0.80), the reaction mixture was cooled to room temperature, ice-cold water (20 mL) was added to the reaction mixture and the product was extracted with EtOAc (3×20 mL). The combined organic layers were dried over Na₂SO₄, filtered and concentrated under reduced pressure to afford crude material. The crude compound was purified by column chromatography over silica gel (100-200 mesh) using a solvent gradient of 6-8% EtOAc in hexane as eluent to afford (E)-N-methyl-N-(4-phenyl-6-styrylquinolin-2-yl) cyanamide (80 mg, 30%) as a white solid. MS (ESI) m/z: 362 [M+H]⁺.

### Step 4: Synthesis of (E)-N-methyl-4-phenyl-6-styryl-N-(1H-tetrazol-5-yl) quinolin-2-amine

To a stirred solution of (E)-N-methyl-4-phenyl-6-styryl-N-(1H-tetrazol-5-yl) quinolin-2-amine (80 mg, 0.222 mmol) in DMF (2 mL) at room temperature was added ammonium chloride (23.7 mg, 0.443 mmol) and Sodium azide (28.8 mg, 0.443 mmol). The reaction mixture was heated at 120° C. for 12 h. After completion of reaction (monitored by T.L.C, Eluent: 100% EtOAc/Hexane, R.f: 0.60), the reaction mixture was cooled to room temperature, ice-cold water (10 mL) was added to the reaction mixture and the product was extracted with EtOAc (3×10 mL). The combined organic layers were dried over Na₂SO₄, filtered and concentrated under reduced pressure to afford (E)-N-methyl-4-phenyl-6-styryl-N-(1H-tetrazol-5-yl) quinolin-2-amine (80 mg) as a pale brown liquid and taken forward for the next step. MS (ESI) m/z: 405 [M+H]⁺.

### Step 5: Synthesis of N-methyl-6-phenethyl-4-phenyl-N-(1H-tetrazol-5-yl) quinolin-2-amine

The process of this step was adopted from step-1 of Example #2 and the title compound was obtained as pale pink solid (18 mg, 22%). ¹H NMR (400 MHz, DMSO-d6): δ 15.72 (s, 1H), 8.27 (d, J=8.00 Hz, 1H), 7.67 (d, J=8.40 Hz, 1H), 7.55-7.51 (m, 4H), 7.44-7.42 (m, 2H), 7.37 (s, 1H), 7.26-7.23 (m, 2H), 7.20-7.18 (m, 1H), 7.13 (d, J=7.20 Hz, 2H), 3.82 (s, 3H), 2.99-2.97 (m, 2H), 2.92-2.90 (m, 2H), MS (ESI) m/z: 405 (M−H)⁻.

### Example #18: Synthesis of N-methyl-N-(4-phenyl-6-(3-propylphenyl) quinolin-2-yl) glycine

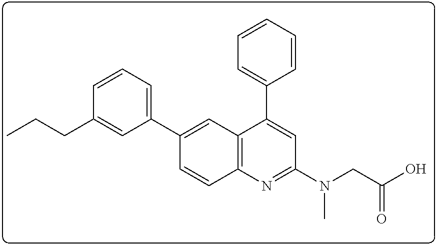 [SMILES: CCCc1cccc(-c2ccc3nc(N(C)CC(=O)O)cc(-c4ccccc4)c3c2)c1]

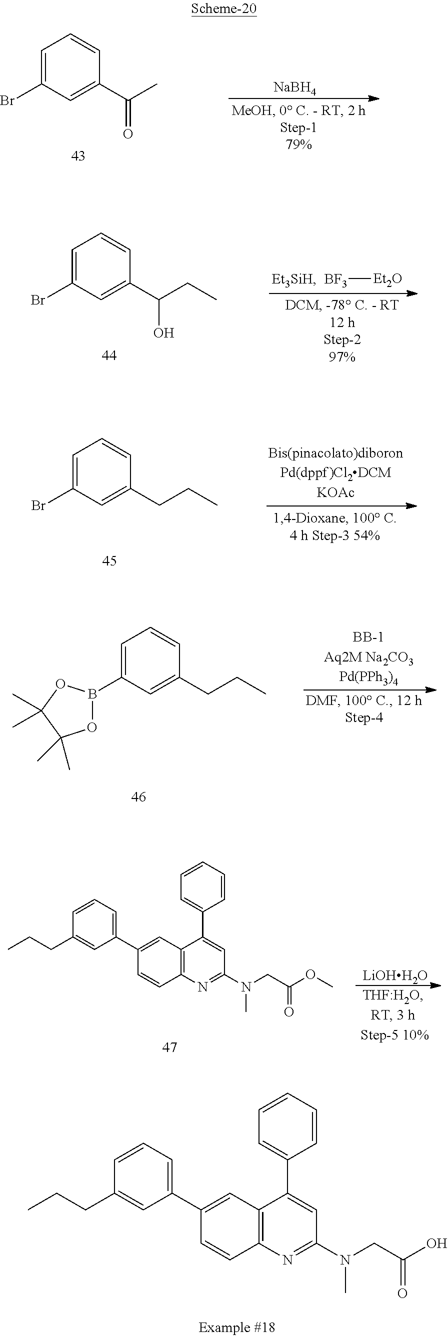

### Step 1: Synthesis of 1-(3-bromophenyl) propan-1-ol

To a stirred solution of 1-(3-bromophenyl) propan-1-one (2.5 g, 11.733 mmol) in MeOH (25 mL) at 0° C., was added NaBH₄ (0.488 g, 12.906 mmol), the reaction mixture was slowly warmed to room temperature and stirred at RT for 2 h. After completion of the reaction (monitored by TLC, Eluent: 10% EtOAc/Hexane, R.f: 0.3), the reaction mixture was cooled to room temperature, diluted with ice cold water (30 mL) and the product was extracted with ethyl acetate (2×50 mL). The combined organic layer was washed with brine (30 mL) and dried over Na₂SO₄, filtered and concentrated under reduced pressure to obtain crude compound. The crude compound was purified by column chromatography over silica gel (100-200 mesh) using a solvent gradient of 15-20% EtOAc in hexane as eluent to afford 1-(3-bromophenyl) propan-1-ol (2 g, 79%) as colorless liquid. ¹H-NMR (400 MHz, DMSO-d6): δ 7.51 (t, J=2.00 Hz, 1H), 7.40 (dt, J=2.00, 7.60 Hz, 1H), 7.26-7.22 (m, 2H), 4.62-4.55 (m, 1H), 1.86 (d, J=3.60 Hz, 1H), 1.82-1.72 (m, 2H), 0.92 (t, J=7.60 Hz, 3H).

### Step 2: Synthesis of 1-bromo-3-propylbenzene

To a stirred solution of 1-(3-bromophenyl) propan-1-ol (2.0 g, 9.345 mmol) in DCM (20 mL) at −78° C., were added Et₃SiH (22 mL, 140.175 mmol) and BF₃Et₂O (4.6 mL, 37.38 mmol). The reaction mixture was slowly warmed to room temperature and continued the stirring for a period of 12 h at room temperature. After completion of the reaction (monitored by T.L.C, Eluent: 10% EtOAc/Hexane, R.f: 0.6), the reaction mixture was poured onto ice-water (50 mL) and the product was extracted with DCM (2×75 mL), washed with brine (20 mL). The combined organic layer was dried over Na₂SO₄ filtered and concentrated under reduced pressure afforded 1-bromo-3-propylbenzene Int-02 (1.8 g, 97%) as Pale green liquid. ¹H-NMR (400 MHz, DMSO-d6): δ 7.33-7.29 (m, 2H), 7.16-7.08 (m, 2H), 2.55 (t, J=7.60 Hz, 2H), 1.65-1.58 (m, 2H), 0.95 (t, J=7.20 Hz, 3H).

### Step 3: Synthesis of 4,4,5,5-tetramethyl-2-(3-propylphenyl)-1,3,2-dioxaborolane

In a sealed tube was charged 1-bromo-3-propylbenzene (0.6 g, 3.030 mmol), bis(pinacolato)diboron (1 g, 3.930 mmol), potassium acetate (0.594 g, 6.060 mmol) and dppf (0.168 g, 0.303 mmol) in 1,4-dioxane (10 mL). The reaction mixture was bubbled with nitrogen for 10 minutes, Pd(dppf)Cl₂·DCM (0.123 g, 0.151 mmol) was added to the reaction mixture the sealed tube was heated at 100° C. for 4 h. After completion of the reaction (monitored by T.L.C, Eluent: 10% EtOAc/Hexane, R.f: 0.4), the reaction mixture was cooled to room temperature and filtered through celite pad, the celite pad was washed with EtOAc (150 mL) and concentrated under reduced pressure afforded a crude material. The obtained crude material was purified by column chromatography over silica gel (100-200 mesh) using a solvent gradient of 15-20% EtOAc in Hexane as eluent to afford 4,4,5,5-tetramethyl-2-(3-propylphenyl)-1,3,2-dioxaborolane (400 mg, 54%) as pale yellow liquid. ¹H-NMR (400 MHz, DMSO-d6): δ 7.64-7.62 (m, 2H), 7.29-7.28 (m, 2H), 2.61-2.57 (m, 2H), 1.68-1.59 (m, 2H), 1.35 (s, 12H), 0.94 (t, J=3.60 Hz, 3H).

### Step 4: Synthesis methyl N-methyl-N-(4-phenyl-6-(3-propylphenyl) quinolin-2-yl) glycinate

A sealed tube was charged with methyl N-(6-iodo-4-phenylquinolin-2-yl)-N-methylglycinate Int #3 (0.235 g, 0.543 mmol), 4,4,5,5-tetramethyl-2-(3-propylphenyl)-1,3,2-dioxaborolane (0.2 g, 0.815 mmol), tetrakis(triphenylphosphine)palladium(0) (0.031 g, 0.027 mmol), Aqueous 2M Na₂CO₃ (0.543 mL, 1.086 mmol) and DMF (6 mL). The reaction mixture was degassed by bubbling nitrogen for 15 min and heated the sealed tube at 100° C. for a period of 12 h. After completion of the reaction (monitored by T.L.C, Eluent: 10% EtOAc/Hexane, R.f: 0.3). The reaction mixture was cooled to room temperature and filtered through a celite pad and washed with EtOAc (50 mL). The organic layer was washed with water (20 mL), dried over Na₂SO₄ and concentrated under reduced pressure to afford a crude product. The obtained crude material was purified by column chromatography over silica gel (100-200 mesh) using a solvent gradient of 15-20% EtOAc in hexane as eluent to afford methyl N-methyl-N-(4-phenyl-6-(3-propylphenyl)quinolin-2-yl)glycinate (90 mg) as orange colour liquid and carried out for the next step without purification. MS (ESI) m/z: 425 [M+H]⁺

### Step 5: Synthesis of N-methyl-N-(4-phenyl-6-(3-propylphenyl) quinolin-2-yl) glycine

To a stirred solution of methyl N-methyl-N-(4-phenyl-6-(3-propylphenyl) quinolin-2-yl) glycinate (90 mg, 0.212 mmol) in THF (4 mL) and water (2 mL) at room temperature, was added LiOH·H₂O (10 mg, 0.254 mmol) and the reaction mixture was stirred at room temperature for 3 h. After completion of the reaction (monitored by T.L.C, Eluent: 10% MeOH:DCM, R.f: 0.4), the solvents were evaporated and the obtained residue was acidified with 1N HCl and excess of water was evaporated under reduced vacuum to obtain 90 mg of crude compound. Crude compound was purified by Prep HPLC, the relevant fractions containing the product were combined and kept for Lyophilization to afford N-methyl-N-(4-phenyl-6-(3-propylphenyl) quinolin-2-yl) glycine (18 mg, 10%) as pale yellow solid. ¹H-NMR (400 MHz, DMSO-d6): δ 7.86 (dd, J=2.00, 8.60 Hz, 1H), 7.75 (d, J=2.00 Hz, 1H), 7.70 (d, J=8.80 Hz, 1H), 7.60-7.53 (m, 5H), 7.39-7.36 (m, 1H), 7.34-7.33 (m, 2H), 7.72-7.32 (m, 1H), 6.99 (s, 1H), 4.45 (s, 2H), 3.23 (s, 3H), 2.59-2.61 (m, 2H), 1.63-1.57 (m, 2H), 0.89 (t, J=7.20 Hz, 3H), MS (ESI) M/z: 411 [M+H]⁺.

### Example #19 Synthesis of N-methyl-N-(6-(2-morpholinoethyl)-4-phenylquinolin-2-yl) glycine

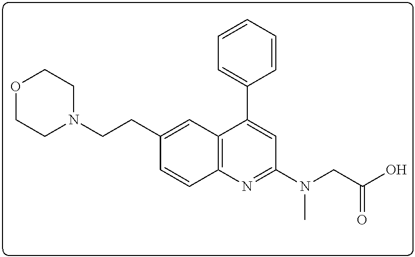 [SMILES: CN(CC(=O)O)c1cc(-c2ccccc2)c2cc(CCN3CCOCC3)ccc2n1]

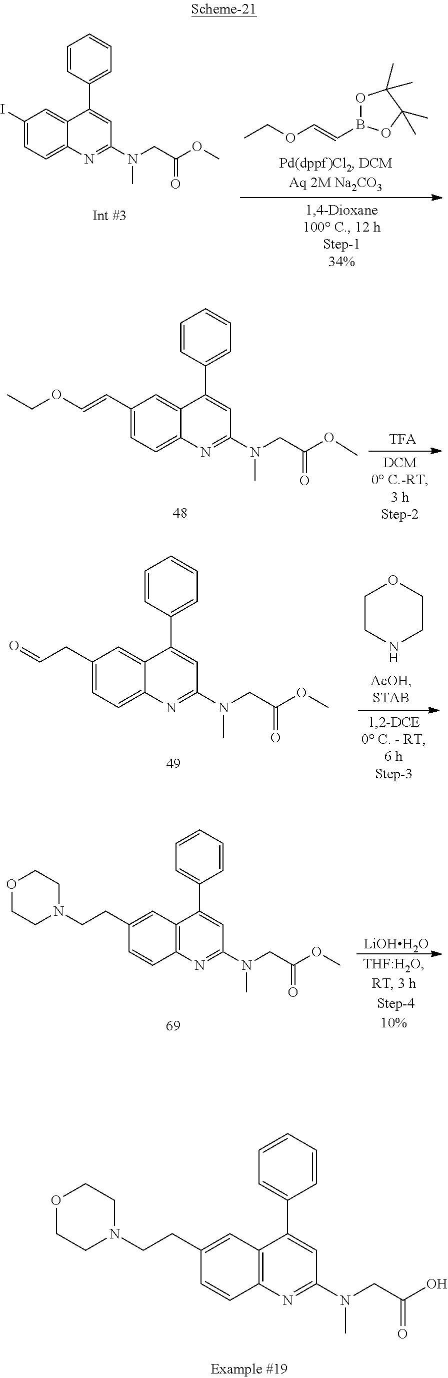

### Step 1: Synthesis of methyl (E)-N-(6-(2-ethoxyvinyl)-4-phenylquinolin-2-yl)-N-methylglycinate

A sealed tube was charged with methyl N-(6-iodo-4-phenylquinolin-2-yl)-N-methylglycinate Int #3 (1 g, 2.314 mmol), (E)-2-(2-ethoxyvinyl)-4,4,5,5-tetramethyl-1,3,2-dioxaborolane (0.687 g, 3.471 mmol) and Aq 2M Sodium carbonate solution (2.314 mL, 4.628 mmol) in 1,4-dioxane (10 mL). The reaction mixture was bubbled with nitrogen for 10 minutes and then Pd(dppf)Cl₂·DCM (0.094 g, 0.115 mmol) was added to the reaction mixture, the sealed tube was tightened and heated at 100° C. for 12 h. After completion of the reaction (monitored by T.L.C, Eluent: 25% EtOAc/Hexane, R.f: 0.5), the reaction mixture was cooled to room temperature and filtered through celite pad, the celite pad was washed with EtOAc (150 mL) and concentrated under reduced pressure afforded a crude material. The obtained crude material was purified by column chromatography over silica gel (100-200 mesh) using a solvent gradient of 10-15% EtOAc in Hexane as eluent to afford methyl (E)-N-(6-(2-ethoxyvinyl)-4-phenylquinolin-2-yl)-N-methylglycinate (300 mg, 34%) as off-white solid. MS (ESI) m/z: 377 [M+H]⁺

### Step 2: Synthesis of methyl N-methyl-N-(6-(2-oxoethyl)-4-phenylquinolin-2-yl) glycinate

To a stirred solution of (E)-N-(6-(2-ethoxyvinyl)-4-phenylquinolin-2-yl)-N-methylglycinate (0.3 g, 0.797 mmol) in DCM (5 mL) at 0° C., was added TFA (0.3 ml) under nitrogen atmosphere and the reaction mixture was stirred at room temperature for 3 h. After completion of the reaction (monitored by T.L.C, Eluent: 25% EtOAc/Hexane, R.f: 0.3), the reaction mixture was basified with Aqueous Na₂CO₃ solution, extracted with DCM (3×30 mL) and washed with brine (10 mL). The combined organic layer was dried over Na₂SO₄, filtered and concentrated under reduced pressure to obtain methyl N-methyl-N-(6-(2-oxoethyl)-4-phenylquinolin-2-yl) glycinate (200 mg) as brown solid. The material was taken forward for the next step without purification. ¹H-NMR (400 MHz, DMSO-d6): δ 9.71 (t, J=2.40 Hz, 1H), 7.74 (d, J=8.40 Hz, 1H), 7.54-7.44 (m, 6H), 7.36 (dd, J=2.00, 8.40 Hz, 1H), 6.86 (s, 1H), 4.55 (s, 2H), 3.76 (s, 3H), 3.69 (d, J=2.40 Hz, 2H), 3.26 (s, 3H).

### Step 3: Synthesis of methyl N-methyl-N-(6-(2-morpholinoethyl)-4-phenylquinolin-2-yl) glycinate

To a stirred solution of methyl N-methyl-N-(6-(2-oxoethyl)-4-phenylquinolin-2-yl)glycinate (200 mg, 0.531 mmol) in 1,2-DCE (8 mL) at 0° C. were added morpholine (80 mg, 0.797 mmol and acetic acid (cat. amount) and stirred the reaction mixture at room temperature for 1 h. After 1 h, sodium triacetoxyborohydride (169 mg, 0.797 mmol) was added and stirred the reaction mixture at room temperature for 5 h (monitored by T.L.C, Eluent: 50% EtOAc/Hexane, R.f: 0.1). After completion of reaction, the reaction mixture was basified with aqueous Na₂CO₃ solution and extracted with DCM (3×20 mL), washed with brine (10 mL). The combined organic layer was dried over Na₂SO₄, filtered and concentrated under reduced pressure to obtain crude compound methyl N-methyl-N-(6-(2-morpholinoethyl)-4-phenylquinolin-2-yl) glycinate (200 mg) as brown liquid. The material was taken forward for the next step without purification. MS (ESI) m/z: 420 [M+H]⁺

### Step 4: Synthesis of N-methyl-N-(6-(2-morpholinoethyl)-4-phenylquinolin-2-yl) glycine

To a stirred solution of methyl N-methyl-N-(6-(2-morpholinoethyl)-4-phenylquinolin-2-yl) glycinate (250 mg, 0.595 mmol) in THF (4 mL) and water (2 mL) at room temperature, was added LiOH·H₂O (30 mg, 0.715 mmol) and the reaction mixture was stirred at room temperature for 3 h. After completion of the reaction (monitored by T.L.C, Eluent: 10% MeOH:DCM, R.f: 0.4), the solvents were evaporated and the obtained residue was acidified with 1N HCl and excess of water was evaporated under reduced vacuum to obtain 250 mg of crude compound. Crude compound was purified by Prep HPLC, and isolated to afford N-methyl-N-(6-(2-morpholino ethyl)-4-phenylquinolin-2-yl) glycine (18 mg, 10%) as off-white solid. ¹H-NMR (400 MHz, DMSO-d6): δ 7.59-7.50 (m, 6H), 7.44 (d, J=2.00 Hz, 1H), 7.41 (s, 1H), 6.91 (s, 1H), 4.41 (s, 2H), 3.52 (t, J=4.40 Hz, 4H), 3.19 (s, 3H), 2.74 (t, J=7.60 Hz, 2H), 2.45-2.46 (m, 2H), 2.33-2.36 (m, 4H), MS (ESI) m/z: 406.4 [M+H]⁺

### Example #20 Synthesis of N-methyl-N-(6-(2-(pyridin-2-yl) ethyl)-4-(pyridin-4-yl) quinolin-2-yl) glycine

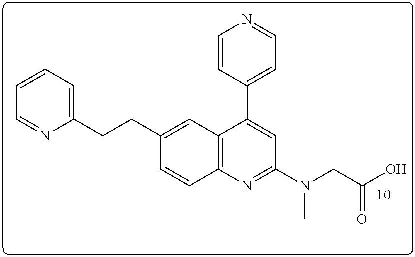 [SMILES: CN(CC(=O)O)c1cc(-c2ccncc2)c2cc(CCc3ccccn3)ccc2n1]

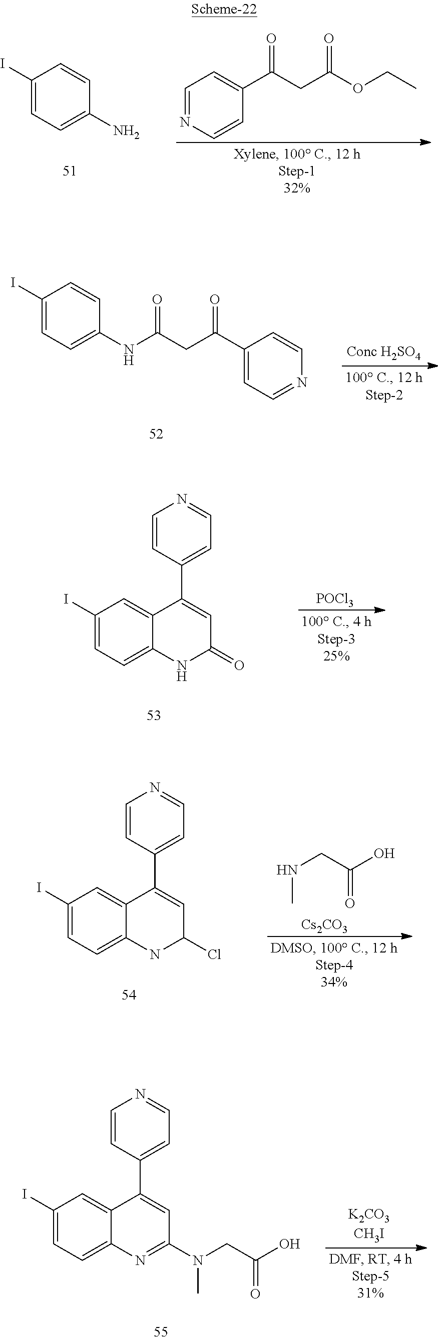

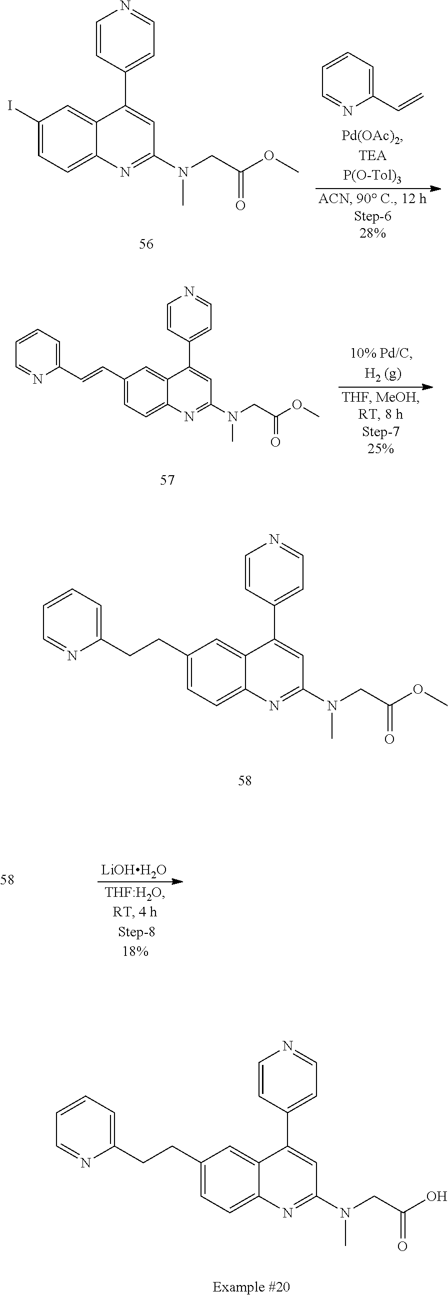

### Step 1: Synthesis of N-(4-iodophenyl)-3-oxo-3-(pyridin-4-yl) propenamide

To a stirred solution of 4-iodoaniline (5 g, 22.828 mmol) in Xylene (100 mL) at room temperature was added ethyl 3-oxo-3-(pyridin-4-yl)propanoate (6.61 g, 34.242 mmol) and the reaction mixture was heated at 100° C. for 12 h. After completion of the reaction (monitored by T.L.C, Eluent: 50% EtOAc/Hexane, R.f: 0.3), the reaction mixture was cooled to room temperature and the solvent was concentrated under reduced pressure afforded crude material. The obtained crude material was purified by column chromatography over silica gel (100-200 mesh) using a solvent gradient of 25-30% EtOAc in Hexane as eluent to afford N-(4-iodophenyl)-3-oxo-3-(pyridin-4-yl) propenamide (2.7 g, 32%) as Yellow solid. MS (ESI) m/z: 367 [M+H]⁺

### Step 2: Synthesis of 6-iodo-4-(pyridin-4-yl) quinolin-2(1H)-one

To the stirred solution of Conc H₂SO₄ (50 mL) at room temperature, was added N-(4-iodophenyl)-3-oxo-3-(pyridin-4-yl) propenamide (7 g, 19.125 mmol) and the reaction mixture was stirred at 100° C. for 12 h. After completion of the reaction (monitored by T.L.C, Eluent: 100% EtOAc/Hexane, R.f: 0.3), the reaction mixture was basified with Aqueous Na₂CO₃ solution and the precipitated product was filtered under vacuum to obtain 6-iodo-4-(pyridin-4-yl) quinolin-2(1H)-one Int-02 (3 g) as brown solid. The material was taken forward for the subsequent step MS (ESI) m/z: 349 [M+H]⁺

### Step 3: Synthesis of 2-chloro-6-iodo-4-(pyridin-4-yl) quinoline

To a stirred solution of POCl₃ (10 mL) at room temperature, was added 6-iodo-4-(pyridin-4-yl) quinolin-2(1H)-one (1.2 g, 3.44 mmol) and stirred the reaction mixture at 100° C. for 4 h. After completion of the reaction (monitored by T.L.C, Eluent: 25% EtOAc/Hexane, R.f: 0.5), the reaction mixture was basified with Aqueous Na₂CO₃ solution and the precipitated product was filtered under vacuum to obtain to obtain Crude material. The obtained crude material was purified by column chromatography over silica gel (100-200 mesh) using a solvent gradient of 10-15% EtOAc in hexane as an eluent to afford 2-chloro-6-iodo-4-(pyridin-4-yl) quinoline (300 mg, 25%) as Off-white solid. MS (ESI) m/z: 367 [M+H]⁺

### Step 4: Synthesis of N-(6-iodo-4-(pyridin-4-yl) quinolin-2-yl)-N-methyl glycine

To a stirred solution of 2-chloro-6-iodo-4-(pyridin-4-yl) quinoline (300 mg, 0.819 mmol) in DMSO (5 mL) at room temperature were added sarcosine (90 mg, 0.983 mmol) and Cs₂CO₃ (634 mg, 1.639 mmol), and the resultant reaction mixture was stirred at 100° C. for 12 h. After completion of the reaction (monitored by T.L.C, Eluent: 100% EtOAc: hexane, R.f: 0.3)). The reaction mixture was cooled to room temperature and acidified with 1N HCl and extracted with 10% MeOH/DCM (3×30 mL), washed with brine (10 mL). The combined organic layer was dried over Na₂SO₄, filtered and concentrated under reduced pressure to obtain N-(6-iodo-4-(pyridin-4-yl) quinolin-2-yl)-N-methyl glycine (300 mg, 34%) as pale green solid. M S (ESI) m/z: 420 [M+H]⁺

### Step 5: Synthesis of methyl N-(6-iodo-4-(pyridin-4-yl) quinolin-2-yl)-N-methyl glycinate

To a stirred solution of N-(6-iodo-4-(pyridin-4-yl) quinolin-2-yl)-N-methyl glycine (300 mg, 0.715 mmol) in DMF (5 mL) at room temperature, were added K₂CO₃ (194 mg, 1.431 mmol) and CH₃I (0.089 mL, 1.431 mmol) and the reaction mixture was stirred at room temperature for 4 h. After completion of the reaction (monitored by T.L.C, Eluent: 10% EtOAc/Hexane, R.f: 0.4), ice cold water (10 mL) was added to the reaction mixture and the precipitated compound was filtered under vacuum to afford methyl N-(6-iodo-4-(pyridin-4-yl) quinolin-2-yl)-N-methyl glycinate (200 mg, 31%) as pale blue solid. MS (ESI) m/z: 434 [M+H]⁺

### Step 6: Synthesis of methyl (E)-N-methyl-N-(6-(2-(pyridin-2-yl) vinyl)-4-(pyridin-4-yl) quinolin-2-yl) glycinate

A sealed tube was charged with methyl N-(6-iodo-4-(pyridin-4-yl)quinolin-2-yl)-N-methyl glycinate (300 mg, 0.692 mmol), 2-vinylpyridine (72.7 mg, 0.692 mmol), triethylamine (0.287 mL, 2.078 mmol) and Tris(o-tolyl)phosphine (63 mg, 0.207 mmol) in ACN (7 mL). The reaction mixture was bubbled with nitrogen for 10 minutes and finally Pd (OAc)₂ (15.5 mg, 0.069 mmol) was added to the reaction mixture and heated at 90° C. for 12 h. After completion of reaction (monitored by T.L.C, Eluent: 50% EtOAc/Hexane, R.f: 0.2), the reaction mixture was cooled to room temperature, added water (30 mL) and extracted with EtOAc (3×40 mL). Combined organic layer was dried over sodium sulphate and concentrated under reduced pressure afforded a crude material. The obtained crude material was purified by column chromatography over silica gel (100-200 mesh) using a solvent gradient of 60-70% EtOAc in Hexane as eluent to afford methyl (E)-N-methyl-N-(6-(2-(pyridin-2-yl) vinyl)-4-(pyridin-4-yl) quinolin-2-yl) glycinate (250 mg, 28%) as a Pale yellow solid. M S (ESI) m/z: 411.1 [M+H]⁺

### Step 7: Synthesis of methyl N-methyl-N-(6-(2-(pyridin-2-yl) ethyl)-4-(pyridin-4-yl) quinolin-2-yl) glycinate

To a stirred solution of (methyl (E)-N-methyl-N-(6-(2-(pyridin-2-yl) vinyl)-4-(pyridin-4-yl) quinolin-2-yl) glycinate (250 mg 0.609 mmol) in Methanol (5 mL) & THF (5 mL) at room temperature, was added 10% Pd/C (250 mg) under nitrogen atmosphere. The reaction mixture was stirred under hydrogen atmosphere for 8 h. After completion of reaction (monitored by T.L.C, Eluent: 80% EtOAc/Hexane, R.f: 0.3), the reaction mixture was filtered through celite pad, and washed the celite pad with methanol (100 mL) and the filtrate was distilled under reduced pressure to afford methyl N-methyl-N-(6-(2-(pyridin-2-yl) ethyl)-4-(pyridin-4-yl) quinolin-2-yl) glycinate (230 mg, 25%) as orange color liquid. MS (ESI) m/z: 413.2 [M+H]⁺

### Step 8: Synthesis of N-methyl-N-(6-(2-(pyridin-2-yl) ethyl)-4-(pyridin-4-yl) quinolin-2-yl) glycine

To a stirred solution of N-methyl-6-phenethyl-4-phenyl-N-(1H-tetrazol-5-yl) quinolin-2-amine (230 mg, 0.558 mmol) in THF (4 mL) and water (2 mL) at room temperature, was added LiOH·H₂O (47 mg, 1.116 mmol) and the reaction mixture was stirred at room temperature for 4 h. After completion of the reaction (monitored by T.L.C, Eluent: 10% MeOH:DCM, R.f: 0.4), the solvents were evaporated and the obtained residue was acidified with 1N HCl and excess of water was evaporated under reduced vacuum to obtain 250 mg of crude compound. Crude compound was purified by Prep HPL to afford N-methyl-N-(6-(2-(pyridin-2-yl) ethyl)-4-(pyridin-4-yl) quinolin-2-yl) glycine (40 mg, 18%) as off-white solid. ¹H NMR (400 MHz, DMSO-d6): δ 8.73 (dd, J=1.60, 4.00 Hz, 2H), 8.47-8.45 (m, 1H), 7.62 (td, J=1.60, 7.60 Hz, 1H), 7.54 (d, J=8.80 Hz, 1H), 7.45 (dd, J=2.00, 8.40 Hz, 1H), 7.39 (td, J=1.60, 4.40 Hz, 2H), 7.20-7.17 (m, 1H), 7.13-7.09 (m, 2H), 6.94 (s, 1H), 4.37 (s, 2H), 3.18 (s, 3H), 3.02-2.99 (m, 4H), MS (ESI) m/z: 399.4 [M+H]⁺

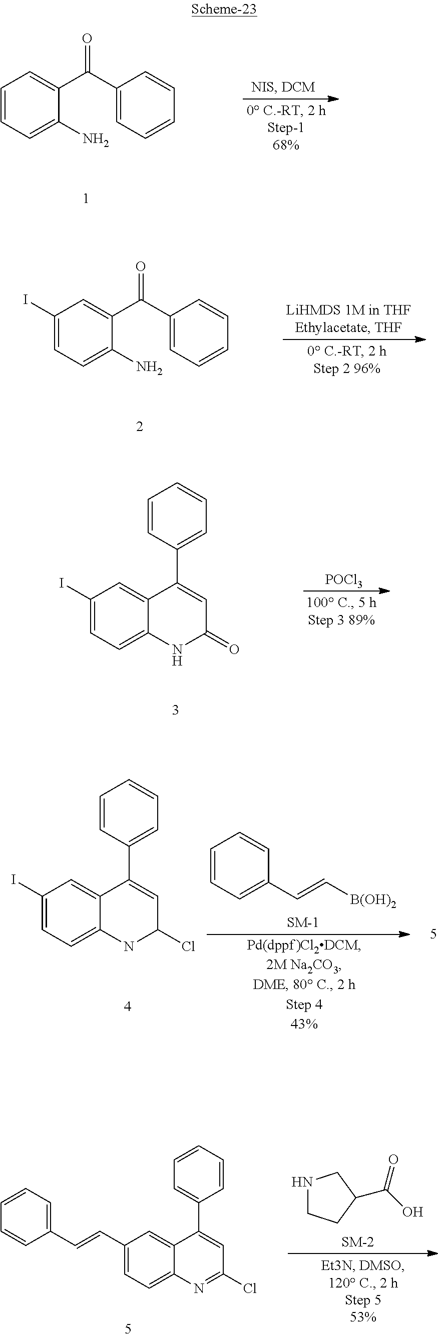

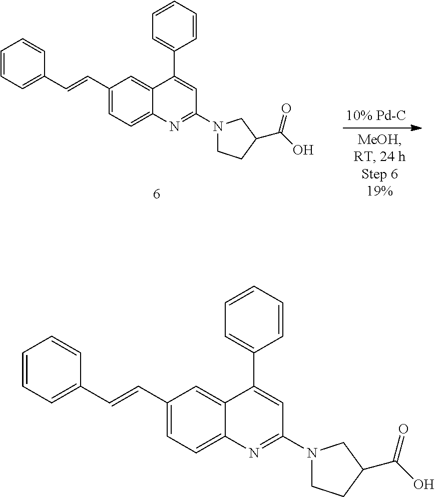 [SMILES: O=C(O)C1CCN(c2cc(-c3ccccc3)c3cc(/C=C/c4ccccc4)ccc3n2)C1 ; O=C(O)C1CCN(c2cc(-c3ccccc3)c3cc(/C=C/c4ccccc4)ccc3n2)C1]

### Example #1: Synthesis of 1-(6-phenethyl-4-phenylquinolin-2-yl)pyrrolidine-3-carboxylic acid

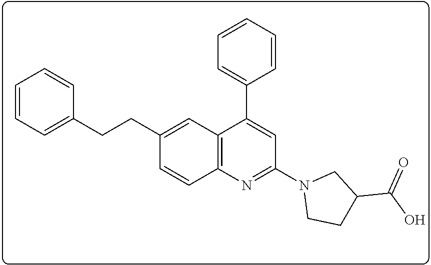 [SMILES: O=C(O)C1CCN(c2cc(-c3ccccc3)c3cc(CCc4ccccc4)ccc3n2)C1]

### Step 1: Synthesis of (2-amino-5-iodophenyl) (Phenyl)methanone

To a stirred solution of (2-aminophenyl) (phenyl)methanone (25 g, 0.126 mol) in DCM (250 mL) was added N-iodosuccinimide portion wise at 0° C. The resultant reaction mixture was warmed to room temperature and stirred for a period of 2 h. After completion of reaction (monitored by TLC, Eluent: 10% EtOAc/Hexane, R f; 0.3), the reaction mixture was poured into ice-water (100 mL) and extracted with EtOAc (2×200 mL), washed with brine (50 mL). The combined organic layer was dried over Na₂SO₄, filtered and concentrated under reduced pressure to afford a crude material. The crude obtained was purified by column chromatography over silica gel (100-200 mesh) using a solvent gradient of 15-20% EtOAc in hexane as an eluent to afford (2-amino-5-iodophenyl) (Phenyl)methanone (28 g, 68%). MS (ESI) m/z: 324 [M+H]⁺

### Step 2: Synthesis of 6-iodo-4-phenylquinolin-2(1H)-one

To a stirred solution of (2-amino-5-iodophenyl) (Phenyl)methanone (28 g, 0.086 mol) in THF (280 mL) was added LiHMDS (520 mL, 0.519 mol, 1M in THF) at 0° C. and the reaction mixture was stirred for a period of 0.5 h. Ethyl acetate (28 mL, 1 Vol) was added and the reaction mixture was warmed to room temperature and stirred for a period of 2 h. After completion of reaction (monitored by TLC, Eluent: 30% EtOAc/Hexane, R.f; 0.15), the reaction mixture was quenched with sat. NH₄Cl solution (100 mL). The precipitated solid was collected by filtration and dried under Vacuum to afford 6-iodo-4-phenylquinolin-2(1H)-one (29 g, 96%). MS (ESI) m/z: 348 [M+H]⁺

### Step 3: Synthesis of 2-chloro-6-iodo-4-Phenylquinoline

To a stirred solution of 6-iodo-4-phenylquinolin-2(1H)-one (29 g, 0.083 mol) in POCl₃ (200 mL) was added N, N-dimethyl aniline (29 mL, 1 Vol) and heated to 110° C. for a period of 4 h. After completion of reaction (monitored by TLC, Eluent: 10% EtOAc/Hexane, R.f; 0.7), the reaction mixture was concentrated under reduced pressure. The resulting residue was poured on to ice-water (100 mL), and the precipitated solid was collected by filtration and dried under vacuum to afford 2-chloro-6-iodo-4-Phenylquinoline (27.3 g, 89.5%). MS (ESI) m/z: 366 [M+H]⁺

### Step 4: Synthesis of (E)-2-chloro-4-phenyl-6-styrylquinoline

To a solution of 2-chloro-6-iodo-4-phenylquinoline (500 mg, 1.36 mmol) and DME (5 mL) in a sealed tube was added (E)-styryl boronic acid (243 mg, 1.64 mmol), 2M solution of sodium carbonate (360 mg, 3.4 mmol) (degassed under nitrogen atmosphere for 10 mins), and Pd (dppf)Cl₂. DCM adduct (55 mg, 0.068 mmol) and heated the reaction mixture at 80° C. for 2 h. After completion of the reaction (monitored by T.L.C, Eluent: 10% EtOAc: Hexane, R.f: 0.5), the reaction mixture was cooled to room temperature, diluted the reaction mixture with water (10 mL) and extracted with ethyl acetate (2×15 mL). The combined organic layer was dried over sodium sulphate, filtered and concentrated under reduced pressure to obtain crude product. The crude compound was purified by column chromatography over silica gel using a solvent gradient of 10% EtOAC in Hexane as eluent to afford (E)-2-chloro-4-phenyl-6-styrylquinoline (200 mg, 43%). MS (ESI) m/z: 342 [M+H]⁺

### Step 5: Synthesis of (E)-1-(4-phenyl-6-styrylquinolin-2-yl)pyrrolidine-3-carboxylic acid

To a solution of (E)-2-chloro-4-phenyl-6-styrylquinoline (200 mg, 0.586 mmol) and DMSO (5 mL) in a sealed tube was added triethyl amine (0.25 ml, 1.76 mmol) and pyrrolidine-3-carboxylic acid (101 mg, 0.879 mmol). The reaction mixture was heated at 120° C. for 2 h. After completion of the reaction (monitored by T.L.C, Eluent: 10% MeOH:DCM, R.f: 0.4), the reaction mixture was cooled to room temperature, diluted the reaction mixture with water (10 mL) and extracted with ethyl acetate (2×15 mL). The combined organic layer was dried over sodium sulphate, filtered and concentrated under reduced pressure to obtain (E)-1-(4-phenyl-6-styrylquinolin-2-yl)pyrrolidine-3-carboxylic acid (130 mg, 53%). MS (ESI) m/z: 421.0 [M+H]⁺

### Step 6: Synthesis of 1-(6-phenethyl-4-phenylquinolin-2-yl)pyrrolidine-3-carboxylic acid (Example #1)

To a stirred solution of (E)-1-(4-phenyl-6-styrylquinolin-2-yl)pyrrolidine-3-carboxylic acid (130 mg, 0.309 mmol) in MeOH (10 mL) under nitrogen atmosphere was added 10% Pd/C (30 mg). The reaction mixture was hydrogenated under hydrogen balloon pressure at room temperature for 24 h. After completion of the reaction (monitored by T.L.C, Eluent: 10% MeOH:DCM, R.f: 0.4), the reaction mixture was filtered through a celite pad, washed with MeOH (20 mL). Filterate was concentrated under reduced pressure to obtain crude product. The crude compound was purified by preparative HPLC and isolated 1-(6-phenethyl-4-phenylquinolin-2-yl)pyrrolidine-3-carboxylic acid (25 mg, 19%) as an off white solid. ¹H-NMR (400 MHz, DMSO-d6): δ 12.45 (bs, 1H), 7.58-7.55 (m, 1H), 7.52-7.50 (m, 2H), 7.45-7.43 (m, 1H), 7.38-7.36 (m, 2H), 7.25-7.17 (m, 4H), 7.11 (d, J=6.80 Hz, 2H), 6.68 (s, 1H), 6.52 (s, 1H), 3.79-3.74 (m, 2H), 3.62-3.56 (m, 2H), 3.22-3.20 (m, 1H), 2.87-2.84 (m, 4H), 2.23-2.18 (m, 2H). MS (ESI) m/z: 423 [M+H]⁺
Prep. HPLC Conditions

### Solubility: Acetonitrile+Methanol+Water (40+40+20)

### Mobile Phase-A: 0.1% Formic Acid in Water

### Mobile Phase-B: Acetonitrile+Methanol (70+30)

Column: Sunfire C-18 (21.2*250 mm), 5μ
Flow Rate: 14 mL/minute

### Gradient: 0/10, 20/55, 30/75.

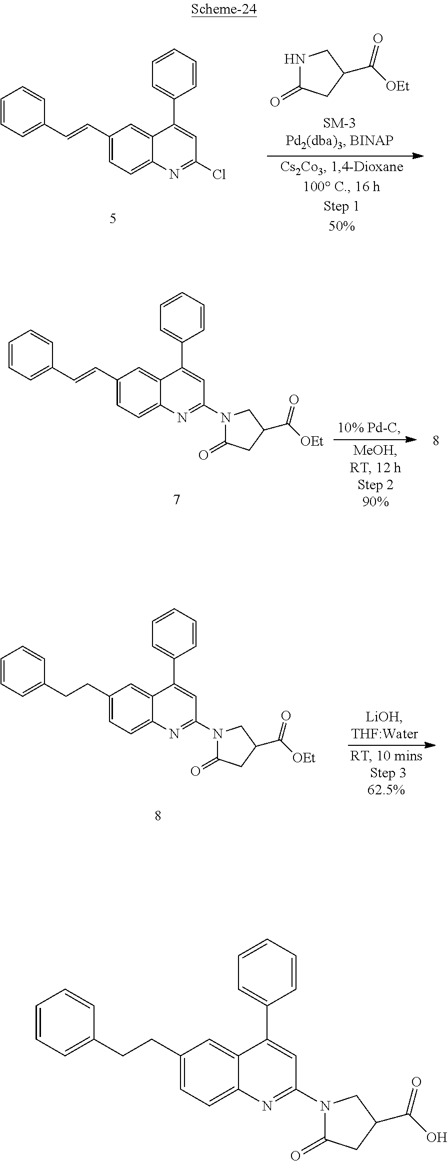 [SMILES: Clc1cc(-c2ccccc2)c2cc(/C=C/c3ccccc3)ccc2n1 ; CCOC(=O)C1CNC(=O)C1 ; CCOC(=O)C1CC(=O)N(c2cc(-c3ccccc3)c3cc(/C=C/c4ccccc4)ccc3n2)C1 ; CCOC(=O)C1CC(=O)N(c2cc(-c3ccccc3)c3cc(CCc4ccccc4)ccc3n2)C1 ; O=C(O)C1CC(=O)N(c2cc(-c3ccccc3)c3cc(CCc4ccccc4)ccc3n2)C1]

### Example #2: Synthesis of 5-oxo-1-(6-phenethyl-4-phenylquinolin-2-yl)pyrrolidine-3-carboxylic acid

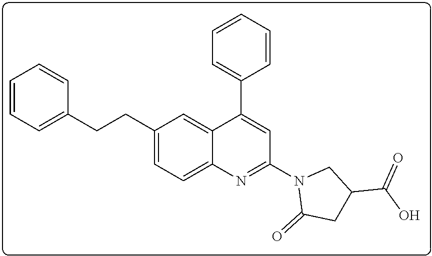 [SMILES: O=C(O)C1CC(=O)N(c2cc(-c3ccccc3)c3cc(CCc4ccccc4)ccc3n2)C1]

### Step 1: Synthesis of ethyl (E)-5-oxo-1-(4-phenyl-6-styrylquinolin-2-yl)pyrrolidine-3-carboxylate

To a solution of (E)-2-chloro-4-phenyl-6-styrylquinoline (0.3 g 0.879 mmol) and 1,4-Dioxane (10 mL) in sealed tube was added Cs₂CO₃ (710 mg, 2.199 mmol) followed by the addition of BINAP (32 mg, 0.0527 mol), and Pd₂(dba)₃ (21.3 mg, 0.0263 mmol), under continuous nitrogen purging. After stirring for 10 min, ethyl 5-oxopyrrolidine-3-carboxylate (165 mg, 1.055 mmol) was added and the reaction mixture was heated to 100° C. for 16 h. After completion of the reaction (monitored by T.L.C, Eluent: 10% EtOAc/Hexane, R.f: 0.5), it was cooled to room temperature and filtered over celite bed. The celite bed was further washed with ethyl acetate (2×30 mL), and the combined organic layers washed with water (25 mL), followed by brine (20 mL). The organic layer was separated, dried over Na₂SO₄, filtered and concentrated under reduced pressure to obtain crude compound. The crude compound was purified by column chromatography over silica gel (100-200 mesh) using a solvent gradient of 10% EtOAC in hexane as eluent to afford ethyl (E)-5-oxo-1-(4-phenyl-6-styrylquinolin-2-yl)pyrrolidine-3-carboxylate (200 mg, 50%) as a brown liquid. MS (ESI) m/z: 463.0 [M+H]⁺.

### Step 2: Synthesis of ethyl 5-oxo-1-(6-phenethyl-4-phenylquinolin-2-yl)pyrrolidine-3-carboxylate

The process of this step was obtained from Step-6 of Example #1 and the desired compound obtained as (180 mg, 90%) brown color solid, MS (ESI) m/z: 465 [M+H]⁺

### Step-3: Synthesis of 5-oxo-1-(6-phenethyl-4-phenylquinolin-2-yl)pyrrolidine-3-carboxylic acid (Example #2)

To a stirred solution of ethyl 5-oxo-1-(6-phenethyl-4-phenylquinolin-2-yl)pyrrolidine-3-carboxylate (0.18 g, 0.3879 mmol) in THF:H₂O (10 mL, 4:1) was added LiOH·H₂O (48.8 mg, 1.1637 mmol) and stirred at room temperature for 10 mins. After completion of the reaction (monitored by T.L.C, Eluent: 30% EtOAc/Hexane, R.f: 0.1), it was diluted with water (10 mL) and acidified with 2M HCl (pH˜5-6) and extracted with 10% MeOH/DCM (2×20 mL). The combined organic layer was washed with water (20 mL), dried over Na₂SO₄, filtered and concentrated under reduced pressure to obtain crude. The crude compound was purified by column chromatography over silica gel (100-200 mesh) using a solvent of 3% MeOH in DCM to afford 5-oxo-1-(6-phenethyl-4-phenylquinolin-2-yl)pyrrolidine-3-carboxylic acid (106.9 g 62.5%) as a white solid. ¹H-NMR (400 MHz, DMSO-d6): δ 12.85 (bs, 1H), 8.38 (s, 1H), 7.88 (d, J=8.40 Hz, 1H), 7.68 (dd, J=2.00, 8.80 Hz, 1H), 7.57-7.53 (m, 3H), 7.38-7.34 (m, 3H), 7.24-7.22 (m, 3H), 7.14-7.11 (m, 2H), 4.36-4.32 (m, 2H), 3.41-3.40 (m, 1H), 3.01-2.95 (m, 2H), 2.92-2.82 (m, 4H).

MS (ESI) m/z: 437 [M+H]⁺

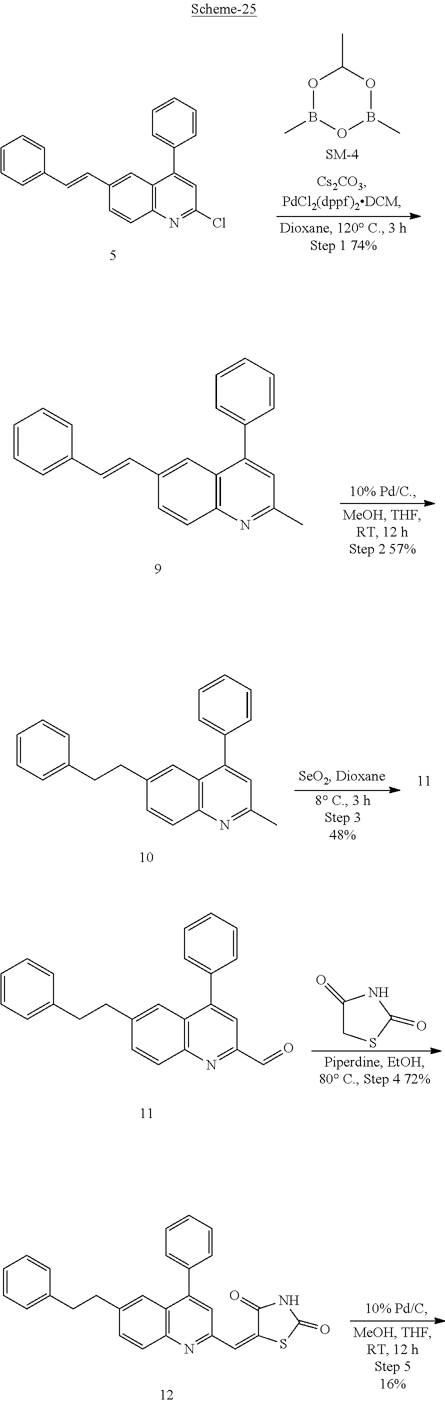

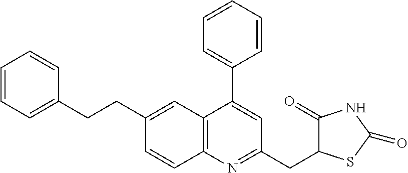 [SMILES: O=C1NC(=O)C(Cc2cc(-c3ccccc3)c3cc(CCc4ccccc4)ccc3n2)S1]

### Example #3: Synthesis of 5-((6-phenethyl-4-phenylquinolin-2-yl)methyl)thiazolidine-2,4-dione

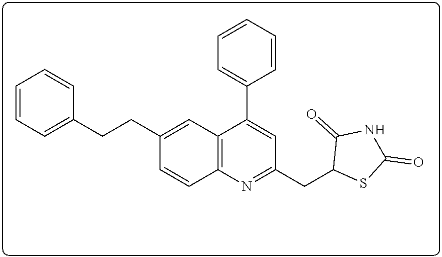 [SMILES: O=C1NC(=O)C(Cc2cc(-c3ccccc3)c3cc(CCc4ccccc4)ccc3n2)S1]

### Step 1: Synthesis of (E)-2-methyl-4-phenyl-6-styrylquinoline

To a solution of (E)-2-chloro-4-phenyl-6-styrylquinoline (1 g, 2.932 mol) and 1,4-Dioxane (10 mL) in a sealed tube was added 2,4,6-trimethyl-1,3,5,2,4,6-trioxatriborinane (0.439 g, 3.5190 mmol), Cs₂CO₃ (2.85 g, 8.797 mmol), (degassed under nitrogen atmosphere for 10 mins), and Pd (DPPf)Cl₂. DCM adduct (120 mg, 0.146 mmol) and heated the reaction mixture at 100° C. for 3 h. After completion of the reaction (monitored by T.L.C, Eluent: 10% EtOAc: Hexane, R.f: 0.3), the reaction mixture was cooled to room temperature, diluted the reaction mixture with water (20 mL) and extracted with ethyl acetate (2×30 mL). The combined organic layer was dried over sodium sulphate, filtered and concentrated under reduced pressure to obtain crude product. The crude compound was purified by column chromatography over silica gel using a solvent gradient of 10% EtOAC in Hexane as eluent to afford (E)-2-methyl-4-phenyl-6-styrylquinoline (700 mg, 74.4%). MS (ESI) m/z: 322.0 [M+H]⁺

### Step 2: Synthesis of 2-methyl-6-phenethyl-4-phenylquinoline

The process of this step was obtained from Step-6 of Example #1 and the desired compound obtained as (400 mg, 57%) as a green color solid, MS (ESI) m/z: 324 [M+H]⁺

### Step 3: Synthesis of 6-phenethyl-4-phenylquinoline-2-carbaldehyde

To a stirred solution of 2-methyl-6-phenethyl-4-phenylquinoline (0.4 g, 1.234 mmol) in 1,4-dioxane was added (0.163 g, 1.48 mmol) and heated at 80° C. for 3 h. After completion of the reaction (monitored by T.L.C, Eluent: 30% EtOAc/Hexane, R.f: 0.6), it was cooled to room temperature, diluted with ice-water (25 mL) and extracted with ethyl acetate (2×25 mL). The combined organic layer was dried over Na₂SO₄, filtered and concentrated under reduced pressure to obtain crude compound. The crude compound was purified by column chromatography over silica gel (100-200 mesh) using a solvent gradient of 20% EtOAc in Hexane to afford 6-phenethyl-4-phenylquinoline-2-carbaldehyde (0.2 g 48.7%) as a light-yellow solid. MS (ESI) m/z: 338 [M−H]⁺

### Step 4: Synthesis of (E)-5-((6-phenethyl-4-phenylquinolin-2-yl)methylene)thiazolidine-2,4-dione

A mixture of (E)-5-((6-phenethyl-4-phenylquinolin-2-yl)methylene)thiazolidine-2,4-dione (200 mg, 0.5934 mmol) and thiazolidine-2,4-dione (73.4 mg, 0.7121 mmol) in EtOH (10 mL) at RT, was added piperidine (0.1 mL, 0.4747 mmol) and heated up to reflux for 4 h. After completion of the reaction (monitored by T.L.C, Eluent: 50% EtOAc/Hexane, R.f: 0.3), it was cooled to room temperature and the solvent was removed under reduced pressure. The residue was diluted with water (10 mL), the compound was extracted with ethyl acetate (3×10 mL). The combined organic layer was dried over Na₂SO₄ filtered and concentrated under reduced pressure to obtain crude compound. The crude compound was purified by column chromatography over silica gel (100-200 mesh) using a solvent gradient of 25% EtOAc in hexane as eluent to afford (E)-5-((6-phenethyl-4-phenylquinolin-2-yl)methylene)thiazolidine-2,4-dione (120 mg, 72%) as a beige solid. MS (ESI) m/z: 437 [M+H]⁺

### Step-5: Synthesis of 5-((6-phenethyl-4-phenylquinolin-2-yl)methyl)thiazolidine-2,4-dione (Example #3)

The process of this step was obtained from Step-6 of Example #1 and the desired compound obtained as (16.1 mg, 16%) as a Pale pink solid. ¹H-NMR (400 MHz, DMSO-d6): δ 12.15 (bs, 1H), 7.90 (d, J=8.80 Hz, 1H), 7.70 (dd, J=2.00, 8.80 Hz, 1H), 7.56-7.52 (m, 3H), 7.44 (d, J=1.20 Hz, 1H), 7.38-7.35 (m, 3H), 7.27-7.22 (m, 2H), 7.20-7.18 (m, 1H), 7.14-7.12 (m, 2H), 5.04-5.00 (m, 1H), 3.85 (dd, J=4.00, 17.00 Hz, 1H), 3.61-3.54 (m, 1H), 3.02 (t, J=7.20 Hz, 2H), 2.90 (t, J=7.60 Hz, 2H). MS (ESI) m/z: 439 [M+H]⁺
Prep. HPLC Conditions

### Solubility: Acetonitrile+Water+THF (60+30+10)

### Mobile Phase-A: Ammonium Acetate in Water

### Mobile Phase-B: Acetonitrile

Column: X-BRIDGE C18 (21.2*250 mm), 5μ
Flow Rate: 14 mL/minute

### Gradient: 0/35, 10/70, 25/90.

### Abbreviations

d doublet
dd doublet of doublet
m multiplet
s singlet
t triplet
DCM Dichloromethane (Methylene chloride)
DIPEA N,N-diisopropylethylamine amine
DMF N, N-dimethyl formamide
DMSO Dimethyl sulphoxide

### eq Equivalent (s)

g gram (s)
h hour (s)
K₂CO₃ Potassium carbonate
THF tetrahydrofuran
EtOAc Ethyl acetate
TLC Thin layer chromatography
RT Room temperature

### EtOH Ethanol

### MeOH Methanol

Cs₂CO₃ Cesium carbonate
LiOH·H₂O Lithium hydroxide monohydrate
Na₂CO₃ Sodium carbonate
PPA Polyphosphoric acid
POCl₃ Phosphorus oxychloride
POBr₃ Phosphorus oxybromide

### NBS N-Bromosuccinimide

### NIS N-Iodosuccinimide

### General Analytical Methods

### LC-MS Conditions

| S.NO | Conditions |
|---|---|
| 1 | Column: GeminiC-18, (50 mm × 2 mm), 3 μm, Mobile Phase-A: 0.01% FA in Water |
| | MobilePhase-B: 100%, ACN, Flow rate: 0.6 mL/Min, Gradient: (T/% B): 0.01/5, 0.8/95, |
| | 3/95 |
| 2 | Column: EclipseRRHD, C18 (50 mm × 2.1 mm), 3 μm, Mobile Phase-A: 0.01% TFA in |
| | Water |
| | Mobile Phase-B: 100% ACN, Flowrate: 0.6 mL/Min, Gradient: (T/% B): 0.01/5, 2/90, 5/90 |
| 3 | Column: X-BridgeC-18(150 mm × 4.6 mm), 3.5 μm, Mobile Phase-A: 5 mM ABC in Water |
| | Mobile Phase-B: 100% ACN, Flowrate: 0.6 mL/Min, Gradient: (T/% B): 0.01/5, 2/90, 5/90 |

### Prep. HPLC Conditions

| S.NO | Conditions |
|---|---|
| 1 | Column: Luna C18 (250 mm × 21.2 mm), 5 μm Mobile Phase-A: 0.01% TFA in Water, Mobile |
| | Phase-B: 100% ACN, Flow rate: 12, Gradient: (T/% B): 0/5, 10/20, 20/60, 30/90. |
| 2 | Column: Xbridge C-18 (250 mm × 19 mm), 5 μm, Mobile Phase-A: 10 mM ABC in |
| | Water, Mobile Phase-B: 100% ACN, Flow rate: 14, Gradient: (T/% B): 0/5, 5/5, 10/30, 20/70, |
| | 30/90. |
| 3 | Column: Ge miniC-18 (250 mm × 21.2 mm), 5 μm, Mobile Phase-A: 0.01% FA in Water |
| | Mobile Phase-B: 100% ACN, Flow rate: 14, Gradient: (T/% B): 0/20, 10/30, 20/50, 30/90. |

Technical outcomes of the above are reflected below in Table 2.

TABLE 2

| | | LCMS method/rt | |
|---|---|---|---|
| | | Observed mass | Synthetic |
| IUPAC Name | NMR | Exact mass | Method |
| 3-{[6-butyl-4-(4- | 400 MHz-DMSO-d6: 12.4 (s, | LCMS5.5/2.09 | method A: R1 = |
| fluorophenyl)quinolin- | 1H), 7.58-7.54 (m, 3H), 7.42- | Observed mass: | n-Butyl; |
| 2- | 7.37(m, 3H), 7.27(d, J = | Exact mass: 394.21 | R2 = 4-Fluoro- |
| yl](methyl)amino}- | 1.6 Hz, 1H), 6.90(s, 1H), | | Phenyl; R3 = 2- |
| 2-methylpropanoic | 3.79(d, J = 7.2 Hz, 2H), 3.15 | | methyl- |
| acid | (s, 3H), 2.91 (q, J = 10.4, 7.2 | | propionic |
| | Hz, 1H), 2.58(t, J = 7.2 Hz, | | acid; |
| | 2H), 1.57-1.49(m, 2H), 1.30- | | R4 = methyl |
| | 1.23(m, 4m), 1.09( d, J = | | |
| | 7.6 Hz, 3H), 0.83(t, J = 6.8 Hz, | | |
| | 3 Hz) | | |
| 3-{[4-(4- | 400 MHz-DMSO-d6: 12.4 (s, | LCMS5.5/2.16 | method A: R1 = |
| fluorophenyl)-6- | 1H), 7.57-7.54 (m, 3H), 7.41- | Observed mass [M − H]: | n-hexyl; |
| hexylquinolin-2- | 7.37(m, 3H), 7.27(s, 1H), | 421.24 | R2 = 4-Fluoro- |
| yl](methyl)amino}- | 6.90(s, 1H), 3.79 (d, J = | Exact mass: 422.24 | Phenyl; R3 = 2- |
| 2-methylpropanoic | 7.6 Hz, 2H), 3.15 (s, 3H), 2.90 | | methyl- |
| acid | (q, J = 14.4, 6.8 Hz, 1H), | | propionic |
| | 2.57(t, J = 7.6 Hz, 2H), 1.57- | | acid; |
| | 1.49(m, 2H), 1.30-1.23(m, | | R4 = methyl |
| | 6m), 1.08 (d, J = 7.6 Hz, 3H), | | |
| | 0.82(t, J = 6.8 Hz, 3 Hz) | | |
| 2-{[4-(4- | 400 MHz-DMSO-d6: 12.4 (s, | LCMS5.5/2.11 | method A: R1 = |
| fluorophenyl)-6- | 1H), 7.57-7.54 (m, 3H), 7.45- | Observed mass [M − H]: | n-pentyl; |
| pentylquinolin-2- | 7.37(m, 3H), 7.27(s, 1H), | 379.29 | R2 = 4-Fluoro- |
| yl](methyl)amino} | 6.90(br s, 1H), 4.49(s, 2H), | Exact mass: 380.19 | Phenyl; |
| acetic acid | 3.25-3.15 (m, 3H), 2.80- | | R3 = ethyl |
| | 2.57(m, 2H), 1.57-1.49(m, | | carboxylic |
| | 2H), 1.30-1.22(m, 4H), 0.82(t, | | acid; |
| | J = 6.8 Hz, 3 Hz) | | R4 = methyl |
| 2-{[4-(4- | 400 MHz-DMSO-d6: 12.4 (s, | LCMS5.5/2.19 | method A: R1 = |
| fluorophenyl)-6- | 1H), 7.57-7.54 (m, 3H), 7.45- | Observed mass [M − H]: | n-hexyl; |
| hexylquinolin-2- | 7.38(m, 3H), 7.30(s, 1H), | 393.38 | R2 = 4-Fluoro- |
| yl](methyl)amino} | 6.97(br s, 1H), 4.47(s, 2H), | Exact mass: 394.21 | Phenyl; |
| acetic acid | 3.25-3.16 (m, 3H), 2.67- | | R3 = ethyl |
| | 2.57(m, 2H), 1.57-1.49(m, | | carboxylic |
| | 2H), 1.30-1.22(m, 6H), 0.82(t, | | acid; |
| | J = 6.0 Hz, 3 Hz) | | R4 = methyl |
| 2-{[6-hexyl-3- | 500 MHz-DMSO-d6: 12.4 (s, | LCMS_5.5 min/2.13 | |
| methyl-4- | 1H), 7.72 (s, 1H), 7.49 (d, J = | Observed mass [M + H]: | |
| (morpholin-4- | 8.4 Hz, 1H), 7.34 (dd, J = 8.4, | 400.50 | |
| yl)quinolin-2- | 2 Hz, 1H), 3.96 (s, 2H), | Exact mass: 399.25 | |
| yl](methyl)amino} | 3.83(m, 4H), 3.20 (m, 4H), | | |
| acetic acid | 2.96 (s, 3H), 2.72 (t, J = | | |
| | 7.2 Hz, 2H), 2.27 (s, 3H), | | |
| | 1.63-1.55(m, 2H), 1.35- | | |
| | 1.26(m, 8H), 0.85 (t, J = | | |
| | 7.2 Hz, 3H) | | |
| 2-([4-(4- | 500 MHz-DMSO-d6: 12.4 (s, | RND-FA-4.51/2.491 | method A′″: |
| fluorophenyl)-6- | 1H), 7.60 (d, J = 7.5 Hz, 1H), | Observed mass [M + H]: | R1 = n-hexyl; |
| hexyl-3- | 7.42-7.38(m, 3H), 7.36- | 409.10 | R1′ = H, R1″ = |
| methylquinolin-2- | 7.33(m, 2H), 6.87(br s, 1H), | Exact mass: 408.22 | Methyl |
| yl](methyl)amino} | 4.01(s, 2H), 3.04(s, 3H), 2.67- | | R2 = 4-Fluoro- |
| acetic acid | 2.57(m, 2H), 2.07 (s, 3H), | | Phenyl; |
| | 1.49-1.47(m, 2H), 1.24- | | R3 = ethyl |
| | 1.20(m, 8H), 0.81(t, J = | | carboxylic |
| | 6.0 Hz, 3 Hz) | | acid; |
| | | | R4 = methyl |
| 2-{[4,6-bis(4- | 400 MHz-DMSO-d6: 12.4 (s, | LCMS_5 MIN/2.72 | method A: R1 = |
| fluorophenyl) | 1H), 7.83 (dd, J = 8.0, 2 Hz, | Observed mass [M + H]: | 4-Fluoro- |
| quinolin-2- | 1H), 7.69-7.59(m, 6H), 7.04 | 405.30 | Phenyl; |
| yl](methyl)amino} | (t, J-9.2 Hz, 2H), 7.25 (t, J- | Exact mass: 404.13 | R2 = 4-Fluoro- |
| acetic acid | 9.2 Hz, 2H), 6.97(br s, 1H), | | Phenyl; |
| | 4.44(s, 2H), 3.226 (s, 3H) | | R3 = ethyl |
| | | | carboxylic |
| | | | acid; |
| | | | R4 = methyl |
| 2-{[4-(4- | 400 MHz-DMSO-d6: 12.4 (s, | LCMS_5 MIN/3.13 | method A: R1 = |
| fluorophenyl)-6- | 1H), 7.55-7.49 (m, 3H), 7.41- | Observed mass [M + H]: | n-hexyl; |
| hexylquinolin-2- | 7.37(m, 3H), 7.26(s, 1H), | 437.46 | R2 = 4-Fluoro- |
| yl](2- | 6.88(s, 1H), 4.32(s, 2H), 3.42 | Exact mass: 436.25 | Phenyl; |
| methylpropyl)amino} | (d, J = 7.6 Hz, 2H), 3.15(s, | | R3 = ethyl |
| acetic acid | 3H), 2.58 (t, J = 7.2 Hz, 2H), | | carboxylic |
| | 2.03 (m, 1H), 1.52(m, 2H), | | acid; R4 = 2- |
| | 1.30-1.23(m, 6H), 0.92 (d, J = | | methyl-propyl |
| | 6.4 Hz, 6H), 0.82(t, J = 6.8 Hz, | | |
| | 3 Hz) | | |
| 2-{[4-(4- | 400 MHz-DMSO-d6: 12.4 (s, | LCMS_5 MIN/3.00 | method A: R1 = |
| fluorophenyl)-6- | 1H), 7.55-7.49 (m, 3H), 7.41- | Observed mass [M − H]: | n-hexyl; |
| hexylquinolin-2- | 7.37(m, 3H), 7.26(s, 1H), | 421.35 | R2 = 4-Fluoro- |
| yl](propyl)amino} | 6.86(s, 1H), 4.33 (s, 2H), 3.56 | Exact mass: 422.24 | Phenyl; |
| acetic acid | (t, J = 7.2 Hz, 2H), 2.58 | | R3 = ethyl |
| | (m, 2H), 1.66-1.62(m, 2H), | | carboxylic |
| | 1.52(m, 2H), 1.30-1.23(m, | | acid; R4 = n- |
| | 6H), 0.90 (t, J = 7.6 Hz, 3H), | | propyl |
| | 0.82(t, J = 6.8 Hz, 3 Hz) | | |
| 2-([4-(4- | | Observed mass: | method A: R1 = |
| fluorophenyl)-6- | | Exact mass: 380.19 | n-hexyl; |
| hexylquinolin-2- | | | R2 = 4-Fluoro- |
| yl]amino}acetic | | | Phenyl; |
| acid | | | R3 = ethyl |
| | | | carboxylic |
| | | | acid; |
| | | | R4 = hydrogen |
| 2-{ethyl[4-(4- | 400 MHz-DMSO-d6: 12.4 (s, | LCMS_5.5/2.25 | method A: R1 = |
| fluorophenyl)-6- | 1H), 7.55-7.50 (m, 3H), 7.41- | Observed mass [M + H]: | n-hexyl; |
| hexylquinolin-2- | 7.37(m, 3H), 7.27(s, 1H), | 409.48 | R2 = 4-Fluoro- |
| yl]amino}acetic | 6.85(s, 1H), 4.33 (s, 2H), 3.66 | Exact mass: 408.22 | Phenyl; |
| acid | (m, 2H), 2.59-2.54 (m, 2H), | | R3 = ethyl |
| | 1.52-1.50 (m, 2H), 1.30- | | carboxylic |
| | 1.23(m, 8H), 1.15 (t, J = | | acid; R4 = n- |
| | 6.8 Hz, 3H), 0.82(t, J = 6.8 Hz, | | ethyl |
| | 3H) | | |
| 2-{[6-hexyl-4- | 400 MHz-DMSO-d6: 12.4 (s, | LCMS_5 min/2.71 | method C: R1 = |
| (pyridin-3- | 1H), 8.55(d, J = 2.4 Hz, 1H), | Observed mass [M + H]: | n-hexyl; |
| yloxy)quinolin-2- | 8.50(d, J = 4.0 Hz, 1H), | 394.38 | R2 = 3- |
| yl](methyl)amino} | 7.70(s, 1H), 7.67-7.64(m, | Exact mass: 393.21 | hydroxy- |
| acetic acid | 1H), 7.53-7.43 (m, 3H), | | pyridyl; |
| | 6.22(s, 1H), 4.21 (s, 2H), | | R3 = ethyl |
| | 3.03(s, 3H), 2.67(t, J = 7.6 Hz, | | carboxylic |
| | 2H), 1.62-1.58 (m, 2H), 1.33- | | acid; |
| | 1.20(m, 6H), 0.84(t, J = | | R4 = methyl |
| | 6.8 Hz, 3H) | | |
| 2-{[6-hexyl-4- | 400 MHz-DMSO-d6: 12.4 (s, | RND X-bridge 5.0 min/ | method C: R1 = |
| (pyridin-4- | 1H), 7.83(d, J = 8 Hz, 2H), | 1.949 | n-hexyl; |
| yloxy)quinolin-2- | 7.54 (d, J = 8.8 Hz, 1H), | Observed mass: | R2 = 4- |
| yl](methyl)amino} | 7.43(d, J = 8.8 Hz, 1H), 7.06 | Exact mass: 393.21 | hydroxy- |
| acetic acid | (s, 1H), 7.02 (s, 1H), 6.26 d, J = | | pyridyl; |
| | 8 Hz, 2H), 4.05 (s, 2H), | | R3 = ethyl |
| | 3.16(s, 3H), 2.62(t, J = 7.6 Hz, | | carboxylic |
| | 2H), 1.57-1.53 (m, 2H), 1.33- | | acid; |
| | 1.20(m, 6H), 0.84 (t, J = | | R4 = methyl |
| | 6.8 Hz, 3H) | | |
| 2-{[4-(3- | 400 MHz-DMSO-d6: 12.4 (s, | LCMS_5.5 min/2.21 | method C: R1 = |
| fluorophenoxy)-6- | 1H), 7.61(s, 1H), 7.52- | Observed mass [M − H]: | n-hexyl; |
| hexylquinolin-2- | 7.46(m, 2H), 7.43-7.40 (m, | 409.22 | R2 = 3-fluoro- |
| yl](methyl)amino} | 1H), 7.11-7.07(m, 2H), 7.01- | Exact mass: 410.20 | 1-hydroxy- |
| acetic acid | 6.99 (m, 1H), 6.3(s, 1H), 4.15 | | phenyl; |
| | (s, 2H), 3.02(s, 3H), 2.65(t, J = | | R3 = ethyl |
| | 7.6 Hz, 2H), 1.62-1.56 (m, | | carboxylic |
| | 2H), 1.33-1.18(m, 6H), 0.83(t, | | acid; |
| | J = 6.8 Hz, 3H) | | R4 = methyl |
| 2-([4-(4- | 400 MHz-DMSO-d6: 12.4 (s, | LCMS_5 min/2.87 | method C: R1 = |
| fluorophenoxy)-6- | 1H), 7.71(s, 1H), 7.48- | Observed mass [M + H]: | n-hexyl; |
| hexylquinolin-2- | 7.40(m, 2H), 7.34-7.24 (m, | 411.38 | R2 = 4-fluoro- |
| yl](methyl)amino} | 4H), 6.08(s, 1H), 4.16 (s, 2H), | Exact mass: 410.20 | 1-hydroxy- |
| acetic acid | 2.97(s, 3H), 2.67(t, J = 7.6 Hz, | | phenyl; |
| | 2H), 1.62-1.56 (m, 2H), 1.33- | | R3 = ethyl |
| | 1.18(m, 6H), 0.84(t, J = | | carboxylic |
| | 6.8 Hz, 3H) | | acid; |
| | | | R4 = methyl |
| 2-{[4-(4- | 400 MHz-DMSO-d6-D20: | LCMS_5 min/3.34 | method A: R1 = |
| fluorophenyl)-6- | 12.4 (s, 1H), 7.74 (m, 1H), | Observed mass [M + H]: | n-octyl; |
| octylquinolin-2- | 7.58-7.56 (m, 3H), 7.43(t, J = | 465.53 | R2 = 4-Fluoro- |
| yl](2- | 7.2 Hz, 2H), 7.3 l(s, 1H), | Exact mass: 464.28 | Phenyl; |
| methylpropyl)amino} | 7.06(s, 1H), 4.56 (s, 2H), 3.66 | | R3 = ethyl |
| acetic acid | (m, 2H), 2.66-2.59 (m, 2H), | | carboxylic |
| | 2.05-2.01 (m, 2H), 1.62- | | acid; R4 = 2- |
| | 1.50(m, 2H), 1.23-1.20(m, | | methyl-propyl |
| | 12H), 0.94 (d, J = 7.5 Hz, 6H), | | |
| | 0.83 (t, J = 7.0 Hz, 3H) | | |
| 2-{[4-(4- | 400 MHz-DMSO-d6: 12.4 (s, | LCMS_5 min/3.16 | method A: R1 = |
| fluorophenyl)-6- | 1H), 7.54-7.49 (m, 3H), 7.47- | Observed mass [M + H]: | n-octyl; |
| octylquinolin-2- | 7.35(m, 3H), 7.24(s, 1H), | 451.53 | R2 = 4-Fluoro- |
| yl](propyl)amino} | 6.84(s, 1H), 4.32 (s, 2H), 3.54 | Exact mass: 450.27 | Phenyl; |
| acetic acid | (m, 2H), 2.57-2.54 (m, 2H), | | R3 = ethyl |
| | 1.62-1.50(m, 2H), 1.53- | | carboxylic |
| | 1.49(m, 2H), 1.30-1.20(m, | | acid; R4 = n- |
| | 10H), 0.88 (t, J = 7.6 Hz, 6H), | | propyl |
| | 0.82 (t, J = 7.2 Hz, 3H) | | |
| 2-{ethyl[4-(4- | 400 MHz-DMSO-d6: 12.4 (s, | RND_FA_4.01/2.37 | method A: R1 = |
| fluorophenyl)-6- | 1H), 7.56-7.50 (m, 3H), 7.41- | Observed mass [M + H]: | n-octyl; |
| octylquinolin-2- | 7.37(m, 3H), 7.26(s, 1H), | 437.10 | R2 = 4-Fluoro- |
| yl]amino}acetic | 6.85(s, 1H), 4.32 (s, 2H), | Exact mass: 436.25 | Phenyl; |
| acid | 3.68-3.64 (m, 2H), 2.57-2.54 | | R3 = ethyl |
| | (m, 2H), 1.55-1.50(m, 2H), | | carboxylic |
| | 1.53-1.49(m, 2H), 1.30- | | acid; R4 = ethyl |
| | 1.20(m, 10H), 1.17 (t, J = | | |
| | 7.2 Hz, 3H), 0.83 (t, J = 7.2 Hz, | | |
| | 3H) | | |
| 2-{methyl [6-octyl- | 400 MHz-DMSO-d6: 12.4 (s, | LCMS_5 min/2.84 | method C: R1 = |
| 4-(pyridin-3- | 1H), 8.56(d, J = 2.4 Hz, 1H), | Observed mass [M + H]: | n-octyl; |
| yloxy)quinolin-2- | 8.50(d, J = 4.0 Hz, 1H), | 422.44 | R2 = 3- |
| yl]amino}acetic | 7.71(s, 1H), 7.66-7.65(m, | Exact mass: 421.24 | hydroxy- |
| acid | 1H), 7.53-7.46 (m, 3H), | | pyridyl; |
| | 6.22(s, 1H), 4.31 (s, 2H), | | R3 = ethyl |
| | 3.00(s, 3H), 2.67(t, J = 7.6 Hz, | | carboxylic |
| | 2H), 1.62-1.58 (m, 2H), 1.33- | | acid; |
| | 1.15(m, 10H), 0.84 (t, J = | | R4 = methyl |
| | 6.8 Hz, 3H) | | |
| 2-{methyl [6-octyl- | 400 MHz-DMSO-d6: 12.4 (s, | LCMS_5 min/2.86 | method C: R1 = |
| 4-(pyridin-4- | 1H), 7.81(d, J = 8 Hz, 2H), | Observed mass [M + H]: | n-octyl; |
| yloxy)quinolin-2- | 7.54 (d, J = 8.8 Hz, 1H), | 422.39 | R2 = 4- |
| yl]amino}acetic | 7.43(d, J = 8 Hz, 1H), 7.06 (s, | Exact mass: 421.24 | hydroxy- |
| acid | 1H), 7.00 (s, 1H), 6.26 (d, J = | | pyridyl; |
| | 8 Hz, 2H), 3.99 (br s, 2H), | | R3 = ethyl |
| | 3.16(s, 3H), 2.63(t, J = 7.6 Hz, | | carboxylic |
| | 2H), 1.57-1.53 (m, 2H), 1.33- | | acid; |
| | 1.15(m, 10H), 0.84 (t, J = | | R4 = methyl |
| | 6.8 Hz, 3H) | | |
| 2-{[4-(3- | 400 MHz-DMSO-d6: 12.4 (s, | LCMS_5.5 min/2.39 | method C: R1 = |
| fluorophenoxy)-6- | 1H), 7.64(s, 1H), 7.53- | Observed mass [M − H]: | n-octyl; |
| octylquinolin-2- | 7.47(m, 2H), 7.45-7.42 (m, | 437.28 | R2 = 3-fluoro- |
| yl](methyl)amino} | 1H), 7.13-7.09(m, 2H), 7.03- | Exact mass: 438.23 | 1-hydroxy- |
| acetic acid | 7.01 (m, 1H), 6.32(s, 1H), | | phenyl; |
| | 4.30 (s, 2H), 3.02(s, 3H), | | R3 = ethyl |
| | 2.66(t, J = 7.6 Hz, 2H), 1.62- | | carboxylic |
| | 1.56 (m, 2H), 1.33-1.18(m, | | acid; |
| | 10H), 0.84 (t, J = 6.8 Hz, 3H) | | R4 = methyl |
| 2-{[4-(4- | 400 MHz-DMSO-d6: 12.4 (s, | LCMS_5 min/2.99 | method C: R1 = |
| fluorophenoxy)-6- | 1H), 7.74(s, 1H), 7.46(d, J = | Observed mass [M + H]: | n-octyl; |
| octylquinolin-2- | 8.8 Hz, 1H), 7.44(d, J = 8.8 Hz, | 439.45 | R2 = 4-fluoro- |
| yl](methyl)amino} | 1H), 7.35-7.26(m, 3H), 6.09 | Exact mass: 438.23 | 1-hydroxy- |
| acetic acid | (s, 1H), 4.29 (s, 2H), 2.97(s, | | phenyl; |
| | 3H), 2.67(t, J = 7.6 Hz, 2H), | | R3 = ethyl |
| | 1.65-1.53 (m, 2H), 1.33- | | carboxylic |
| | 1.20(m, 10H), 0.84 (t, J = | | acid; |
| | 6.8 Hz, 3H) | | R4 = methyl |
| 2-{[6-decyl-4-(4- | 400 MHz-DMSO-d6: 12.4 (s, | AA_9.0/4.1 | method A: R1 = |
| fluorophenyl)quinolin- | 1H), 7.56-7.53 (m, 3H), 7.41- | Observed mass [M + H]: | n-decyl; |
| 2- | 7.36(m, 3H), 7.28 (s, 1H), | 451.51 | R2 = 4-Fluoro- |
| yl](methyl)amino} | 6.89(br s, 1H), 4.39(s, | Exact mass: 450.27 | Phenyl; |
| acetic acid | 2H), 3.16(s, 3H), 2.67-2.57(m, | | R3 = ethyl |
| | 2H), 1.57-1.49(m, 2H), 1.30- | | carboxylic |
| | 1.22(m, 14H), 0.82(t, J = | | acid; |
| | 6.0 Hz, 3 Hz) | | R4 = methyl |
| 2-([4-(4- | 400 MHz-DMSO-d6: 12.4 (s, | LCMS_5.5/2.28 | method A: R1 = |
| fluorophenyl)-6- | 1H), 7.56-7.52 (m, 3H), 7.41- | Observed mass [M + H]: | n-septyl; |
| heptylquinolin-2- | 7.36 (m, 3H), 7.28 (s, 1H), | 409.47 | R2 = 4-Fluoro- |
| yl](methyl)amino} | 6.89 (s, 1H), 4.35 (s, 2H), | Exact mass: 408.22 | Phenyl; |
| acetic acid | 3.04 (s, 3H), 2.57-2.54 | | R3 = ethyl |
| | (m, 2H), 1.55-1.50(m, 2H), | | carboxylic |
| | 1.30-1.20(m, 8H), 0.83 (t, J = | | acid; |
| | 7.2 Hz, 3H) | | R4 = methyl |
| 2-{[4-(4- | 400 MHz-DMSO-d6: 12.4 (s, | LCMS_5.0/3.02 | method A: R1 = |
| fluorophenyl)-6- | 1H), 7.57-7.53 (m, 3H), 7.41- | Observed mass [M + H]: | n-octyl; |
| octylquinolin-2- | 7.37 (m, 3H), 7.28 (s, 1H), | 423.44 | R2 = 4-Fluoro- |
| yl](methyl)amino} | 6.91 (s, 1H), 4.41 (s, 2H), | Exact mass: 422.24 | Phenyl; |
| acetic acid | 3.19 (s, 3H), 2.60-2.54 | | R3 = ethyl |
| | (m, 2H), 1.55-1.50(m, 2H), | | carboxylic |
| | 1.30-1.20(m, 10H), 0.83 (t, J = | | acid; |
| | 9.6 Hz, 3H) | | R4 = methyl |
| 2-[(6- | | Observed mass: | |
| hexylquinolin-2- | | Exact mass: 300.18 | |
| yl)(methyl)amino] | | | |
| acetic acid | | | |
| 2-{2- | 400 MHz-DMSO-d6: 12.5 (s, | RND_FA_3.5/1.815 | method A: R1 = |
| [(carboxymethyl) | 2H), 7.97 (dd, J = 8.0, 1.2 Hz, | Observed mass [M + H]: | n-hexyl; |
| (methyl)amino]-6- | 1H), 7.69 (dt, J = 7.6, 1.2 Hz, | 421.00 | R2 = 2- |
| hexylquinolin-4- | 1H), 7.61 (dt, J = 7.6, 1.2 Hz, | Exact mass: 420.20 | carboxylic |
| yl}benzoic acid | 1H), 7.49 (d, J = 8.4 Hz, 1H), | | acid-Phenyl; |
| | 7.37-7.32 (m, 2H), 6.91 (s, | | R3 = ethyl |
| | 1H), 6.85 (s, 1H), 4.45 (d, J = | | carboxylic |
| | 18 Hz, 1H), 4.35 (d, J = | | acid; |
| | 18 Hz, 1H), 3.16 (s, 3H), | | R4 = methyl |
| | 1.52-1.40(m, 2H), 1.30- | | |
| | 1.20(m, 6H), 0.81 (t, J = | | |
| | 6.4 Hz, 3H) | | |
| 2-{[4-(4, 4- | 400 MHz-DMSO-d6: 12.4 (s, | LCMS_5 min/2.79 | method C: R1 = |
| difluoropiperidin-1- | 1H), 7.52 (s, 1H), 7.41 (d, J = | Observed mass [M + H]: | n-hexyl; |
| yl)-6-hexylquinolin- | 8.4 Hz, 1H), 7.31 (dd, J = 8.4, | 420.44 | R2 = 4, 4-di- |
| 2- | 2 Hz, 1H), 6.46 (s, 1H), 4.29 | Exact mass: 419.24 | fluoro- |
| yl](methyl)amino} | (s, 2H), 3.30-3.17(m, 4H), | | piperidine; |
| acetic acid | 3.14 (s, 3H), 2.68 (t, J = | | R3 = ethyl |
| | 7.2 Hz, 2H), 2.33-2.21(m, | | carboxylic |
| | 4H), 1.65-1.58(m, 2H), 1.35- | | acid; |
| | 1.26(m, 6H), 0.86 (t, J = | | R4 = methyl |
| | 7.2 Hz, 3H) | | |
| 2-{[4-(3, 3- | 400 MHz-DMSO-d6: 12.4 (s, | LCMS_5 min/2.75 | method C: R1 = |
| difluoropyrrolidin- | 1H), 7.62 (s, 1H), 7.38 (d, J = | Observed mass [M + H]: | n-hexyl; |
| 1-yl)-6- | 8.4 Hz, 1H), 7.29 (dd, J = 8.4, | 406.40 | R2 = 3,3′-di- |
| hexylquinolin-2- | 2 Hz, 1H), 6.20 (s, 1H), 4.33 | Exact mass: 405.22 | fluoro- |
| yl](methyl)amino} | (s, 2H), 3.88(t, J = 13.2 Hz, | | pyrrolidine; |
| acetic acid | 2H), 3.66 (t, J = 6.8 Hz, 2H), | | R3 = ethyl |
| | 3.17 (s, 3H), 2.67 (t, J = | | carboxylic |
| | 7.2 Hz, 2H), 2.59-2.50(m, | | acid; |
| | 2H), 1.63-1.55(m, 2H), 1.32- | | R4 = methyl |
| | 1.26(m, 6H), 0.85 (t, J = | | |
| | 7.2 Hz, 3H) | | |
| 2-{[6-hexyl-4- | 400 MHz-DMSO-d6: 12.4 (s, | LCMS_5 min/2.67 | method C: R1 = |
| (morpholin-4- | 1H), 7.50 (s, 1H), 7.40 (d, J = | Observed mass [M + H]: | n-hexyl; |
| yl)quinolin-2- | 8.4 Hz, 1H), 7.40 (dd, J = 8.4, | 386.10 | R2 = N- |
| yl](methyl)amino} | 2 Hz, 1H), 6.39 (s, 1H), 4.24 | Exact mass: 385.24 | morpholine; |
| acetic acid | (s, 2H), 3.86(m, 4H), 3.14(s, | | R3 = ethyl |
| | 3H), 3.09(m, 4H), 2.66 (t, J = | | carboxylic |
| | 7.2 Hz, 2H), 1.63-1.55(m, | | acid; |
| | 2H), 1.35-1.26(m, 6H), 0.85 | | R4 = methyl |
| | (t, J = 7.2 Hz, 3H) | | |
| 2-{[6-butyl-4-(2- | 400 MHz-DMSO-d6: 12.5(s, | LCMS5MIN/1.92 | method A: R1 = |
| methyl-pyridin-4- | 1H), 8.62(s, 1H), 8.55(d, J = | Observed mass [M + H]: | n-hexyl; |
| yl)quinolin-2- | 4 Hz, 1HH), 7.55(d, J = | 392.25 | R2 = 4-(2- |
| yl](methyl)amino} | 8.8 Hz, 1H), 7.40 (d, J = | Exact mass: 391.23 | methyl)pyridyl; |
| acetic acid | 8.0 Hz, 2H), 7.26(d, J = 4 Hz, | | R3 = ethyl |
| | 1H), 6.90 (s, 1H), 6.81(s, | | acid; |
| | 1H), 4.42-4.35 (m, 2H), 3.17 | | R4 = methyl |
| | (s, 3H), 2.62 (t, J = 8 Hz, 2H), | | |
| | 2.01(s, 3H), 1.58-1.51 (m, | | |
| | 2H), 1.35-1.26(m, 6H), 0.90 | | |
| | (t, J = 6.5 Hz, 3H) | | |
| 2-{[4-(3, 5- | 400 MHz-DMSO-d6: 12.4 (s, | LCMS_5 min/2.76 | method A: R1 = |
| dimethyl-1,2- | 1H), 7.50 (d, J = 8.4 Hz, 1H), | Observed mass [M + H]: | n-hexyl; |
| oxazol-4-yl)-6- | 7.36 (dd, J = 8.8, 1.6 Hz, 1H), | 396.42 | R2 = 3, 5- |
| hexylquinolin-2- | 7.03 (s, 1H), 6.81 (s, 1H), | Exact mass: 395.22 | methyl-4- |
| yl](methyl)amino} | 4.04 (s, 2H), 3.15 (s, 3H), | | oxazole; |
| acetic acid | 2.60 (t, J = 7.2 Hz, 2H), | | R3 = ethyl |
| | 2.28(s, 3H), 2.06(s, 3H), 1.55- | | carboxylic |
| | 1.50(m, 2H), 1.30-1.20(m, | | acid; |
| | 6H), 0.83 (t, J = 7.2 Hz, 3H) | | R4 = methyl |
| 2-{[4-(3- | 500 MHz-DMSO-d6: 12.5 (s, | RND X-bridge 5.0/ | method A: R1 = |
| cyanophenyl)-6- | 1H), 7.99-7.96 (m, 2H), 7.81 | 2.369 | n-hexyl; |
| hexylquinolin-2- | (d, J = 6.0 Hz, 1H), 7.75(t, J = | Observed mass [M + H]: | R2 = 3-cyano- |
| yl](methyl)amino} | 8 Hz, 1H), 7.52 (d, J = 8.5 Hz, | 401.90 | Phenyl; |
| acetic acid | 1H), 7.38 (d, J = 8.5 Hz, 1H), | Exact mass: 401.21 | R3 = ethyl |
| | 7.18 (s, 1H), 6.87 (s, 1H), | | carboxylic |
| | 4.17 (s, 1H), 3.18 (s, 3H), | | acid; |
| | 2.57 (m, 2H), 1.52-1.40(m, | | R4 = methyl |
| | 2H), 1.52-1.50(m, 2H), 1.23 | | |
| | (m, 6H), 0.81 (t, J = 6.4 Hz, | | |
| | 3H) | | |
| 3-{[4-(3- | 400 MHz-DMSO-d6: 12.5 (s, | LCMS_5 min/2.79 | method A: R1 = |
| cyanophenyl)-6- | 1H), 7.99-7.96 (m, 2H), 7.83 | Observed mass [M + H]: | n-hexyl; |
| hexylquinolin-2- | (d, J = 7.5 Hz, 1H), 7.74(t, J = | 430.43 | R2 = 3-cyano- |
| yl](methyl)amino} | 8 Hz, 1H), 7.54 (d, J = 8.5 Hz, | Exact mass: 429.24 | Phenyl; R3 = 2- |
| butanoic acid | 1H), 7.37 (d, J = 8.5 Hz, 1H), | | methyl- |
| | 7.69 (dt, J = 7.6, 1.2 Hz, 1H), | | propionic |
| | 7.17 (s, 1H), 6.98 (s, 1H), | | acid; |
| | 5.13 (s, 1H), 2.96 (s, 3H), | | R4 = methyl |
| | 2.59-2.55(m, 2H), 2.4-23.3(m, | | |
| | 2H), 1.52-1.40(m, 2H), 1.30- | | |
| | 1.20(m, 6H), 1.18 (d, J = 6 Hz, | | |
| | 3H), 0.81 (t, J = 6.4 Hz, 3H) | | |
| 3-[(6-hexyl-4- | 400 MHz-DMSO-d6: 12.5 (s, | LCMS_5 min/2.90 | method A: R1 = |
| phenylquinolin-2- | 1H), 7.56-7.48 (m, 6H), 7.38 | Observed mass [M + H]: | n-hexyl; |
| yl)(methyl)amino]- | (d, J = 8.5 Hz, 1H), 7.30 (s, | 405.40 | R2 = Phenyl; |
| 2-methylpropanoic | 1H), 6.89 (s, 1H), 3.77(d, J = | Exact mass: 404.25 | R3 = 2-methyl- |
| acid | 8 Hz, 2H), 3.15 (s, 3H), 2.90- | | propionic |
| | 2.80 (m, 1H), 2.57 (t, J = | | acid; |
| | 7.2 Hz, 2H), 1.52-1.40(m, | | R4 = methyl |
| | 2H), 1.30-1.20(m, 6H), 1.07 | | |
| | (d, J = 6.8 Hz, 3H), 0.82 (t, J = | | |
| | 5.6 Hz, 3H) | | |
| 2-[methyl(6- | 400 MHz-DMSO-d6: 12.5 (s, | LCMS_LCMS_41/ | method A: R1 = |
| pentanamido-4- | 1H), 9.90(s, 1H), 7.85-7.80 | 1.80 | n-pentyl |
| phenylquinolin-2- | (m, 2H), 7.65-7.45 (m, 4H), | Observed mass [M + H]: | amide; R2 = 3- |
| yl)amino]acetic | 6.89 (s, 1H), 4.50 (m, 2H), | 392.42 | cyano-Phenyl; |
| acid | 3.15 (s, 3H), 2.25 (m, 2H), | Exact mass: 391.19 | R3 = ethyl acid; |
| | 1.52-1.40(m, 2H), 1.30- | | R4 = methyl |
| | 1.20(m, 2H), 0.82 (t, J = | | |
| | 5.6 Hz, 3H) | | |
| 2-{methyl [6- | 400 MHz-DMSO-d6: 12.5 (s, | RND-FA-4.10 MIN/ | method A: R1 = |
| (pentyloxy)-4- | 1H), 9.90(s, 1H), 7.58-7.52 | 1.96 | n-pentyl |
| phenylquinolin-2- | (m, 6H), 7.22(dd, J = 9.2, | Observed mass [M + H]: | ether; R2 = 3- |
| yl]amino}acetic | 2.8 Hz, 1H), 6.93-6.91 (m, | 379.10 | cyano-Phenyl; |
| acid | 2H), 4.38 (2, 2H), 3.849(t, J = | Exact mass: 378.19 | R3 = ethyl acid; |
| | 6.8 Hz, 2H), 3.18 (s, 3H), | | R4 = methyl |
| | 2.25 (m, 2H), 1.69-1.65(m, | | |
| | 2H), 1.33-1.25(m, 4H), 0.86 | | |
| | (t, J = 5.6 Hz, 3H) | | |
| 2-[(7-bromo-4- | 400 MHz-DMSO-d6: 12.5 (s, | LCMS_5 min/2.73 | method A″: |
| phenylquinolin-2- | 1H), 7.73(s, 1H), 7.57-7.49 | Observed mass [M + H]: | R1 = bromo;; |
| yl)(methyl)amino] | (m, 6H), 7.27(dd, J = 8.8, | 371.45 | R2 = Phenyl; |
| acetic acid | 2 Hz, 1H), 6.92(s, 1H), 4.28 | Exact mass: 370.03 | R3 = ethyl acid; |
| | (s, 2H), 3.19(s, 3H), | | R4 = methyl |
| 2-[(7-hexyl-4- | 400 MHz-DMSO-d6: 12.5 (s, | LCMS_5 min/2.86 | method A″: |
| phenylquinolin-2- | 1H), 7.57-7.49 (m, 5H), 7.45 | Observed mass [M + H]: | R1 = n-hexyl;; |
| yl)(methyl)amino] | (d, J = 8.8 Hz, 1H), 7.40(s, | 377.40 | R2 = Phenyl; |
| acetic acid | 1H), 7.03(dd, J = 8.8, 1.6 Hz, | Exact mass: 376.22 | R3 = ethyl acid; |
| | 1H), 6.87(s, 1H), 4.42 (s, 2H), | | R4 = methyl |
| | 3.18 (s, 3H), 2.68(t, J = | | |
| | 7.2 Hz, 2H), 1.65-1.59(m, | | |
| | 2H), 1.35-1.20(m, 6H), 0.86 | | |
| | (t, J = 7.2 Hz, 3H) | | |
| 2-[methyl(6-octyl- | 400 MHz-DMSO-d6: 12.5 (s, | RND-FA-3.5/2.515 | method A: R1 = |
| 4-phenylquinolin-2- | 1H), 7.58-7.49 (m, 6H), 7.41- | Observed mass [M + H]: | n-octyl;; |
| yl)amino]acetic | 7.36 (m, 3H), 7.39(dd, J = 8.8, | 405.10 | R2 = Phenyl; |
| acid | 1.6 Hz, 1H), 6.91(s, 1H), 4.42 | Exact mass: 404.25 | R3 = ethyl acid; |
| | (s, 2H), 3.19 (s, 3H), 2.57- | | R4 = methyl |
| | 2.54 (m, 2H), 1.55-1.50(m, | | |
| | 2H), 1.30-1.20(m, 10H), | | |
| | 0.83 (t, J = 7.2 Hz, 3H) | | |
| 3-{[6-hexyl-4- | 400 MHz-DMSO-d6: 12.5 (s, | RND-FA-3.5 min/1.772 | method A: R1 = |
| (pyridin-3- | 1H), 8.71(m, 2H), 7.95(d, J = | Observed mass [M + H]: | n-hexyl; |
| yl)quinolin-2- | 8 Hz, 1H), 7.60-7.54(m, 2H), | 406.10 | R2 = 3-pyridyl; |
| yl](methyl)amino}- | 7.39(d, J = 8.4 Hz, 1H), 7.20 | Exact mass: 405.24 | R3 = 2-methyl- |
| 2-methylpropanoic | (s, 1H), 6.97(s, 1H), 3.75(s, | | propionic |
| acid | 2H), 3.17 (s, 3H), 2.58 (t, J = | | acid; |
| | 7.6 Hz, 2H), 1.55-1.50(m, | | R4 = methyl |
| | 2H), 1.30-1.20(m, 6H), | | |
| | 1.04(d, J = 7.2 Hz, 3H), 0.82 | | |
| | (t, J = 6.8 Hz, 3H) | | |
| 2-{[6-hexyl-4- | 400 MHz-DMSO-d6: 12.5 (s, | LCMS_5 min/2.64 | method A: R1 = |
| (pyridin-3- | 1H), 8.70-8.67(m, 2H), | Observed mass [M + H]: | n-hexyl; |
| yl)quinolin-2- | 7.91(d, J = 8 Hz, 1H), 7.57(dd, | 378.42 | R2 = 3-pyridyl; |
| yl](methyl)amino} | J = 7.6, 4.8 Hz, 1H), 7.51(d, J = | Exact mass: 377.21 | R3 = ethyl acid; |
| acetic acid | 8.4 Hz, 1H), 7.36(m, 1H), | | R4 = methyl |
| | 7.20 (s, 1H), 6.79(s, 1H), 4.00 | | |
| | (s, 2H), 3.17 (s, 3H), 2.57 (t, | | |
| | J = 7.6 Hz, 2H), 1.55-1.50(m, | | |
| | 2H), 1.30-1.20(m, 6H), 0.82 | | |
| | (t, J = 6.8 Hz, 3H) | | |
| 2-{[4-(3- | 400 MHz-DMSO-d6: 13.2 (s, | LCMS_5 min/3.04 | method B: R1 = |
| cyanophenyl)-6- | 1H), 8.02-7.99 (m, 2H), 7.83 | Observed mass [M + H]: | n-hexyl; |
| hexylquinolin-2- | (d, J = 8.0 Hz, 1H), 7.76(t, J = | 389.31 | R2 = 3-cyano- |
| yl]oxy}acetic acid | 8 Hz, 1H), 7.52 (dd, J = 8.5, | Exact mass: 388.18 | phenyl; R3 = |
| | 1.6 Hz, 1H), 7.42 (d, J = | | ethyl acid |
| | 8.5 Hz, 1H), 7.12 (s, 1H), 6.60 | | |
| | (s, 1H), 5.05(s, 2H), 2.56 (t, J = | | |
| | 7.2 Hz, 2H), 1.52-1.40(m, | | |
| | 2H), 1.23 (m, 6H), 0.82 (t, J = | | |
| | 6.4 Hz, 3H) | | |
| 3-{[4-(3- | 400 MHz-DMSO-d6: 12.2 (s, | LCMS_5 min/2.80 | method A: R1 = |
| cyanophenyl)-6- | 1H), 8.01-7.97 (m, 2H), 7.84 | Observed mass [M + H]: | n-hexyl; |
| hexylquinolin-2- | (d, J = 8.0 Hz, 1H), 7.76 (t, J = | 430.43 | R2 = 3-cyano- |
| yl](methyl)amino}- | 8 Hz, 1H), 7.57 (d, J = 8.4 Hz, | Exact mass: 429.24 | phenyl; R3 = 2- |
| 2-methylpropanoic | 1H), 7.41 (d, J = 8.5 Hz, 1H), | | methyl |
| acid | 7.19 (s, 1H), 6.98 (s, 1H), | | propionic |
| | 3.81(d, J = 7.2 Hz, 2H), | | acid; |
| | 3.16(s, 3H), 2.94-2.89(m, | | R4 = methyl |
| | 1H), 2.56 (t, J = 7.2 Hz, 2H), | | |
| | 1.52-1.40(m, 2H), 1.23 (m, | | |
| | 6H), 1.10(d, J = 7.2 Hz, 3H), | | |
| | 0.82 (t, J = 6.4 Hz, 3H) | | |
| 2-{[4-(3- | 400 MHz-DMSO-d6: 12.5(s, | RND X-bridge 5.0 min/ | method A: R1 = |
| cyanophenyl)-6- | 1H), 7.98-7.96(m, 2H), | 2.385 | n-hexyl; |
| hexylquinolin-2- | 7.80(d, J = 7.6 Hz, 1H), | Observed mass: | R2 = 3-cyano- |
| yl](methyl)amino} | 7.74(t, J = 8.4 Hz, 1H), 7.50(d, | Exact mass: 401.21 | phenyl; |
| acetic acid | J = 8.8 Hz, 1H), 7.36(dd, J = | | R3 = ethyl acid; |
| | 8.8, 1.6 Hz, 1H), 7.16 (s, 1H), | | R4 = methyl |
| | 6.80 (s, 1H), 4.01 (s, 2H), | | |
| | 3.16 (s, 3H), 2.57 (t, J = 8 Hz, | | |
| | 2H), 1.55-1.47 (m, 2H), 1.36- | | |
| | 1.22(m, 6H), 0.82 (t, J = | | |
| | 6.5 Hz, 3H) | | |
| 1-[6-hexyl-4- | 400 MHz-DMSO-d6: 12.5(s, | LCMS_5 MIN/2.66 | method A: R1 = |
| (pyridin-3- | 1H), 8.72-8.70(m, 2H), 7.95 | Observed mass [M + H]: | n-hexyl; |
| yl)quinolin-2- | (d, J = 7.6 Hz, 1H), 7.62- | 418.43 | R2 = 3-pyridyl; |
| yl]piperidine-3- | 7.58(m, 2H), 7.42(d, J = | Exact mass: 417.24 | R3-R4 = 2- |
| carboxylic acid | 8.4 Hz, 1H), 7.23(s, 1H), | | carboxylic |
| | 7.18(s, 1H), 4.55(d, J = | | acid piperidyl |
| | 12.8 Hz, 1H), 4.27(d, J = | | |
| | 12.8 Hz, 1H), 3.21-3.10 (m, | | |
| | 2H), 2.59 (t, J = 8 Hz, 2H), | | |
| | 2.07-1.97(m, 1H), 1.75-1.64 | | |
| | (m, 2H), 1.54-1.48 (m, 2H), | | |
| | 1.32-1.26(m, 6H), 0.82 (t, J = | | |
| | 6.5 Hz, 3H) | | |
| 1-(6-hexyl-4- | 400 MHz-DMSO-d6: 12.5(s, | RND-FA-3.5 min/2.049 | method A: R1 = |
| phenylquinolin-2- | 1H), 7.59-7.49(m, 6H), 7.40 | Observed mass [M + H]: | n-hexyl; |
| yl)piperidine-3- | (dd, J = 8.4, 1.6 Hz, 1H), | 417.10 | R2 = phenyl; |
| carboxylic acid | 7.30(s, 1H), 7.06(s, 1H), | Exact mass: 416.25 | R3-R4 = 2- |
| | 4.52(d, J = 12.8 Hz, 1H), | | carboxylic |
| | 4.23(d, J = 12.8 Hz, 1H), | | acid piperidyl |
| | 3.31-3.11 (m, 2H), 2.59 (t, J = | | |
| | 8 Hz, 2H), 2.07-1.97(m, 1H), | | |
| | 1.75-1.64 (m, 2H), 1.54-1.48 | | |
| | (m, 2H), 1.32-1.26(m, 6H), | | |
| | 0.82 (t, J = 6.5 Hz, 3H) | | |
| 2-{[6-butyl-4-(4- | 400 MHz-DMSO-d6: 12.5(s, | RND-FA-3.5 min/1.823 | method A: R1 = |
| hydroxyphenyl) | 1H), 9.72 (s, 1H), 7.52 (d, J = | Observed mass [M + H]: | n-butyl; |
| quinolin-2- | 8.4 Hz, 1H), 7.40-7.32(m, | 365.00 | R2 = 4- |
| yl](methyl)amino} | 4H), 6.94 (d, J = 8.4 Hz, 1H), | Exact mass: 364.18 | hydroxyl- |
| acetic acid | 6.84(s, 1H) 4.41 (s, 2H), 3.18 | | phenyl; |
| | (s, 3H), 2.60 (t, J = 8 Hz, 2H), | | R3 = ethyl acid; |
| | 1.54-1.48 (m, 2H), 1.32- | | R4 = methyl |
| | 1.26(m, 2H), 0.87 (t, J = | | |
| | 6.5 Hz, 3H) | | |
| 2-{[6-butyl-4-(3- | 400 MHz-DMSO-d6: 12.5(s, | LCMS_5 min/2.59 | method A: R1 = |
| hydroxyphenyl) | 1H), 9.65 (s, 1H), 7.52 (d, J = | Observed mass [M + H]: | n-butyl; |
| quinolin-2- | 8.0 Hz, 1H), 7.40-7.32(m, | 365.31 | R2 = 3- |
| yl](methyl)amino} | 3H), 7.30 (d, J = 8.0 Hz, 1H), | Exact mass: 364.18 | hydroxyl- |
| acetic acid | 6.90-6.87(m, 4H) 4.41 (s, | | phenyl; |
| | 2H), 3.18 (s, 3H), 2.59 (t, J = | | R3 = ethyl acid; |
| | 8 Hz, 2H), 1.54-1.48 (m, 2H), | | R4 = methyl |
| | 1.32-1.26(m, 2H), 0.86 (t, J = | | |
| | 6.5 Hz, 3H) | | |
| 2-{[6-butyl-4-(2- | 400 MHz-DMSO-d6: 12.5(s, | LCMS_5 min/2.59 | method A: R1 = |
| hydroxyphenyl) | 1H), 9.46 (s, 1H), 7.50 (d, J = | Observed mass [M + H]: | n-butyl; |
| quinolin-2- | 8.4 Hz, 1H), 7.36-7.28(m, | 365.37 | R2 = 2- |
| yl](methyl)amino} | 2H), 7.30 (d, J = 8.0 Hz, 1H), | Exact mass: 364.18 | hydroxyl- |
| acetic acid | 7.16(d, J = 6.4 Hz, 1H), | | phenyl; |
| | 7.09(s, 1H), 7.00(d, J = 6.8 | | R3 = ethyl acid; |
| | Hz, 1H), 6.94(t, J = 7.2 Hz, 1H), | | R4 = methyl |
| | 6.84(s, 1H), 4.39(d, | | |
| | J = 4.4 Hz, 2H), 3.16 (s, 3H), | | |
| | 2.54 (t, J = 8 Hz, 2H), 1.54- | | |
| | 1.48 (m, 2H), 1.31-1.26(m, | | |
| | 2H), 0.85 (t, J = 6.5 Hz, 3H) | | |
| 2-{[6-butyl-4-(4- | 400 MHz-DMSO-d6: 12.5(s, | RND-FA-10/2.218 | method A: R1 = |
| fluorophenyl) | 1H), 7.55-7.52(m, 3H), 7.41- | Observed mass [M + H]: | n-butyl; |
| quinolin-2- | 7.37(m, 3H), 7.27 (s, 1H), | 367.00 | R2 = 4-fluoro- |
| yl](methyl)amino} | 6.84(s, 1H), 4.25 (s, 2H), | Exact mass: 366.17 | phenyl; |
| acetic acid | 3.18 (s, 3H), 2.58 (t, J = 8 Hz, | | R3 = ethyl acid; |
| | 2H), 1.55-1.49 (m, 2H), 1.32- | | R4 = methyl |
| | 1.26(m, 2H), 0.86 (t, J = | | |
| | 6.5 Hz, 3H) | | |
| 2-{[6-butyl-4-(3- | | Observed mass: | |
| fluorophenyl) | | Exact mass: 366.17 | |
| quinolin-2- | | | |
| yl](methyl)amino} | | | |
| acetic acid | | | |
| 2-{[6-butyl-4-(2- | | Observed mass: | |
| fluorophenyl) | | Exact mass: 366.17 | |
| quinolin-2- | | | |
| yl](methyl)amino} | | | |
| acetic acid | | | |
| 2-{[6-butyl-4-(4- | 400 MHz-DMSO-d6: 12.5(s, | RND-FA-10 min/2.279 | method A: R1 = |
| methylphenyl) | 1H), 7.49(d, J = 8.4 Hz, 1H), | Observed mass [M + H]: | n-butyl; |
| quinolin-2- | 7.37-7.30(m, 6H), 6.69(s, | 363.00 | R2 = 4-methyl- |
| yl](methyl)amino} | 1H), 3.93 (s, 2H), 3.16 (s, | Exact mass: 362.20 | phenyl; |
| acetic acid | 3H), 2.56 (t, J = 8 Hz, 2H), | | R3 = ethyl acid; |
| | 2.40(s, 3H), 1.53-1.47 (m, | | R4 = methyl |
| | 2H), 1.32-1.26(m, 2H), 0.86 | | |
| | (t, J = 7.2 Hz, 3H) | | |
| 2-{[6-butyl-4-(3- | | Observed mass: | |
| methylphenyl) | | Exact mass: 362.20 | |
| quinolin-2- | | | |
| yl](methyl)amino} | | | |
| acetic acid | | | |
| 2-{[6-butyl-4-(2- | 500 MHz-DMSO-d6: 12.5(s, | LCMS_5 MIN/2.95 | method A: R1 = |
| methylphenyl) | 1H), 7.54(d, J = 8.5 Hz, 1H), | Observed mass [M + H]: | n-butyl; |
| quinolin-2- | 7.40-7.34(m, 4H), 7.21(d, J = | 363.42 | R2 = 2-methyl- |
| yl](methyl)amino} | 6.5 Hz, 1H), 6.86 (s, 1H), | Exact mass: 362.20 | phenyl; |
| acetic acid | 6.85 (s, 1H), 4.44(d, 1H), 4.37 | | R3 = ethyl acid; |
| | (d, 1H), 3.16 (s, 3H), 2.60 (m, | | R4 = methyl |
| | 2H), 2.00 (s, 3H), 1.53-1.47 | | |
| | (m, 2H), 1.26-1.16(m, 2H), | | |
| | 0.83 (t, J = 7.2 Hz, 3H) | | |
| 2-{[6-butyl-4-(4- | 500 MHz-DMSO-d6: 12.5(s, | LCMS_5 MIN/2.74 | method A: R1 = |
| cyanophenyl) | 1H), 8.04(d, J = 6.5 Hz, 2H), | Observed mass [M + H]: | n-butyl; |
| quinolin-2- | 7.72(d, J = 6.5 Hz, 2H), 7.56 | 374.30 | R2 = 4-cy ano- |
| yl](methyl)amino} | (d, J = 8.5 Hz, 1H), 7.43 (d, J = | Exact mass: 373.18 | phenyl; |
| acetic acid | 8.5 Hz, 1H), 7.22(s, 1H), | | R3 = ethyl acid; |
| | 6.97 (s, 1H), 4.42(s, 2H), | | R4 = methyl |
| | 3.19 (s, 3H), 2.60 (m, 2H), | | |
| | 1.53-1.47 (m, 2H), 1.26- | | |
| | 1.16(m, 2H), 0.86 (t, J = | | |
| | 7.2 Hz, 3H) | | |
| 2-{[6-butyl-4-(4- | 500 MHz-DMSO-d6: 12.5(s, | RND-FA-3.5/1.657 | method A: R1 = |
| carbamoylphenyl) | 1H), 8.10(s, 1H), 8.04(d, J = | Observed mass [M + H]: | n-butyl; |
| quinolin-2- | 8.4 Hz, 2H), 8.04(d, J = 6.4 | 392.00 | R2 = 4-amide- |
| yl](methyl)amino} | Hz, 2H), 7.58-7.71(m, 3H), | Exact mass: 391.19 | phenyl; |
| acetic acid | 7.48(s, 1H), 7.38 (d, J = 8.4 | | R3 = ethyl acid; |
| | Hz, 1H), 7.27(s, 1H), 6.84 (s, | | R4 = methyl |
| | 1H), 4.19 (s, 2H), 3.18 (s, | | |
| | 3H), 2.57 (t, J = 7.6 Hz, 2H), | | |
| | 1.53-1.47 (m, 2H), 1.26- | | |
| | 1.16(m, 2H), 0.86 (t, J = | | |
| | 7.2 Hz, 3H) | | |
| 2-{[6-butyl-4- | 500 MHz-DMSO-d6: 12.5(s, | ANL_MCL5/6.317 | method A: R1 = |
| (pyridin-4- | 1H), 8.70(d, J = 4.5 Hz, 2H), | Observed mass [M + H]: | n-butyl; |
| yl)quinolin-2- | 7.65(d, J = 9 Hz, 1H), 7.57 (d, | 350.31 | R2 = 4-pyridyl; |
| yl](methyl)amino} | J = 4.5 Hz, 2H), 7.41(d, J = | Exact mass: 349.18 | R3 = ethyl acid; |
| acetic acid | 9 Hz, 1H), 7.25 (s, 1H), | | R4 = methyl |
| | 6.88(s, 1H), 4.27 (s, 2H), | | |
| | 3.30 (s, 3H), 2.62 (t, J = 8 Hz, | | |
| | 2H), 1.58-1.51 (m, 2H), 1.35- | | |
| | 1.26(m, 2H), 0.90 (t, J = | | |
| | 6.5 Hz, 3H) | | |
| 6-butyl-2- | 400 MHz-DMSO-d6: 13.2(s, | LCMS_5 MIN/2.85 | |
| (carboxymethoxy)- | 1H), 7.58-7.51 (m, 4H), | Observed mass [M + H]: | |
| 4-phenylquinoline- | 7.44(d, J = 8.8 Hz, 1H), 7.37- | 380.22 | |
| 3-carboxylic acid | 7.35(m, 2H), 6.94 (s, 1H), 5.02 | Exact mass: 379.14 | |
| | (s, 2H), 2.52 (t, J = 7.6 Hz, | | |
| | 2H), 1.55-1.51(m, 2H), 1.30- | | |
| | 1.26(m, 2H), 0.82 (t, J = 8 Hz, | | |
| | 3H) | | |
| 2-{[6-butyl-4- | 500 MHz-DMSO-d6: 12.5(s, | LCMS_5 MIN/2.49 | method A: R1 = |
| (pyridin-3- | 1H), 8.67(m, 2H), 8.00 (d, J = | Observed mass [M + H]: | n-butyl; |
| yl)quinolin-2- | 7.6 Hz, 1H), 7.69 (d, J = | 350.40 | R2 = 3-pyridyl; |
| yl](methyl)amino} | 8.5 Hz, 1H), 7.64-7.61 (m, | Exact mass: 349.18 | R3 = ethyl acid; |
| acetic acid | 1H), 7.43(d, J = 8.5 Hz, 1H), | | R4 = methyl |
| | 7.24(s, 1H), 6.90(s, 1H), | | |
| | 4.28(s, 2H), 3.30 (s, 3H), 2.59 | | |
| | (t, J = 8 Hz, 2H), 2.07-1.97(s, | | |
| | 1H), 1.75-1.64 (m, 2H), 1.54- | | |
| | 1.48 (m, 2H), 0.82 (t, J = | | |
| | 6.5 Hz, 3H) | | |
| 1-[6-butyl-4-(3- | 400 MHz-DMSO-d6: 12.5(s, | RND-FA-3.5/1.986 | method A: R1 = |
| cyanophenyl)quinol | 1H), 8.05-7.95(m, 2H), 7.85 | Observed mass [M + H]: | n-hexyl; |
| in-2-yl]piperidine- | (d, J = 7.6 Hz, 1H), 7.76(t, | 414.00 | R2 = 3-cyano- |
| 3-carboxylic acid | J = 7, 6 Hz, 1), 7.69 (d, J = | Exact mass: 413.21 | phenyl; R3- |
| | 8.8 Hz, 1H), 7.69 (dd, J = 8.4, | | R4 = 3- |
| | 1.6 Hz, 1H), 7.21(s, 1H), | | carboxylic |
| | 7.17(s, 1H), 4.55(d, J = | | acid piperidyl |
| | 12.8 Hz, 1H), 4.27(d, J = | | |
| | 12.8 Hz, 1H), 3.21-3.10 (m, | | |
| | 2H), 2.59 (t, J = 8 Hz, 2H), | | |
| | 2.07-1.97(m, 1H), 1.75-1.64 | | |
| | (m, 2H), 1.54-1.48 (m, 2H), | | |
| | 1.32-1.26(m, 2H), 0.86 (t, J = | | |
| | 6.5 Hz, 3H) | | |
| 4-(6-butyl-4- | 400 MHz-DMSO-d6: 12.4(br | LCMS_5 min/2.84 | method A: R1 = |
| phenylquinolin-2- | s, 1H), 7.61(d, J = 8.0 Hz, | Observed mass [M + H]: | n-hexyl; |
| yl)morpholine-2- | 1H), 7.58-7.45(m, 5H), | 391.37 | R2 = phenyl; |
| carboxylic acid | 7.41(d, J = 7.6 Hz, 1H), | Exact mass: 390.19 | R3-R4 = 3- |
| | 7.32(s, 1H), 7.01(s, 1H), | | carboxylic |
| | 4.53(m, 1H), 4.23(m, 2H), | | acid |
| | 3.78(m, 1H), 3.52(m, 1H), | | morpholino |
| | 3.06(m, 1H), 2.94(m, 1H), | | |
| | 2.60-2.56 (m, 2H), 1.53-1.47 | | |
| | (m, 2H), 1.32-1.26(m, 2H), | | |
| | 0.86 (t, J = 7.2 Hz, 3H) | | |
| 1-(6-butyl-4- | 400 MHz-DMSO-d6: 12.4(br | RND-FA-3.5 min/2.051 | method A: R1 = |
| phenylquinolin-2- | s, 1H), 7.85(d, J = 8.0 Hz, | Observed mass [M + H]: | n-hexyl; |
| yl)piperidine-3- | 1H), 7.70-7.45(m, 5H), | 389.10 | R2 = phenyl; |
| carboxylic acid | 7.41(d, J = 7.6 Hz, 1H), | Exact mass: 388.22 | R3-R4 = 3- |
| | 7.31(s, 1H), 7.07(s, 1H), | | carboxylic |
| | 4.53(d, J = 13.2 Hz, 1H), | | acid piperidyl |
| | 4.23(d, J = 13.2 Hz, 1H), 3.22- | | |
| | 3.10 (m, 2H), 2.60-2.56 (m, | | |
| | 2H), 2.05-1.96(m, 1H), 1.80- | | |
| | 1.60(m, 2H), 1.53-1.47 (m, | | |
| | 2H), 1.32-1.26(m, 3H), 0.86 | | |
| | (t, J = 7.2 Hz, 3H) | | |
| 3-{[6-butyl-4-(3- | 400 MHz-DMSO-d6: 12.4(br | LCMS_5 min/2.73 | method A: R1 = |
| cyanophenyl) | s, 1H), 8.00-7.97(m, 2H), | Observed mass [M + H]: | n-butyl; |
| quinolin-2- | 7.85(d, J = 8.0 Hz, 1H), 7.76(t, | 402.30 | R2 = 3-cyano- |
| yl](methyl)amino}- | J = 8 Hz, 1H), 7.56(d, J = | Exact mass: 401.21 | phenyl; R3 = 2- |
| 2-methylpropanoic | 8.8 Hz, 2H), 7.41(dd, J = 8.4, | | methyl |
| acid | 2 Hz, 1H), 7.19(d, J = 1.6 Hz, | | propionic |
| | 1H), 6.98(s, 1H), 3.78 (d, | | acid; |
| | J = 6.8 Hz, 2H), 3.16(s, 3H), | | R4 = methyl |
| | 2.95-2.82 (m, 1H), 2.60-2.54 | | |
| | (m, 2H), 1.53-1.47 (m, 2H), | | |
| | 1.32-1.26(m, 2H), 1.08 (d, J = | | |
| | 7.2 Hz, 3H), 0.86 (t, J = 7.2 Hz, | | |
| | 3H) | | |
| 3-{[6-butyl-4- | 400 MHz-DMSO-d6: 8.70(s, | LCMS_5 min/2.54 | method A: R1 = |
| (pyridin-3- | 2H), 7.95(d, J = 7.6 Hz, 1H), | Observed mass [M + H]: | n-butyl; |
| yl)quinolin-2- | 7.60-7.53(m, 2H), 7.38(d, J = | 378.31 | R2 = 3-pyridyl; |
| yl](methyl)amino}- | 8.4 Hz, 1H), 7.20(s, 1H), | Exact mass: 377.21 | R3 = 2-methyl |
| 2-methylpropanoic | 6.97(s, 1H), 3.73 (s, 2H), | | propionic |
| acid | 3.16 (s, 3H), 2.59-2.54 (m, | | acid; |
| | 3H), 1.53-1.47 (m, 2H), 1.32- | | R4 = methyl |
| | 1.26(m, 2H), 1.00 (d, J = | | |
| | 6.4 Hz, 3H), 0.86 (t, J = 7.2 Hz, | | |
| | 3H) | | |
| 3-[(6-butyl-4- | 400 MHz-DMSO-d6-D2O | RND-FA-3.5 min/2.085 | method A: R1 = |
| phenylquinolin-2- | exchange: 7.80(br s, 1H), | Observed mass [M + H]: | n-butyl; |
| yl)(methyl)amino]- | 7.64-7.43(m, 6H), 7.37(s, | 377.10 | R2 = phenyl; |
| 2-methylpropanoic | 1H), 7.15 (br s, 1H), 4.00(s, | Exact mass: 376.22 | R3 = 2-methyl |
| acid | 1H), 3.83-3.78 (m, 1H), 3.28 | | propionic |
| | (s, 3H), 2.94(dd, J = 14.8, | | acid; |
| | 7.8 Hz, 1H), 2.62(t, J = 8 Hz, | | R4 = methyl |
| | 2H), 1.53-1.47 (m, 2H), 1.36- | | |
| | 1.19(m, 3H), 1.13 (d, J = | | |
| | 6.4 Hz, 3H), 0.86 (t, J = 7.2 Hz, | | |
| | 3H) | | |
| 3-[(6-butyl-4- | 400 MHz-DMSO-d6: 7.64- | ANL-MCL3/2.03 | method A: R1 = |
| phenylquinolin-2- | 7.43(m, 6H), 7.3 8(d, J = | Observed mass [M + H]: | n-butyl; |
| yl)(methyl)amino] | 8.4 Hz, 1H), 7.37(s, 1H), | 377.24 | R2 = phenyl; |
| butanoic acid | 7.29(s, 1H), 6.91 (br s, 1H), | Exact mass: 376.22 | R3 = 1-methyl |
| | 5.20 (s, 1H), 2.96 (s, 3H), | | propionic |
| | 2.94(dd, J = 14.8, 7.8 Hz, 1H), | | acid; |
| | 2.57(t, J = 8 Hz, 2H), 1.53- | | R4 = methyl |
| | 1.47 (m, 2H), 1.32-1.22(m, | | |
| | 3H), 1.20 (d, J = 6.8 Hz, 3H), | | |
| | 0.86 (t, J = 7.2 Hz, 3H) | | |
| 3-[(6-butyl-4- | 400 MHz-DMSO-d6: 7.64- | RND-FA-3.5 min/1.992 | method A: R1 = |
| phenylquinolin-2- | 7.43(m, 6H), 7.37(d, J = | Observed mass [M + H]: | n-butyl; |
| yl)(methyl)amino] | 8.8 Hz, 1H), 7.28(s, 1H), 6.86 | 363.10 | R2 = phenyl; |
| propanoic acid | (br s, 1H), 5.20 (s, 1H), 3.8(s, | Exact mass: 362.20 | R3 = n- |
| | 2H), 3.14 (s, 3H), 2.57(t, J = | | propionic |
| | 8 Hz, 2H), 2.328(s, 2H), 1.53- | | acid; |
| | 1.47 (m, 2H), 1.32-1.22(m, | | R4 = methyl |
| | 3H), 0.85 (t, J = 7.2 Hz, 3H) | | |
| N-(6-butyl-4-(3- | 400 MHz-DMSO-d6: 11.8(s, | LCMS_5 MIN/3.07 | method A: R1 = |
| cyanophenyl)quinol | 1H), 8.01-7.97(m, 2H), 7.84- | Observed mass [M + H]: | n-butyl; |
| in-2-yl)-N- | 7.81(m, 1H), 7.76(t, J = | 416.28 | R2 = 3-cyano- |
| methylvaline? | 8.4 Hz, 1H), 7.51(d, J = 7.2 Hz, | Exact mass: 415.23 | phenyl; R3 = l- |
| | 1H), 7.43-7.35 (m1H), 7.15 | | isopropyl- |
| | (s, 1H), 7.05 (s, 1H), 4.42 (s, | | ethyl acid; |
| | 2H), 3.11 (s, 3H), 2.54 (t, J = | | R4 = methyl |
| | 8 Hz, 2H), 2.33-2.19(m, 1H), | | |
| | 1.55-1.47 (m, 2H), 1.32- | | |
| | 1.22(m, 2H), 1.01 (d, J = | | |
| | 6.4 Hz, 3H), 0.87(t, J = 6.4 Hz, | | |
| | 3Hh), 0.78 (d, J = 6.4 Hz, 3H) | | |
| 2-{[4-(3- | 400 MHz-DMSO-d6: 8.00(dd, | ANL_MCL5/6.615 | method B: R1 = |
| cyanophenyl)-6- | J = 7.6, 1.6 Hz, 1H), 7.96(s, | Observed mass [M + H]: | n-pentyl; |
| pentylquinolin-2- | 1H), 7.82-7.74 (m, 2H), | 375.23 | R2 = 3-cyano- |
| yl]oxy}acetic acid | 7.42(d, J = 8.0 Hz, 1H), | Exact mass: 374.16 | phenyl; R3 = |
| | 7.28(d, J = 8.0 Hz, 1H), | | ethyl acid |
| | 7.07(d, J = 1.2 Hz, 1H), 6.53 | | |
| | (s, 1H), 4.65 (s, 2H), 2.54(t, J = | | |
| | 8 Hz, 2H), 1.50-1.45 (m, | | |
| | 2H), 1.27-1.19(m, 4H), 0.82 | | |
| | (t, J = 7.2 Hz, 3H) | | |
| 2-{[4-(3- | 400 MHz-DMSO-d6: 13.2 (s, | RND-FA-3.5/1.996 | method B: R1 = |
| carbamoylphenyl)- | 1H), 8.08(s, 1H), 8.04- | Observed mass [M + H]: | n-pentyl; |
| 6-pentylquinolin-2- | 8.01(m, 1H), 7.99(s, 1H), | 393.00 | R2 = 3-amido- |
| yl]oxy}acetic acid | 7.67-7.64 (m, 2H), 7.51- | Exact mass: 392.17 | phenyl; R3 = |
| | 7.41(m, 3H), 7.20(d, J = | | ethyl acid |
| | 1.2 Hz, 1H), 6.59 (s, 1H), 5.07 | | |
| | (s, 2H), 2.55(t, J = 8 Hz, 2H), | | |
| | 1.53-1.45 (m, 2H), 1.28- | | |
| | 1.19(m, 4H), 0.82 (t, J = | | |
| | 7.2 Hz, 3H) | | |
| 2-([4-(3- | 400 MHz-DMSO-d6: 8.01- | RND-FA-3.5/2.142 | method B: R1 = |
| cyanophenyl)-6- | 7.97(m, 2H), 7.83-7.74 (m, | Observed mass [M + H]: | n-propyl; |
| propylquinolin-2- | 2H), 7.44(d, J = 8.0 Hz, 1H), | 347.00 | R2 = 3-cyano- |
| yl]oxy}acetic acid | 7.31(d, J = 8.0 Hz, 1H), | Exact mass: 346.13 | phenyl; R3 = |
| | 7.08(d, J = 1.2 Hz, 1H), 6.55 | | ethyl acid |
| | (s, 1H), 4.74 (s, 2H), 2.52(t, J = | | |
| | 8 Hz, 2H), 1.50-1.45 (m, | | |
| | 2H), 0.84 (t, J = 7.2 Hz, 3H) | | |
| 2-{[4-(3- | 400 MHz-DMSO-d6: 13.2 (s, | RND-FA-3.5/1.826 | method B: R1 = |
| carbamoylphenyl)- | 1H), 8.08(s, 1H), 8.04- | Observed mass [M + H]: | n-propyl; |
| 6-propylquinolin-2- | 8.01(m, 1H), 7.99(s, 1H), | 365.00 | R2 = 3-amido- |
| yl]oxy}acetic acid | 7.67-7.64 (m, 2H), 7.51- | Exact mass: 364.14 | phenyl; R3 = |
| | 7.40(m, 3H), 7.20(d, J = | | ethyl acid |
| | 1.6 Hz, 1H), 6.59 (s, 1H), 5.03 | | |
| | (s, 2H), 2.55(t, J = 8 Hz, 2H), | | |
| | 1.53-1.45 (m, 2H), 0.84 (t, J = | | |
| | 7.2 Hz, 3H) | | |
| 2-{[4-(3- | 400 MHz-DMSO-d6: 13.2 (s, | RND-FA-6.0/1.128 | method B: R1 = |
| carbamoylphenyl)- | 1H), 8.09(s, 1H), 8.04- | Observed mass [M + H]: | ethyl; R2 = 3- |
| 6-ethylquinolin-2- | 8.02(m, 1H), 7.99(s, 1H), | 351.00 | amido-phenyl; |
| yl]oxy}acetic acid | 7.66-7.64 (m, 2H), 7.52 (d, J = | Exact mass: 350.13 | R3 = ethyl acid |
| | 7.6 Hz, 1H), 7.46-7.42(m, | | |
| | 2H), 7.22(s 1H), 6.59 (s, 1H), | | |
| | 5.06 (s, 2H), 2.55(q, J = | | |
| | 7.6 Hz, 2H), 1.11(t, J = 7.2 Hz, | | |
| | 3H) | | |
| 2-{[6-bromo-4-(3- | 400 MHz-DMSO-d6: 13.2(s, | RND-FA-3.5/1.975 | method B: R1 = |
| cyanophenyl)quinol | 1H), 8.05-8.02(m, 2H), 7.87- | Observed mass [M + H]: | bromo; |
| in-2-yl]oxy}acetic | 7.76 (m, 3H), 7.51(d, J = | 382.90 | R2 = 3-cyano- |
| acid | 8.8 Hz, 1H), 7.38 (d, J = | Exact mass: 382.00 | phenyl; R3 = |
| | 1.2 Hz, 1H), 6.70 (s, 1H), 5.07 | | ethyl acid |
| | (s, 2H) | | |
| 2-{[6-bromo-4-(3- | 400 MHz-DMSO-d6: 13.2 (s, | RND-FA-3.5/1.652 | method B: R1 = |
| carbamoylphenyl) | 1H), 8.10(s, 1H), 8.06- | Observed mass [M + H]: | bromo; |
| quinolin-2- | 8.02(m, 1H), 7.99(s, 1H), | 400.90 | R2 = 3-amido- |
| yl]oxy}acetic acid | 7.80 (dd, J = 8.8, 2 Hz, 1H), | Exact mass: 400.01 | phenyl; R3 = |
| | 7.80 (d, J = 5.2 Hz, 2H), 7.5 l(d, | | ethyl acid |
| | J = 8.8 Hz, 1H), 7.48(s, 1H), | | |
| | 7.44(d, J = 2 Hz, 1H), 6.69 (s, | | |
| | 1H), 5.07(s, 2H) | | |
| 2-{[6-butyl-4-(3- | 400 MHz-DMSO-d6: 12.5(s, | RND-FA-3.5 min/2.471 | method B: R1 = |
| cyanophenyl)quinol | 1H), 8.03-8.01(m, 2H), 7.84 | Observed mass [M + H]: | n-butyl; |
| in-2-yl]oxy}acetic | (m, 1H), 7.79 (t, J = 7.6 Hz, | 361.00 | R2 = 3-cyano- |
| acid | 1H), 7.73(d, J = 8.4 Hz, 1H), | Exact mass: 360.15 | phenyl; R3 = |
| | 7.58(dd, J = 8.8, 2.0 Hz, 1H), | | ethyl acid |
| | 7.37 (s, 1H), 7.06 (s, 1H), 4.97 | | |
| | (s, 2H), 2.66 (t, J = 7.6 Hz, | | |
| | 2H), 1.55-1.51(m, 2H), 1.30- | | |
| | 1.26(m, 2H), 0.87 (t, J = 8 Hz, | | |
| | 3H) | | |
| 2-{[6-butyl-4-(3- | 400 MHz-DMSO-d6: 13.2 (s, | RND-FA-3.5 min/2.179 | method B: R1 = |
| carbamoylphenyl) | 1H), 8.13(s, 1H), 8.04- | Observed mass [M + H]: | n-butyl; |
| quinolin-2- | 8.01(m, 2H), 7.69-7.66 (m, | 379.00 | R2 = 3-amido- |
| yl]oxy}acetic acid | 3H), 7.51-7.40(m, 2H), | Exact mass: 378.16 | phenyl; R3 = |
| | 7.39(s, 1H), 6.92 (s, 1H), | | ethyl acid |
| | 4.63 (s, 2H), 2.63(t, J = 8 Hz, | | |
| | 2H), 1.53-1.45 (m, 2H), 1.30- | | |
| | 1.26(m, 2H), 0.85 (t, J = | | |
| | 7.2 Hz, 3H) | | |
| 2-{[4-(3- | 400 MHz-DMSO-d6: 12.5(s, | LCMS_5 min/2.85 | method A: R1 = |
| cyanophenyl)-6- | 1H), 8.01-7.98(m, 2H), 7.84 | Observed mass: | n-pentyl; |
| pentylquinolin-2- | (d, J = 7.6 Hz, 1H), 7.76 (t, J = | Exact mass: 387.19 | R2 = 3-cyano- |
| yl](methyl)amino} | 7.6 Hz, 1H), 7.55(d, J = 8.4 Hz, | | phenyl; R3 = |
| acetic acid | 1H), 7.40(dd, J = 8.8, 2.0 Hz, | | ethyl acid; |
| | 1H), 7.21 (s, 1H), 6.95 (s, | | R4 = methyl |
| | 1H), 4.32 (s, 2H), 3.18 (s, | | |
| | 3H), 2.56 (t, J = 7.6 Hz, 2H), | | |
| | 1.55-1.51(m, 2H), 1.30- | | |
| | 1.26(m, 4H), 0.83 (t, J = 8 Hz, | | |
| | 3H) | | |
| 2-{[4-(3- | 400 MHz-CDCl3: 12.5(s, 1H), | LCMS_5 min/2.59 | method A: R1 = |
| carbamoylphenyl)- | 7.97-7.94(m, 2H), 7.71(d, J = | Observed mass [M + H]: | n-pentyl; |
| 6-pentylquinolin-2- | 8 Hz, 1H), 7.65-7.63(m, 2H), | 406.26 | R2 = 3-amido- |
| yl](methyl)amino} | 7.52-7.48 (m, 1H), 7.44 (s, | Exact mass: 405.21 | phenyl; R3 = |
| acetic acid | 1H), 7.31 (s, 1H), 6.86 (s, | | ethyl acid; |
| | 1H), 4.34 (s, 2H), 3.30 (s, | | R4 = methyl |
| | 3H), 2.63 (t, J = 7.6 Hz, 2H), | | |
| | 1.57-1.51(m, 2H), 1.30- | | |
| | 1.26(m, 4H), 0.86 (t, J = | | |
| | 6.5 Hz, 3H) | | |
| 2-([4-(3- | 400 MHz-DMSO-d6: 12.5(s, | RND-FA-3.5/1.875 | method A: R1 = |
| cyanophenyl)-6- | 1H), 8.01-7.98(m, 2H), | Observed mass [M + H]: | n-propyl; |
| propylquinolin-2- | 7.82(d, J = 7.6 Hz, 1H), 7.75 | 360.00 | R2 = 3-cyano- |
| yl](methyl)amino} | (t, J = 7.6 Hz, 1H), 7.53(d, J = | Exact mass: 359.16 | phenyl; R3 = |
| acetic acid | 9.2 Hz, 1H), 7.38(dd, J = 8.8, | | ethyl acid; |
| | 2.0 Hz, 1H), 7.19 (s, 1H), 6.88 | | R4 = methyl |
| | (s, 1H), 4.16(s, 2H), 3.17(s, | | |
| | 3H), 2.55 (t, J = 7.6 Hz, 2H), | | |
| | 1.57-1.51(m, 2H), 0.85 (t, J = | | |
| | 8 Hz, 3H) | | |
| 2-{[4-(3- | 400 MHz-DMSO-d6: 12.5(s, | LCMS_5 min/2.41 | method A: R1 = |
| carbamoylphenyl)- | 1H), 8.05(m, 1H), 8.01- | Observed mass [M + H]: | n-propyl; |
| 6-propylquinolin-2- | 7.99(m, 2H), 7.65-7.61(m, | 378.23 | R2 = 3-amido- |
| yl](methyl)amino} | 2H), 7.55(d, J = 8 Hz, 1H), | Exact mass: 377.17 | phenyl; R3 = |
| acetic acid | 7.44 (s, 1H), 7.39 (d, J = | | ethyl acid; |
| | 9.6 Hz, 1H), 7.26 (s, 1H), 6.92 | | R4 = methyl |
| | (s, 1H), 4.32 (s, 2H), 3.19(s, | | |
| | 3H), 2.55 (m, 2H), 1.57- | | |
| | 1.51(m, 2H), 0.85 (t, J = | | |
| | 6.5 Hz, 3H) | | |
| 2-{[4-(3- | 400 MHz-DMSO-d6: 12.5(s, | RND-FA-3.5/1.765 | method A: R1 = |
| cyanophenyl)-6- | 1H), 8.01-7.98(m, 2H), | Observed mass [M + H]: | ethyl; |
| ethylquinolin-2- | 7.84(d, J = 7.6 Hz, 1H), 7.76 | 346.00 | R2 = 3-cyano- |
| yl](methyl)amino} | (t, J = 7.6 Hz, 1H), 7.55(d, J = | Exact mass: 345.15 | phenyl; R3 = |
| acetic acid | 9.2 Hz, 1H), 7.42(dd, J = 8.8, | | ethyl acid; |
| | 2.0 Hz, 1H), 7.22(s, 1H), 6.92 | | R4 = methyl |
| | (s, 1H), 4.24 (s, 2H), 3.19(s, | | |
| | 3H), 2.61(dd, J = 15.2, 7.6 | | |
| | Hz, 2H), 1.14(t, J = 8 Hz, 3H) | | |
| 2-{[4-(3- | 400 MHz-DMSO-d6: 12.5(s, | Observed mass: | method A: R1 = |
| carbamoylphenyl)- | 1H), 8.08(m, 1H), 8.01- | Exact mass: 363.16 | ethyl; |
| 6-ethylquinolin-2- | 7.97(m, 2H), 7.65-7.61(m, | | R2 = 3-amido- |
| yl](methyl)amino} | 2H), 7.52(d, J = 8 Hz, 1H), | | phenyl; R3 = |
| acetic acid | 7.49 (s, 1H), 7.37 (d, J = | | ethyl acid; |
| | 9.6 Hz, 1H), 7.23 (s, 1H), 6.81 | | R4 = methyl |
| | (s, 1H), 4.20 (s, 2H), 3.16(s, | | |
| | 3H), 2.57 (m, 2H), 1.53(m, | | |
| | 2H), 1.29-1.22(m, 2H), 0.85 | | |
| | (t, J = 6.5 Hz, 3H) | | |
| 2-{[6-bromo-4-(3- | 400 MHz-DMSO-d6: 12.5(s, | RND-FA-3.5/1.977 | method A: R1 = |
| cyanophenyl) | 1H), 8.05(m, 1H), 8.03- | Observed mass [M + H]: | bromo; |
| quinolin-2- | 8.01(m, 1H), 7.87(d, J = | 395.90 | R2 = 3-cyano- |
| yl](methyl)amino} | 7.6 Hz, 1H), 7.78(t, J = 7.6 Hz, | Exact mass: 395.03 | phenyl; R3 = |
| acetic acid | 1H), 7.66(dd, J = 8.8, 2.4 Hz, | | ethyl acid; |
| | 1H), 7.56(d, J = 8 Hz, 1H), | | R4 = methyl |
| | 7.51(d, J = 2 Hz, 1H), 7.11 (s, | | |
| | 1H), 6.81 (s, 1H), 4.44 (s, | | |
| | 2H), 3.21 (s, 3H) | | |
| 2-{[6-bromo-4-(3- | 400 MHz-DMSO-d6: 12.5(s, | LCMS_5 min/2.36 | method A: R1 = |
| carbamoylphenyl) | 1H), 8.05(m, 1H), 8.03- | Observed mass [M − H]: | ethyl; |
| quinolin-2- | 7.98(m, 2H), 7.67-7.60(m, | 412.50 | R2 = 3-amido- |
| yl](methyl)amino} | 3H), 7.54-7.50(m, 3H), 6.92 | Exact mass: 413.04 | phenyl; R3 = |
| acetic acid | (s, 1H), 4.44 (s, 2H), 3.18(s, | | ethyl acid; |
| | 3H) | | R4 = methyl |
| 2-{[6-butyl-4-(3- | 400 MHz-DMSO-d6: 12.5(s, | RND-FA-3.5/1.985 | method A: R1 = |
| cyanophenyl) | 1H), 8.01-7.97(m, 2H), | Observed mass [M + H]: | n-butyl; |
| quinolin-2- | 7.85(d, J = 7.6 Hz, 1H), | 374.00 | R2 = 3-cyano- |
| yl](methyl)amino} | 7.77(t, J = 8.4 Hz, 1H), 7.55(d, | Exact mass: 373.18 | phenyl; R3 = |
| acetic acid | J = 8.8 Hz, 1H), 7.55(dd, J = | | ethyl acid; |
| | 8.8, 1.6 Hz, 1H), 7.22 (s, 1H), | | R4 = methyl |
| | 7.01 (s, 1H), 4.42 (s, 2H), | | |
| | 3.16 (s, 3H), 2.57 (t, J = 8 Hz, | | |
| | 2H), 1.55-1.47 (m, 2H), 1.36- | | |
| | 1.22(m, 2H), 0.86 (t, J = | | |
| | 6.5 Hz, 3H) | | |
| 2-{[6-butyl-4-(3- | 400 MHz-DMSO-d6: 12.5(s, | LCMS_5 min/2.51 | method A: R1 = |
| carbamoylphenyl) | 1H), 8.08(m, 1H), 8.01- | Observed mass [M + H]: | n-butyl; |
| quinolin-2- | 7.97(m, 2H), 7.65-7.61(m, | 392.21 | R2 = 3-amidoo- |
| yl](methyl)amino} | 2H), 7.52(d, J = 8 Hz, 1H), | Exact mass: 391.19 | phenyl; R3 = |
| acetic acid | 7.49 (s, 1H), 7.37 (d, J = | | ethyl acid; |
| | 9.6 Hz, 1H), 7.23 (s, 1H), 6.81 | | R4 = methyl |
| | (s, 1H), 4.20 (s, 2H), 3.16(s, | | |
| | 3H), 2.57(m, 2H), 1.53(m, | | |
| | 2H), 1.29-1.22(m, 2H), 0.85 | | |
| | (t, J = 6.5 Hz, 3H) | | |
| 2-{2-[(6-chloro-4- | | Observed mass: | |
| phenylquinolin-2- | | Exact mass: 383.10 | |
| yl)(methyl)amino] | | | |
| acetamido}acetic | | | |
| acid | | | |
| 2-[methyl(6-pentyl- | 500 MHz-DMSO-d6: 12.5(s, | RND-FA-3.5/2.088 | method A: R1 = |
| 4-phenylquinolin-2- | 1H), 7.57-7.49 (m, 6H), 7.39 | Observed mass [M + H]: | n-pentyl; |
| yl)amino]acetic | (dd, J = 6.8, 1.6 Hz, 1H), 7.31 | 363.10 | R2 = phenyl; |
| acid | (s, 1H), 6.87 (s, 1H), 4.34 (s, | Exact mass: 362.20 | R3 = ethyl |
| | 2H), 3.17 (s, 3H), 2.57 (t, J = | | acid; |
| | 7.5 Hz, 2H), 1.53(dd, J = | | R4 = methyl |
| | 14.8, 7.2 Hz, 2H), 1.29- | | |
| | 1.22(m, 4H), 0.83 (t, J = | | |
| | 6.5 Hz, 3H) | | |
| 2-[methyl(4-phenyl- | 400 MHz-DMSO-d6: 12.5(s, | RND-FA-3.5/1.880 | method A: R1 = |
| 6-propylquinolin-2- | 1H), 7.58-7.49 (m, 6H), 7.39 | Observed mass [M + H]: | n-propyl; |
| yl)amino]acetic | (d, J = 7.6 Hz, 1H), 7.32 (s, | 335.10 | R2 = phenyl; |
| acid | 1H), 6.88 (s, 1H), 4.35 (s, | Exact mass: 334.17 | R3 = ethyl |
| | 2H), 3.19(s, 3H), 2.54 (dd, J = | | acid; |
| | 14.4, 7.2 Hz, 2H), 1.54(dd, | | R4 = methyl |
| | J = 14.8, 7.2 Hz, 2H), 0.85 (t, | | |
| | J = 6.8 Hz, 3 Hz) | | |
| 2-[(6-ethyl-4- | 400 MHz-DMSO-d6: 12.5(s, | RND-FA-3.5/1.780 | method A: R1 = |
| phenylquinolin-2- | 1H), 7.58-7.52 (m, 6H), 7.41 | Observed mass [M + H]: | ethyl; |
| yl)(methyl)amino] | (d, J = 8.8 Hz, 1H), 7.34 (s, | 321.00 | R2 = phenyl; |
| acetic acid | 1H), 6.88 (s, 1H), 4.35 (s, | Exact mass: 320.15 | R3 = ethyl |
| | 2H), 3.19(s, 3H), 2.61 (dd, J = | | acid; |
| | 14.4, 7.2 Hz, 2H), 1.12 (t, J = | | R4 = methyl |
| | 6.8 Hz, 3 Hz) | | |
| 1-(6-hexyl-4- | 500 MHz-DMSO-d6: 12.5(s, | RND-FA-3.5/2.238 | method A: R1 = |
| phenylquinolin-2- | 1H), 8.28(br s, 1H), 7.58-7.48 | Observed mass [M + H]: | n-hexyl; |
| yl)pyrrolidine-2- | (m, 6H), 7.38 (d, J = 10 | 403.10 | R2 = phenyl; |
| carboxylic acid | Hz, 1H), 7.31 (s, 1H), 6.69 (s, | Exact mass: 402.23 | R3-R4 = 2- |
| | 1H), 4.55 (s, 1H), 3.70- | | carboxylic |
| | 3.55(m, 2H), 2.57 (t, J = | | acid proline; |
| | 9.0 Hz, 2H), 2.30-2.23(m, 1H), | | |
| | 2.18-2.00(m, 3H), 1.54- | | |
| | 1.50(m, 2H), 1.24-1.21(m, | | |
| | 6H), 0.82 (t, J = 6.8 Hz, 3 Hz) | | |
| 6-hexyl-4-phenyl-2- | 400 MHz-CDCl3: 7.68(d, J = | RND-FA-3.5/2.238 | method A: R1 = |
| (piperidin-1- | 8.8 Hz, 1H), 7.50-7.45(m, 5H), | Observed mass [M + H]: | n-hexyl; |
| yl)quinoline | 7.38-7.34 (m, 2H), 6.88 (br s, | 373.10 | R2 = phenyl; |
| | 1H), 3.71(s, 4H), 2.60 (t, J = | Exact mass: 372.26 | R3-R4 = |
| | 7.2 Hz, 2H), 1.68(m, 6H), | | Piperidinyl |
| | 1.58-1.54(m, 2H), 1.35- | | |
| | 1.24(m, 6H), 0.85 (t, J = | | |
| | 6.8 Hz, 3 Hz) | | |
| 2-[(6-hexyl-4- | 400 MHz-DMSO-d6: 8.3(s, | RND-FA-3.5/2.197 | method A: R1 = |
| phenylquinolin-2- | 1H), 7.57-7.47 (m, 6H), | Observed mass [M + H]: | n-hexyl; |
| yl)(methyl)amino] | 7.38(dd, J = 8.4, 1.2 Hz, 1H), | 377.10 | R2 = phenyl; |
| acetic acid | 7.30(s, 1H), 6.84 (br s, 1H), | Exact mass: 376.22 | R3 = ethyl |
| | 4.27 (s, 2H), 3.18 (s, 3H), | | acid; |
| | 2.57 (t, J = 7.2 Hz, 2H), 1.55- | | R4 = methyl |
| | 1.49(m, 2H), 1.30-1.22(m, | | |
| | 6H), 0.82 (t, J = 6.8 Hz, 3 Hz) | | |
| 1-(6-butyl-4- | 400 MHz-DMSO-d6: 12.5(s, | RND-FA-3.5/2.001 | method A: R1 = |
| phenylquinolin-2- | 1H), 7.58-7.47 (m, 6H), 7.38 | Observed mass [M + H]: | n-butyl; |
| yl)pyrrolidine-2- | (d, J = 8.8 Hz, 1H), 7.3 l(s, | 375.10 | R2 = phenyl; |
| carboxylic acid | 1H), 6.69 (br s, 1H), 4.55 (s, | Exact mass: 374.20 | R3-R4 = 2- |
| | 2H), 3.68-3.55(m, 2H), 2.57 | | carboxylic |
| | (t, J = 7.2 Hz, 2H), 2.24- | | acid proline; |
| | 2.20(m, 1H), 2.08-1.97(m, | | |
| | 3H), 1.55-1.49(m, 2H), 1.30- | | |
| | 1.22(m, 2H), 0.86 (t, J = | | |
| | 6.8 Hz, 3 Hz) | | |
| 6-butyl-4-phenyl-2- | 400 MHz-CDCl3: 7.68(d, J = | RND-FA-3.5/2.074 | method A: R1 = |
| (piperidin-1- | 8.8 Hz, 1H), 7.50-7.45(m, 5H), | Observed mass [M + H]: | n-butyl; |
| yl)quinoline | 7.38-7.34 (m, 2H), 6.88 (br s, | 345.10 | R2 = phenyl; |
| | 1H), 3.72(s, 4H), 2.61 (t, J = | Exact mass: 344.23 | R3-R4 = |
| | 7.2 Hz, 2H), 1.60-1.52 (m, | | Piperidinyl |
| | 8H), 1.35-1.24(m, 2H), 0.89 | | |
| | (t, J = 6.8 Hz, 3 Hz) | | |
| 2-[(6-bromo-4- | 400 MHz-DMSO-d6: 13.2 (s, | RND-FA-3.5/2.129 | method B: R1 = |
| phenylquinolin-2- | 1H), 7.79(dd, J = 8.8, 2.4 Hz, | Observed mass [M + H]: | bromo; |
| yl)oxy]acetic acid | 1H), 7.62-7.56 (m, 3H), 7.54- | 357.90 | R2 = phenyl; |
| | 7.48(m, 4H), 6.61 (s, 1H), | Exact mass: 357.00 | R3 = ethyl acid |
| | 5.05 (s, 2H) | | |
| 2-[(6-bromo-4- | 400 MHz-DMSO-d6: 12.6 (s, | RND-FA-3.5/1.96 | method A: R1 = |
| phenylquinolin-2- | 1H), 7.66-7.53 (m, 8H), 7.02 | Observed mass [M + H]: | bromo; |
| yl)(methyl)amino] | (br s, 1H), 4.44 (s, 2H), 3.21 | 370.90 | R2 = phenyl; |
| acetic acid | (s, 3H) | Exact mass: 370.03 | R3 = ethyl |
| | | | acid; |
| | | | R4 = methyl |
| 2-[(6-pentyl-4- | 400 MHz-DMSO-d6: 13.1 (s, | Observed mass [M + H]: | method B: R1 = |
| phenylquinolin-2- | 1H), 7.59-7.54 (m, 3H), 7.57- | 350.00 | n-pentyl; |
| yl)oxy]acetic acid | 7.47 (m, 3H), 7.39 (d, J = 8.8 | Exact mass: 349.17 | R2 = phenyl; |
| | Hz, 1H), 7.23 (d, J = 1.6 Hz, | | R3 = ethyl acid |
| | 1H), 6.51 (br s, 1H), 5.03 (s, | | |
| | 2H), 2.55 (m, 2H), 1.53-1.46 | | |
| | (m, 2H), 1.28-1.19 (m, 4H), | | |
| | 0.82 (t, J = 6.8 Hz, 3 Hz) | | |
| 2-[(4-phenyl-6- | 400 MHz-DMSO-d6: 13.1 (s, | RND-FA-3.5/2.24 | method B: R1 = |
| propylquinolin-2- | 1H), 7.57-7.53 (m, 3H), 7.49- | Observed mass [M + H]: | n-propyl; |
| yl)oxy]acetic acid | 7.42 (m, 3H), 7.31 (d, J = 8.8 | 322.10 | R2 = phenyl; |
| | Hz, 1H), 7.20 (d, J = 1.6 Hz, | Exact mass: 321.14 | R3 = ethyl acid |
| | 1H), 6.47 (br s, 1H), 4.80 (s, | | |
| | 2H), 2.50 (dd, J = 14.8, | | |
| | 7.8 Hz, 2H), 1.50 (m, 2H), | | |
| | 0.84 (t, J = 6.8 Hz, 3 Hz) | | |
| 2-[(6-ethyl-4- | 400 MHz-DMSO-d6: 13.1 (s, | ANL-MCL3/2.17 | method B: R1 = |
| phenylquinolin-2- | 1H), 7.60-7.48 (m, 6H), 7.41 | Observed mass [M + H]: | ethyl; |
| yl)oxy]acetic acid | (d, J = 8.0 Hz, 1H), 7.20 (d, J = | 308.30 | R2 = phenyl; |
| | 1.2 Hz, 1H), 6.52 (br s, 1H), | Exact mass: 307.12 | R3 = ethyl acid |
| | 5.06 (s, 2H), 2.59 (dd, J = | | |
| | 14.8, 7.8 Hz, 2H), 1.13 (t, J = | | |
| | 6.8 Hz, 3 Hz) | | |
| 2-[(6-chloro-4- | 400 MHz-DMSO-d6: 13.4 (s, | RND-FA-3.5/2.084 | method B: R1 = |
| phenylquinolin-2- | 1H), 7.68(dd, J = 8.8, 2.4 Hz, | Observed mass [M + H]: | chloro; |
| yl)oxy]acetic acid | 1H), 7.62-7.50 (m, 6H), 7.34 | 314.00 | R2 = phenyl; |
| | (d, J = 2.4 Hz, 1H), 6.63 (s, | Exact mass: 313.05 | R3 = ethyl acid |
| | 1H), 5.06 (s, 2H), 3.19 (s, | | |
| | 3H), 2.53 (t, J = 7.2 Hz, 2H), | | |
| | 1.49-1.43(m, 2H), 1.29- | | |
| | 1.24(m, 2H), 0.84 (t, J = | | |
| | 6.8 Hz, 3 Hz) | | |
| 2-[(6-butyl-4- | 400 MHz-DMSO-d6: 12.5 (s, | ANL-MCL3/2.413 | method B: R1 = |
| phenylquinolin-2- | 1H), 7.59-7.51 (m, 3H), 7.48- | Observed mass [M + H]: | n-butyl; |
| yl)oxy]acetic acid | 7.42 (m, 3H), 7.30 (d, J = 8.0 | 336.32 | R2 = phenyl; |
| | Hz, 1H), 7.20 (d, J = 1.2 Hz, | Exact mass: 335.15 | R3 = ethyl acid |
| | 1H), 6.47 (br s, 1H), 4.80 (s, | | |
| | 2H), 3.19 (s, 3H), 2.53 (t, J = | | |
| | 7.2 Hz, 2H), 1.49-1.43(m, 2H), | | |
| | 1.29-1.24(m, 2H), 0.84 (t, J = | | |
| | 6.8 Hz, 3 Hz) | | |
| 2-[(6-butyl-4- | 400 MHz-DMSO-d6: 12.5 (s, | RND-FA-3.5/1.993 | method A: R1 = |
| phenylquinolin-2- | 1H), 7.59-7.51 (m, 6H), 7.41 | Observed mass [M + H]: | n-butyl; |
| yl)(methyl)amino] | (dd, J = 8.8, 2.0 Hz, 1H), 7.30 | 349.10 | R2 = phenyl; |
| acetic acid | (d, J = 1.2 Hz, 1H), 6.91 (br s, | Exact mass: 348.18 | R3 = ethyl |
| | 1H), 4.42 (s, 2H), 3.19 (s, | | acid; |
| | 3H), 2.58(t, J = 7.2 Hz, 2H), | | R4 = methyl |
| | 1.55-1.47(m, 2H), 1.29- | | |
| | 1.24(m, 2H), 0.86 (t, J = | | |
| | 6.8 Hz, 3 Hz) | | |
| 1-(6-chloro-4- | 500 MHz-CDCl3: 7.71 (d, J = | Observed mass: | method A: R1 = |
| phenylquinolin-2- | 9 Hz, 1H), 7.63(d, J = 2.5 Hz, | Exact mass: 352.10 | chloro; |
| yl)pyrrolidine-2- | 1H), 7.57-7.53(m, 4H), 7.47- | | R2 = phenyl; |
| carboxylic acid | 7.45(m, 2H), 6.93 (br s, 1H), | | R3-R4 = 2- |
| | 5.02(q, J = 7 Hz, 1H), 3.15 (s, | | carboxylic |
| | 3H), 1.59(d, J = 7 Hz, 3H), | | acid-proline |
| 2-[(6-chloro-4- | 500 MHz-CDCl3: 7.71 (d, J = | RND-X-bridge-5.0 MIN/ | method A: R1 = |
| phenylquinolin-2- | 9 Hz, 1H), 7.63(d, J = 2.5 Hz, | 2.306 | n-butyl; |
| yl)(methyl)amino] | 1H), 7.57-7.53(m, 4H), 7.47- | Observed mass [M + H]: | R2 = phenyl; |
| propanoic acid | 7.45(m, 2H), 6.93 (br s, 1H), | 341.10 | R3 = 1-methyl- |
| | 5.02(q, J = 7 Hz, 1H), 3.15 (s, | Exact mass: 340.10 | ethyl acid; |
| | 3H), 1.59(d, J = 7 Hz, 3H), | | R4 = methyl |
| 2-[(6-chloro-4- | 400 MHz-DMSO-d6: 12.5 (s, | RND-FA-3.5 MIN/ | method A: R1 = |
| phenylquinolin-2- | 1H), 7.63-7.51 (m, 7H), 7.45 | 1.865 | chloro; |
| yl)(methyl)amino] | (d, J = 2.4 Hz, 1H), 7.02 (br s, | Observed mass [M + H]: | R2 = phenyl; |
| acetic acid | 1H), 4.42 (s, 2H), 3.21 (s, 3H) | 327.00 | R3 = ethyl |
| | | Exact mass: 326.08 | acid; |
| | | | R4 = methyl |
| 6-chloro-4-phenyl- | | Observed mass: | method A: R1 = |
| 2-(piperidin-1- | | Exact mass: 322.12 | chloro; |
| yl)quinoline | | | R2 = phenyl; |
| | | | R3-R4 = |
| | | | piperidinyl |
| 2-({6-[(1E)-hex-1- | 1H-NMR (400 MHz, DMSO- | G-LCMS-6 min-001; | Scheme 6: |
| en-1-yl]-4- | d6): _ 12.58 (s, 1H), 7.84-7.72 | r.t. = 3.164 min; exp. | Suzuki |
| phenylquinolin-2- | (m, 2H), 7.62-7.54 (m, 5H), | (M + H)+ = 375.2; obs | reaction with |
| yl}(methyl)amino) | 7.43 (s, 1H), 7.10 (s, 1H), | (M + H)+ = 375.1 | (1E)-hexenyl |
| acetic acid | 6.42(d, J = 16.0 Hz, 1H), 6.24 | | boronic acid |
| | (m, 1h), 4.56(s, 2H), 3.30(s, | | |
| | 3H), 2.16 (q, J = 6.8 Hz, 2H), | | |
| | 1.42-1.35(m, 2H), 1.34- | | |
| | 1.28(m, 2H), 0.88 (t, J = 7.20 | | |
| | Hz, 3H) | | |
| 2-[methyl({4- | 1H-NMR (400 MHz, DMSO- | G-LCMS-6 min-001; | Scheme 6: |
| phenyl-6-[(1E)-2- | d6): _ 12.57 (bs, 1H), 7.97 (d, | r.t. = 3.380 min; exp. | +suzuki with |
| phenylethenyl] | J = 7.20 Hz, 1H), 7.63-7.55 | (M + H)+ = 395.2; obs | (1E)-2- |
| quinolin-2- | (m, 9H), 7.35-7.31 (m, 2H), | (M + H)+ = 395.1 | phenyl-vinyl |
| yl})amino]acetic | 7.27-7.20 (m, 2H), 7.18-7.13 | | boronic acid |
| acid | (m, 1H), 6.95 (s, 1H), 4.46 (s, | | |
| | 2H), 3.22 (s, 3H) | | |
| 2-{methyl [4- | 1H-NMR (400 MHz, DMSO- | G-LCMS-6 min-001; | Scheme 6: |
| phenyl-6-(2- | d6): _12.53 (bs, 1H), 7.55- | r.t. = 2.770 min; exp. | Suzuki with |
| phenylethyl)quinolin- | 7.49 (m, 4H), 7.45 (dd, J = | (M + H)+ = 397.2; obs | (1E)-2- |
| 2-yl]amino}acetic | 1.60, 8.60 Hz, 1H), 7.37-7.34 | (M + H)+ = 397.0 | phenyl-vinyl |
| acid | (m, 2H), 7.26-7.22 (m, 2H), | | boronic acid |
| | 7.19-7.15 (m, 2H), 7.11 (d, J = | | then |
| | 6.80 Hz, 2H), 6.88 (s, 1H), | | hydrogenation |
| | 4.41 (s, 2H), 3.19 (s, 3H), | | |
| | 2.89 (d, J = 6.00 Hz, 2H), | | |
| | 2.85 (d, J = 5.60 Hz, 2H), | | |
| 2-[(6-hexyl-3- | 1H-NMR (400 MHz, DMSO- | G-LCMS-12 min-001; | Scheme 4 |
| methyl-4- | d6): 7.81 (s, 1H), 7.65-7.53 | r.t. = 4.660 min; exp. | |
| phenylquinolin-2- | (m, 4H), 7.33-7.28 (m, 2H), | (M + H)+ = 391.2; obs | |
| yl)(methyl)amino] | 6.93 (s, 1H), 4.25 (s, 2H), | (M + H)+ = 391.0 | |
| acetic acid | 3.19 (s, 3H), 2.57-2.54 (m, | | |
| | 2H), 2.09 (s, 3H), 1.50-1.47 | | |
| | (m, 2H), 1.15-1.25 (m, 6H), | | |
| | 0.81 (t, J = 6.8 Hz, 3H), | | |
| 6-hexyl-N-methyl- | 1H-NMR (400 MHz, DMSO- | G-LCMS-12 min-001; | Scheme 5 with |
| 4-phenyl-N-[(2H- | d6): _ 7.59-7.54 (m, 4H), | r.t. = 7.451 min; exp. | N-methyl-1- |
| 1,2,3,4-tetrazol-5- | 7.52-7.49 (m, 2H), 7.42-7.39 | (M + H)+ = 401.2; obs | (2H-tetrazol- |
| yl)methyl]quinolin- | (m, 1H), 7.33 (br.s, 1H), 7.02 | (M + H)+ = 401.0 | 5- |
| 2-amine | (s, 1H), 5.22 (s, 2H), 3.27 (s, | | yl)methanamine |
| | 3H), 2.67-2.56 (m, 2H), 1.51- | | |
| | 1.49 (m, 2H), 1.23-1.15 (m, | | |
| | 7H), 0.82 (t, J = 6.80 Hz, 3H) | | |
| 6-hexyl-N-methyl- | 1H-NMR (400 MHz, DMSO- | G-LCMS-6 min-001; | Scheme 9 |
| 4-phenyl-N-(2H- | d6): _ 15.71 (s, 1H), 8.26 (d, J = | r.t. = 4.050 min; exp. | |
| 1,2,3,4-tetrazol-5- | 8.40 Hz, 1H), 7.64-7.55 (m, | (M + H)+ = 387.2; obs | |
| yl)quinolin-2-amine | 7H), 7.50 (s, 1H), 3.83 (s, | (M + H)+ = 387.0 | |
| | 3H), 2.67 (t, J = 15.20 Hz, | | |
| | 2H), 1.62-1.55 (m, 2H), 1.31- | | |
| | 1.20 (m, 6H), 0.83 (t, J = 7.2 | | |
| | Hz, 3H) | | |
| 5-[(6-hexyl-4- | 1H-NMR (400 MHz, CDCl3): | G-LCMS-6 min-001; | Scheme 11 |
| phenylquinolin-2- | _ 7.99 (s, 1H), 7.97 (s, 1H), | r.t. = 4.075 min; exp. | |
| yl)methyl]-1,3- | 7.61 (br.s, 1H), 7.58-7.26 (m, | (M + H)+ = 419.2; obs | |
| thi azolidine-2,4- | 6H), 7.18 (s, 1H), 5.061 (dd, J = | (M + H)+ = 419.0 | |
| dione | 3.6 Hz, 1H), 4.04-3.99 (m, | | |
| | 1H), 3.60-3.53 (m, 1H), 2.70 | | |
| | (t, J = 7.6 Hz, 2H), 1.63-1.55 | | |
| | (m, 2H), 1.33-1.26 (m, 6H) | | |
| | 0.88-0.84 (t, J = 6.8 Hz, 3H) | | |
| 5-[(6-hexyl-4- | 1H-NMR (400 MHz, DMSO- | G-LCMS-12 min-001; | Scheme 10 |
| phenylquinolin-2- | d6): _ 12.49 (s, 1H), 8.12 (d, J = | r.t. = 8.988 min; exp. | |
| yl)methylidene]- | 8.8 Hz, 1H), 7.96 (br.s, 1H), | (M + H)+ = 417.2; obs | |
| 1,3-thiazolidine- | 7.86 (s, 1H), 7.75-7.72 (m, | (M + H)+ = 417.4 | |
| 2,4-dione | 1H), 7.61-7.56 (m, 6H), 2.72 | | |
| | (t, J = 14.4 Hz, 2H), 1.62-1.55 | | |
| | (m, 2H), 1.31-1.20 (m, 6H), | | |
| | 0.84-0.82 (m, 3H) | | |
| 2-[(6-hexyl-4- | 1H-NMR (400 MHz, DMSO- | G-LCMS-12 min-001; | Scheme 12 |
| phenylquinolin-2- | d6): _ 7.69-7.67 (m, 1H), | r.t. = 5.062 min; exp. | |
| yl)oxy]propanoic | 7.61-7.51 (m, 6H), 7.46-7.45 | (M + H)+ = 378.2; obs | |
| acid | (m, 1H), 6.93 (s, 1H), 5.50 | (M + H)+ = 378.0 | |
| | (m, 1H), 2.68-2.64 (m, 2H), | | |
| | 1.57-1.52 (m, 5H), 1.29-1.20 | | |
| | (m, 6H), 0.82 (t, J = 6.8 Hz, | | |
| | 3H) | | |
| 3-(6-hexyl-4- | 1H-NMR (400 MHz, DMSO- | G-LCMS-6 min-001; | Scheme 13 |
| phenylquinolin-2- | d6): _ 8.03 (d, J = 8.40 Hz, | r.t. = 3.910 min; exp. | |
| yl)butanoic acid | 1H), 7.66-7.63 (m, 2H), 7.58- | (M + H)+ = 376.2; obs | |
| | 7.54 (m, 3H), 7.52-7.49 (m, | (M + H)+ = 376.0 | |
| | 2H), 7.29 (s, 1H), 3.52-3.48 | | |
| | (m, 1H), 3.15 (dd, J = 1.60, | | |
| | 14.80 Hz, 1H), 2.91 (dd, J = | | |
| | 7.20, 14.80 Hz, 1H), 2.73 (t, J = | | |
| | 8.00 Hz, 2H), 1.64-1.60 (m, | | |
| | 2H), 1.54 (d, J = 7.20 Hz, | | |
| | 3H), 1.33-1.22 (m, 6H), 0.88- | | |
| | 0.85 (m, 3H), | | |
| 2-{[6-hexyl-4-(3- | 1H-NMR (400 MHz, DMSO- | G-LCMS-6 min-001; | Method C |
| methylphenyl) | d6): _ 12.58 (s, 1H), 7.54-7.52 | r.t. = 3.217 min; exp. | with R1 = |
| quinolin-2- | (m, 1H), 7.46-7.42 (m, 1H), | (M + H)+ = 391.2; obs | hexyl, R2 = 3- |
| yl](methyl)amino} | 7.40-7.40 (m, 1H), 7.38-7.27 | (M + H)+ = 391.0 | tolyl, R3 = |
| acetic acid | (m, 4H), 6.88 (s, 1H), 4.40 (s, | | ethyl acid, |
| | 2H), 3.19 (s, 3H), 2.68-2.60 | | R4 = methyl |
| | (m, 2H), 2.41 (s, 3H), 1.54- | | |
| | 1.51 (m, 2H), 1.29-1.20 (m, | | |
| | 6H), 0.82 (t, J = 7.20 Hz, 3H) | | |
| 2-[(6-heptyl-4- | 1H-NMR (400 MHz, DMSO- | G-LCMS-6 min-001; | Method C |
| phenylquinolin-2- | d6): _ 12.52 (s, 1H), 7.59-7.50 | r.t. = 3.290 min; exp. | with R1 = |
| yl)(methyl)amino] | (m, 6H), 7.46-7.42 (m, 1H), | (M + H)+ = 391.2; obs | heptyl, R2 = |
| acetic acid | 7.32 (m, 1H), 6.92 (s, 1H), | (M + H)+ = 391.0 | phenyl, R3 = |
| | 4.43 (s, 2H), 3.20 (s, 3H), | | ethyl acid, |
| | 2.60-2.56 (m, 2H), 1.54-1.51 | | R4 = methyl |
| | (m, 2H), 1.29-1.20 (m, 8H), | | |
| | 0.83 (t, J = 7.20 Hz, 3H) | | |
| 2-{[6-(4- | 1H-NMR (400 MHz, DMSO- | G-LCMS-6 min-001; | Scheme 6: |
| ethylphenyl)-4- | d6): _ 12.57 (s, 1H), 7.87-7.83 | r.t. = 3.125 min; exp. | Suzuki with 4- |
| phenylquinolin-2- | (m, 1H), 7.74-7.74 (m, 1H), | (M + H)+ = 397.2; obs | ethylphenyl |
| yl](methyl)amino} | 7.70-7.68 (m, 1H), 7.60-7.58 | (M + H)+ = 397.1 | boronic acid |
| acetic acid | (m, 4H), 7.57-7.51 (m, 1H), | | |
| | 7.46 (d, J = 8.40 Hz, 2H), | | |
| | 7.26 (d, J = 8.40 Hz, 2H), | | |
| | 6.99 (s, 1H), 4.47 (s, 2H), | | |
| | 3.23 (s, 3H), 2.68-2.58 (m, | | |
| | 2H), 1.18 (t, J = 7.20 Hz, 3H) | | |
| 2-{methyl[4- | 1H-NMR (400 MHz, DMSO- | G-LCMS-12 min-001; | Scheme 6: |
| phenyl-6-(3- | d6): 7.86 (dd, J = 2.00, 8.60 | r.t. = 5.632 min; exp. | Suzuki with 3- |
| propylphenyl) | Hz, 1H), 7.75 (d, J = 2.00 Hz, | (M + H)+ = 411.2; obs | propylphenyl |
| quinolin-2- | 1H), 7.70 (d, J = 8.80 Hz, | (M + H)+ = 411.0 | boronic acid |
| yl]amino}acetic | 1H), 7.60-7.53 (m, 5H), 7.39- | | |
| acid | 7.36 (m, 1H), 7.34-7.33 (m, | | |
| | 2H), 7.72-7.32 (m, 1H), 6.99 | | |
| | (s, 1H), 4.45 (s, 2H), 3.23 (s, | | |
| | 3H), 2.59-2.61 (m, 2H), 1.63- | | |
| | 1.57 (m, 2H), 0.89 (t, J = 7.20 | | |
| | Hz, 3H), | | |
| 1-(6-hexyl-4- | 1H-NMR (400 MHz, DMSO- | G-LCMS-5 min-002; | Scheme 5 with |
| phenylquinolin-2- | d6): _ 12.58 (s, 1H), 7.58-7.50 | r.t. = 2.400 min; exp. | pyrrolidine-3- |
| yl)pyrrolidine-3- | (m, 6H), 7.39 (dd, J = 8.0, | (M + H)+ = 403.2; obs | carboxylic |
| carboxylic acid | 1.6 Hz, 1H), 7.31 (m, 1H), | (M + H)+ = 403.4 | acid |
| | 6.70 (s, 1H), 3.64-3.60 (m, | | |
| | 2H), 3.58-3.54 (m, 2H), 3.23- | | |
| | 3.16 (m, 1H), 2.59-2.51 (m, | | |
| | 2H), 2.23-2.15 (m, 2H), 1.52- | | |
| | 1.50 (m, 2H), 1.29-1.20 (m, | | |
| | 6H), 0.82 (t, J = 6.8 Hz, 3H) | | |
| 1-(6-hexyl-4- | 1H-NMR (400 MHz, DMSO- | G-LCMS-6 min-001; | Scheme 5 with |
| phenylquinolin-2- | d6): _ 12.58 (s, 1H), 7.62-7.49 | r.t. = 2.727 min; exp. | azetidine-3- |
| yl)azetidine-3- | (m, 6H), 7.42 (dd, J = 8.4, | (M + H)+ = 389.2; obs | carboxylic |
| carboxylic acid | 1.6 Hz, 1H), 7.34 (s, 1H), 6.61 | (M + H)+ = 397.1 | acid |
| | (s, 1H), 4.28 (t, J = 8.4 Hz, | | |
| | 2H), 4.15 (t, J = 8.4 Hz, 2H), | | |
| | 3.59-3.54 (m, 1H), 2.59-2.51 | | |
| | (m, 2H) 1.52-1.50 (m, 2H), | | |
| | 1.29-1.20 (m, 6H), 0.82 (t, J = | | |
| | 6.80 Hz, 3H) | | |
| 2-[(6-hexyl-4- | 1H-NMR (400 MHz, DMSO- | G-LCMS-12 min-001; | Scheme 5 with |
| phenylquinolin-2- | d6): 7.91 (t, J = 5.6 Hz, 1H), | r.t. = 3.251 min; exp. | (methyl)amino- |
| yl)(methyl)amino]- | 7.59-7.50 (m, 6H), 7.39 (dd, J | (M + H)+ = 420.3; obs | N-(2- |
| N-(2- | = 8.8, 2.0 Hz, 1H), 7.31 (s, | (M + H)+ = 420.0 | hydroxy ethyl) |
| hydroxyethyl) | 1H), 6.86 (s, 1H), 4.64 (t, J = | | acetamide |
| acetamide | 5.6 Hz, 1H), 4.28 (s, 2H), | | |
| | 3.41-3.27(m, 2H), 3.18 (s, | | |
| | 3H), 3.14 (t, J = 6 Hz, 2H), | | |
| | 2.60-2.56 (m, 2H), 1.54-1.51 | | |
| | (m, 2H), 1.29-1.20 (m, 6H), | | |
| | 0.82 (t, J = 7.20 Hz, 3H) | | |
| 2-[(6-hexyl-4- | 1H-NMR (400 MHz, DMSO- | G-LCMS-12 min-001; | |
| phenyl-5,6,7,8- | d6): _ 7.46-7.38 (m, 3H), | r.t. = 7.249 min; exp. | |
| tetrahydroquinolin- | 7.33-7.31 (m, 2H), 6.26 (s, | (M + H)+ = 381.2; obs | |
| 2- | 1H), 4.23 (s, 1H), 3.00 (s, | (M + H)+ = 381.0 | |
| yl)(methyl)amino] | 3H), 3.78( dd, J = 8.4, 4.4 Hz, | | |
| acetic acid | 1H), 2.33-2.26(m, 1H), 1.89- | | |
| | 1.70 (m, 2H), 1.40-1.25(m, | | |
| | 10H), 1.18-1.14 (m, 1H), 0.87 | | |
| | (t, J = 7.20 Hz, 3H) | | |
| cis-2-(6-hexyl-4- | 1H-NMR (400 MHz, DMSO- | G-LCMS-12 min-001; | Scheme 18 |
| phenylquinolin-2- | d6): 8.08 (d, J = 8.40 Hz, | r.t. = 9.293 min; exp. | |
| yl)cyclopropane-1- | 1H), 7.83 (d, J = 8.80 Hz, | (M + H)+ = 374.2; obs | |
| carboxylic acid | 1H), 7.77 (s, 1H), 7.67 (s, | (M + H)+ = 374.5 | |
| | 1H), 7.63-7.57 (m, 5H), 3.03 | | |
| | (q, J = 8.40 Hz, 1H), 2.78 (t, J = | | |
| | 8.00 Hz, 2H), 2.43 (q, J = | | |
| | 8.40 Hz, 1H), 2.02-1.98 (m, | | |
| | 1H), 1.77-1.72 (m, 1H), 1.67- | | |
| | 1.61 (m, 2H), 1.39-1.25 (m, | | |
| | 6H), 0.89-0.85 (m, 3H), | | |
| trans-2-(6-hexyl-4- | | G-LCMS-12 min-001; | Scheme 18 |
| phenylquinolin-2- | | r.t. = 6.932 min; exp. | |
| yl)cyclopropane-1- | | (M + H)+ = 374.2; obs | |
| carboxylic acid | | (M + H)+ = 374.5 | |
| 2-{methyl[4- | 1H-NMR (400 MHz, DMSO- | G-LCMS-12 min-001; | Scheme 6: |
| phenyl-6-(3- | d6): _ 12.54 (s, 1H), 7.59-7.50 | r.t. = 4.372 min; exp. | Suzuki with 3- |
| phenylpropyl) | (m, 6H), 7.42 (dd, J = 8.8, 2.0 | (M + H)+ = 411.2; obs | phenyl-1-vinyl |
| quinolin-2- | Hz, 1H), 7.31 (d, J = 1.6 Hz, | (M + H)+ = 411.0 | boronic acid |
| yl]amino}acetic | 1H), 7.24 (t, J = 8 Hz, 2H), | | |
| acid | 7.15 (m, 3H), 6.91 (s, 1H), | | |
| | 4.42 (s, 2H), 3.19(s, 3H), | | |
| | 2.64-2.55 (m, 4H), 1.85 (q, J | | |
| | = 7.2 Hz, 2H) | | |
| 2-{[6-(benzyloxy)- | 1H-NMR (400 MHz, DMSO- | G-LCMS-10 min-001; | Scheme 14 |
| 4-phenylquinolin-2- | d6): _ 7.57-7.51 (m, 4H), | r.t. = 3.800 min; exp. | |
| yl](methyl)amino} | 7.44-7.42 (m, 2H), 7.37-7.36 | (M − H)− = 397.2; obs | |
| acetic acid | (m, 4H), 7.34-7.29 (m, 2H), | (M − H)− = 397.0 | |
| | 7.00 (d, J = 2.40 Hz, 1H), | | |
| | 6.88 (s, 1H), 5.02 (s, 2H), | | |
| | 4.32 (s, 2H), 3.16(s, 3H), | | |
| 2-[(6-methoxy-4- | 1H-NMR (400 MHz, DMSO- | | Scheme 14 |
| phenylquinolin-2- | d6): _ 12.54 (s, 1H), 7.57-7.50 | | |
| yl)(methyl)amino] | (m, 6H), 7.23 (dd, J = 11.2, | | |
| acetic acid | 2.8 Hz, 1H), 6.95(d, J = 2.8 Hz, | | |
| | 1H), 6.91 (s, 1H), 4.37 (s, | | |
| | 2H), 3.70 (s, 3H), 3.15 (s, | | |
| | 3H), | | |
| 2-[methyl({6-[2-(2- | 1H-NMR (400 MHz, DMSO- | G-LCMS-12 min-001; | Scheme 6: |
| methylphenyl)ethyl]- | d6): _ 12.57 (s, 1H), 7.55-7.54 | r.t. = 5.315 min; exp. | Suzuki with 2- |
| 4-phenylquinolin- | (m, 2H), 7.52-7.49 (m, 2H), | (M + H)+ = 411.2; obs | methylphenyl- |
| 2-yl})amino]acetic | 7.45(dd, J = 8.4, 2.0 Hz, 1H), | (M + H)+ = 411.0 | 1-vinyl |
| acid | 7.36-7.34 (m, 2H), 7.18 (d, J = | | boronic acid |
| | 1.6 Hz, 1H), 7.12-7.02 | | |
| | (m, 4H), 6.88 (s, 1H), 4.41 (s, | | |
| | 2H), 3.19(s, 3H), 2.84(br s, | | |
| | 4H), 2.13 (s, 3H) | | |
| 2-[methyl({6-[2-(3- | 1H-NMR (400 MHz, DMSO- | G-LCMS-12 min-001; | Scheme 6: |
| methylphenyl)ethyl]- | d6): _ 12.50 (s, 1H), 7.55-7.48 | r.t. = 7.37 min; exp. | Suzuki with |
| 4-phenylquinolin- | (m, 4H), 7.46-7.44 (m, 1H), | (M + H)+ = 411.2; obs | 3- |
| 2-yl})amino]acetic | 7.42-7.35 (m, 2H), 7.22 (s, | (M + H)+ = 411.0 | methylphenyl- |
| acid | 1H), 7.11 (t, J = 15.60 Hz, | | 1-vinyl |
| | 1H), 7.01-6.95 (m, 2H), 6.92- | | boronic acid |
| | 6.86 (m, 2H), 4.41 (s, 2H), | | |
| | 3.19 (s, 3H), 2.90-2.86 (m, | | |
| | 2H), 2.81-2.67 (m, 2H), 2.24 | | |
| | (s, 3H) | | |
| 2-[methyl({6-[2-(4- | 1H-NMR (400 MHz, DMSO- | G-LCMS-12 min-001; | Scheme 6: |
| methylphenyl)ethyl]- | d6): _ 12.57 (s, 1H), 7.55-7.50 | r.t. = 5.463 min; exp. | Suzuki with 4- |
| 4-phenylquinolin- | (m, 2H), 7.43 (dd, J = 8.8, 2.0 | (M + H)+ = 411.2; obs | methylphenyl- |
| 2-yl})amino]acetic | Hz, 1H), 7.36-7.34 (m, 2H), | (M + H)+ = 411.0 | 1-vinyl |
| acid | 7.17 (d, J = 1.6 Hz, 1H), 7.04 | | boronic acid |
| | (d, J = 8.0 Hz, 2H), 6.97 (d, J = | | |
| | 8.0 Hz, 2H), 6.87 (s, 1H), | | |
| | 4.39 (s, 2H), 3.18(s, 3H), | | |
| | 2.88-2.84 (m, 2H), 2.81-2.78 | | |
| | (m, 2H), 2.25 (s, 3H) | | |
| 2-[methyl({4- | 1H-NMR (400 MHz, DMSO- | G-LCMS-12 min-002; | Scheme 6: |
| phenyl-6-[2- | d6): _ 12.57 (s, 1H), 8.73 (d, J = | r.t. = 2.81 min; exp. | Suzuki with 1- |
| (pyridin-4- | 6.0 Hz, 2H), 7.81 (br s, 1H), | (M + H)+ = 398.2; obs | (pyridin-4- |
| yl)ethyl]quinolin-2- | 7.73 (d, J = 6.0 Hz, 2H), 7.64 | (M + H)+ = 398.0 | yl)vinyl |
| yl})amino]acetic | (d, J = 6.0 Hz, 1H), 7.59-7.57 | | boronic acid |
| acid | (m, 3H), 7.42-7.39 (m, 2H), | | |
| | 7.25 (s, 1H), 7.11 (s, 1H), | | |
| | 4.58 (s, 2H), 3.30 (s, 3H), | | |
| | 3.14-3.11 (m, 2H), 3.07-3.04 | | |
| | (m, 2H) | | |
| 2-[methyl({4- | 1H-NMR (400 MHz, DMSO- | G-LCMS-12 min-001; | Scheme 6: |
| phenyl-6-[2- | d6): _ 12.57 (s, 1H), 8.38 (d, J = | r.t. = 2.804 min; exp. | Suzuki with 1- |
| (pyridin-3- | 7.2 Hz, 2H), 8.29 (br s, 1H), | (M − H)− = 396.2; obs | (pyridin-3- |
| yl)ethyl]quinolin-2- | 7.56-7.51 (m, 4H), 7.44 (d, J = | (M − H)− = 396.0 | yl)vinyl |
| yl})amino]acetic | 8.4 Hz, 1H), 7.34 (d, J = 7.6 | | boronic acid |
| acid | Hz, 2H), 7.26 (dd, J = 7.6, | | |
| | 5.2 Hz, 1H), 7.16(s, 1H), | | |
| | 6.87(s, 1H), 4.39 (s, 2H), 3.19 | | |
| | (s, 3H), 2.91-2.87 (m, 4H) | | |
| 2-[methyl({4- | 1H-NMR (400 MHz, DMSO- | G-LCMS-12 min-001; | Scheme 6: |
| phenyl-6-[2- | d6): _ 12.52 (s, 1H), 8.46 (d, J = | r.t. = 2.614 min; exp. | Suzuki with 1- |
| (pyridin-2- | 7.2 Hz, 2H), 7.62-7.60 (m, | (M + H)+ = 398.2; obs | (pyridin-2- |
| yl)ethyl]quinolin-2- | 1H), 7.56-7.51 (m, 4H), 7.44 | (M + H)+ = 398.0 | yl)vinyl |
| yl})amino]acetic | (dd, J = 8.4, 1.2 Hz, 1H), 7.36 | | boronic acid |
| acid | (d, J = 7.6 Hz, 2H), 7.09 (d, J = | | |
| | 8.0 Hz, 1H), 6.87(s, 1H), | | |
| | 4.40 (s, 2H), 3.18(s, 3H), | | |
| | 2.99 (m, 4H) | | |
| 2-({6-[2-(2- | 1H-NMR (400 MHz, DMSO- | G-LCMS-12 min-001; | Scheme 6: |
| chl orophenyl)ethyl]- | d6):_13.0(s, 1H), 7.94 (d, J = | r.t. = 5.227 min; exp. | Suzuki with 1- |
| 4-phenylquinolin- | 7.6 Hz, 1H), 7.71 (dd, J = | (M + H)+ = 431.2; obs | (2- |
| 2- | 8.4, 1.6 Hz, 1H), 7.60-7.55 (m, | (M + H)+ = 431.0 | chlorophenyl) |
| yl}(methyl)amino) | 3H), 7.40 (dd, J = 8.4, 1.2 Hz, | | vinyl boronic |
| acetic acid | 1H), 7.35-7.33 (m, 2H), 7.28- | | acid |
| | 7.21 (m, 3H), 7.19-7.12 (m, | | |
| | 2H), 4.65 (s, 2H), 3.36 (s, | | |
| | 3H), 2.99-2.96 (m, 4H) | | |
| 2-({6-[2-(3- | 1H-NMR (400 MHz, DMSO- | G-LCMS-12 min-001; | Scheme 6: |
| chlorophenyl)ethyl]- | d6): _ 7.56-7.51 (m, 4H), 7.47 | r.t. = 5.184 min; exp. | Suzuki with 1- |
| 4-phenylquinolin- | (m, 1H), 7.38-7.35 (m, 2H), | (M + H)+ = 431.1; obs | (3- |
| 2- | 7.25-7.21 (m, 3H), 7.17 (d, J = | (M + H)+ = 431.3 | chlorophenyl) |
| yl}(methyl)amino) | 1.6 Hz, 1H), 7.08-7.03(m, | | vinyl boronic |
| acetic acid | 1H), 6.88(s, 1H), 4.41 (s, 2H), | | acid |
| | 3.19 (s, 3H), 2.90-2.85 (m, | | |
| | 4H) | | |
| 2-({6-[2-(4- | 1H-NMR (400 MHz, DMSO- | G-LCMS-12 min-001; | Scheme 6: |
| chl orophenyl)ethyl]- | d6): _ 12.54 (s, 1H), 7.55-7.49 | r.t. = 5.949 min; exp. | Suzuki with 1- |
| 4-phenylquinolin- | (m, 4H), 7.44 (dd, J = 8.8, | (M + H)+ = 431.1; obs | (4- |
| 2- | 2.0 Hz, 1H), 7.33-7.27 (m, | (M + H)+ = 431.0 | chlorophenyl) |
| yl}(methyl)amino) | 4H), 7.11-7.08 (m, 3H), 6.87 | | vinyl boronic |
| acetic acid | (s, 1H), 4.41 (s, 2H), 3.19(s, | | acid |
| | 3H), 2.88-2.83 (m, 4H) | | |
| 2-[methyl({6-[2- | 1H-NMR (400 MHz, DMSO- | G-LCMS-12 min-001; | Scheme 21: |
| (morpholin-4- | d6): _ 7.59-7.50 (m, 6H), 7.44 | r.t. = 1.698 min; exp. | reductive |
| yl)ethyl]-4- | (d, J = 2.00 Hz, 1H), 7.41 (s, | (M + H)+ = 406.2; obs | a mination |
| phenylquinolin-2- | 1H), 6.91 (s, 1H), 4.41 (s, | (M + H)+ = 406.4 | with |
| yl})amino]acetic | 2H), 3.52 (t, J = 4.40 Hz, 4H), | | morpholine |
| acid | 3.19 (s, 3H), 2.74 (t, J = 7.60 | | |
| | Hz, 2H), 2.45-2.46 (m, 2H), | | |
| | 2.33-2.36 (m, 4H), | | |
| 2-[methyl((6-[2-(4- | 1H-NMR (400 MHz, DMSO- | G-LCMS-12 min-001; | Scheme 21: |
| methylpiperazin-1- | d6): _ 7.59-7.50 (m, 6H), 7.44 | r.t. = 1.793 min; exp. | reductive |
| yl)ethyl]-4- | (d, J = 1.6 Hz, 1H), 7.40 (s, | (M + H)+ = 419.2; obs | a mination |
| phenylquinolin-2- | 1H), 6.90 (s, 1H), 4.41 (s, | (M + H)+ = 419.5 | with 4- |
| yl})amino]acetic | 2H), 3.19 (s, 3H), 2.72 (t, J = | | methylpiperazine |
| acid | 7.20 Hz, 2H), 2.45-2.46 (m, | | |
| | 2H), 2.33-2.36 (m, 4H), 2.17 | | |
| | (s, 3H) | | |
| 2-({6-[2-(2- | 1H-NMR (400 MHz, DMSO- | G-LCMS-6 min-001; | Scheme 6: |
| methoxyphenyl) | d6): _ 12.5 (s, 1H), 7.60-7.48 | r.t. = 3.475 min; exp. (M- | Suzuki with 1- |
| ethyl]-4- | (m, 4H), 7.43-7.35 (m, 3H), | H)− = 425.2; obs (M- | (2- |
| phenylquinolin-2- | 7.18-7.13 (m, 3H), 6.94(d, J = | H)− = 425.0 | methoxyphenyl) |
| yl}(methyl)amino) | 8.0 Hz, 2H), 6.78(t, J = 7.6 Hz, | | vinyl |
| acetic acid | 1H), 4.41 (s, 2H), 3.71 (s, | | boronic acid |
| | 3H), 3.19 (s, 3H), 2.85-2.75 | | |
| | (br s, 4H) | | |
| 2-({6-[2-(3- | 1H-NMR (400 MHz, DMSO- | G-LCMS-12 min-001; | Scheme 6: |
| methoxyphenyl) | d6): _ 12.51 (s, 1H), 7.60-7.45 | r.t. = 5.065 min; exp. | Suzuki with 1- |
| ethyl]-4- | (m, 5H), 7.43-7.35 (m, 2H), | (M − H)− = 425.2; obs | (3- |
| phenylquinolin-2- | 7.25-7.18 (m, 1H), 7.15-7.10 | (M − H)− = 425.0 | methoxyphenyl) |
| yl}(methyl)amino) | (m, 1H), 6.88(m, 1H), 6.78- | | vinyl |
| acetic acid | 6.66 (m, 3H), 4.41 (s, 2H), | | boronic acid |
| | 3.68 (s, 3H), 3.19 (s, 3H), | | |
| | 2.92-2.75 (m, 4H) | | |
| 2-({6-[2-(4- | 1H-NMR (400 MHz, DMSO- | G-LCMS-12 min-001; | Scheme 6: |
| methoxyphenyl) | d6): _ 12.54 (s, 1H), 7.55-7.49 | r.t. = 7.184 min; exp. | Suzuki with 1- |
| ethyl]-4- | (m, 4H), 7.44 (d, J = 8.8, 1H), | (M + H)+ = 427.2; obs | (4- |
| phenylquinolin-2- | 7.35-7.30 (m, 2H), 7.14 (s, | (M + H)+ = 427.0 | methoxyphenyl) |
| yl}(methyl)amino) | 1H), 7.00 (d J = 8, 4 hz, 2H), | | vinyl |
| acetic acid | 6.82-6.77 (m, 3H), 4.29 (s, | | boronic acid |
| | 2H), 3.71 (s, 3H), 3.18 (s, | | |
| | 3H), 2.85-2.80 (m, 2H), 2.78- | | |
| | 2.75 (m, 2H) | | |
| 1-[4-phenyl-6-(2- | 1H-NMR (400 MHz, DMSO- | G-LCMS-6 min-001; | scheme 31 |
| phenylethyl)quinolin- | d6): _ 12.53 (bs, 1H), 7.57 (d, | r.t. = 2.485 min; exp. | |
| 2-yl]pyrrolidine- | J = 8.8 Hz, 1H), 7.52-7.47(m, | (M + H)+ = 423.2; obs | |
| 3-carboxylic acid | 2H), 7.44 (d, J = 8.4 Hz, 1H), | (M + H)+ = 423.0 | |
| | 7.36 (m, 2H), 7.26-7.22 (m, | | |
| | 4H), 7.11 (d, J = 6.8 Hz, 2H), | | |
| | 6.68 (s, 1H), 6.52 (s, 1H), | | |
| | 3.79-3.70 (m, 2H), 3.67-3.55 | | |
| | (m, 2H), 3.22-3.19 (m, 1H), | | |
| | 2.89-2.80 (m, 4H), 2.25-2.15 | | |
| | (m, 2H) | | |
| 2-{[4-(2- | 1H-NMR (400 MHz, MeOD): | G-LCMS-6 min-001; | Method A |
| fluorophenyl)-6- | _ 12.58 (s, 1H), 7.78 (d, J = | r.t. = 3.166 min; exp. | |
| hexylquinolin-2- | 8.4 Hz, 1H), 7.62-7.56 (m, | (M + H)+ = 395.2; obs | |
| yl](methyl)amino} | 1H), 7.54-7.51 (m, 1H), 7.47 | (M + H)+ = 395.0 | |
| acetic acid | (dt, J = 7.2, 1.6 Hz, 1H), 7.38 | | |
| | (dt, J = 7.6, 1.2 Hz, 1H), | | |
| | 7.34-7.29 (m, 1H), 7.18 (br s, | | |
| | 1H), 7.05 (s, 1H), 4.40 (s, | | |
| | 2H), 3.35 (s, 3H), 2.63 (t, J = | | |
| | 7.6 Hz, 2H), 1.59-1.54 (m, | | |
| | 2H), 1.29-1.20 (m, 6H), 0.86 | | |
| | (t, J = 6.8 Hz, 3H) | | |
| 5-oxo-1-[4-phenyl- | 1H-NMR (400 MHz, DMSO- | G-LCMS-12 min-001; | Scheme 16 |
| 6-(2- | d6): _ 12.53 (bs, 1H), 8.38 ( s, | r.t. = 7.108 min; exp. | |
| phenylethyl)quinolin- | 1H), 7.88 (d, J = 8.4 Hz, 1H), | (M + H)+ = 437.2; obs | |
| 2-yl]pyrrolidine- | 7.67 (dd, J = 8.4, 2.0 Hz, 1H), | (M + H)+ = 437.0 | |
| 3-carboxylic acid | 7.57-7.53(m, 3H), 7.38-7.35 | | |
| | (m, 3H), 7.24-7.22 (m, 3H), | | |
| | 7.11 (m, 2H), 4.38-4.30 (m, | | |
| | 2H), 3.42-3.35 (m, 1H), 3.10- | | |
| | 2.94 (m, 2H), 2.92-2.85 (m, | | |
| | 4H) | | |
| 1-(6-hexyl-4- | 1H-NMR (400 MHz, MeOD): | G-LCMS-12 min-001; | Scheme 16 |
| phenylquinolin-2- | _ 8.39 (s, 1H), 7.92-7.90 (m, | r.t. = 7.882 min; exp. | |
| yl)-5- | 1H), 7.59-7.51 (m, 7H), 4.52- | (M + H)+ = 417.2; obs | |
| oxopyrrolidine-3- | 4.48 (m, 2H), 3.49-3.40 (m, | (M + H)+ = 417.0 | |
| carboxylic acid | 1H), 2.99-2.97 (m, 2H), 2.70 | | |
| | (t, J = 15.20 Hz, 2H), 1.69- | | |
| | 1.59 (m, 2H), 1.32-1.27 (m, | | |
| | 6H), 0.87 (t, J = 14.00 Hz, | | |
| | 3H), | | |
| 4-(6-hexyl-4- | 1H-NMR (400 MHz, MeOD): | G-LCMS-12 min-001; | Scheme 5 + |
| phenylquinolin-2- | _ 12.53 (bs, 1H), 7.72 (d, J = | r.t. = 7.433 min; exp. | morpholine-3- |
| yl)morpholine-2- | 8.8 Hz, 1H), 7.58-7.47 (m, | (M + H)+ = 419.2; obs | carboxylic |
| carboxylic acid | 6H), 7.38 (m, 1H), 7.11 (s, | (M + H)+ = 419.0 | acid |
| | 1H), 4.47 (d, J = 13.2 Hz, | | |
| | 1H), 4.19 (d, J = 7.2 Hz, 1H), | | |
| | 4.14-4.08 (m, 2H), 3.80- | | |
| | 3.74 (m, 1H), 3.39-3.33 (m, | | |
| | 2H), 2.64 (t, J = 7.2 Hz, 2H), | | |
| | 1.61-1.57 (m, 2H), 1.35-1.25 | | |
| | (m, 6H), 0.87 (t, J = 7.2 Hz, | | |
| | 3H) | | |
| 1-(6-hexyl-4- | 1H-NMR (400 MHz, | | Scheme 5 with |
| phenylquinolin-2- | MeOD): _ 7.97 (d, J = 8.40 | | 1H-imidazole- |
| yl)-1H-imidazole-4- | Hz, 1H), 7.71-7.67 (m, 1H), | | 4-carboxylic |
| carboxylic acid | 7.67 (s, 1H), 7.63-7.57 (m, | | acid |
| | 5H), 7.35(s, 1H), 2.88-2.85 | | |
| | (m, 1H), 2.73 (t, J = 8.00 Hz, | | |
| | 2H), 2.41 (m, 1H), 1.84-1.79 | | |
| | (m, 1H), 1.75-1.70 (m, 1H), | | |
| | 1.65-1.61 (m, 2H), 1.39-1.25 | | |
| | (m, 6H), 0.89-0.85 (m, 3H), | | |
| 1-(6-hexyl-4- | 1H NMR (400 MHz, DMSO- | G-LCMS-12 min-001; | Scheme 17 |
| phenylquinolin-2- | d6): _ 12.20 (bs, 1H), 8.49 (s, | r.t. = 8.293 min; exp. | with 1H- |
| yl)-1H-pyrrole-3- | 1H), 7.96-8.00 (m, 3H), 7.70 | (M + H)+ = 399.2; obs | pyrrole-3- |
| carboxylic acid | (dd, J = 1.60, 8.60 Hz, 1H), | (M + H)+ = 399.0 | carboxylic |
| | 7.66-7.58 (m, 6H), 6.65 (dd, J | | acid |
| | 1.60, 3.20 Hz, 1H), 2.72- = | | |
| | 2.71 (m, 2H), 1.60-1.57 (m, | | |
| | 2H), 1.30-1.20 (m, 6H), 0.83 | | |
| | (t, J = 6.80 Hz, 3H), | | |
| l-(6-hexyl-4- | 1H-NMR (400 MHz, MeOD): | G-LCMS-12 min-001; | Scheme 5 + 3- |
| phenylquinolin-2- | _ 12.75 (s, 1H), 7.92 (d, J = | r.t. = 5.920 min; exp. | methylpyrrolidine- |
| yl)-3- | 8.8 Hz, 1H), 7.70 (dd, J = | (M + H)+ = 415.2; obs | 3- |
| methylpyrrolidine- | 8.4, 1.6 Hz, 1H), 7.63-7.56( | (M + H)+ = 415.0 | carboxylic |
| 3-carboxylic acid | m, 5H), 7.50 (s, 1H), 7.06 (s, | | acid |
| | 1H), 4.31 ( d, J = 10.8 Hz, | | |
| | 1H), 4.00-3.94 (m, 1H), 3, 92- | | |
| | 3.86(m, 1H), 3.65 (d, J = 10.8 | | |
| | Hz, 1H) 2.69( t, J = 7.6 Hz, | | |
| | 2H), 2.70-2.54(m, 1H), 2.18- | | |
| | 2.15(m, 1H), 1.61-1.55(m, | | |
| | 2H), 1.52 (s, 3H), 1.29-1.20 | | |
| | (m, 6H), 0.87 (t, J = 6.8 Hz, | | |
| | 3H) | | |
| 2-{[4-(3- | 1H-NMR (400 MHz, | G-LCMS-12 min-001; | Method B |
| cyanophenyl)-6- | DMSO-d6): _ 12.75(br s, 1H), | r.t. = 7.635 min; exp. | with lactic |
| hexylquinolin-2- | 8.02-8.01(m, 2H), 7.87( d, JK = | (M + H)+ = 402.2; obs | acid |
| yl]oxy}propanoic | 7.6 Hz, 1H), 7.79(t, J = 7.2 | (M + H)+ = 403.5 | |
| acid | hz, 1H), 7.70 (d, J = 8.4 Hz, | | |
| | 1H), 7.57 (dd, J = 7.6, 1.6 | | |
| | Hz, 1H), 7.36 (s, 1H), 7.04 (s, | | |
| | 1H), 5.50 (q, J = 7.2 Hz, 1H), | | |
| | 2.65 (t, J = 7.6 Hz, 2H), 1.58 | | |
| | (d, J = 6.8 Hz, 3H) 1.57-1.52 | | |
| | (m, 2H), 1.29-1.20 (m, 6H), | | |
| | 0.82 (t, J = 6.8 Hz, 3H) | | |
| 2-{[6-butyl-4-(3- | 1H-NMR (400 MHz, | G-LCMS-12 min-001; | Method B |
| cyanophenyl)quinolin- | DMSO-d6): _ 12.86 (br s, | r.t. = 5.959 min; exp. | with lactic |
| 2- | 1H), 8.04-8.02(m, 2H), 7.88 | (M + H)+ = 375.2; obs | acid |
| yl]oxy}propanoic | (d, J = 8.0 Hz, 1H), 7.79 (t, J = | (M + H)+ = 375.0 | |
| acid | 8.0 hz, 1H), 7.70 (d, J = 8.4 | | |
| | Hz, 1H), 7.58 (dd, J = 8.4, | | |
| | 1.6 Hz, 1H), 7.37 (s, 1H), | | |
| | 7.05 (s, 1H), 5.41 (q, J = | | |
| | 7.2 Hz, 1H), 2.66 (t, J = 7.6 | | |
| | Hz, 2H), 1.57 ( d, J = 6.8 Hz, | | |
| | 3H) 1.57-1.52 (m, 2H), 1.31- | | |
| | 1.24 (m, 2H), 0.87 (t, J = 6.8 | | |
| | Hz, 3H) | | |
| 2-[(6-butyl-4- | | G-LCMS-12 min-001; | |
| phenylquinolin-2- | | r.t. = 4.900 min; exp. | |
| yl)(methyl)amino] | | (M + H)+ = 349.2; obs | |
| acetic acid | | (M + H)+ = 349.1 | |
| 2-{[4-phenyl-6-(2- | 1H-NMR (400 MHz, DMSO- | G-LCMS-5 min-001; | Method B |
| phenylethyl) | d6): _ 12.70 (bs, 1H), 7.68 (d, | r.t. = 3.141 min; exp. | with lactic |
| quinolin-2- | J = 7.6 Hz, 1H), 7.59 (dd, J = | (M + H)+ = 398.2; obs | acid |
| yl]oxy}propanoic | 8.4, 2.0 Hz, 1H), 7.53-7.50 | (M + H)+ = 398.0 | |
| acid | (m, 3H), 7.37-7.35(m, 2H), | | |
| | 7.32 (s, 1H), 7.26-7.24 (m, | | |
| | 2H), 7.24-7.19 (m, 1H), 7.12- | | |
| | 7.10 (m, 2H), 6.91 (s, 1H), | | |
| | 5.40 (q, J = 6.8 Hz, 1H), 2.98- | | |
| | 2.94(m, 2H), 2.88-2.84(m, | | |
| | 2H), 1.57 (d, J = 6.8 Hz, | | |
| | 3H) | | |
| N-methyl-4-phenyl- | 1H NMR (400 MHz, DMSO- | G-LCMS-12 min-001; | Scheme 5+ |
| 6-(2-phenylethyl)- | d6): _ 15.72 (s, 1H), 8.27 (d, J = | r.t. = 7.069 min; exp. | Scheme 9 |
| N-(1H-1,2,3,4- | 8.00 Hz, 1H), 7.67 (d, J = | (M + H)+ = 405.2; obs | |
| tetrazol-5- | 8.40 Hz, 1H), 7.55-7.51 (m, | (M + H)+ = 405.0 | |
| yl)quinolin-2-amine | 4H), 7.44-7.42 (m, 2H), 7.37 | | |
| | (s, 1H), 7.26-7.23 (m, 2H), | | |
| | 7.20-7.18 (m, 1H), 7.13 (d, J = | | |
| | 7.20 Hz, 2H), 3.82 (s, 3H), | | |
| | 2.99-2.97 (m, 2H), 2.92-2.90 | | |
| | (m, 2H), | | |
| 4-phenyl-6-(2- | 1H NMR (400 MHz, DMSO- | G-LCMS-12 min-001; | |
| phenylethyl)-2-[2- | d6): _ 15.72 (s, 1H), 7.90 (d, J = | r.t. = 6.948 min; exp. | |
| (1H-1,2,3,4- | 8.4 Hz, 1H), 7.68 (dd, J = | (M + H)+ = 420.2; obs | |
| tetrazol-5- | 8.4, 1.6 Hz, 1H), 7.55-7.51 | (M + H)+ = 420.0 | |
| yl)propan-2- | (m, 3H), 7.43 (s, 1H), 7.34- | | |
| yl]quinoline | 7.31 (m, 3H), 7.26-7.23 (m, | | |
| | 2H), 7.20-7.18 (m, 1H), 7.11 | | |
| | (d, J = 6.8 Hz, 2H), 3.03 (t, J = | | |
| | 7.6 Hz, 2H), 2.89(t, J = | | |
| | 7.6 Hz, 2H), 1.91 (s, 6H), | | |
| 5-{[4-phenyl-6-(2- | 1H NMR (400 MHz, DMSO- | G-LCMS-12 min-001; | Scheme 33 |
| phenylethyl)quinolin- | d6): _ 12.10 (s, 1H), 7.90 (d, J = | r.t. = 6.110 min; exp. | |
| 2-yl]methyl}-1,3- | 8.4 Hz, 1H), 7.69 (dd, J = | (M + H)+ = 439.2; obs | |
| thiazolidine-2,4- | 8.4, 1.6 Hz, 1H), 7.55-7.51 | (M + H)+ = 439.0 | |
| dione | (m, 3H), 7.43 (s, 1H), 7.38- | | |
| | 7.35 (m, 3H), 7.27-7.22 (m, | | |
| | 2H), 7.20-7.18 (m, 1H), 7.15- | | |
| | 7.12 (m, 2H), 5.02 (dd, J = | | |
| | 9.2,3.6 Hz, 1H), 3.86(dd, | | |
| | 16.8, 3.6 Hz, 1H), 3.57(dd, | | |
| | 14.0, 10.4 Hz, 1H), 3.03 (t, J = | | |
| | 7.6 Hz, 2H), 2.89(t, J = | | |
| | 7.6 Hz, 2H) | | |
| N-cyclopropyl-6- | 1H-NMR (400 MHz, DMSO- | G-LCMS-12 min-001; | Scheme 5 + |
| hexyl-4-phenyl-N- | d6): _ 15.66 (s, 1H), 8.18 (d, J = | r.t. = 7.130 min; exp. | Scheme 9 |
| (1H-1,2,3,4- | 8.80 Hz, 1H), 7.64-7.55 (m, | (M + H)+ = 413.2; obs | |
| tetrazol-5- | 6H), 7.50 (br s, 2H), 2.66 (t, J = | (M + H)+ = 413.0 | |
| yl)quinolin-2-amine | 7.20 Hz, 2H), 1.58-1.53 (m, | | |
| | 2H), 1.31-1.20 (m, 6H), 1.18- | | |
| | 1.15(m, 2H), 0.83 (t, J = 7.2 | | |
| | Hz, 3H), 0.8-0.7(m, 2H) | | |
| 2-[methyl({4- | 1H NMR (400 MHz, DMSO- | G-LCMS-12 min-001; | Scheme 6: |
| phenyl-6-[2- | d6): _ 12.54 (s, 1H), 8.54-8.53 | r.t. = 3.58 min; exp. | Suzuki with 2- |
| (pyrazin-2- | (m, 1H), 8.45 (d, J = 2.8 Hz, | (M + H)+ = 399.2; obs | (pyrazin-2- |
| yl)ethyl]quinolin-2- | 1H), 8.39 (d, J = 1.2 Hz, 1H), | (M + H)+ = 399.0 | yl)vinyl |
| yl})amino]acetic | 7.58-7.51 (m, 4H), 7.44 (dd, J = | | boronic acid |
| acid | 8.4, 2.0 Hz, 1H), 7.38-7.35 | | |
| | (m, 2H), 7.16(s, 1H), 6.89 (s, | | |
| | 1H), 4.42 (s, 2H), 3.19(s, 3H), | | |
| | 3.04(s, 4H) | | |
| 2-[methyl((4- | 1H NMR (400 MHz, DMSO- | G-LCMS-12 min-001; | Scheme 6: |
| phenyl-6-[2- | d6): _ 12.54 (s, 1H), 9.01 (s, | r.t. = 3.455 min; exp. | Suzuki with 2- |
| (pyrimidin-5- | 1H), 8.55 (s, 2H), 7.57-7.50 | (M + H)+ = 399.2; obs | (pyrimidin-5- |
| yl)ethyl]quinolin-2- | (m, 4H), 7.46 (dd, J = 8.4, 2.0 | (M + H)+ = 399.0 | yl)vonyl |
| yl})amino]acetic | Hz, 1H), 7.38-7.35 (m, 2H), | | boronic acid |
| acid | 7.16(d, J = 1.2 Hz, 1H), 6.89 | | |
| | (s, 1H), 4.41 (s, 2H), 3.19(s, | | |
| | 3H), 2.95-2.92(m, 2H), 2.89- | | |
| | 2.86(m, 2H) | | |
| 2-[methyl({4- | 1H NMR (400 MHz, DMSO- | G-LCMS-10 min-001; | Scheme 6: |
| phenyl-6-[2- | d6): _ 12.54 (s, 1H), 8.69 (s, | r.t. = 4.287 min; exp. | Suzuki with 2- |
| (quinoxalin-2- | 1H), 8.08-8.05 (m, 1H), 8.01- | (M + H)+ = 449.2; obs | (quinoxalin-2- |
| yl)ethyl]quinolin-2- | 7.99 (m, 1H), 7.86-7.79(m, | (M + H)+ = 449.0 | yl)vinyl |
| yl})amino]acetic | 2H), 7.56 (d, J = 8.4 Hz, 1H), | | boronic acid |
| acid | 7.52(dd, J = 8.8, 2.0 Hz, 1H), | | |
| | 7.43 (t, J = 8.0 Hz, 1H), 7.34 | | |
| | (t, J = 8.0 Hz, 2H), 7.22-7.19 | | |
| | (m, 2H), 7.16 (s, 1H), 6.85 (s, | | |
| | 1H), 4.41 (s, 2H), 3.29- | | |
| | 3.26(m, 2H), 3.17(s, 3H), | | |
| | 3.17-3.15 (m, 2H) | | |
| 2-[methyl({4- | 1H NMR (400 MHz, DMSO- | G-LCMS-12 min-001; | Scheme 6: |
| phenyl-6-[2- | d6): _ 12.53 (s, 1H), 7.54- | r.t. = 3.740 min; exp. | Suzuki with 2- |
| (1,2,3,4- | 7.50 (m, 6H), 7.45(dd, J = 8.4, | (M + H)+ = 453.2; obs | (1,2,3,4- |
| tetrahydroquinoxalin- | 1.6 Hz, 1H), 7.39 (s, 1H), | (M + H)+ = 453.1 | tetrahydroquin |
| 2- | 6.91 (s, 1H), 6.39-6.37(m, | | oxalin-2- |
| yl)ethyl]quinolin-2- | 1H), 6.33-6.3 l(m, 3H), | | yl)vinyl |
| yl})amino]acetic | 5.28(br s, 2), 4.42 (s, 2H), | | boronic acid |
| acid | 3.19(s, 3H), 3.17-3.15 (m, | | |
| | 2H), 2.89-2.84(m, 1H), 2.75- | | |
| | 2.67(m, 2H), 1.70-1.60(m, | | |
| | 2H) | | |
| 2-[methyl({4- | 1H-NMR (400 MHz, DMSO- | G-LCMS-12 min-001; | Scheme 6: |
| phenyl-6-[2- | d6): _ 12.66 (s, 1H), 8.67 (d, J = | r.t. = 3.161 min; exp. | Suzuki with 2- |
| (pyrimidin-2- | 4.8 Hz, 1H), 7.58-7.50 (m, | (M + H)+ = 399.2; obs | (pyrimidin-2- |
| yl)ethyl]quinolin-2- | 4H), 7.47-7.43(m, 1H), 7.38- | (M + H)+ = 399.1 | yl)vinyl |
| yl})amino]acetic | 7.35 (m, 2H), 7.20(d, J = | | boronic acid |
| acid | 1.6 Hz, 1H), 6.86(s, 1H), 4.36 | | |
| | (s, 2H), 3.17(s, 3H), 3.16-3.10 | | |
| | (m, 4H) | | |
| 2-[methyl((4- | 1H-NMR (400 MHz, DMSO- | G-LCMS-12 min-001; | Scheme 6: |
| phenyl-6-[2- | d6): _ 12.66 (s, 1H), 8.85 (dd, | r.t. = 3.272 min; exp. | Suzuki with 2- |
| (quinolin-6- | J = 4.0, 1.6 Hz, 1H), 8.21 (d, J = | (M + H)+ = 448.2; obs | (quinolin-6-yl) |
| yl)ethyl]quinolin-2- | 6.8 Hz, 1H), 7.91 (d, J = | (M + H)+ = 448.0 | vinyl boronic |
| yl})amino]acetic | 8.4 Hz, 1H), 7.62(s, 1H), 7.58- | | acid |
| acid | 7.54(m, 2H), 7.49-7.46(m, | | |
| | 2H), 7.41(t, J = 7.6 Hz, 1H), | | |
| | 7.30(t, J = 8.0 Hz, 2H), 7.14- | | |
| | 7.12(m, 2H), 7.07(d, J = | | |
| | 1.2 Hz, 1H), 6.80 (s, 1H), | | |
| | 4.32 (s, 2H), 3.17(s, 3H), | | |
| | 3.05-3.00 (m, 4H) | | |
| 2-[methyl({4- | 1H-NMR (400 MHz, DMSO- | G-LCMS-6 min-001; | Scheme 6: |
| phenyl-6-[2- | d6): _ 12.55 (s, 1H), 7.56- | r.t. = 3.259 min; exp. | Suzuki with 2- |
| (1,2,3,4- | 7.48(m, 4H), 7.48-7.36(m, | (M + H)+ = 452.2; obs | (1,2,3,4- |
| tetrahydroquinolin- | 3H), 7.21(d, J = 1.6 Hz, 1H), | (M + H)+ = 452.2 | tetrahydroquin |
| 6-yl)ethyl]quinolin- | 6.89 (s, 1H), 6.61-6.55 (m, | | olin-6-yl) |
| 2-yl})amino]acetic | 2H), 6.30(d, J = 8.0 Hz, 1H), | | vinyl boronic |
| acid | 5.41 (s, 1H), 4.41(s, 2H), 3.33 | | acid |
| | (s, 3H), 3.15-3.11 (m, 2H), | | |
| | 2.78 (t, J = 7.2 Hz, 2H), 2.62 | | |
| | (t, J = 8.0 Hz, 2H), 2.56 (t, J = | | |
| | 6.4 Hz, 2H), 1.78-1.72 (m, 2H) | | |
| 2-[methyl({4- | 1H-NMR (400 MHz, DMSO- | G-LCMS-12 min-001; | Scheme 6: |
| phenyl-6-[2- | d6): _ 12.50 (s, 1H), 8.84 (dd, | r.t. = 3.396 min; exp. | Suzuki with 2- |
| (quinolin-7- | J = 4.4, 1.6 Hz, 1H), 8.34 (d, J = | (M + H)+ = 448.2; obs | (quinolin-7- |
| yl)ethyl]quinolin-2- | 7.2 Hz, 1H), 7.86 (d, J = | (M + H)+ = 448.4 | yl)vinyl |
| yl})amino]acetic | 8.4 Hz, 1H), 7.69(s, 1H), 7.58- | | boronic acid |
| acid | 7.46(m, 3H), 7.44-7.39(m, | | |
| | 2H), 7.30(t, J = 8.0 Hz, 2H), | | |
| | 7.16 (d, J = 6.8 Hz, 2H), | | |
| | 7.12(s, 1H), 6.85 (s, 1H), | | |
| | 4.42 (s, 2H), 3.18(s, 3H), | | |
| | 3.11-3.07 (m, 2H), 3.04-3.00 | | |
| | (m, 2H) | | |
| 2-[methyl({4- | 1H-NMR (400 MHz, DMSO- | G-LCMS-12 min-001; | Scheme 6: |
| phenyl-6-[2- | d6): _ 12.50 (s, 1H), 8.89 (s, | r.t. = 3.768 min; exp. | Suzuki with 2- |
| (quinoxalin-6- | 2H), 7.99 (d, J = 8.4 Hz, 1H), | (M + H)+ = 449.2; obs | (quinoxalin-6- |
| yl)ethyl]quinolin-2- | 7.75(s, 1H), 7.67 (dd, J = 8.4, | (M + H)+ = 449.2 | yl)vinyl |
| yl})amino]acetic | 1.6 Hz, 1H), 7.55 (d, J = | | boronic acid |
| acid | 8.4 Hz, 1H), 7.51 (d, J = | | |
| | 8.4 Hz, 1H), 7.42 (t, J = | | |
| | 7.6 Hz, 1H), 7.32 (t, J = | | |
| | 7.6 Hz, 1H), 7.15 (d, J = | | |
| | 7.2 Hz, 1H), 7.09(s, 1H), | | |
| | 6.81 (s, 1H), 4.35 (s, 2H), | | |
| | 3.17 (s, 3H), 3.13 (t, J = | | |
| | 8.0 Hz, 2H), 3.04 (d, J = | | |
| | 8.0 Hz, 2H) | | |
| 2-{methyl [4- | 1H-NMR (400 MHz, DMSO- | G-LCMS-6 min-001; | Scheme 6: |
| phenyl-6-(2-{1H- | d6): _ 11.83 (s, 1H), 8.12 (d, J = | r.t. = 2.069 min; exp. | Suzuki with |
| pyrrolo[2,3- | 5.2 Hz, 1H), 8.02-7.98(br s, | (M + H)+ = 437.2; obs | 1H- |
| b]pyridin-4- | 1H), 7.79-7.70 (br s, 1H), | (M + H)+ = 437.1 | pyrrolo[2,3- |
| yl}ethyl)quinolin-2- | 7.55-7.48 (m, 3H), 7.43 (t, J = | | b]pyridin-4- |
| yl]amino}acetic | 2.8 Hz, 1H), 7.24-7.21 (m, | | vinyl boronic |
| acid | 1H), 7.12 (s, 1H), 6.85 (d, J = | | acid |
| | 3.2 Hz, 1H), 6.48 (s, 1H), | | |
| | 4.60 (s, 2H), 3.39(s, 3H), | | |
| | 3.19 (t, J = 8.0 Hz, 2H), 3.10 | | |
| | (d, J = 8.0 Hz, 2H) | | |
| 2-({6-[2-(1,3- | 1H-NMR (400 MHz, DMSO- | G-LCMS-12 min-001; | Scheme 6: |
| benzothiazol-2- | d6): _ 12.6 (s, 1H), 8.04 (dd, J = | r.t. = 4.610 min; exp. | Suzuki 1,3- |
| yl)ethyl]-4- | 8.0, 0.8 Hz, 1H), 7.91 (d, J = | (M + H)+ = 454.2; obs | benzthiazol-2- |
| phenylquinolin-2- | 8.0 Hz, 1H), 7.57-7.37 (m, | (M + H)+ = 454.3 | vinyl boronic |
| yl}(methyl)amino) | 7H), 7.28-7.26 (m, 3H), 6.86 | | acid |
| acetic acid | (s, 1H), 3.39 (t, J = 7.2 Hz, | | |
| | 2H), 3.18 (s, 3H), 3.15 (d, J = | | |
| | 7.2 Hz, 2H) | | |
| 2-({6-[2-(1,3- | 1H-NMR (400 MHz, DMSO- | G-LCMS-8.5m; r.t. = | Scheme 6: |
| benzoxazol-2- | d6): _ 12.6 (s, 1H), 7.65-7.62 | 3.464 min; exp. | Suzuki with |
| yl)ethyl]-4- | (m, 2H), 7.51 (d, J = 8.4 Hz, | (M + H)+ = 438.2; obs | 1,3- |
| phenylquinolin-2- | 1H) , 7.47-7.42 (m, 4H), | (M + H)+ = 438.1 | benzoxazol-2- |
| yl}(methyl)amino) | 7.37-7.31 (m, 2H), 7.28-7.26 | | vinyl boronic |
| acetic acid | (m, 3H), 6.73 (s, 1H), 4.05 | | acid |
| | (s, 1H), 3.22 (t, J = 7.2 Hz, | | |
| | 2H), 3.16 (s, 3H), 3.13 (d, J = | | |
| | 7.2 Hz, 2H) | | |
| 2-({6-[2-(1H-1,3- | 1H-NMR (400 MHz, DMSO- | G-LCMS-10 min-001; | Scheme 6: |
| benzodiazol-2- | d6): _ 12.12 (s, 1H), 7.54 (d, | r.t. = 2.018 min; exp. | Suzuki with 2- |
| yl)ethyl]-4- | J = 8.8 Hz, 1H), 7.49-7.46 | (M + H)+ = 437.2; obs | (1H-1,3- |
| phenylquinolin-2- | (m, 3H), 7.45-7.38 (m, 3H), | (M + H)+ = 437.0 | benzodiazol- |
| yl}(methyl)amino) | 7.32-7.29 (m, 3H), 7.14-7.09 | | 2-yl)vinyl |
| acetic acid | (m, 2H), 6.25 (s, 1H), 4.33 | | boronic acid |
| | (s, 2H), 3.18 (s, 3H), 3.10- | | |
| | 3.06 (m, 4H) | | |
| 2-[methyl({6-[2-(1- | 1H-NMR (400 MHz, DMSO- | G-LCMS-10 min-001; | 1 + suzuki with |
| methyl-1H-1,3- | d6): _ 12.59 (s, 1H), 7.57- | r.t. = 3.100 min; exp. | 2-(1-methyl- |
| benzodiazol-2- | 7.50 (m, 3H), 7.45-7.41 (m, | (M + H)+ = 451.2; obs | 1,3- |
| yl)ethyl]-4- | 2H), 7.34 (t, J = 7.6 Hz, 2H), | (M + H)+ = 451.0 | benzodi azol- |
| phenylquinolin-2- | 7.24-7.21 (m, 3H), 7.19-7.17 | | 2-yl)vinyl |
| yl})amino]acetic | (m, 2H), 6.86 (s, 1H), 4.40 | | boronic acid |
| acid | (s, 2H), 3.48(s, 3H), 3.18 (s, | | |
| | 3H), 3.16-3.11 (m, 4H) | | |
| 2-[methyl(6-{2- | 1H-NMR (400 MHz, DMSO- | G-LCMS-10 min-001; | Scheme 21: |
| [methyl(phenyl) | d6): _ 12.59 (s, 1H), 7.57- | r.t. = 3.887 min; exp. | redcutive |
| amino]ethyl}-4- | 7.50 (m, 3H), 7.45-7.41 (m, | (M − H)− = 424.2; obs | a mination |
| phenylquinolin-2- | 2H), 7.34 (t, J = 7.6 Hz, 2H), | (M − H)− = 424.0 | with N- |
| yl)amino]acetic | 7.24-7.21 (m, 3H), 7.19-7.17 | | Methyl aniline |
| acid | (m, 2H), 6.86 (s, 1H), 4.40 | | |
| | (s, 2H), 3.53 (t, J = 7.2 Hz, | | |
| | 2H), 3.19(s, 3H), 2.78 (t, J = | | |
| | 7.2 Hz, 2H), 2.75 (s, 3H) | | |
| 2-[methyl({4- | 1H-NMR (400 MHz, DMSO- | G-LCMS-10 min-001; | Scheme 21: |
| phenyl-6-[2- | d6): _ 12.3 (s, 1H), 7.56- | r.t. = 2.581 min; exp. | redcutive |
| (1,2,3,4- | 7.54 (m, 1H), 7.52-7.48 (m, | (M − H)− = 450.2; obs | a mination |
| tetrahydroisoquinolin- | 6H), 7.46-7.44 (m, 1H), | (M − H)− = 450.0 | with 1,2,3,4- |
| 2- | 7.11-7.06 (m, 3H), 7.03-7.00 | | tetrahydroisoq |
| yl)ethyl]quinolin-2- | (m, 1H), 6.90 (s, 1H), 4.40 | | uinoline |
| yl})amino]acetic | (s, 2H), 3.58 (s, 2H), 3.19(s, | | |
| acid | 3H), 2.85(t, J = 7.2 Hz, 2H), | | |
| | 2.74(t, J = 7.2 Hz, 2H), 2.69- | | |
| | 2.64(m, 4H) | | |
| 2-({6-[2-(2,3- | 1H-NMR (400 MHz, DMSO- | G-LCMS-10 min-001; | Scheme 21: |
| dihydro-1H-indol- | d6): _ 12.3 (s, 1H), 7.58-7.44 | r.t. = 5.317 min; exp. | redcutive |
| 1-yl)ethyl]-4- | (m, 8H), 7.00 (d, J = 7.6 Hz, | (M − H)− = 436.2; obs | a mination |
| phenylquinolin-2- | 1H), 6.92(d, J = 7.6 Hz, 1H), | (M − H)− = 436.0 | with 2,3- |
| yl}(methyl)amino) | 6.90( s, 1H), 6.52(t, J = | | dihydro-1H- |
| acetic acid | 7.2 Hz, 1H), 6.38(d, J = 7.6 Hz, | | indole |
| | 1H), 7.03-7.00 (m, 1H), | | |
| | 4.40 (s, 2H), 3.31-3.25 (m, | | |
| | 4H), 3.19 (s, 3H), 2.85-2.81 | | |
| | (m, 4H) | | |
| 2-[methyl({6-[2- | 1H NMR (400 MHz, | G-LCMS-12 min-001; | scheme 22 |
| (pyridin-2-yl)ethyl]- | DMSO-d6): _ 8.73 (dd, J = | r.t. = 1.717 min; exp. | with 2-vinyl- |
| 4-(pyridin-4- | 1.60, 4.00 Hz, 2H), 8.47-8.45 | (M + H)+ = 399.2; obs | pyridine |
| yl)quinolin-2- | (m, 1H), 7.62 (td, J = 1.60, | (M + H)+ = 399.4 | |
| yl})amino]acetic | 7.60 Hz, 1H), 7.54 (d, J = | | |
| acid | 8.80 Hz, 1H), 7.45 (dd, J = | | |
| | 2.00, 8.40 Hz, 1H), 7.39 (td, J = | | |
| | 1.60, 4.40 Hz, 2H), 7.20- | | |
| | 7.17 (m, 1H), 7.13-7.09 (m, | | |
| | 2H), 6.94 (s, 1H), 4.37 (s, | | |
| | 2H), 3.18 (s, 3H), 3.02-2.99 | | |
| | (m, 4H) | | |
| 2-[methyl({6-[2- | 1H NMR (400 MHz, DMSO- | G-LCMS-12 min-001; | scheme 22 |
| (pyridin-3-yl)ethyl]- | d6): 8.71 (d, J = 4.4 Hz, | r.t. = 2.373 min; exp. | with 3-vinyl- |
| 4-(pyridin-4- | 2H), 8.39 (d, J = 4.4 Hz, 1H), | (M + H)+ = 399.2; obs | pyridine |
| yl)quinolin-2- | 8.30 (s, 1H), 7.57-7.53 (m, | (M + H)+ = 399.0 | |
| yl})amino]acetic | 1H), 7.46 (d, J = 8.4 Hz, 1H), | | |
| acid | 7.65 (d, J = 4.4 Hz, 2H), 7.28- | | |
| | 7.25 (m, 1H), 7.09(s, 1H), | | |
| | 6.91 (s, 1H), 4.29 (s, 2H), | | |
| | 3.18 (s, 3H), 2.92-2.86 (m, | | |
| | 4H) | | |
| 2-{[6-hexyl-4- | 1H NMR (400 MHz, DMSO- | G-LCMS-10 min-001; | Method C |
| (pyridin-4- | d6): _8.74 (d, J = 6.0 Hz, | r.t. = 3.868 min; exp. | with R1 = |
| yl)quinolin-2- | 2H), 7.57-7.53 (m, 3H), 7.43 | (M − H)− = 376.2; obs | hexyl, R2 = 4- |
| yl](methyl)amino} | (dd, J = 8.8, 2.0 Hz, 1H), 7.25 | (M − H)− = 376.0 | pyridyl, R3 = |
| acetic acid | (d, J = 2.0 Hz, 1H), 6.98 (s, | | ethyl acid, |
| | 1H), 4.40 (s, 2H), 3.19 (s, | | R4 = methyl |
| | 3H), 2.59 (t, J = 7.2 Hz, 2H), | | |
| | 1.54-1.50(m, 2H), 1.25- | | |
| | 1.21(m, 6H), 0.82(t, J = | | |
| | 6.8 Hz, 3H) 4H) | | |
| 2-{[6-hexyl-4- | 1H-NMR (400 MHz, | G-LCMS-10 min-001; | Scheme 12 |
| (pyridin-4- | DMSO-d6): _ 8.77(dd, 4.4, | r.t. = 5.555 min; exp. | plus Method C |
| yl)quinolin-2- | 1.6 Hz, 2H), 7.70(d, J = 8.4 Hz, | (M + H)+ = 379.2; obs | |
| yl]oxy}propanoic | 1H), 7.59-7.55(m, 3H), 7.40 | (M + H)+ = 379.0 | |
| acid | (s, 1H), 7.02 (s, 1H), 5.40 | | |
| | (m, 1H), 2.68-2.65 (m, 2H), | | |
| | 1.57-1.52 (m, 5H), 1.29-1.20 | | |
| | (m, 6H), 0.82 (t, J = 6.8 Hz, | | |
| | 3H) | | |
| 2-({6-[2-(4- | 1H-NMR (400 MHz, | G-LCMS-10 min-001; | Scheme 21: |
| hydroxypiperidin-1- | DMSO-d6): _ 7.59-7.55(m, | r.t. = 2.467 min; exp. | redcutive |
| yl)ethyl]-4- | 5H), 7.41-7.38 (m, 2H), 6.90 | (M + H)+ = 420.2; obs | amination |
| phenylquinolin-2- | (s, 1H), 4.51(s, 1H), 4.39 | (M + H)+ = 420.0 | with 4- |
| yl}(methyl)amino) | (s, 1H), 2.68-2.65 (m, 2H), | | hydroxy- |
| acetic acid | 2.09-2.04(m, 2H), 1.68- | | piperidine |
| | 1.65(m, 2H), 1.35-1.32(m, | | |
| | 2H) | | |
| 2-[methyl({4- | 1H-NMR (400 MHz, | G-LCMS-6 min-001; | Scheme 21: |
| phenyl-6-[2- | DMSO-d6): _ 7.58-7.50 (m, | r.t. = 2.073 min; exp. | redcutive |
| (piperidin-1- | 5H), 7.42-7.39 (m, 2H), 6.89 | (M + H)+ = 404.2; obs | amination |
| yl)ethyl]quinolin-2- | (s, 1H), 4.39(s, 1H), 3.19(s, | (M + H)+ = 404.1 | with |
| yl})amino]acetic | 3H), 2.73 (t, J = 6.8 Hz, 2H), | | piperidine |
| acid | 2.49-2.43(m, 2H), 2.38- | | |
| | 2.33(m, 4H), 1.45-1.43(m, | | |
| | 4H), 1.37-1.35(m, 2H) | | |
| 2-[methyl({4- | 1H-NMR (400 MHz, | G-LCMS-10 min-001; | Scheme 21: |
| phenyl-6-[2- | DMSO-d6): _ 7.59-7.49 (m, | r.t. = 1.601 min; exp. | redcutive |
| (pyrrolidin-1- | 5H), 7.42-7.39 (m, 2H), 6.90 | (M − H)− = 388.2; obs | amination |
| yl)ethyl]quinolin-2- | (s, 1H), 4.39(s, 1H), 3.19(s, | (M − H)− = 388.0 | with |
| yl})amino]acetic | 3H), 2.79 (s, 4H), 2.67- | | pyrrolidine |
| acid | 2.65(m, 4H), 1.75-1.67(m, | | |
| | 4H), | | |
| 2-({6-[2- | 1H-NMR (400 MHz, | G-LCMS-10 min-001; | Scheme 21: |
| (diethylamino)ethyl]- | DMSO-d6): _ 7.59-7.49 (m, | r.t. = 2.372 min; exp. | redcutive |
| 4-phenylquinolin- | 5H), 7.45-7.39 (m, 2H), 6.91 | (M − H)− = 390.2; obs | amination |
| 2- | (s, 1H), 4.40 (s, 1H), 3.19(s, | (M − H)− = 390.0 | with N, N- |
| yl}(methyl)amino) | 3H), 2.73 (s, 2H), 2.67- | | diethylamine |
| acetic acid | 2.60(m, 2H), 2.54-2.52(m, | | |
| | 2H) 0.95(t, J = 7.2 Hz, 6H) | | |
| 2-({6-[2-(2,3- | 1H-NMR (400 MHz, | G-LCMS-10 min-001; | Scheme 21: |
| dihydro-1H- | DMSO-d6): _ 7.56-7.45 (m, | r.t. = 2.879 min; exp. | redcutive |
| isoindol-2-yl)ethyl]- | 8H), 7.22-7.16 (m, 4H), 6.90 | (M − H)− = 436.2; obs | amination |
| 4-phenylquinolin-2- | (s, 1H), 4.40 (s, 1H), 3.86 (s, | (M − H)− = 436.0 | with 2,3- |
| yl}(methyl)amino) | 4H), 3.19(s, 3H), 2.89- | | dihydro-1H- |
| acetic acid | 2.87(m, 2H), 2.84-2.82(m, | | isoindoline |
| | 2H), 2.54-2.52(m, 2H) | | |

Activity of compounds on adipocyte glucose consumption was tested in differentiated 3T3-L1 mouse adipocyte cells. 3T3-L1 preadipocytes (ATCC) were routinely cultured in a growth medium composed of DMEM high-glucose (Sigma), 10% FBS (Gibco), 10 U/ml penicillin and 10 μg/ml streptomycin (P/S; Gibco). To induce adipogenic differentiation, a confluent layer of 3T3-L1 cells were incubated with the growth medium containing 2 μM rosiglitazone, 1 μM dexamethasone, 500 μM IBMX, and 1 μg/ml insulin (Sigma). Forty-eight (48) hours later (on day 2) and on days 4 and 6, medium of the cells was replaced with fresh medium containing 1 μg/ml insulin. On days 8 and 10, the medium was refreshed with regular growth medium and addition of insulin was omitted. On day 11, medium of the cells were 10 replaced with fresh medium containing either the indicated compounds (10 μM) or the vehicle in which the compounds were dissolved (DMSO). The final concentration of DMSO was 0.1% (v/v). Growth medium containing 0.1% DMSO was incubated in culture wells containing no cells and used as control. 24 hours later, medium was harvested and subjected to centrifugation at 10,000 g for 5 min. Glucose concentration in the supernatants was determined using a 15 colorimetric assay (Glucose Assay Kit I, Eton Biosciences). Glucose consumption in compound and vehicle treated cells was measured as loss of glucose from the culture medium and represented as mean fold change (compound/DMSO)±standard deviation (SD).

Activity of compounds with FABP4 was profiled using a Terbium (Tb) based time resolved fluorescence energy transfer (TR-FRET) assay. The assay measures the compound mediated displacement of the fluorescent fatty acid BODIPY FL C12 (Thermo Fisher; catalog number D3822) from His6 tagged human recombinant FABP4 (His6-FABP4; Cayman Chemicals, catalog number 10009549) via recording the energy transfer from TB donor molecule on anti-His6-tag antibody to acceptor BODIPY moiety. Briefly, compounds and BODIPY FL C12 were prepared at a concentration of 1.085 mM and 4.2 μM, respectively, in DMSO. 1.2 μL of each compound or DMSO (vehicle control) and 1.2 μL of BODIPY FL C12 were added into the wells of a 384-well black polypropylene plate. His6-FABP4 and Tb anti-His6 antibody were prepared in the assay buffer (25 mM Tris/HCl, pH 7.4, 0.4 mg/ml γ-globulins, 0.010% NP-40, 1 mM DTT) at a concentration of 83 nM and 49.6 nM, respectively. The protein and antibody solutions were then mixed at a ratio of 34:7 (v/v) and incubated on ice for 30 minutes. The assay was initiated by adding 41 μL of the resulting protein/antibody solution into the wells containing the compounds and BODIPY FL C12. The plate was centrifuged and incubated at room temperature for 10 min. The TR-FRET signals were recorded using an EnVision Multilabel plate reader (PerkinElmer; TB excitation 320 nm, BODIPY FL C12 emission 520 nm; TB emission 615 nm). Relative fluorescence ratio (520 nm*10,000/615 nm) were used to calculate the compound mediated inhibition of BODIPY C12 FL fatty acid binding to FABP4. The compounds were tested in triplicates and the results were represented as mean percent inhibition (compound*100/DMSO)±standard deviation (SD). Glucose consumption and FABP4 inhibition is shown below in Table 3 below. The compounds showing no inhibition of activity in the TR-FRET assay are marked as N.A. N.D. stands for “not determined”.

TABLE 3

| | Glucose | | FABP4 | |
|---|---|---|---|---|
| | consumption | | inhibition | |
| | (fold change) | | (%) | |
| IUPAC | Mean | SD | Mean | SD |
| 3-{[6-butyl-4-(4-fluorophenyl)quinolin- | 1.34 | 0.05 | 73.99 | 6.38 |
| 2-yl](methyl)amino}-2-methylpropanoic | | | | |
| acid | | | | |
| 3-{[4-(4-fluorophenyl)-6-hexylquinolin- | 1.31 | 0.00 | 92.30 | 5.06 |
| 2-yl](methyl)amino}-2-methylpropanoic | | | | |
| acid | | | | |
| 2-{[4-(4-fluorophenyl)-6-pentylquinolin- | 1.34 | 0.04 | 80.87 | 5.95 |
| 2-yl](methyl)amino}acetic acid | | | | |
| 2-{[4-(4-fluorophenyl)-6-hexylquinolin- | 1.30 | 0.05 | 81.01 | 6.80 |
| 2-yl](methyl)amino}acetic acid | | | | |
| 2-{[6-hexyl-3-methyl-4-(morpholin-4- | 1.17 | 0.06 | 93.85 | 6.28 |
| yl)quinolin-2-yl](methyl)amino}acetic | | | | |
| acid | | | | |
| 2-{[4-(4-fluorophenyl)-6-hexyl-3 - | 1.19 | 0.05 | 110.67 | 1.95 |
| methylquinolin-2- | | | | |
| yl](methyl)amino}acetic acid | | | | |
| 2-{ [4,6-bis(4-fluorophenyl)quinolin-2- | 1.28 | 0.01 | 42.03 | 6.39 |
| yl](methyl)amino}acetic acid | | | | |
| 2-{[4-(4-fluoropheny1)-6-hexylquinolin- | 1.18 | 0.00 | 78.68 | 3.08 |
| 2-yl](2-methylpropyl)amino} acetic acid | | | | |
| 2-{[4-(4-fluoropheny1)-6-hexylquinolin- | 1.13 | 0.06 | 57.12 | 4.67 |
| 2-yl](propyl)amino}acetic acid | | | | |
| 2-{[4-(4-fluorophenyl)-6-hexylquinolin- | 1.12 | 0.06 | N.D. | |
| 2-yl]amino}acetic acid | | | | |
| 2-{ethyl[4-(4-fluorophenyl)-6- | 1.27 | 0.01 | 78.33 | 5.12 |
| hexylquinolin-2-yl]amino}acetic acid | | | | |
| 2-{[6-hexyl-4-(pyridin-3-yloxy)quinolin- | 1.33 | 0.00 | 84.99 | 10.33 |
| 2-yl](methyl)amino}acetic acid | | | | |
| 2-{[6-hexyl-4-(pyridin-4-yloxy)quinolin- | 1.02 | 0.00 | 10.72 | 5.32 |
| 2-yl](methyl)amino}acetic acid | | | | |
| 2-{[4-(3 -fluorophenoxy)-6- | 1.21 | 0.01 | 98.75 | 2.69 |
| hexylquinolin-2-yl](methyl)amino}acetic | | | | |
| acid | | | | |
| 2-{[4-(4-fluorophenoxy)-6- | 1.27 | 0.04 | 65.16 | 4.00 |
| hexylquinolin-2-yl](methyl)amino}acetic | | | | |
| acid | | | | |
| 2-{[4-(4-fluorophenyl)-6-octylquinolin- | 1.28 | 0.00 | 68.01 | 5.83 |
| 2-yl](2-methylpropyl)amino}acetic acid | | | | |
| 2-{[4-(4-fluorophenyl)-6-octylquinolin- | 1.20 | 0.07 | 53.10 | 5.12 |
| 2-yl](propyl)amino}acetic acid | | | | |
| 2-{ethyl[4-(4-fluorophenyl)-6- | 1.25 | 0.00 | 65.82 | 2.62 |
| octylquinolin-2-yl]amino}acetic acid | | | | |
| 2-{methyl[6-octyl-4-(pyridin-3- | 1.47 | 0.04 | 83.58 | 1.42 |
| yloxy)quinolin-2-yl]amino}acetic acid | | | | |
| 2-{methy1[6-octyl-4-(pyridin-4- | 1.14 | 0.02 | 19.43 | 1.38 |
| yloxy)quinolin-2-yl]amino}acetic acid | | | | |
| 2-{[4-(3 -fluorophenoxy)-6- | 1.32 | 0.03 | 74.59 | 3.45 |
| octylquinolin-2-yl](methyl)amino}acetic | | | | |
| acid | | | | |
| 2-{[4-(4-fluorophenoxy)-6- | 1.41 | 0.00 | 78.69 | 0.23 |
| octylquinolin-2-yl](methyl)amino}acetic | | | | |
| acid | | | | |
| 2-{[6-decyl-4-(4-fluorophenyl)quinolin- | 1.37 | 0.01 | N.D. | |
| 2-yl](methyl)amino}acetic acid | | | | |
| 2-{[4-(4-fluorophenyl)-6-heptylquinolin- | 1.25 | 0.03 | 75.57 | 1.70 |
| 2-yl](methyl)amino}acetic acid | | | | |
| 2-{[4-(4-fluorophenyl)-6-octylquinolin- | 1.28 | 0.01 | 53.83 | 6.02 |
| 2-yl](methyl)amino}acetic acid | | | | |
| 2-[(6-hexylquinolin-2- | 1.15 | 0.03 | 53.26 | 3.86 |
| yl)(methyl)amino]acetic acid | | | | |
| 2-{2-[(carboxymethyl)(methyl)amino]-6- | 1.17 | 0.02 | N.D. | |
| hexylquinolin-4-yl}benzoic acid | | | | |
| 2-{[4-(4,4-difluoropiperidin-1 -y l)-6- | 1.23 | 0.06 | 53.66 | 6.71 |
| hexylquinolin-2-yl](methyl)amino}acetic | | | | |
| acid | | | | |
| 2-{[4-(3,3 -difluoropyrrolidin-1 -y l)-6 - | 1.28 | 0.02 | 50.49 | 6.87 |
| hexylquinolin-2-yl](methyl)amino}acetic | | | | |
| acid | | | | |
| 2-{[6-hexyl-4-(morpholin-4-yl)quinolin- | 1.31 | 0.04 | 26.54 | 8.69 |
| 2-yl](methyl)amino}acetic acid | | | | |
| 2-{[6-butyl-4-(2-methyl-pyridin-4- | 1.23 | 0.05 | 58.46 | 1.93 |
| yl)quinolin-2-yl](methyl)amino}acetic | | | | |
| acid | | | | |
| 2-{[4-(3,5-dimethyl-1,2-oxazol-4-yl)-6- | 1.22 | 0.01 | N.D. | |
| hexylquinolin-2-yl](methyl)amino}acetic | | | | |
| acid | | | | |
| 2-{[4-(3-cyanophenyl)-6-hexylquinolin- | 1.27 | 0.05 | 59.08 | 6.13 |
| 2-yl](methyl)amino}acetic acid | | | | |
| 3-{[4-(3 -cy anophenyl)-6-hexylquinolin- | 1.03 | 0.07 | 77.91 | 1.59 |
| 2-yl](methyl)amino}butanoic acid | | | | |
| 3-[(6-hexyl-4-phenylquinolin-2- | 1.31 | 0.09 | 89.70 | 3.72 |
| yl)(methyl)amino]-2-methylpropanoic | | | | |
| acid | | | | |
| 2-[methyl(6-pentanamido-4- | 0.98 | 0.02 | 28.70 | 0.70 |
| phenylquinolin-2-yl)amino]acetic acid | | | | |
| 2-{methyl[6-(pentyloxy)-4- | 1.27 | 0.05 | 102.18 | 2.02 |
| phenylquinolin-2-yl]amino}acetic acid | | | | |
| 2-[(7-bromo-4-phenylquinolin-2- | 1.19 | 0.06 | 84.48 | 4.78 |
| yl)(methyl)amino]acetic acid | | | | |
| 2-[(7-hexyl-4-phenylquinolin-2- | 1.26 | 0.05 | 25.29 | 3.26 |
| yl)(methyl)amino]acetic acid | | | | |
| 2-[methyl(6-octyl-4-phenylquinolin-2- | 1.52 | 0.04 | 99.27 | 2.48 |
| yl)amino]acetic acid | | | | |
| 3-{[6-hexyl-4-(pyridin-3-yl)quinolin-2- | 1.16 | 0.04 | 54.72 | 1.99 |
| yl](methyl)amino}-2-methylpropanoic | | | | |
| acid | | | | |
| 2-{[6-hexyl-4-(pyridin-3-yl)quinolin-2- | 1.28 | 0.09 | 71.35 | 4.63 |
| yl](methyl)amino}acetic acid | | | | |
| 2-{[4-(3-cyanophenyl)-6-hexylquinolin- | 1.02 | 0.00 | 39.42 | 1.53 |
| 2-yl] oxy} acetic acid | | | | |
| 3-{[4-(3-cy anophenyl)-6-hexylquinolin- | 1.30 | 0.03 | 56.52 | 4.57 |
| 2-yl](methyl)amino}-2-methylpropanoic | | | | |
| acid | | | | |
| 2-{[4-(3-cyanophenyl)-6-hexylquinolin- | 1.26 | 0.03 | 36.12 | 3.42 |
| 2-yl](methyl)amino}acetic acid | | | | |
| 1-[6-hexyl-4-(pyridin-3-yl)quinolin-2- | 1.09 | 0.03 | 91.62 | 4.04 |
| yl]piperidine-3-carboxylic acid | | | | |
| 1-(6-hexyl-4-phenylquinolin-2- | 1.16 | 0.05 | 88.33 | 7.35 |
| yl)piperidine-3-carboxylic acid | | | | |
| 2-{[6-butyl-4-(4- | 1.26 | 0.03 | 60.45 | 8.44 |
| hydroxyphenyl)quinolin-2- | | | | |
| yl](methyl)amino}acetic acid | | | | |
| 2-{[6-butyl-4-(3- | 1.28 | 0.14 | 83.75 | 3.96 |
| hydroxyphenyl)quinolin-2- | | | | |
| yl](methyl)amino}acetic acid | | | | |
| 2-{[6-butyl-4-(2- | 1.19 | 0.05 | 93.24 | 5.92 |
| hydroxyphenyl)quinolin-2- | | | | |
| yl](methyl)amino}acetic acid | | | | |
| 2-{[6-butyl-4-(4-fluorophenyl)quinolin- | 1.49 | 0.13 | 58.65 | 6.01 |
| 2-yl](methyl)amino}acetic acid | | | | |
| 2-{[6-butyl-4-(3 -fluorophenyl)quinolin- | 1.43 | 0.05 | 58.92 | 3.52 |
| 2-yl](methyl)amino}acetic acid | | | | |
| 2-{[6-butyl-4-(2-fluorophenyl)quinolin- | 1.33 | 0.03 | 67.03 | 1.70 |
| 2-yl](methyl)amino}acetic acid | | | | |
| 2-{[6-butyl-4-(4-methylphenyl)quinolin- | 1.36 | 0.06 | 30.80 | 5.86 |
| 2-yl](methyl)amino}acetic acid | | | | |
| 2-{[6-butyl-4-(3-methylphenyl)quinolin- | 1.40 | 0.02 | 82.42 | 6.14 |
| 2-yl](methyl)amino}acetic acid | | | | |
| 2-{[6-butyl-4-(2-methylphenyl)quinolin- | 1.27 | 0.00 | 99.65 | 5.63 |
| 2-yl](methyl)amino}acetic acid | | | | |
| 2-{[6-butyl-4-(4-cyanophenyl)quinolin- | 1.18 | 0.02 | 16.77 | 7.49 |
| 2-yl](methyl)amino}acetic acid | | | | |
| 2-{[6-butyl-4-(4- | 1.26 | 0.04 | 33.38 | 3.82 |
| carbamoylphenyl)quinolin-2- | | | | |
| yl](methyl)amino}acetic acid | | | | |
| 2-{[6-butyl-4-(pyridin-4-yl)quinolin-2- | 1.28 | 0.02 | 15.01 | 7.07 |
| yl](methyl)amino}acetic acid | | | | |
| 6-butyl-2-(carboxymethoxy)-4- | 0.99 | 0.02 | 44.59 | 1.95 |
| phenylquinoline-3-carboxylic acid | | | | |
| 2-{[6-butyl-4-(pyridin-3-yl)quinolin-2- | 1.19 | 0.02 | 39.18 | 1.69 |
| yl](methyl)amino}acetic acid | | | | |
| 1-[6-butyl-4-(3-cyanophenyl)quinolin-2- | 0.91 | 0.11 | 24.28 | 7.86 |
| yl]piperidine-3-carboxylic acid | | | | |
| 4-(6-butyl-4-phenylquinolin-2- | 1.07 | 0.17 | 21.07 | 4.35 |
| yl)morpholine-2-carboxylic acid | | | | |
| 1-(6-butyl-4-phenylquinolin-2- | 1.25 | 0.00 | 98.11 | 6.80 |
| yl)piperidine-3-carboxylic acid | | | | |
| 3-{[6-butyl-4-(3 -cy anophenyl)quinolin- | 1.21 | 0.06 | 38.74 | 8.86 |
| 2-yl](methyl)amino}-2-methylpropanoic | | | | |
| acid | | | | |
| 3-{[6-butyl-4-(pyridin-3-yl)quinolin-2- | 1.13 | 0.00 | 46.39 | 6.28 |
| yl](methyl)amino}-2-methylpropanoic | | | | |
| acid | | | | |
| 3-[(6-butyl-4-phenylquinolin-2- | 1.34 | 0.05 | 74.72 | 4.32 |
| yl)(methyl)amino]-2-methylpropanoic | | | | |
| acid | | | | |
| 3-[(6-butyl-4-phenylquinolin-2- | 1.04 | 0.00 | 103.16 | 5.07 |
| yl)(methyl)amino]butanoic acid | | | | |
| 3-[(6-butyl-4-phenylquinolin-2- | 1.10 | 0.11 | N.D. | |
| yl)(methyl)amino]propanoic acid | | | | |
| N-(6-butyl-4-(3-cyanophenyl)quinolin-2- | 1.18 | 0.06 | 21.03 | 1.34 |
| yl)-N-methylvaline? | | | | |
| 2-{[4-(3-cyanophenyl)-6-pentylquinolin- | 1.01 | 0.07 | 10.59 | 2.45 |
| 2-yl]oxy}acetic acid | | | | |
| 2-{[4-(3-carbamoylphenyl)-6- | 1.02 | 0.03 | 74.19 | 7.26 |
| pentylquinolin-2-yl]oxy}acetic acid | | | | |
| 2-{[4-(3-cyanophenyl)-6-propylquinolin- | 1.06 | 0.00 | 34.76 | 6.09 |
| 2-yl]oxy}acetic acid | | | | |
| 2-{[4-(3-carbamoylphenyl)-6- | 1.00 | 0.01 | 42.92 | 6.74 |
| propyl quinolin-2-yl ] oxy } aceti c aci d | | | | |
| 2-{[4-(3-carbamoylphenyl)-6- | 0.93 | 0.01 | 30.38 | 8.26 |
| ethylquinolin-2-yl]oxy}acetic acid | | | | |
| 2-{[6-bromo-4-(3-cyanophenyl)quinolin- | 0.99 | 0.03 | 19.85 | 4.50 |
| 2-yl]oxy}acetic acid | | | | |
| 2-{[6-bromo-4-(3- | 1.00 | 0.01 | 19.14 | 5.23 |
| carbamoylphenyl)quinolin-2- | | | | |
| yl]oxy}acetic acid | | | | |
| 2-{[6-butyl-4-(3 -cyanophenyl)quinolin- | 1.12 | 0.05 | 36.18 | 5.42 |
| 2-yl]oxy}acetic acid | | | | |
| 2-{[6-butyl-4-(3- | 1.15 | 0.00 | 54.54 | 5.67 |
| carbamoylphenyl)quinolin-2- | | | | |
| yl]oxy}acetic acid | | | | |
| 2-{[4-(3-cyanopheny1)-6-pentylquinolin- | 1.23 | 0.00 | 65.10 | 7.41 |
| 2-yl](methyl)amino}acetic acid | | | | |
| 2-{[4-(3-carbamoylphenyl)-6- | 1.04 | 0.01 | 60.44 | 4.25 |
| pentylquinolin-2- | | | | |
| yl](methyl)amino}acetic acid | | | | |
| 2-{[4-(3-cyanophenyl)-6-propylquinolin- | 1.31 | 0.05 | 27.73 | 8.15 |
| 2-yl](methyl)amino}acetic acid | | | | |
| 2-{[4-(3-carbamoylphenyl)-6- | 1.07 | 0.04 | 47.99 | 4.35 |
| propylquinolin-2- | | | | |
| yl](methyl)amino}acetic acid | | | | |
| 2-{[4-(3-cyanophenyl)-6-ethylquinolin- | 1.16 | 0.00 | 50.82 | 6.00 |
| 2-yl](methyl)amino}acetic acid | | | | |
| 2-{[4-(3-carbamoylphenyl)-6- | 1.07 | 0.06 | 78.02 | 3.58 |
| ethylquinolin-2-yl](methyl)amino}acetic | | | | |
| acid | | | | |
| 2-{[6-bromo-4-(3-cyanophenyl)quinolin- | 1.16 | 0.07 | 91.14 | 2.49 |
| 2-yl](methyl)amino}acetic acid | | | | |
| 2-{[6-bromo-4-(3- | 1.10 | 0.01 | 93.76 | 2.81 |
| carbamoylphenyl)quinolin-2- | | | | |
| yl](methyl)amino}acetic acid | | | | |
| 2-{[6-butyl-4-(3 -cy anophenyl)quinolin- | 1.33 | 0.04 | 33.16 | 4.62 |
| 2-yl](methyl)amino}acetic acid | | | | |
| 2-{[6-butyl-4-(3- | 1.13 | 0.01 | 26.06 | 4.72 |
| carbamoylphenyl)quinolin-2- | | | | |
| yl](methyl)amino}acetic acid | | | | |
| 2-{2-[(6-chloro-4-phenylquinolin-2- | 1.08 | 0.03 | 103.39 | 1.66 |
| yl)(methyl)amino]acetamido}acetic acid | | | | |
| 2-[methyl(6-pentyl-4-phenylquinolin-2- | 1.32 | 0.03 | 90.55 | 2.70 |
| yl)amino]acetic acid | | | | |
| 2-[methyl(4-phenyl-6-propylquinolin-2- | 1.36 | 0.11 | 104.01 | 2.07 |
| yl)amino]acetic acid | | | | |
| 2-[(6-ethyl-4-phenylquinolin-2- | 1.12 | 0.01 | 103.15 | 1.72 |
| yl)(methyl)amino]acetic acid | | | | |
| 1-(6-hexyl-4-phenylquinolin-2- | 1.09 | 0.03 | 67.55 | 2.51 |
| yl)pyrrolidine-2-carboxylic acid | | | | |
| 6-hexyl-4-phenyl-2-(piperidin-1 - | 1.18 | 0.00 | 46.32 | 2.83 |
| yl)quinoline | | | | |
| 2-[(6-hexyl-4-phenylquinolin-2- | 1.41 | 0.11 | 88.24 | 6.33 |
| yl)(methyl)amino]acetic acid | | | | |
| 1-(6-butyl-4-phenylquinolin-2- | 1.12 | 0.01 | 92.47 | 6.57 |
| yl)pyrrolidine-2-carboxylic acid | | | | |
| 6-butyl-4-phenyl-2-(piperidin-1 - | 0.99 | 0.02 | 56.45 | 10.68 |
| yl)quinoline | | | | |
| 2-[(6-bromo-4-phenylquinolin-2- | 1.01 | 0.02 | 34.96 | 4.29 |
| yl)oxy]acetic acid | | | | |
| 2-[(6-bromo-4-phenylquinolin-2- | 1.26 | 0.04 | 109.33 | 7.62 |
| yl)(methyl)amino]acetic acid | | | | |
| 2-[(6-pentyl-4-phenylquinolin-2- | 1.05 | 0.01 | 55.06 | 4.59 |
| yl)oxy]acetic acid | | | | |
| 2-[(4-phenyl-6-propylquinolin-2- | 0.99 | 0.02 | 33.84 | 8.83 |
| yl)oxy]acetic acid | | | | |
| 2-[(6-ethyl-4-phenylquinolin-2- | 0.97 | 0.00 | 31.94 | 5.02 |
| yl)oxy]acetic acid | | | | |
| 2-[(6-chloro-4-phenylquinolin-2- | 1.10 | 0.01 | 34.35 | 7.55 |
| yl)oxy]acetic acid | | | | |
| 2-[(6-butyl-4-phenylquinolin-2- | 1.12 | 0.01 | 11.31 | 2.19 |
| yl)oxy]acetic acid | | | | |
| 2-[(6-butyl-4-phenylquinolin-2- | 1.37 | 0.08 | 73.53 | 6.24 |
| yl)(methyl)amino]acetic acid | | | | |
| 1-(6-chloro-4-phenylquinolin-2- | 1.08 | 0.03 | 104.56 | 0.93 |
| yl)pyrrolidine-2-carboxylic acid | | | | |
| 2-[(6-chloro-4-phenylquinolin-2- | 1.18 | 0.01 | 97.41 | 3.14 |
| yl)(methyl)amino]propanoic acid | | | | |
| 2-[(6-chloro-4-phenylquinolin-2- | 1.14 | 0.00 | 99.69 | 2.10 |
| yl)(methyl)amino]acetic acid | | | | |
| 6-chloro-4-phenyl-2-(piperidin-1 - | 0.99 | 0.03 | 64.30 | 8.35 |
| yl)quinoline | | | | |
| 2-({6-[(1E)-hex-1-en-1-yl]-4- | 1.32 | 0.03 | N.D. | |
| phenylquinolin-2- | | | | |
| yl}(methyl)amino)acetic acid | | | | |
| 2-[methyl({4-phenyl-6-[(lE)-2- | 1.21 | 0.11 | N.D. | |
| phenylethenyl]quinolin-2- | | | | |
| yl})amino]acetic acid | | | | |
| 2-{methyl[4-phenyl-6-(2- | 1.48 | 0.10 | N.D. | |
| phenylethyl)quinolin-2-yl]amino}acetic | | | | |
| acid | | | | |
| 2-[(6-hexyl-3-methyl-4-phenylquinolin- | N.D. | | N.D. | |
| 2-yl)(methyl)amino]acetic acid | | | | |
| 6-hexyl-N-methyl-4-phenyl-N-[(2H- | 1.52 | 0.02 | 37.80 | 8.32 |
| 1,2,3,4-tetrazol-5-yl)methyl]quinolin-2- | | | | |
| amine | | | | |
| 6-hexy l -N-methy 1-4-pheny l -N -(2H- | 1.11 | 0.02 | N.D. | |
| 1,2,3,4-tetrazol-5-yl)quinolin-2-amine | | | | |
| 5-[(6-hexyl-4-phenylquinolin-2- | 1.33 | 0.00 | N.D. | |
| yl)methyl]-1,3 -thi azolidine-2,4-dione | | | | |
| 5-[(6-hexyl-4-phenylquinolin-2- | 1.29 | 0.01 | N.D. | |
| yl)methylidene]-1,3-thi azolidine-2,4- | | | | |
| dione | | | | |
| 2-[(6-hexyl-4-phenylquinolin-2- | 1.25 | 0.00 | N.D. | |
| yl)oxy]propanoic acid | | | | |
| 3-(6-hexyl-4-phenylquinolin-2- | 1.30 | 0.01 | N.D. | |
| yl)butanoic acid | | | | |
| 2-{[6-hexyl-4-(3-methylphenyl)quinolin- | 1.11 | 0.04 | N.D. | |
| 2-yl](methyl)amino}acetic acid | | | | |
| 2-[(6-heptyl-4-phenylquinolin-2- | 1.40 | 0.03 | N.D. | |
| yl)(methyl)amino]acetic acid | | | | |
| 2-{[6-(4-ethylphenyl)-4-phenylquinolin- | 1.06 | 0.09 | N.D. | |
| 2-yl](methyl)amino}acetic acid | | | | |
| 2-{ methyl [4-phenyl-6-(3- | 1.07 | 0.14 | 61.18 | 4.99 |
| propylphenyl)quinolin-2-yl]amino}acetic | | | | |
| acid | | | | |
| 1-(6-hexyl-4-phenylquinolin-2- | 1.24 | 0.05 | N.D. | |
| yl)pyrrolidine-3-carboxylic acid | | | | |
| 1-(6-hexyl-4-phenylquinolin-2- | 1.27 | 0.07 | N.D. | |
| yl)azetidine-3-carboxylic acid | | | | |
| 2-[(6-hexyl-4-phenylquinolin-2- | 1.11 | 0.05 | N.D. | |
| yl)(methyl)amino] -N-(2- | | | | |
| hydroxyethyl)acetamide | | | | |
| 2-[(6-hexyl-4-phenyl-5,6,7,8- | 1.34 | 0.01 | 64.52 | 3.05 |
| tetrahydroquinolin-2- | | | | |
| yl)(methyl)amino]acetic acid | | | | |
| cis-2-(6-hexyl-4-phenylquinolin-2- | 1.36 | 0.01 | N.D. | |
| yl)cyclopropane-1-carboxylic acid | | | | |
| trans-2-(6-hexyl-4-phenylquinolin-2- | 1.14 | 0.02 | N.D. | |
| yl)cyclopropane-1-carboxylic acid | | | | |
| 2-{methyl [4-phenyl-6-(3- | 1.31 | 0.06 | 97.03 | 3.79 |
| phenylpropyl)quinolin-2-yl]amino}acetic | | | | |
| acid | | | | |
| 2-{[6-(benzyloxy)-4-phenylquinolin-2- | 1.27 | 0.06 | 84.23 | 6.86 |
| yl](methyl)amino}acetic acid | | | | |
| 2-[(6-methoxy-4-phenylquinolin-2- | 1.12 | 0.00 | 101.40 | 4.98 |
| yl)(methyl)amino]acetic acid | | | | |
| 2-[methyl({6-[2-(2-methylphenyl)ethyl]- | 1.40 | 0.05 | 38.16 | 10.74 |
| 4-phenylquinolin-2-yl})amino]acetic | | | | |
| acid | | | | |
| 2-[methyl({6-[2-(3-methylphenyl)ethyl]- | 1.53 | 0.00 | 50.23 | 2.14 |
| 4-phenylquinolin-2-yl})amino]acetic | | | | |
| acid | | | | |
| 2-[methyl({6-[2-(4-methylphenyl)ethyl]- | 1.55 | 0.00 | 67.22 | 8.54 |
| 4-phenylquinolin-2-yl})amino]acetic | | | | |
| acid | | | | |
| 2-[methyl({4-phenyl-6-[2-(pyridin-4- | 1.26 | 0.03 | N.A. | |
| yl)ethyl]quinolin-2-yl})amino]acetic acid | | | | |
| 2-[methyl({4-phenyl-6-[2-(pyridin-3- | 1.41 | 0.07 | 32.05 | 11.12 |
| yl)ethyl]quinolin-2-yl})amino]acetic acid | | | | |
| 2-[methyl({4-phenyl-6-[2-(pyridin-2- | 1.39 | 0.11 | N.D. | |
| yl)ethyl]quinolin-2-yl})amino]acetic acid | | | | |
| 2-({6-[2-(2-chlorophenyl)ethyl]-4- | 1.26 | 0.03 | 30.05 | 6.31 |
| phenylquinolin-2- | | | | |
| yl}(methyl)amino)acetic acid | | | | |
| 2-({6-[2-(3-chlorophenyl)ethyl]-4- | 1.47 | 0.00 | 57.09 | 5.92 |
| phenylquinolin-2- | | | | |
| yl}(methyl)amino)acetic acid | | | | |
| 2-({6-[2-(4-chlorophenyl)ethyl]-4- | 1.43 | 0.01 | 56.37 | 2.87 |
| phenylquinolin-2- | | | | |
| yl}(methyl)amino)acetic acid | | | | |
| 2-[methyl({6-[2-(morpholin-4-yl)ethyl]- | 1.13 | 0.00 | 6.78 | 5.99 |
| 4-phenylquinolin-2-yl})amino]acetic | | | | |
| acid | | | | |
| 2-[methyl({6-[2-(4-methylpiperazin-1- | 0.99 | 0.02 | 60.30 | 7.62 |
| yl)ethyl]-4-phenylquinolin-2- | | | | |
| yl})amino]acetic acid | | | | |
| 2-({6-[2-(2-methoxyphenyl)ethyl]-4- | 1.11 | 0.02 | 65.11 | 7.87 |
| phenylquinolin-2- | | | | |
| yl}(methyl)amino)acetic acid | | | | |
| 2-({6-[2-(3-methoxyphenyl)ethyl]-4- | 1.31 | 0.04 | 93.23 | 2.52 |
| phenylquinolin-2- | | | | |
| yl}(methyl)amino)acetic acid | | | | |
| 2-({6-[2-(4-methoxyphenyl)ethyl]-4- | 1.26 | 0.02 | 14.97 | 3.16 |
| phenylquinolin-2- | | | | |
| yl}(methyl)amino)acetic acid | | | | |
| 1-[4-phenyl-6-(2-phenylethyl)quinolin-2- | 1.21 | 0.02 | N.D. | |
| yl]pyrrolidine-3-carboxylic acid | | | | |
| 2-{[4-(2-fluorophenyl)-6-hexylquinolin- | 1.26 | 0.09 | N.D. | |
| 2-yl](methyl)amino}acetic acid | | | | |
| 5-oxo-1-[4-phenyl-6-(2- | 1.24 | 0.05 | 7.89 | 8.64 |
| phenylethyl)quinolin-2-yl]pyrrolidine-3- | | | | |
| carboxylic acid | | | | |
| 1-(6-hexyl-4-phenylquinolin-2-yl)-5- | 1.11 | 0.06 | 6.12 | 6.11 |
| oxopyrrolidine-3-carboxylic acid | | | | |
| 4-(6-hexyl-4-phenylquinolin-2- | 1.22 | 0.04 | 36.09 | 10.05 |
| yl)morpholine-2-carboxylic acid | | | | |
| l -(6-hexyl-4-phenylquinolin-2-yl)-1H- | ND. | | N.D. | |
| imidazole-4-carboxylic acid | | | | |
| 1-(6-hexyl-4-phenylquinolin-2-yl)-1H- | 1.09 | 0.01 | N.A. | |
| pyrrole-3-carboxylic acid | | | | |
| 1-(6-hexyl-4-phenylquinolin-2-yl)-3- | 1.36 | 0.02 | 36.35 | 7.84 |
| methylpyrrolidine-3-carboxylic acid | | | | |
| 2-{[4-(3-cyanophenyl)-6-hexylquinolin- | 1.19 | 0.00 | 3.30 | 1.92 |
| 2-yl]oxy}propanoic acid | | | | |
| 2-{[6-butyl-4-(3 -cy anophenyl)quinolin- | 1.26 | 0.08 | 12.66 | 10.19 |
| 2-yl]oxy}propanoic acid | | | | |
| 2-[(6-butyl-4-phenylquinolin-2- | 1.27 | 0.02 | 49.34 | 3.62 |
| yl)(methyl)amino]acetic acid | | | | |
| 2-{[4-phenyl-6-(2-phenylethyl)quinolin- | 1.21 | 0.00 | 34.10 | 2.54 |
| 2-yl]oxy}propanoic acid | | | | |
| N-methyl-4-phenyl-6-(2-phenylethyl)-N- | 1.17 | 0.01 | 76.32 | 8.56 |
| (1H-1,2,3,4-tetrazol-5-yl)quinolin-2- | | | | |
| amine | | | | |
| 4-phenyl-6-(2-phenylethyl)-2-[2-(1H- | 1.20 | 0.19 | 75.05 | 3.78 |
| 1,2,3,4-tetrazol-5-yl)propan-2- | | | | |
| yl]quinoline | | | | |
| 5-{[4-phenyl-6-(2-phenylethyl)quinolin- | 1.45 | 0.01 | 73.51 | 10.09 |
| 2-yl]methyl} -1,3 -thi azolidine-2,4-dione | | | | |
| N-cyclopropyl-6-hexyl-4-phenyl-N-(1H- | 1.18 | 0.06 | 84.02 | 8.10 |
| 1,2,3,4-tetrazol-5-yl)quinolin-2-amine | | | | |
| 2-[methyl({4-phenyl-6-[2-(pyrazin-2- | 1.28 | 0.01 | 4.98 | 5.13 |
| yl)ethyl]quinolin-2-yl})amino]acetic acid | | | | |
| 2-[methyl({4-phenyl-6-[2-(pyrimidin-5- | 1.22 | 0.00 | 13.19 | 7.80 |
| yl)ethyl]quinolin-2-yl})amino]acetic acid | | | | |
| 2-[methyl({4-phenyl-6-[2-(quinoxalin-2- | 1.28 | 0.04 | 33.57 | 17.47 |
| yl)ethyl]quinolin-2-yl})amino]acetic acid | | | | |
| 2-[methyl({4-phenyl-6-[2-(1,2,3,4- | 1.26 | 0.05 | 14.92 | 12.55 |
| tetrahydroquinoxalin-2- | | | | |
| yl)ethyl]quinolin-2-yl})amino]acetic acid | | | | |
| 2-[methyl({4-phenyl-6-[2-(pyrimidin-2- | 1.35 | 0.03 | N.A. | |
| yl)ethyl]quinolin-2-yl})amino]acetic acid | | | | |
| 2-[methyl({4-phenyl-6-[2-(quinolin-6- | 1.40 | 0.07 | 34.74 | 6.23 |
| yl)ethyl]quinolin-2-yl})amino]acetic acid | | | | |
| 2-[methyl({4-phenyl-6-[2-(1, 2,3,4- | 1.31 | 0.01 | 44.68 | 1.25 |
| tetrahydroquinolin-6-yl)ethyl]quinolin-2- | | | | |
| yl})amino]acetic acid | | | | |
| 2-[methyl({4-phenyl-6-[2-(quinolin-7- | 1.25 | 0.03 | 43.47 | 4.79 |
| yl)ethyl]quinolin-2-yl})amino]acetic acid | | | | |
| 2-[methyl({4-phenyl-6-[2-(quinoxalin-6- | 1.33 | 0.02 | 33.63 | 2.40 |
| yl)ethyl]quinolin-2-yl})amino]acetic acid | | | | |
| 2-{methyl[4-phenyl-6-(2-{1H- | 1.26 | 0.03 | 29.82 | 10.14 |
| pyrrolo[2,3-b]pyridin-4- | | | | |
| yl}ethyl)quinolin-2-yl]amino}acetic acid | | | | |
| 2-({6-[2-(1,3-benzothiazol-2-yl)ethyl]-4- | 1.31 | 0.02 | 37.88 | 9.68 |
| phenylquinolin-2- | | | | |
| yl}(methyl)amino)acetic acid | | | | |
| 2-({6-[2-(l ,3 -benzoxazol-2-yl)ethyl]-4- | 1.23 | 0.01 | 27.42 | 6.05 |
| phenylquinolin-2- | | | | |
| yl}(methyl)amino)acetic acid | | | | |
| 2-({6-[2-(1H-1,3-benzodiazol-2- | N.D. | | 31.18 | 13.39 |
| yl)ethyl]-4-phenylquinolin-2- | | | | |
| yl}(methyl)amino)acetic acid | | | | |
| 2-[methyl({ 6-[2-( 1 -methyl- 1H- 1,3- | N.D. | | 30.29 | 6.47 |
| benzodiazol-2-yl)ethyl]-4- | | | | |
| phenylquinolin-2-yl})amino]acetic acid | | | | |
| 2-[methyl(6-{2- | 1.17 | 0.02 | 55.90 | 2.62 |
| [methyl(phenyl)amino]ethyl}-4- | | | | |
| phenylquinolin-2-yl)amino]acetic acid | | | | |
| 2-[methyl({4-phenyl-6-[2-(1,2,3,4- | N.D. | | 21.70 | 7.48 |
| tetrahydroisoquinolin-2- | | | | |
| yl)ethyl]quinolin-2-yl})amino]acetic acid | | | | |
| 2-({6-[2-(2,3-dihydro-1H-indol-1- | N.D. | | 76.26 | 6.64 |
| yl)ethyl]-4-phenylquinolin-2- | | | | |
| yl}(methyl)amino)acetic acid | | | | |
| 2-[methyl({6-[2-(pyridin-2-yl)ethyl]-4- | 1.06 | 0.02 | 16.11 | 4.19 |
| (pyridin-4-yl)quinolin-2- | | | | |
| yl})amino]acetic acid | | | | |
| 2-[methyl({6-[2-(pyridin-3-yl)ethyl]-4- | 1.06 | 0.04 | 24.57 | 11.83 |
| (pyridin-4-yl)quinolin-2- | | | | |
| yl})amino]acetic acid | | | | |
| 2-{[6-hexyl-4-(pyridin-4-yl)quinolin-2- | 1.17 | 0.01 | 51.44 | 15.00 |
| yl](methyl)amino}acetic acid | | | | |
| 2-{[6-hexyl-4-(pyridin-4-yl)quinolin-2- | 1.30 | 0.03 | 20.96 | 12.42 |
| yl]oxy}propanoic acid | | | | |
| 2-({6-[2-(4-hydroxypiperidin-1- | N.D. | | 14.34 | 11.78 |
| yl)ethyl]-4-phenylquinolin-2- | | | | |
| yl}(methyl)amino)acetic acid | | | | |
| 2-[methyl({4-phenyl-6-[2-(piperidin-1 - | 0.96 | 0.06 | N.A. | |
| yl)ethyl]quinolin-2-yl})amino]acetic acid | | | | |
| 2-[methyl({4-phenyl-6-[2-(pyrrolidin-1 - | N.D. | | 2.04 | 5.47 |
| yl)ethyl]quinolin-2-yl})amino]acetic acid | | | | |
| 2-({6-[2-(diethylamino)ethyl]-4- | N.D. | | 5.17 | 1.61 |
| phenylquinolin-2- | | | | |
| yl}(methyl)amino)acetic acid | | | | |
| 2-({6-[2-(2,3-dihydro-1H-isoindol-2- | N.D. | | 12.24 | 7.88 |
| yl)ethyl]-4-phenylquinolin-2- | | | | |
| yl}(methyl)amino)acetic acid | | | | |

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A compound of Formula I comprising: 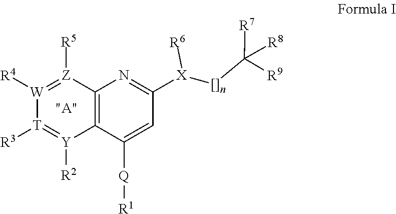
or pharmaceutically acceptable salts or esters thereof, wherein:
R¹-R⁹ are independently hydrogen, CN, COOH, CONH₂, B(ORₐ)₂ (where Rₐ is H or alkyl), an acid isostere, halo, C₁-C₁₀ alkyl, C₁-C₁₀ alkenyl, C₁-C₁₀ alkynyl, aryl, aminoalkyl, haloalkyl, heteroaryl, cycloalkyl, or heterocycloalkyl,
wherein any of R²-R⁹ may be bonded with an adjoining R group thereby forming a fused cycloalkyl, fused heterocycloalkyl, fused aryl, or fused heteroaryl ring having from 4 to 10 carbon atoms;
wherein each of said C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl; —C_{q}—U—C_{q}, where each q is independently 0 to 10 and U is any one of aryl, heteroaryl, cycloalkyl, heterocycloalkyl, O, S, SO₂, or N(R₁)(R₁), wherein each R₁ is independently hydrogen, C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl;
Q is a bond or O;
X is C, N, O, or S, with the proviso that R⁶ is not present if X is O or S;
wherein “A” is a saturated or unsaturated ring depicted by 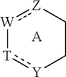
and Y, T, W, and Z are independently a bond, C, N, O, or alkyl or alkenyl having 1 to 4 carbon atoms; and
n is 0, 1, 2, or 3.

2. The compound of claim 1 wherein at least one of: R¹ and R², R² and R³, R³ and R⁴, R⁴ and R⁵, R⁵ and R⁶, R⁷ and R⁸, or R⁸ and R⁹ are bonded forming a fused heteroaryl or fused heterocycloalkyl.

3. The compound of claim 1 wherein R³ is a Cₙ alkyl, and wherein the n is between 3-8.

4. The compound of claim 1 wherein R¹ is the halo.

5. The compound of claim 1 wherein Q comprises the bond.

6. A compound of Formula II comprising: 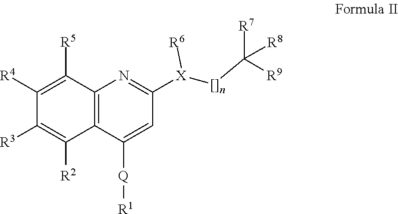
or pharmaceutically acceptable salts or esters thereof wherein:
R¹-R⁹ are independently hydrogen, CN, COOH, CONH₂, B(ORₐ)₂ (where Rₐ is H or alkyl), an acid isostere, halo, C₁-C₁₀ alkyl, C₁-C₁₀ alkenyl, C₁-C₁₀ alkynyl, aryl, aminoalkyl, haloalkyl, heteroaryl, cycloalkyl, or heterocycloalkyl,
wherein any of R²-R⁹ may be bonded with an adjoining R group thereby forming a fused cycloalkyl, fused heterocycloalkyl, fused aryl, or fused heteroaryl ring having from 4 to 10 carbon atoms and wherein each of said C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl; —C_{q}—U—C_{q}, where each q is independently 0 to 10 and U is any one of aryl, heteroaryl, cycloalkyl, heterocycloalkyl, O, S, SO₂, or N(R₁)(R₁), wherein each R₁ is independently hydrogen, C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl;
Q is a bond or O;
X is C, N, O, or S, with the proviso that R⁶ is not present if X is O or S;
X is C, N, O, or S; and
n is 0, 1, 2, or 3.

7. The compound of claim 6 wherein said heterocycloalkyl formed by bonding two of R⁷, R⁸ or R⁹ is

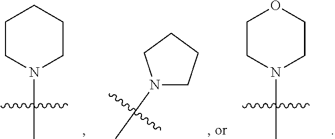 [SMILES: *N1CCCCC1 ; *N1CCCC1 ; *N1CCOCC1]

8. A compound of Formula III comprising: 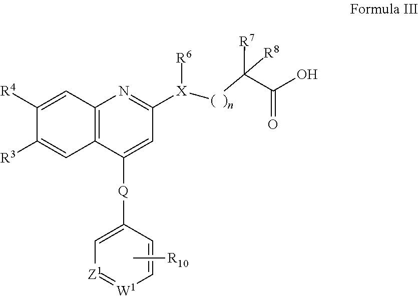
or pharmaceutically acceptable salts or esters thereof, wherein:
R¹ is cyano, alkyl, hydrogen, halo, deuterium, amino, alkoxy, aminoalkyl, (amino)alkoxy, alkenyl, alkynyl, alkoxy, hydroxy, alkylhydroxy, aryloxy, alkyl(aryl), (alkoxyalkyl)amino, aryl, aryl(halo), heteroaryl, hydroxyl-alkyl, hydroxyl-aryl, (aryl)alkyl, C(O)OH, —S(O)₂-alkyl, —S(O)₂-aryl, —C(O)alkyl, or C(O)NH₂;
R³ and R⁴ are independently halo or C₁-C₁₀ alkyl, —C_{q}—U—C_{q}, where each q is independently 0 to 10 and U is any one of O, S, SO₂, or N(R₁)(R₁), wherein each R₁ is independently hydrogen, C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl;
R⁶ is independently H or alkyl;
R⁷ and R⁸ are independently hydrogen, deuterium, fluoro or alkyl;
wherein R⁶-R⁷ may be bonded with an adjoining R group thereby forming a fused cycloalkyl, fused heterocycloalkyl, fused aryl, or fused heteroaryl ring having from 4 to 10 carbon atoms and wherein each of said C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl
n is 0 or 1;
Q is a bond or O;
X is C, N, O, or S, with the proviso that R⁶ is not present if X is O or S; and
Z¹ or W¹ are independently C, N, O, or S.

9. The compound of claim 8 wherein X is N.

10. The compound of claim 8 wherein R⁶ is hydrogen and X is N.

11. The compound of claim 8 wherein R³ is alkyl and R¹ is cyano.

12. The compound of claim 8 wherein X is N, and n=0.

13. The compound of claim 8 wherein X is N, and n=1.

14. The compounds of claim 8 wherein X is N, and Z¹ and W¹ are C.

15. A compound selected from the group consisting of: 3-{[6-butyl-4-(4-fluorophenyl)quinolin-2-yl](methyl)amino}-2-methylpropanoic acid, 3-{[4-(4-fluorophenyl)-6-hexylquinolin-2-yl](methyl)amino}-2-methylpropanoic acid, 2-{[4-(4-fluorophenyl)-6-pentylquinolin-2-yl](methyl)amino}acetic acid, 2-{[4-(4-fluorophenyl)-6-hexylquinolin-2-yl](methyl)amino}acetic acid, 2-{[6-hexyl-3-methyl-4-(morpholin-4-yl)quinolin-2-yl](methyl)amino}acetic acid, 2-{[4-(4-fluorophenyl)-6-hexyl-3-methylquinolin-2-yl](methyl)amino}acetic acid, 2-{[4,6-bis(4-fluorophenyl)quinolin-2-yl](methyl)amino}acetic acid, 2-{[4-(4-fluorophenyl)-6-hexylquinolin-2-yl](2-methylpropyl)amino}acetic acid, 2-{[4-(4-fluorophenyl)-6-hexylquinolin-2-yl](propyl)amino}acetic acid, 2-{[4-(4-fluorophenyl)-6-hexylquinolin-2-yl]amino}acetic acid, 2-{ethyl[4-(4-fluorophenyl)-6-hexylquinolin-2-yl]amino}acetic acid, 2-{[6-hexyl-4-(pyridin-3-yloxy)quinolin-2-yl](methyl)amino}acetic acid, 2-{[6-hexyl-4-(pyridin-4-yloxy)quinolin-2-yl](methyl)amino}acetic acid, 2-{[4-(3-fluorophenoxy)-6-hexylquinolin-2-yl](methyl)amino}acetic acid, 2-{[4-(4-fluorophenoxy)-6-hexylquinolin-2-yl](methyl)amino}acetic acid, 2-{[4-(4-fluorophenyl)-6-octylquinolin-2-yl](2-methylpropyl)amino}acetic acid, 2-{[4-(4-fluorophenyl)-6-octylquinolin-2-yl](propyl)amino}acetic acid, 2-{ethyl[4-(4-fluorophenyl)-6-octylquinolin-2-yl]amino}acetic acid, 2-{methyl[6-octyl-4-(pyridin-3-yloxy)quinolin-2-yl]amino}acetic acid, 2-{methyl[6-octyl-4-(pyridin-4-yloxy)quinolin-2-yl]amino}acetic acid, 2-{[4-(3-fluorophenoxy)-6-octylquinolin-2-yl](methyl)amino}acetic acid, 2-{[4-(4-fluorophenoxy)-6-octylquinolin-2-yl](methyl)amino}acetic acid, 2-{[6-decyl-4-(4-fluorophenyl)quinolin-2-yl](methyl)amino}acetic acid, 2-{[4-(4-fluorophenyl)-6-heptylquinolin-2-yl](methyl)amino}acetic acid, 2-{[4-(4-fluorophenyl)-6-octylquinolin-2-yl](methyl)amino}acetic acid, 2-[(6-hexylquinolin-2-yl)(methyl)amino]acetic acid, 2-{2-[(carboxymethyl)(methyl)amino]-6-hexylquinolin-4-yl}benzoic acid, 2-{[4-(4,4-difluoropiperidin-1-yl)-6-hexylquinolin-2-yl](methyl)amino}acetic acid, 2-{[4-(3,3-difluoropyrrolidin-1-yl)-6-hexylquinolin-2-yl](methyl)amino}acetic acid, 2-{[6-hexyl-4-(morpholin-4-yl)quinolin-2-yl](methyl)amino}acetic acid, 2-{[6-butyl-4-(2-methyl-pyridin-4-yl)quinolin-2-yl](methyl)amino}acetic acid, 2-{[4-(3,5-dimethyl-1,2-oxazol-4-yl)-6-hexylquinolin-2-yl](methyl)amino}acetic acid, 2-{[4-(3-cyanophenyl)-6-hexylquinolin-2-yl](methyl)amino}acetic acid, 3-{[4-(3-cyanophenyl)-6-hexylquinolin-2-yl](methyl)amino}butanoic acid, 3-[(6-hexyl-4-phenylquinolin-2-yl)(methyl)amino]-2-methylpropanoic acid, 2-[methyl(6-pentanamido-4-phenylquinolin-2-yl)amino]acetic acid, 2-{methyl[6-(pentyloxy)-4-phenylquinolin-2-yl]amino}acetic acid, 2-[(7-bromo-4-phenylquinolin-2-yl)(methyl)amino]acetic acid, 2-[(7-hexyl-4-phenylquinolin-2-yl)(methyl)amino]acetic acid, 2-[methyl(6-octyl-4-phenylquinolin-2-yl)amino]acetic acid, 3-{[6-hexyl-4-(pyridin-3-yl)quinolin-2-yl](methyl)amino}-2-methylpropanoic acid, 2-{[6-hexyl-4-(pyridin-3-yl)quinolin-2-yl](methyl)amino}acetic acid, 2-{[4-(3-cyanophenyl)-6-hexylquinolin-2-yl]oxy}acetic acid, 3-{[4-(3-cyanophenyl)-6-hexylquinolin-2-yl](methyl)amino}-2-methylpropanoic acid, 2-{[4-(3-cyanophenyl)-6-hexylquinolin-2-yl](methyl)amino}acetic acid, 1-[6-hexyl-4-(pyridin-3-yl)quinolin-2-yl]piperidine-3-carboxylic acid, 1-(6-hexyl-4-phenylquinolin-2-yl)piperidine-3-carboxylic acid, 2-{[6-butyl-4-(4-hydroxyphenyl)quinolin-2-yl](methyl)amino}acetic acid, 2-{[6-butyl-4-(3-hydroxyphenyl)quinolin-2-yl](methyl)amino}acetic acid, 2-{[6-butyl-4-(2-hydroxyphenyl)quinolin-2-yl](methyl)amino}acetic acid, 2-{[6-butyl-4-(4-fluorophenyl)quinolin-2-yl](methyl)amino}acetic acid, 2-{[6-butyl-4-(3-fluorophenyl)quinolin-2-yl](methyl)amino}acetic acid, 2-{[6-butyl-4-(2-fluorophenyl)quinolin-2-yl](methyl)amino}acetic acid, 2-{[6-butyl-4-(4-methylphenyl)quinolin-2-yl](methyl)amino}acetic acid, 2-{[6-butyl-4-(3-methylphenyl)quinolin-2-yl](methyl)amino}acetic acid, 2-{[6-butyl-4-(2-methylphenyl)quinolin-2-yl](methyl)amino}acetic acid, 2-{[6-butyl-4-(4-cyanophenyl)quinolin-2-yl](methyl)amino}acetic acid, 2-{[6-butyl-4-(4-carbamoylphenyl)quinolin-2-yl](methyl)amino}acetic acid, 2-{[6-butyl-4-(pyridin-4-yl)quinolin-2-yl](methyl)amino}acetic acid, 6-butyl-2-(carboxymethoxy)-4-phenylquinoline-3-carboxylic acid, 2-{[6-butyl-4-(pyridin-3-yl)quinolin-2-yl](methyl)amino}acetic acid, 1-[6-butyl-4-(3-cyanophenyl)quinolin-2-yl]piperidine-3-carboxylic acid, 4-(6-butyl-4-phenylquinolin-2-yl)morpholine-2-carboxylic acid, 1-(6-butyl-4-phenylquinolin-2-yl)piperidine-3-carboxylic acid, 3-{[6-butyl-4-(3-cyanophenyl)quinolin-2-yl](methyl)amino}-2-methylpropanoic acid, 3-{[6-butyl-4-(pyridin-3-yl)quinolin-2-yl](methyl)amino}-2-methylpropanoic acid, 3-[(6-butyl-4-phenylquinolin-2-yl)(methyl)amino]-2-methylpropanoic acid, 3-[(6-butyl-4-phenylquinolin-2-yl)(methyl)amino]butanoic acid, 3-[(6-butyl-4-phenylquinolin-2-yl)(methyl)amino]propanoic acid, N-(6-butyl-4-(3-cyanophenyl)quinolin-2-yl)-N-methylvaline, 2-{[4-(3-cyanophenyl)-6-pentylquinolin-2-yl]oxy}acetic acid, 2-{[4-(3-carbamoylphenyl)-6-pentylquinolin-2-yl]oxy}acetic acid, 2-{[4-(3-cyanophenyl)-6-propylquinolin-2-yl]oxy}acetic acid, 2-{[4-(3-carbamoylphenyl)-6-propylquinolin-2-yl]oxy}acetic acid, 2-{[4-(3-carbamoylphenyl)-6-ethylquinolin-2-yl]oxy}acetic acid, 2-{[6-bromo-4-(3-cyanophenyl)quinolin-2-yl]oxy}acetic acid, 2-{[6-bromo-4-(3-carbamoylphenyl)quinolin-2-yl]oxy}acetic acid, 2-{[6-butyl-4-(3-cyanophenyl)quinolin-2-yl]oxy}acetic acid, 2-{[6-butyl-4-(3-carbamoylphenyl)quinolin-2-yl]oxy}acetic acid, 2-{[4-(3-cyanophenyl)-6-pentylquinolin-2-yl](methyl)amino}acetic acid, 2-{[4-(3-carbamoylphenyl)-6-pentylquinolin-2-yl](methyl)amino}acetic acid, 2-{[4-(3-cyanophenyl)-6-propylquinolin-2-yl](methyl)amino}acetic acid, 2-{[4-(3-carbamoylphenyl)-6-propylquinolin-2-yl](methyl)amino}acetic acid, 2-{[4-(3-cyanophenyl)-6-ethylquinolin-2-yl](methyl)amino}acetic acid, 2-{[4-(3-carbamoylphenyl)-6-ethylquinolin-2-yl](methyl)amino}acetic acid, 2-{[6-bromo-4-(3-cyanophenyl)quinolin-2-yl](methyl)amino}acetic acid, 2-{[6-bromo-4-(3-carbamoylphenyl)quinolin-2-yl](methyl)amino}acetic acid, 2-{[6-butyl-4-(3-cyanophenyl)quinolin-2-yl](methyl)amino}acetic acid, 2-{[6-butyl-4-(3-carbamoylphenyl)quinolin-2-yl](methyl)amino}acetic acid, 2-{2-[(6-chloro-4-phenylquinolin-2-yl)(methyl)amino]acetamido}acetic acid, 2-[methyl(6-pentyl-4-phenylquinolin-2-yl)amino]acetic acid, 2-[methyl(4-phenyl-6-propylquinolin-2-yl)amino]acetic acid, 2-[(6-ethyl-4-phenylquinolin-2-yl)(methyl)amino]acetic acid, 1-(6-hexyl-4-phenylquinolin-2-yl)pyrrolidine-2-carboxylic acid, 6-hexyl-4-phenyl-2-(piperidin-1-yl)quinoline, 2-[(6-hexyl-4-phenylquinolin-2-yl)(methyl)amino]acetic acid, 1-(6-butyl-4-phenylquinolin-2-yl)pyrrolidine-2-carboxylic acid, 6-butyl-4-phenyl-2-(piperidin-1-yl)quinoline, 2-[(6-bromo-4-phenylquinolin-2-yl)oxy]acetic acid, 2-[(6-bromo-4-phenylquinolin-2-yl)(methyl)amino]acetic acid, 2-[(6-pentyl-4-phenylquinolin-2-yl)oxy]acetic acid, 2-[(4-phenyl-6-propylquinolin-2-yl)oxy]acetic acid, 2-[(6-ethyl-4-phenylquinolin-2-yl)oxy]acetic acid, 2-[(6-chloro-4-phenylquinolin-2-yl)oxy]acetic acid, 2-[(6-butyl-4-phenylquinolin-2-yl)oxy]acetic acid, 2-[(6-butyl-4-phenylquinolin-2-yl)(methyl)amino]acetic acid, 1-(6-chloro-4-phenylquinolin-2-yl)pyrrolidine-2-carboxylic acid, 2-[(6-chloro-4-phenylquinolin-2-yl)(methyl)amino]propanoic acid, 2-[(6-chloro-4-phenylquinolin-2-yl)(methyl)amino]acetic acid, 6-chloro-4-phenyl-2-(piperidin-1-yl)quinoline, 3-{[6-butyl-4-(4-fluorophenyl)quinolin-2-yl](methyl)amino}-2-methylpropanoic acid, 2-[methyl(6-octyl-4-phenylquinolin-2-yl)amino]acetic acid, 2-{[6-butyl-4-(4-fluorophenyl)quinolin-2-yl](methyl)amino}acetic acid, 2-{methyl[6-octyl-4-(pyridin-3-yloxy)quinolin-2-yl]amino}acetic acid, 2-{[6-butyl-4-(3-fluorophenyl)quinolin-2-yl](methyl)amino}acetic acid, 2-[(6-hexyl-4-phenylquinolin-2-yl)(methyl)amino]acetic acid, 2-{[4-(4-fluorophenoxy)-6-octylquinolin-2-yl](methyl)amino}acetic acid, 2-{[6-butyl-4-(3-methylphenyl)quinolin-2-yl](methyl)amino}acetic acid, 2-[(6-butyl-4-phenylquinolin-2-yl)(methyl)amino]acetic acid, 2-{[6-decyl-4-(4-fluorophenyl)quinolin-2-yl](methyl)amino}acetic acid, 2-[methyl(4-phenyl-6-propylquinolin-2-yl)amino]acetic acid, 2-{[6-butyl-4-(4-methylphenyl)quinolin-2-yl](methyl)amino}acetic acid, 3-[(6-butyl-4-phenylquinolin-2-yl)(methyl)amino]-2-methylpropanoic acid, 2-{[4-(4-fluorophenyl)-6-pentylquinolin-2-yl](methyl)amino}acetic acid, 2-{[6-butyl-4-(3-cyanophenyl)quinolin-2-yl](methyl)amino}acetic acid, 2-{[6-butyl-4-(2-fluorophenyl)quinolin-2-yl](methyl)amino}acetic acid, 2-{[6-hexyl-4-(pyridin-3-yloxy)quinolin-2-yl](methyl)amino}acetic acid, 2-{[4-(3-fluorophenoxy)-6-octylquinolin-2-yl](methyl)amino}acetic acid, 2-[methyl(6-pentyl-4-phenylquinolin-2-yl)amino]acetic acid, 3-[(6-hexyl-4-phenylquinolin-2-yl)(methyl)amino]-2-methylpropanoic acid, 2-{[4-(3-cyanophenyl)-6-propylquinolin-2-yl](methyl)amino}acetic acid, 2-{[6-hexyl-4-(morpholin-4-yl)quinolin-2-yl](methyl)amino}acetic acid, 3-{[4-(4-fluorophenyl)-6-hexylquinolin-2-yl](methyl)amino}-2-methylpropanoic acid, 2-{[4-(4-fluorophenyl)-6-hexylquinolin-2-yl](methyl)amino}acetic acid, 3-{[4-(3-cyanophenyl)-6-hexylquinolin-2-yl](methyl)amino}-2-methylpropanoic acid, 2-[methyl({6-[2-(4-methylphenyl)ethyl]-4-phenylquinolin-2-yl})amino]acetic acid, 2-[methyl({6-[2-(3-methylphenyl)ethyl]-4-phenylquinolin-2-yl})amino]acetic acid, 6-hexyl-N-methyl-4-phenyl-N-[(2H-1,2,3,4-tetrazol-5-yl)methyl]quinolin-2-amine, 2-{methyl[4-phenyl-6-(2-phenylethyl)quinolin-2-yl]amino}acetic acid, 2-({6-[2-(3-chlorophenyl)ethyl]-4-phenylquinolin-2-yl}(methyl)amino)acetic acid, 5-{[4-phenyl-6-(2-phenylethyl)quinolin-2-yl]methyl}-1,3-thiazolidine-2,4-dione, 2-({6-[2-(4-chlorophenyl)ethyl]-4-phenylquinolin-2-yl}(methyl)amino)acetic acid, 2-[methyl({4-phenyl-6-[2-(pyridin-3-yl)ethyl]quinolin-2-yl})amino]acetic acid, 2-[methyl({4-phenyl-6-[2-(quinolin-6-yl)ethyl]quinolin-2-yl})amino]acetic acid, 2-[(6-heptyl-4-phenylquinolin-2-yl)(methyl)amino]acetic acid, 2-[methyl({6-[2-(2-methylphenyl)ethyl]-4-phenylquinolin-2-yl})amino]acetic acid, 2-[methyl({4-phenyl-6-[2-(pyridin-2-yl)ethyl]quinolin-2-yl})amino]acetic acid, cis-2-(6-hexyl-4-phenylquinolin-2-yl)cyclopropane-1-carboxylic acid, 1-(6-hexyl-4-phenylquinolin-2-yl)-3-methylpyrrolidine-3-carboxylic acid, 2-[methyl({4-phenyl-6-[2-(pyrimidin-2-yl)ethyl]quinolin-2-yl})amino]acetic acid, 2-[(6-hexyl-4-phenyl-5,6,7,8-tetrahydroquinolin-2-yl)(methyl)amino]acetic acid, 2-[methyl({4-phenyl-6-[2-(quinoxalin-6-yl)ethyl]quinolin-2-yl})amino]acetic acid, 5-[(6-hexyl-4-phenylquinolin-2-yl)methyl]-1,3-thiazolidine-2,4-dione, 2-({6-[(1E)-hex-1-en-1-yl]-4-phenylquinolin-2-yl}(methyl)amino)acetic acid, 2-{methyl[4-phenyl-6-(3-phenylpropyl)quinolin-2-yl]amino}acetic acid, 2-[methyl({4-phenyl-6-[2-(1,2,3,4-tetrahydroquinolin-6-yl)ethyl]quinolin-2-yl})amino]acetic acid, 2-({6-[2-(3-methoxyphenyl)ethyl]-4-phenylquinolin-2-yl}(methyl)amino)acetic acid, 2-({6-[2-(1,3-benzothiazol-2-yl)ethyl]-4-phenylquinolin-2-yl}(methyl)amino)acetic acid, 2-{[6-hexyl-4-(pyridin-4-yl)quinolin-2-yl]oxy}propanoic acid, and 3-(6-hexyl-4-phenylquinolin-2-yl)butanoic acid.

16. A method of inhibiting the fatty acid binding protein FABP4 in a mammal, which comprises administering to a mammal an effective amount of a compound of claim 1.

17. A method according to claim 16, wherein the subject is a human.

18. The compound according to claim 1 for use in the prophylaxis or treatment of disorders acting on the fatty acid binding protein FABP4.

19. The compound according to claim 16, wherein the disorders are selected from type 2 diabetes, hyperglycemia, metabolic syndrome, obesity, atherosclerosis, intracranial atherosclerotic disease, non-alcoholic steatohepatitis, asthma, multiple sclerosis, Alzheimer's disease, other chronic inflammatory and autoimmune/inflammatory diseases, chronic heart disease, polycystic ovary syndrome, preeclampsia, and cancer.

20. A pharmaceutical composition comprising a compound according to claim 1 as the active ingredient.

21. The pharmaceutical composition according claim 20, further comprising at least one additional active ingredient or a pharmaceutically acceptable carrier.

22. A method for the prophylaxis or treatment of disorders acting on the fatty acid binding protein FABP4, which comprises administering to a subject in need of such treatment an effective amount of a compound according to claim 1.

23. A method according to claim 22, wherein the subject is a human.
